(19) United States
(12) Patent Application Publication
(10) Pub. No.: US 2025/0026765 A1
(43) Pub. Date: Jan. 23, 2025

(54) SPIROHETEROCYCLE DERIVATIVE HAVING SEROTONIN RECEPTOR BINDING ACTIVITY

(71) Applicant: Shionogi & Co., Ltd., Osaka-shi, Osaka (JP)

(72) Inventors: Tatsuhiko UENO, Osaka-shi, Osaka (JP); Rina YASUI, Osaka-shi, Osaka (JP)

(73) Assignee: Shionogi & Co., Ltd., Osaka-shi, Osaka (JP)

(21) Appl. No.: 18/693,319

(22) PCT Filed: Sep. 21, 2022
(86) PCT No.: PCT/JP2022/035044

§ 371 Date: Mar. 19, 2024

(30) Foreign Application Priority Data
Sep. 22, 2021 (JP) 2021-153715

(51) Int. Cl. C07D 498/10; A61K 31/438; A61K 31/444; A61K 31/506; A61K 31/527; C07D 471/10

(52) U.S. Cl. CPC C07D 498/10; A61K 31/438; A61K 31/444; A61K 31/506; A61K 31/527; C07D 471/10

(57) ABSTRACT

Provided are compounds having a serotonin 5-HT2A receptor antagonism and/or inverse agonism, pharmaceutically acceptable salts thereof, and a pharmaceutical composition comprising thereof.

A compound represented by Formula (I):

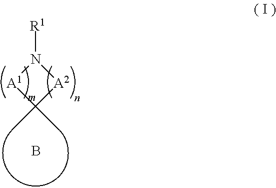

wherein R¹ is a hydrogen atom or the like; A¹ is each independently CR²R^{2′}; A² is each independently CR³R^{3′}, R² is each independently a hydrogen atom or the like; R^{2′} is each independently a hydrogen atom or the like; R³ is each independently a hydrogen atom or the like; R^{3′} is each independently a hydrogen atom or the like; m and n are each independently 1 or the like; ring B is a ring represented by Formula:

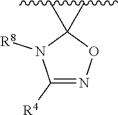

or the like
wherein R⁴ is a group represented by Formula:

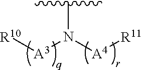

or the like
wherein A³ is each independently CR¹³R^{13′}; A⁴ is each independently CR¹⁴R^{14′}; R¹³ is each independently a hydrogen atom or the like; R^{13′} is each independently a hydrogen atom or the like; R¹⁴ is each independently a hydrogen atom or the like; R^{14′} is each independently a hydrogen atom or the like; q and r are each independently 1 or the like; R¹⁰ and R¹¹ are each independently substituted or unsubstituted aromatic carbocyclyl or the like; R⁸ is a hydrogen atom or the like, or a pharmaceutically acceptable salt thereof.

### TECHNICAL FIELD

The present invention relates to a compound having serotonin 5-HT2A receptor antagonism and/or inverse agonism and useful in the treatment and/or prevention of a disease caused by a serotonin 5-HT2A receptor or a pharmaceutically acceptable salt thereof, and a pharmaceutical composition comprising thereof.

### BACKGROUND ART

Neurodegenerative disorder (ND) is a group of related human diseases that exhibit a common pathophysiological feature, namely progressive degeneration of selective neuronal populations that occurs over time. These neurodegenerative diseases include, but are not limited to, for example, Alzheimer's disease and related dementia, Parkinson's disease, Huntington's disease, Lewy bodies disease and related movement disorders. Each of these disorders has its own unique clinical aspects, such as age of onset, time course of progression, neurologic signs and symptoms, neuropsychiatric symptoms, and susceptibility to known therapeutic agents. In addition, the pathophysiological basis of each of these disorders is caused by a genetic mechanism peculiar to each disease (Non-Patent Document 1).

Despite the considerable progress in elucidating the genetic causes underlying these essentially different disorders, comparatively little is known about the biochemical mechanisms that cause selective neuronal degeneration that are common to all of them. In addition, for the most common disorders of these ones, including Parkinson's disease and Alzheimer's disease, genetic factors that cause these rare familial diseases have been discovered, but for the majority of sporadic cases, the pathophysiological basis is not known yet. Therefore, there is currently no specific therapeutic agent capable of directly altering the progression of the disease. Instead, clinicians utilize a variety of existing agents to achieve symptom relief of the motional manifestations, cognitive manifestations and neuropsychiatric manifestations that characterize these disorders (Non-Patent Document 2 and 3).

Of the various neurological symptoms that characterize ND, the appearance of neuropsychiatric symptoms, including slow motion, abnormal motor function, including dyskinesia and chorea, and emotional symptoms such as psychosis and anxiety and depression, are common symptoms, seriously affects the functional status and quality of life of patients (Non-Patent Documents 4 and 5). Most existing therapeutic agents, including antipsychotics and antidepressants, are often effective in these patients, but their tolerability is significantly poor (Non-Patent Document 6). Also, available Parkinson's disease therapeutic agents, including L-dopa and dopamine agonists, are generally effective, but cause the emergence of treatment-restricting side effects that are currently too severe to be addressed by drug therapy.

Although there has been no ND-specific approved drug for a long time, the 5-HT2A receptor inverse agonist pimavanserin was first approved in the United States in 2016 for the indication of Parkinson's disease-related hallucinations and delusions (Non-Patent Document 7). Unlike existing antipsychotic drugs, this drug has not been reported to have side effects of worsening motor symptoms or cognitive decline. The main pharmacological action of pimavanserin is serotonin 5-HT2A receptor inverse agonism/antagonism, but it also has serotonin 5-HT2C receptor inverse agonism (Non-Patent Document 8). The results of 5-HT2A occupancy measured in the PET test of pimavanserin in humans and the results of clinical trials of pimavanserin suggest that pimavanserin exerts its medicinal effect via 5-HT2A and 5-HT2C (Non-Patent Document 9). In addition, pimavanserin has a large adverse effect on the cardiovascular system, and its use is restricted.

These findings require the development of novel therapeutic agents specifically designed to be not only effective for these specific symptoms which cause physical disability, but also tolerated in these specific patient populations. This can be achieved by improving the selectivity of drug-target interactions of new therapeutic agents. Specifically, it can be achieved by having strong activity and selectivity for target 5-HT2A and 5-HT2C, and reducing adverse effects on the cardiovascular system.

Patent Documents 3 to 14 and 16 to 25 describe compounds having serotonin 5-HT2A receptor antagonism and/or inverse agonism, but any of the documents neither describe nor suggest the compounds related to the present invention.

A quinuclidine derivative having muscarinic M₃ receptor inhibitory activity is disclosed in Patent Document 15, but serotonin 5-HT2A receptor antagonism and/or inverse agonism and therapeutic effects on hallucinations and delusions are not described, and the compounds related to the present invention are neither described nor suggested.

### PRIOR ART REFERENCES

### Patent Documents

[Patent Document 1] International Publication WO2018/131672
[Patent Document 2] US Patent Publication U.S. Pat. No. 8,377,959
[Patent Document 3] International Publication WO2001/066521
[Patent Document 4] International Publication WO2004/064738
[Patent Document 5] International Publication WO2019/040104
[Patent Document 6] International Publication WO2019/040105
[Patent Document 7] International Publication WO2019/040106
[Patent Document 8] International Publication WO2019/040107
[Patent Document 9] International Publication WO2010/111353
[Patent Document 10] International Publication WO2004/000808
[Patent Document 11] International Publication WO2003/057698
[Patent Document 12] China Patent Application Publication CN109111385
[Patent Document 13] International Publication WO2009/039461
[Patent Document 14] International Publication WO2007/124136
[Patent Document 15] International Publication WO2004/000840
[Patent Document 16] International Publication WO2021/147818
[Patent Document 17] International Publication WO2021/147909
[Patent Document 18] International Publication WO2022/145408
[Patent Document 19] China Patent Application Publication CN113214141
[Patent Document 20] China Patent Application Publication CN113214231
[Patent Document 21] China Patent Application Publication CN113214289
[Patent Document 22] International Publication WO2021/218863
[Patent Document 23] China Patent Application Publication CN113549006
[Patent Document 24] International Publication WO2021/193790
[Patent Document 25] International Publication WO2022/017440

### Non-Patent Documents

[Non-patent Document 1] Nature Reviews Neurology volume 10, pages 620-633(2014)
[Non-patent Document 2] Progress in Neurology and Psychiatry Vol. 22, Iss. 1, 30-35, 2018
[Non-patent Document 3] Movement Disorders Vol. 24, No. 11, 2009, pp. 1641-1649
[Non-patent Document 4] Parkisonism and related disorders 1553, 2009, 5105-S109
[Non-patent Document 5] Neurology. 2004; 63(2):293-300.
[Non-patent Document 6] JAMA Neurol. 2016; 73(5):535-541.
[Non-patent Document 7] The Lancet; 383:533-540(2014)
[Non-patent Document 8] Journal of Pharmacology and Experimental Therapeutics May 2006, 317 (2) 910-918
[Non-patent Document 9] CNS Spectrums (2016), 21, 271-275

### SUMMARY OF THE INVENTION

### Problems to be Solved by the Invention

An object of the present invention is to provide a novel compound having serotonin 5-HT2A receptor antagonism and/or inverse agonism. More preferably the present invention is to provide a novel compound having an effect on serotonin-related disease such as Parkinson's disease- and/or dementia-related hallucinations and delusions by having serotonin 5-HT2A receptor antagonism and/or inverse agonism, and a pharmaceutical comprising thereof.

### Means for Solving the Problem

The present invention relates to the following items (1) to (30).

(1) A compound represented by Formula (I):

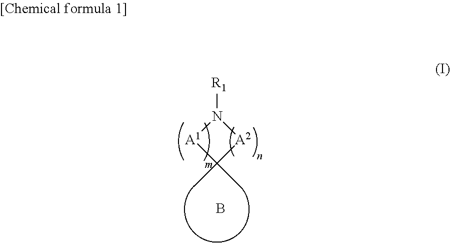

wherein R¹ is a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
A¹ is each independently CR²R^{2′};
A² is each independently CR³R^{3′};
R² is each independently a hydrogen atom, halogen, substituted or unsubstituted alkyl, or substituted or unsubstituted alkyloxy;
R^{2′} is each independently a hydrogen atom, halogen, substituted or unsubstituted alkyl, or substituted or unsubstituted alkyloxy;
R³ is each independently a hydrogen atom, halogen, substituted or unsubstituted alkyl, or substituted or unsubstituted alkyloxy;
R^{3′} is each independently a hydrogen atom, halogen, substituted or unsubstituted alkyl, or substituted or unsubstituted alkyloxy;
R² and R^{2′} and R³ and R^{3′} may be taken together with an identical carbon atom to which they are bonded to form a substituted or unsubstituted non-aromatic carbocycle or a substituted or unsubstituted non-aromatic heterocycle;
m and n are each independently 1, 2, or 3;
ring B is a ring represented by Formula:

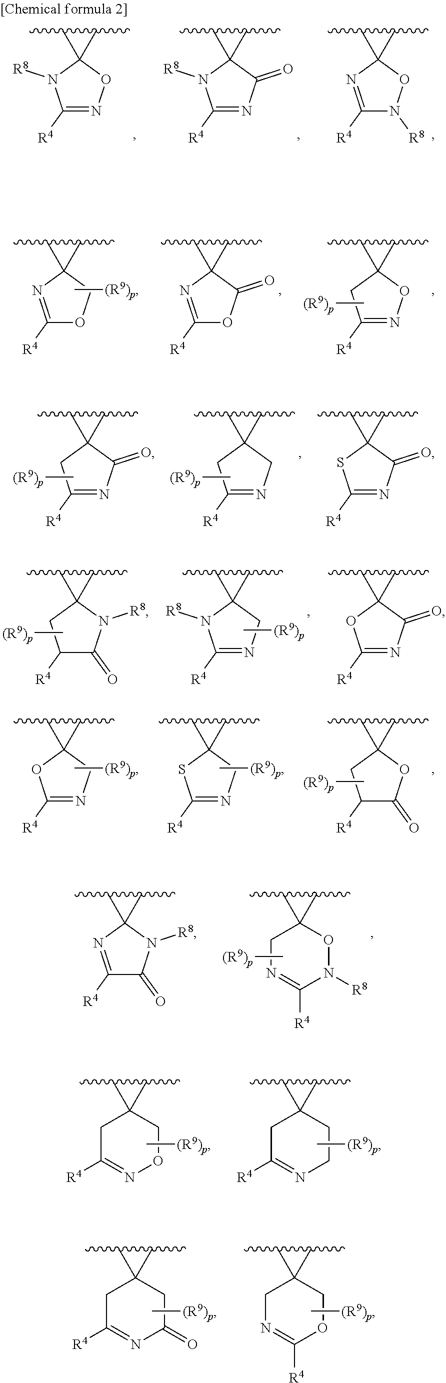

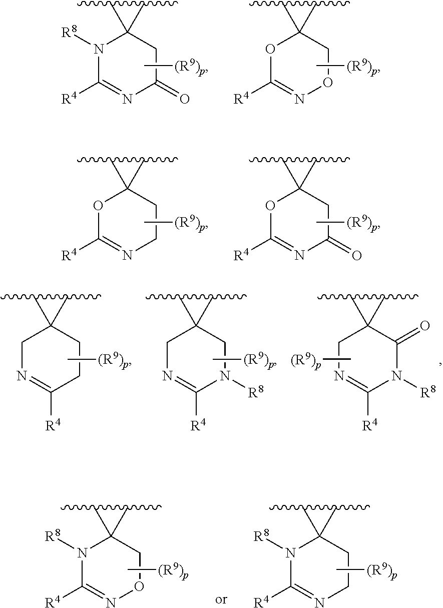

wherein R⁴ is a group represented by Formula:

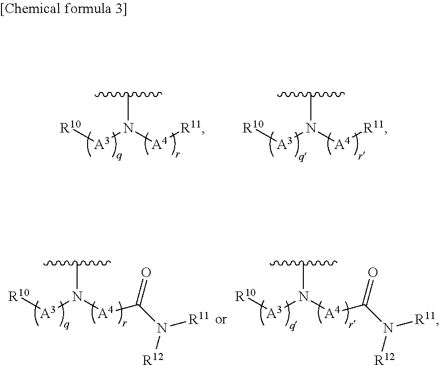

wherein A⁴ is each independently CR¹³R¹³;
A⁴ is each independently CR¹⁴R¹⁴;
R¹³ is each independently a hydrogen atom, substituted or unsubstituted alkyl, or substituted or unsubstituted alkyloxy;
R^{13′} is each independently a hydrogen atom, halogen, substituted or unsubstituted alkyl, or substituted or unsubstituted alkyloxy;
R¹⁴ is each independently a hydrogen atom, halogen, substituted or unsubstituted alkyl, or substituted or unsubstituted alkyloxy;
R^{14′} is each independently a hydrogen atom, halogen, substituted or unsubstituted alkyl, or substituted or unsubstituted alkyloxy;
q and r are each independently 0, 1, or 2;
q′ and r′ are each independently 1 or 2;
R¹⁰ and R¹¹ are each independently substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R¹² is a hydrogen atom or substituted or unsubstituted alkyl;
R⁸ is a hydrogen atom or substituted or unsubstituted alkyl;
R⁹ is each independently halogen or substituted or unsubstituted alkyl;
p is an integer of any of 0 to 6,
   or a pharmaceutically acceptable salt thereof.
   (2) The compound according to the above item (1), wherein R¹ is a hydrogen atom or substituted or unsubstituted alkyl, or a pharmaceutically acceptable salt thereof.
   (3) The compound according to the above item (1) or (2), wherein m and n are each independently 1 or 2, or a pharmaceutically acceptable salt thereof.
   (4) The compound according to the above item (1) or (2), wherein m and n are 2, or a pharmaceutically acceptable salt thereof.
   (5) The compound according to any one of the above items (1) to (4), wherein ring B is a ring represented by Formula:

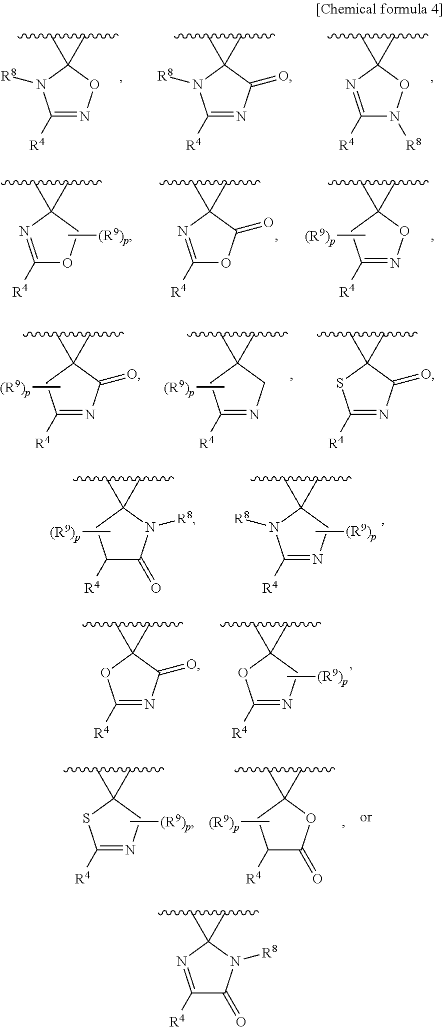

wherein symbols have the same meanings as those in the above item (1), or a pharmaceutically acceptable salt thereof.
(6) The compound according to any one of the above items (1) to (4), wherein ring B is a ring represented by Formula:

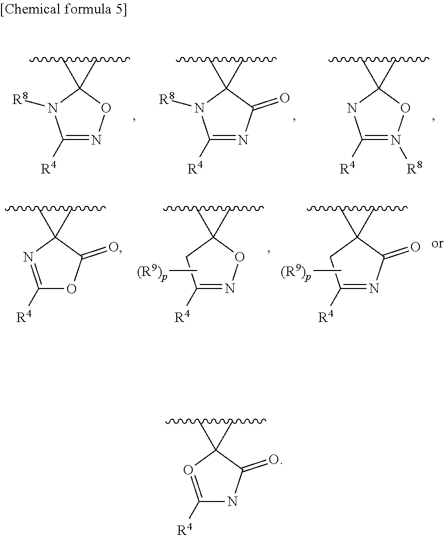

wherein symbols have the same meanings as those in the above item (1), or a pharmaceutically acceptable salt thereof.
(7) The compound according to any one of the above items (1) to (6), wherein R⁴ is a group represented by Formula:

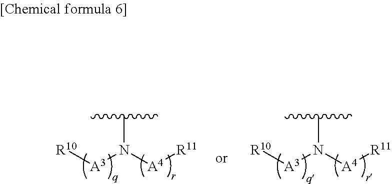

wherein symbols have the same meanings as those in the above item (1), or a pharmaceutically acceptable salt thereof.
(8) The compound according to any one of the above items (1) to (7), wherein R¹⁰ is substituted or unsubstituted aromatic carbocyclyl or substituted or unsubstituted aromatic heterocyclyl, or a pharmaceutically acceptable salt thereof.
(9) The compound according to any one of the above items (1) to (8), wherein R¹⁰ is substituted or unsubstituted aromatic heterocyclyl, or a pharmaceutically acceptable salt thereof.
(10) The compound according to any one of the above items (1) to (9), wherein R¹⁰ is substituted or unsubstituted 5-membered aromatic heterocyclyl, or a pharmaceutically acceptable salt thereof.
(11) The compound according to any one of the above items (1) to (10), wherein R¹¹ is substituted or unsubstituted aromatic carbocyclyl, or a pharmaceutically acceptable salt thereof.
(12) The compound according to any one of the above items (1) to (11), wherein q, r, q′, and r′ are 1, or a pharmaceutically acceptable salt thereof.
(13) The compound according to any one of the above items (1) to (12), wherein the compound is represented by Formula (II):
[Chemical Formula 7] [symbol]
wherein R¹ is a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R² is a hydrogen atom, halogen, or substituted or unsubstituted alkyl;
R^{2′} is a hydrogen atom, halogen, or substituted or unsubstituted alkyl;
R³ is a hydrogen atom, halogen, or substituted or unsubstituted alkyl;
R^{3′} is a hydrogen atom, halogen, or substituted or unsubstituted alkyl, or a pharmaceutically acceptable salt thereof.
   (14) A compound represented by Formula (III):

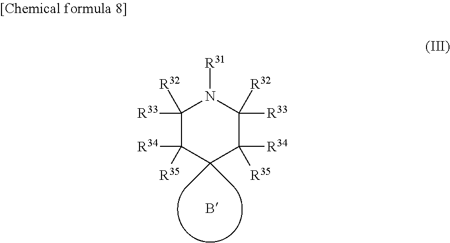

wherein R³¹ is a hydrogen atom or C1-C3 alkyl;
R³² is each independently a hydrogen atom or substituted or unsubstituted alkyl;
R³³ is each independently a hydrogen atom or substituted or unsubstituted alkyl;
R³⁴ is each independently a hydrogen atom, halogen, or substituted or unsubstituted alkyl;
R³⁵ is each independently a hydrogen atom, halogen, or substituted or unsubstituted alkyl;
R³² and R³³ and R³⁴ and R³⁵ may be taken together with an identical carbon atom to which they are bonded to form a substituted or unsubstituted non-aromatic carbocycle or a substituted or unsubstituted non-aromatic heterocycle;
ring B′ is a group represented by Formula:

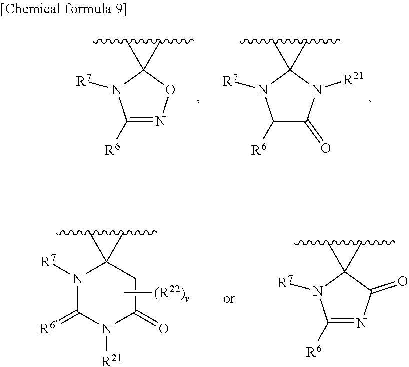

wherein R⁶ is a group represented by Formula:

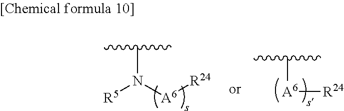

wherein A⁶ is each independently CR²⁵R^{25′};
R²⁵ is each independently a hydrogen atom, halogen, substituted or unsubstituted alkyl, or substituted or unsubstituted alkyloxy;
R^{25′} is each independently a hydrogen atom, halogen, substituted or unsubstituted alkyl, or substituted or unsubstituted alkyloxy;
s is 0 or 1;
s′ is 0, 1, or 2;
R²⁴ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁵ is a hydrogen atom or substituted or unsubstituted alkyl;
R⁶ is a group represented by Formula:

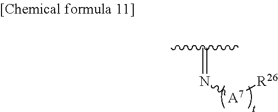

wherein A⁷ is each dependently CR²⁷R^{27′};
R²⁷ is a hydrogen atom, halogen, substituted or unsubstituted alkyl, or substituted or unsubstituted alkyloxy;
R^{27′} is a hydrogen atom, halogen, substituted or unsubstituted alkyl, or substituted or unsubstituted alkyloxy;
t is 0 or 1;
R²⁶ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁷ is a group represented by Formula:

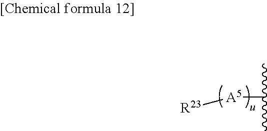

wherein A⁵ is each independently CR²⁸R^{28′};
R²⁸ is each independently a hydrogen atom, halogen, substituted or unsubstituted alkyl, or substituted or unsubstituted alkyloxy;
R^{28′} is each independently a hydrogen atom, halogen, substituted or unsubstituted alkyl, or substituted or unsubstituted alkyloxy;
u is 0, 1, or 2;
R²³ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl,
R²¹ is a hydrogen atom or substituted or unsubstituted alkyl;
R²² is each independently halogen or substituted or unsubstituted alkyl;
v is 0, 1, or 2,
   or a pharmaceutically acceptable salt thereof.
   (15) The compound according to the above item (14), wherein ring B′ is a ring represented by Formula:

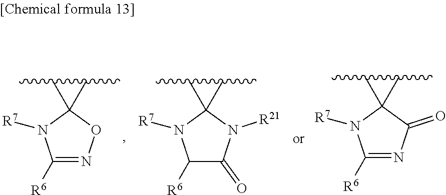

wherein symbols have the same meanings as those in the above item (14), or a pharmaceutically acceptable salt thereof.
(16) The compound according to the above item (14) or (15), wherein R⁶ is a group represented by Formula:

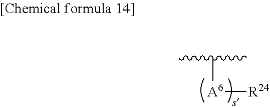

wherein symbols have the same meanings as those in the above item (14), or a pharmaceutically acceptable salt thereof.
(17) The compound according to any one of the above items (14) to (16), wherein s′ is 1, or a pharmaceutically acceptable salt thereof.
(18) The compound according to any one of the above items (14) to (17), wherein R²⁴ is substituted or unsubstituted aromatic carbocyclyl, or a pharmaceutically acceptable salt thereof.
(19) The compound according to any one of the above items (14) to (18), wherein u is 1, or a pharmaceutically acceptable salt thereof.
(20) The compound according to any one of the above items (14) to (19), wherein R²³ is substituted or unsubstituted aromatic heterocyclyl, or a pharmaceutically acceptable salt thereof.
(21) The compound according to any one of the above items (14) to (20), wherein R³² and R³³ are hydrogen atoms, or a pharmaceutically acceptable salt thereof.
(22) A pharmaceutical composition comprising the compound according to any one of the above items (1) to (21) or a pharmaceutically acceptable salt thereof.
(23) The pharmaceutical composition according to the above item (22), wherein the pharmaceutical composition is a serotonin 5-HT2A receptor antagonist and/or inverse agonist.
(24) The pharmaceutical composition according to the above item (22), wherein the pharmaceutical composition is a serotonin 5-HT2A and 5-HT2C receptor antagonist and/or inverse agonist.
(25) A method for treating and/or preventing a disease associated with a 5-HT2A receptor comprising administering the compound according to any one of the above items (1) to (21) or a pharmaceutically acceptable salt thereof.
(26) A method for treating and/or preventing a disease associated with 5-HT2A and 5-HT2C receptors comprising administering the compound according to any one of the above items (1) to (21) or a pharmaceutically acceptable salt thereof.
(27) Use of the compound according to any one of the above items (1) to (21) or a pharmaceutically acceptable salt thereof, for the manufacture of a therapeutic and/or prophylactic agent for a disease associated with a 5-HT2A receptor antagonist and/or inverse agonist.
(28) Use of the compound according to any one of the above items (1) to (21) or a pharmaceutically acceptable salt thereof, for the manufacture of a therapeutic and/or prophylactic agent for a disease associated with a 5-HT2A and 5-HT2C receptor antagonist and/or inverse agonist.
(29) The compound according to any one of the above items (1) to (21) or a pharmaceutically acceptable salt thereof, for use in treating and/or preventing a disease associated with a 5-HT2A receptor antagonist and/or inverse agonist.
(30) The compound according to any one of the above items (1) to (21) or a pharmaceutically acceptable salt thereof, for use in treating and/or preventing a disease associated with a 5-HT2A and 5-HT2C receptor antagonist and/or inverse agonist.
(1′) A compound represented by Formula (I):

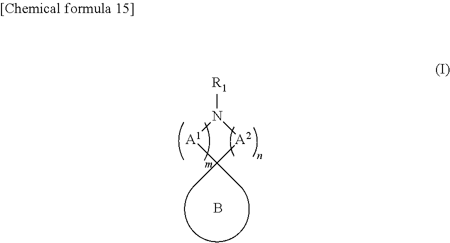

wherein R¹ is a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
A¹ is each independently CR²R^{2′};
A² is each independently CR³R^{3′};
R² is each independently a hydrogen atom, halogen, substituted or unsubstituted alkyl, or substituted or unsubstituted alkyloxy;
R^{2′} is each independently a hydrogen atom, halogen, substituted or unsubstituted alkyl, or substituted or unsubstituted alkyloxy;
R³ is each independently a hydrogen atom, halogen, substituted or unsubstituted alkyl, or substituted or unsubstituted alkyloxy;
R^{3′} is each independently a hydrogen atom, halogen, substituted or unsubstituted alkyl, or substituted or unsubstituted alkyloxy;
R² and R^{2′} and R³ and R^{3′} may be taken together with an identical carbon atom to which they are bonded to form a substituted or unsubstituted non-aromatic carbocycle or a substituted or unsubstituted non-aromatic heterocycle;
m and n are each independently 1, 2, or 3;
ring B is a ring represented by Formula:

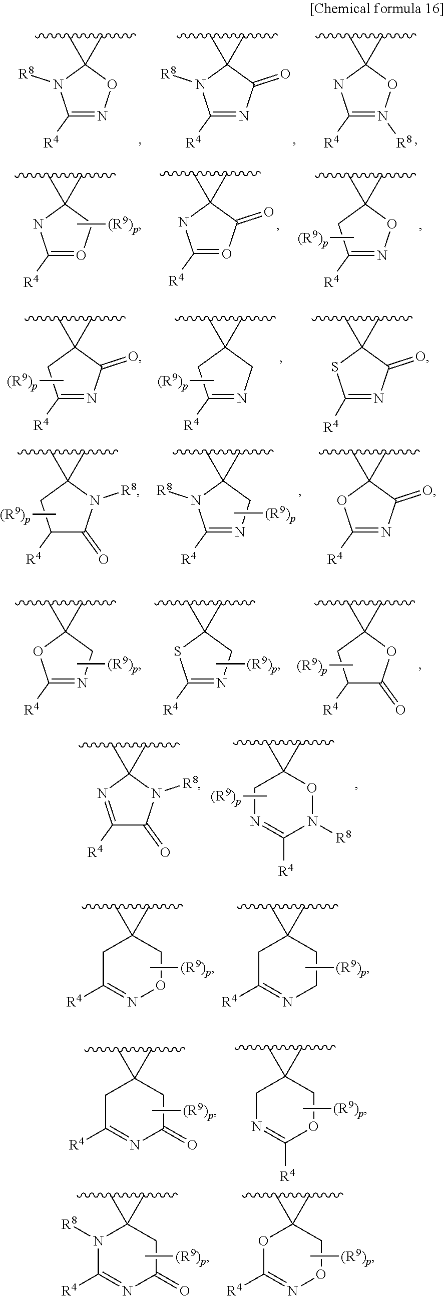

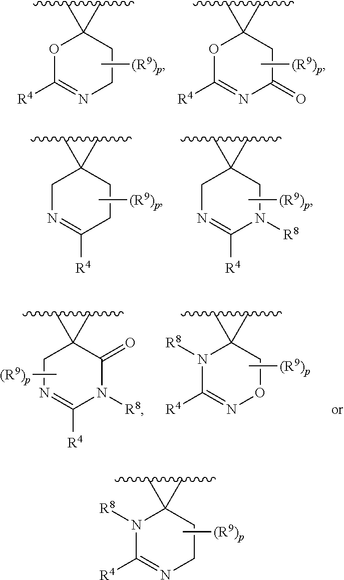

wherein R⁴ is a group represented by Formula:

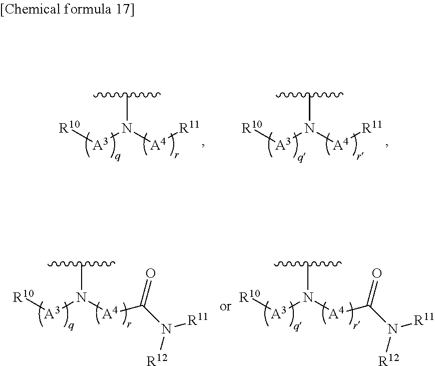

wherein A³ is each CR¹³R^{13′};
A⁴ is each independently CR¹⁴R^{14′};
R¹³ is each independently a hydrogen atom, halogen, substituted or unsubstituted alkyl, or substituted or unsubstituted alkyloxy;
R^{13′} is each independently a hydrogen atom, halogen, substituted or unsubstituted alkyl, or substituted or unsubstituted alkyloxy;
R¹⁴ is each independently a hydrogen atom, halogen, substituted or unsubstituted alkyl, or substituted or unsubstituted alkyloxy;
R^{14′} is each independently a hydrogen atom, halogen, substituted or unsubstituted alkyl, or substituted or unsubstituted alkyloxy;
q and r are each independently 0, 1, or 2;
q′ and r′ are each independently 1 or 2;
R¹⁰ and R¹¹ are each independently substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R¹² is a hydrogen atom or substituted or unsubstituted alkyl;
R⁸ is a hydrogen atom or substituted or unsubstituted alkyl;
R⁹ is each independently halogen or substituted or unsubstituted alkyl;
p is an integer of any of 0 to 6,
   or a pharmaceutically acceptable salt thereof.
   (2′) The compound according to the above item (1′), wherein R¹ is a hydrogen atom or substituted or unsubstituted alkyl, or a pharmaceutically acceptable salt thereof.
   (3′) The compound according to the above item (1′) or (2′), wherein m and n are each independently 1 or 2, or a pharmaceutically acceptable salt thereof.
   (4′) The compound according to the above item (1′) or (2′), wherein m and n are 2, or a pharmaceutically acceptable salt thereof.
   (5′) The compound according to any one of the above items (1′) to (4′), wherein ring B is a ring represented by Formula:

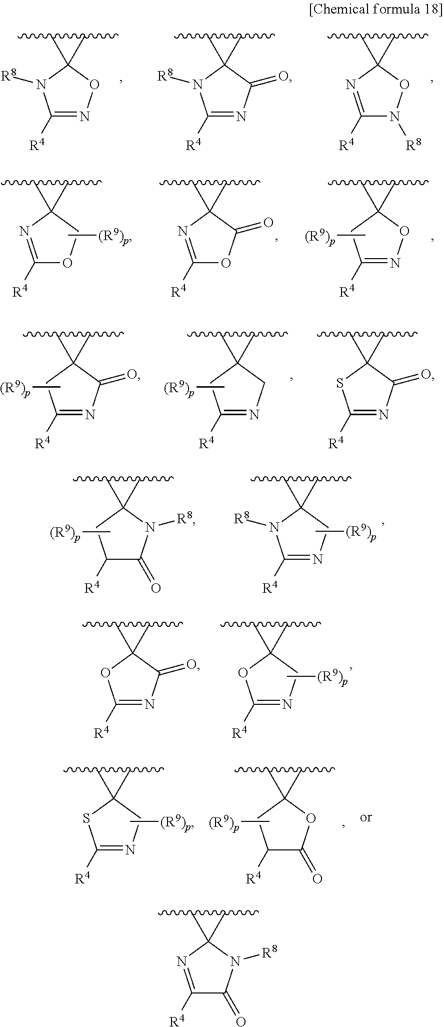

wherein symbols have the same meanings as those in the above item (1′), or a pharmaceutically acceptable salt thereof.
(6′) The compound according to any one of the above items (1′) to (4′), wherein ring B is a ring represented by Formula:

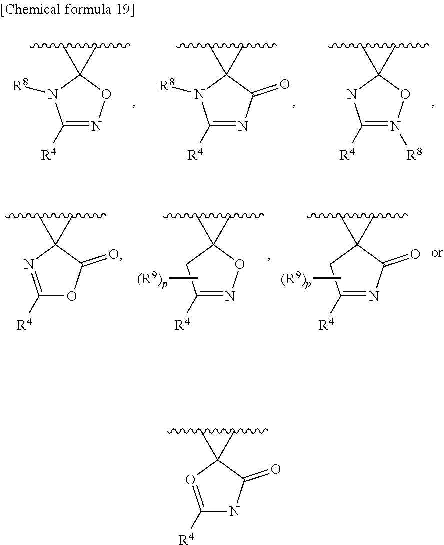

wherein symbols have the same meanings as those in the above item (1′), or a pharmaceutically acceptable salt thereof.
(7′) The compound according to any one of the above items (1′) to (6′), wherein R⁴ is a group represented by Formula:

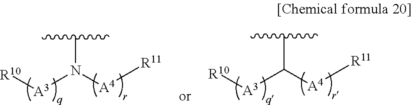

wherein symbols have the same meanings as those in the above item (1′), or a pharmaceutically acceptable salt thereof.
(8′) The compound according to any one of the above items (1′) to (7′), wherein R¹⁰ is substituted or unsubstituted aromatic carbocyclyl or substituted or unsubstituted aromatic heterocyclyl, or a pharmaceutically acceptable salt thereof.
(9′) The compound according to any one of the above items (1′) to (8′), wherein R¹⁰ is substituted or unsubstituted aromatic heterocyclyl, or a pharmaceutically acceptable salt thereof.
(10′) The compound according to any one of the above items (1′) to (9′), wherein R¹⁰ is substituted or unsubstituted 5-membered aromatic heterocyclyl, or a pharmaceutically acceptable salt thereof.
(11′) The compound according to any one of the above items (1′) to (10′), wherein R¹¹ is substituted or unsubstituted aromatic carbocyclyl, or a pharmaceutically acceptable salt thereof.
(12′) The compound according to any one of the above items (1′) to (11′), wherein q, r, q′, and r′ are 1, or a pharmaceutically acceptable salt thereof.
(13′) The compound according to any one of the above items (1′) to (12′), wherein the compound is represented by Formula (II):

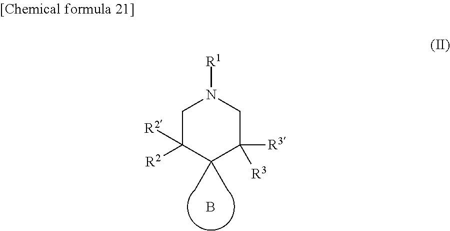

wherein R¹ is a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R² is a hydrogen atom, halogen, or substituted or unsubstituted alkyl;
R^{2′} is a hydrogen atom, halogen, or substituted or unsubstituted alkyl;
R³ is a hydrogen atom, halogen, or substituted or unsubstituted alkyl;
R^{3′} is a hydrogen atom, halogen, or substituted or unsubstituted alkyl; and other symbols have the same meanings as those in the above item (1′), or a pharmaceutically acceptable salt thereof.
   (14′) The compound according to the above item (1′), wherein the compound is represented by Formula (II):
   [Chemical formula 22] [symbol]
wherein R¹ is a hydrogen atom or alkyl;
R² is a hydrogen atom or halogen;
R^{2′} is a hydrogen atom;
R³ is a hydrogen atom;
R^{3′} is a hydrogen atom;
ring B is a ring represented by Formula:

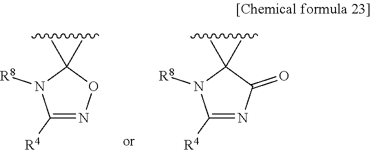

wherein R⁴ is a group represented by Formula:

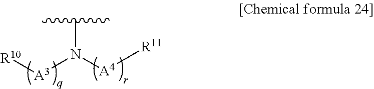

wherein A³ is CR¹³R^{13′};
A⁴ is CR¹⁴R^{14′};
R¹³ is a hydrogen atom;
R^{13′} is a hydrogen atom;
R¹⁴ is a hydrogen atom;
R^{14′} is a hydrogen atom;
q and r are each 1;
R¹⁰ is phenyl substituted with halogen, phenyl, 5-membered aromatic heterocyclyl substituted with one or more substituents selected from substituent group ω (substituent group ω: alkyl, haloalkyl, and non-aromatic carbocyclyl), or 6-membered aromatic heterocyclyl substituted with one or more substituents selected from substituent group ω′ (substituent group ω′: alkyl and halogen);
R¹¹ is a group represented by Formula:

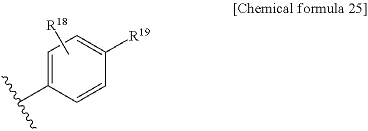

wherein R¹⁸ is a hydrogen atom or halogen;
R¹⁹ is alkyl, haloalkyl, alkyl substituted with aromatic carbocyclyl, alkyloxy, alkyloxy substituted with non-aromatic carbocyclyl, alkyloxy substituted with non-aromatic carbocyclyl substituted with halogen, or haloalkyloxy, 9-membered aromatic heterocyclyl, which is bicyclic, or 9-membered aromatic heterocyclyl, which is bicyclic, substituted with one or more substituents selected from substituent group ψ (substituent group ψ: halogen, alkyl, and alkyloxy);
R⁸ is a hydrogen atom,
   or a pharmaceutically acceptable salt thereof.
   (15′) A compound represented by Formula (III):

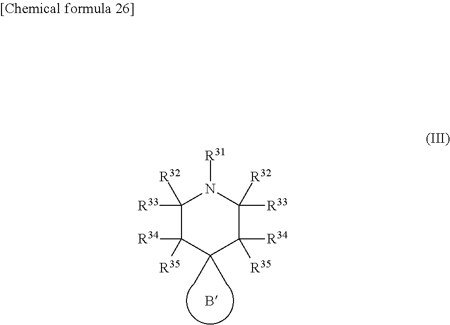

wherein R³¹ is a hydrogen atom or C1-C3 alkyl;
R³² is each independently a hydrogen atom or substituted or unsubstituted alkyl;
R³³ is each independently a hydrogen atom or substituted or unsubstituted alkyl;
R³⁴ is each independently a hydrogen atom, halogen, or substituted or unsubstituted alkyl;
R³⁵ is each independently a hydrogen atom, halogen, or substituted or unsubstituted alkyl;
R³² and R³³ and R³⁴ and R³⁵ may be taken together with an identical carbon atom to which they are bonded to form a substituted or unsubstituted non-aromatic carbocycle or a substituted or unsubstituted non-aromatic heterocycle;
ring B′ is a group represented by Formula:

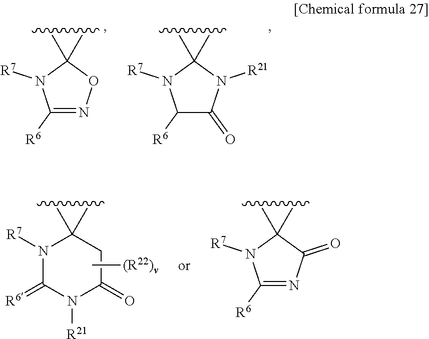

wherein R⁶ is a group represented by Formula:

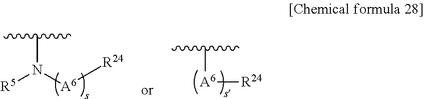

wherein A⁶ is each independently CR²⁵R^{25′};
R²⁵ is each independently a hydrogen atom, halogen, substituted or unsubstituted alkyl, or substituted or unsubstituted alkyloxy;
R^{25′} is each independently a hydrogen atom, halogen, substituted or unsubstituted alkyl, or substituted or unsubstituted alkyloxy;
s is 0 or 1;
s′ is 0, 1, or 2;
R²⁴ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁵ is a hydrogen atom or substituted or unsubstituted alkyl;
R^{6′} is a group represented by Formula:

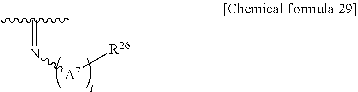

wherein A⁷ is CR²⁷R^{27′};
R²⁷ is a hydrogen atom, halogen, substituted or unsubstituted alkyl, or substituted or unsubstituted alkyloxy;
R^{27′} is a hydrogen atom, halogen, substituted or unsubstituted alkyl, or substituted or unsubstituted alkyloxy;
t is 0 or 1;
R²⁶ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁷ is a group represented by Formula:

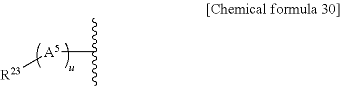

wherein A⁵ is each independently CR²⁸R^{28′};
R²⁸ is each independently a hydrogen atom, halogen, substituted or unsubstituted alkyl, or substituted or unsubstituted alkyloxy;
R^{28′} is each independently a hydrogen atom, halogen, substituted or unsubstituted alkyl, or substituted or unsubstituted alkyloxy;
u is 0, 1, or 2;
R²³ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl,
R²¹ is a hydrogen atom or substituted or unsubstituted alkyl;
R²² is each independently halogen or substituted or unsubstituted alkyl;
v is 0, 1, or 2,
   or a pharmaceutically acceptable salt thereof.
   (16′) The compound according to the above item (15′), wherein ring B′ is a ring represented by Formula:

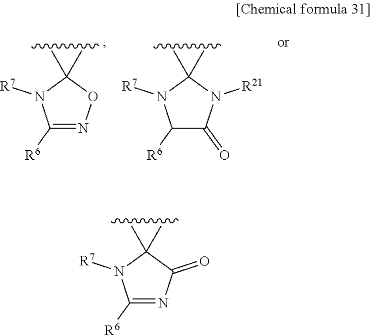

wherein symbols have the same meanings as those in the above item (15′), or a pharmaceutically acceptable salt thereof.
(17′) The compound according to the above item (15′) or (16′), wherein R⁶ is a group represented by Formula:

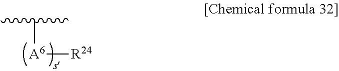

wherein symbols have the same meanings as those in the above item (15′), or a pharmaceutically acceptable salt thereof.
(18′) The compound according to any one of the above items (15′) to (17′), wherein s′ is 1, or a pharmaceutically acceptable salt thereof.
(19′) The compound according to any one of the above items (15′) to (18′), wherein R²⁴ is substituted or unsubstituted aromatic carbocyclyl, or a pharmaceutically acceptable salt thereof.
(20′) The compound according to any one of the above items (15′) to (19′), wherein u is 1, or a pharmaceutically acceptable salt thereof.
(21′) The compound according to any one of the above items (15′) to (20′), wherein R²³ is substituted or unsubstituted aromatic heterocyclyl, or a pharmaceutically acceptable salt thereof.
(22′) The compound according to any one of the above items (15′) to (21′), wherein R³² and R³³ are hydrogen atoms, or a pharmaceutically acceptable salt thereof.
(23′) The compound according to the above item (1′), wherein the compound is selected from the group consisting of compounds I-067, I-080, I-104, I-105, I-113, I-114, I-115, I-125, and I-128, or a pharmaceutically acceptable salt thereof.
(24′) A pharmaceutical composition comprising the compound according to any one of the above items (1′) to (23′) or a pharmaceutically acceptable salt thereof.
(25′) The pharmaceutical composition according to the above item (24′), wherein the pharmaceutical composition is a serotonin 5-HT2A receptor antagonist and/or inverse agonist.
(26′) The pharmaceutical composition according to the above item (24′), wherein the pharmaceutical composition is a serotonin 5-HT2A and 5-HT2C receptor antagonist and/or inverse agonist.
(27′) A method for treating and/or preventing a disease associated with a 5-HT2A receptor comprising administering the compound according to any one of the above items (1′) to (23′) or a pharmaceutically acceptable salt thereof.
(28′) A method for treating and/or preventing a disease associated with 5-HT2A and 5-HT2C receptors comprising administering the compound according to any one of the above items (1′) to (23′) or a pharmaceutically acceptable salt thereof.
(29′) The compound according to any one of the above items (1′) to (23′) or a pharmaceutically acceptable salt thereof, for use in treating and/or preventing a disease associated with a 5-HT2A receptor antagonist and/or inverse agonist.
(30′) The compound according to any one of the above items (1′) to (23′) or a pharmaceutically acceptable salt thereof, for use in treating and/or preventing a disease associated with a 5-HT2A and 5-HT2C receptor antagonist and/or inverse agonist.
(31′) Use of the compound according to any one of the above items (1′) to (23′) or a pharmaceutically acceptable salt thereof, for the manufacture of a therapeutic and/or prophylactic agent for a disease associated with a 5-HT2A receptor antagonist and/or inverse agonist.
(32′) Use of the compound according to any one of the above items (1′) to (23′) or a pharmaceutically acceptable salt thereof, for the manufacture of a therapeutic and/or prophylactic agent for a disease associated with a 5-HT2A and 5-HT2C receptor antagonist and/or inverse agonist.

### Effect of the Invention

The compound according to the present invention has serotonin 5-HT2A receptor antagonism and/or inverse agonism, and is useful as a therapeutic and/or prophylactic agent for Parkinson's disease- and/or dementia-related hallucinations and delusions.

### MODE FOR CARRYING OUT THE INVENTION

Terms used in this description are explained below. Each term, unless otherwise indicated, has the same meaning when it is used alone or together with other terms.

The term of “consisting of” means having only components.

The term of “comprising” means not restricting with components and not excluding undescribed factors.

Hereinafter, the present invention is described with reference to embodiments. It should be understood that, throughout the present description, the expression of a singular form includes the concept of its plural form unless specified otherwise. Accordingly, it should be understood that an article in singular form (for example, in the English language, “a”, “an”, and “the”) includes the concept of its plural form unless specified otherwise.

Furthermore, it should be understood that the terms used herein are used in a meaning normally used in the art unless specified otherwise. Thus, unless defined otherwise, all technical and scientific terms used herein have the same meaning as those generally understood by those skilled in the art in the field to which the present invention pertains. If there is a contradiction, the present description (including definitions) precedes.

The term “halogen” includes a fluorine atom, a chlorine atom, a bromine atom and an iodine atom. A fluorine atom and a chlorine atom are especially preferable.

The term “alkyl” includes a C1 to C15, preferably C1 to C10, more preferably C1 to C6 and further preferably C1 to C4 linear or branched hydrocarbon group. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, and n-decyl.

Preferred embodiments of “alkyl” include methyl, ethyl, n-propyl, isopropyl, n-butyl, isobutyl, sec-butyl, tert-butyl, and n-pentyl. More preferred embodiments include methyl, ethyl, n-propyl, isopropyl, and tert-butyl.

As the “alkyl” moiety in the case where R¹¹, R²⁴, or R²⁶ is aromatic carbocyclyl substituted with alkyl, C2-C5 alkyl is preferable. Examples include ethyl, n-propyl, isopropyl, n-butyl, isobutyl, sec-butyl, tert-butyl, and n-pentyl. Further, C3-C5 alkyl is more preferable. Examples include n-propyl, isopropyl, n-butyl, isobutyl, sec-butyl, tert-butyl, and n-pentyl.

As the “alkyl” moiety of alkyloxy in the case where R¹¹, R²⁴, or R²⁶ is aromatic carbocyclyl substituted with alkyloxy, C2-C5 alkyl is preferable. Examples include ethyl, n-propyl, isopropyl, n-butyl, isobutyl, sec-butyl, tert-butyl, and n-pentyl. Further, C3-C5 alkyl is more preferable. Examples include n-propyl, isopropyl, n-butyl, isobutyl, sec-butyl, tert-butyl, and n-pentyl.

The term “haloalkyl” means the above alkyl substituted with one or more halogen(s). When substituted with two or more halogens, the halogens may be the same or different. Examples include fluoromethyl, difluoromethyl, trifluoromethyl, chlorofluoromethyl, 2,2-difluoroethyl, 2,2,2-trifluoroethyl, 2,2-difluoropropyl, 2,2,3,3-tetrafluoropropyl, and 2,2,3,3,3-pentafluoropropyl.

The term “alkenyl” includes a C2 to C15, preferably a C2 to C10, more preferably a C2 to C6 and further preferably a C2 to C4 linear or branched hydrocarbon group having one or more double bond(s) at any position(s). Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl, and pentadecenyl.

Preferred embodiments of “alkenyl” include vinyl, allyl, propenyl, isopropenyl, and butenyl. More preferred embodiments include vinyl and n-propenyl.

The term “alkynyl” includes a C2 to C10, preferably a C2 to C8, more preferably a C2 to C6 and further preferably a C2 to C4 linear or branched hydrocarbon group having one or more triple bond(s) at any position(s). Furthermore, it may have double bond(s) at any position(s). Examples include ethynyl, propynyl, butynyl, pentynyl, hexynyl, heptynyl, octynyl, nonynyl, and decynyl.

Preferred embodiments of “alkynyl” include ethynyl, propynyl, butynyl, and pentynyl. More preferred embodiments include ethynyl and propynyl.

The term “aromatic carbocyclyl” means a cyclic aromatic hydrocarbon group which is monocyclic or polycyclic having two or more rings. Examples include phenyl, naphthyl, anthryl, and phenanthryl.

Preferred embodiments of the “aromatic carbocyclyl” include phenyl.

The term “aromatic carbocycle” means a ring derived from the above “aromatic carbocyclyl”.

The term “non-aromatic carbocyclyl” means a cyclic saturated hydrocarbon group or a cyclic non-aromatic unsaturated hydrocarbon group, which is monocyclic or polycyclic having two or more rings. The “non-aromatic carbocyclyl” which is polycyclic having two or more rings includes a fused ring group wherein a non-aromatic carbocyclyl, which is monocyclic or polycyclic having two or more rings, is fused with a ring of the above “aromatic carbocyclyl”.

In addition, examples of the “non-aromatic carbocyclyl” also include a group having a bridge or a group to form a spiro ring as follows:

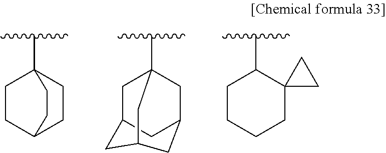 [SMILES: *C12CCC(CC1)CC2 ; *C12CC3CC(CC(C3)C1)C2 ; *C1CCCCC12CC2]

The non-aromatic carbocyclyl which is monocyclic is preferably C3 to C16, more preferably C3 to C12 and further preferably C4 to C8 carbocyclyl. Examples include cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl, and cyclohexadienyl.

The non-aromatic carbocyclyl which is polycyclic having two or more rings is preferably C8 to C20, more preferably C8 to C16 carbocyclyl. Examples include indanyl, indenyl, acenaphthyl, tetrahydronaphthyl, and fluorenyl.

The term “non-aromatic carbocycle” means a ring derived from the above “non-aromatic carbocyclyl”.

The term “aromatic heterocyclyl” means an aromatic cyclyl, which is monocyclic or polycyclic having two or more rings, containing one or more, same or different heteroatom(s) selected independently from O, S and N.

The aromatic heterocyclyl, which is polycyclic having two or more rings, includes a fused ring group wherein an aromatic heterocyclyl, which is monocyclic or polycyclic having two or more rings, is fused with a ring of the above “aromatic carbocyclyl”, the bond may be held in any ring.

The aromatic heterocyclyl, which is monocyclic, is preferably a 5- to 8-membered ring and more preferably a 5- to 6-membered ring. Examples of the 5-membered aromatic heterocyclyl include pyrrolyl, imidazolyl, pyrazolyl, triazolyl, tetrazolyl, furyl, thienyl, isoxazolyl, oxazolyl, oxadiazolyl, isothiazolyl, thiazolyl, and thiadiazolyl. Examples of the 6-membered aromatic heterocyclyl include pyridyl, pyridazinyl, pyrimidinyl, pyrazinyl, triazinyl.

The aromatic heterocyclyl, which is bicyclic, is preferably an 8- to 10-membered ring and more preferably a 9- to 10-membered ring. Examples of aromatic heterocyclyl, which is bicyclic, include indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl, and thiazolopyridyl.

The aromatic heterocyclyl, which is polycyclic having three or more rings, is preferably a 13- to 15-membered ring. Examples of aromatic heterocyclyl, which is polycyclic having three or more rings, include carbazolyl, acridinyl, xanthenyl, phenothiazinyl, phenoxathiinyl, phenoxazinyl, and dibenzofuryl.

The term “aromatic heterocycle” means a ring derived from the above “aromatic heterocyclyl”.

The term “aromatic nitrogen-containing heterocyclyl” means an aromatic heterocyclyl, which is monocyclic or polycyclic having two or more rings, containing one or more N and optionally containing one or more, same or different heteroatom(s) selected independently from O and S. The aromatic nitrogen-containing heterocyclyl, which is polycyclic having two or more rings, includes a fused ring group wherein an aromatic nitrogen-containing heterocyclyl, which is monocyclic or polycyclic having two or more rings, is fused with a ring of the above “aromatic carbocyclyl”, the bond may be held in any ring.

The aromatic nitrogen-containing heterocyclyl, which is monocyclic, is preferably a 5- to 8-membered ring and more preferably a 5- to 6-membered ring. Examples of the 5-membered aromatic nitrogen-containing heterocyclyl include pyrrolyl, imidazolyl, pyrazolyl, triazolyl, tetrazolyl, isoxazolyl, oxazolyl, oxadiazolyl, isothiazolyl, thiazolyl, and thiadiazolyl. Examples of the 6-membered aromatic nitrogen-containing heterocyclyl include pyridyl, pyridazinyl, pyrimidinyl, pyrazinyl, triazinyl.

The aromatic nitrogen-containing heterocyclyl, which is bicyclic, is preferably an 8- to 10-membered ring and more preferably a 9- to 10-membered ring. Examples of aromatic nitrogen-containing heterocyclyl, which is bicyclic, include indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzotriazolyl, imidazopyridyl, triazolopyridyl, imidazothiazolyl, pyrazinopyridazinyl, oxazolopyridyl, and thiazolopyridyl.

The aromatic nitrogen-containing heterocyclyl, which is polycyclic having three or more rings, is preferably a 13- to 15-membered ring. Examples of aromatic nitrogen-containing heterocyclyl, which is polycyclic having three or more rings, include carbazolyl, acridinyl, and phenothiazinyl.

The term “non-aromatic heterocyclyl” means a non-aromatic cyclyl, which is monocyclic or polycyclic having two or more rings, containing one or more, same or different heteroatom(s) selected independently from O, S and N. The “non-aromatic heterocyclyl”, which is polycyclic having two or more rings, includes a non-aromatic heterocyclyl, which is monocyclic or polycyclic having two or more rings, fused with a ring of the above “aromatic carbocyclyl”, “non-aromatic carbocyclyl” and/or “aromatic heterocyclyl”, and further includes a non-aromatic carbocyclyl, which is monocyclic or polycyclic having two or more rings, fused with a ring of the above “aromatic heterocyclyl”, the bond may be held in any ring.

In addition, examples of the “non-aromatic heterocyclyl” also include a group having a bridge or a group to form a spiro ring as follows:

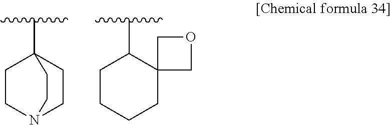

The non-aromatic heterocyclyl, which is monocyclic, is preferably a 3- to 8-membered and more preferably a 5- to 6-membered ring.

Examples of the 3-membered non-aromatic heterocyclyl include thiiranyl, oxiranyl and aziridinyl. Examples of the 4-membered non-aromatic heterocyclyl include oxetanyl and azetidinyl. Examples of the 5-membered non-aromatic heterocyclyl include oxathiolanyl, thiazolidinyl, pyrrolidinyl, pyrrolinyl, imidazolidinyl, imidazolinyl, pyrazolidinyl, pyrazolinyl, tetrahydrofuryl, dihydrothiazolyl, tetrahydroisothiazolyl, dioxolanyl, dioxolyl, and thiolanyl. Examples of the 6-membered non-aromatic heterocyclyl include dioxanyl, thianyl, piperidyl, piperazinyl, morpholinyl, morpholino, thiomorpholinyl, thiomorpholino, dihydropyridyl, tetrahydropyridyl, tetrahydropyranyl, dihydrooxazinyl, tetrahydropyridazinyl, hexahydropyrimidinyl, dioxazinyl, thiinyl, and thiazinyl. Examples of the 7-membered non-aromatic heterocyclyl include hexahydroazepinyl, tetrahydrodiazepinyl, and oxepanyl. Examples of the 8-membered non-aromatic heterocyclyl include azocane, thiocane and oxocane.

The non-aromatic heterocyclyl, which is polycyclic having two or more rings, is preferably an 8- to 20-membered and more preferably an 8- to 10-membered ring. Examples of non-aromatic heterocyclyl, which is polycyclic having two or more rings, include indolinyl, isoindolinyl, chromanyl, and isochromanyl.

The term “non-aromatic nitrogen-containing heterocyclyl” means a non-aromatic heterocyclyl, which is monocyclic or polycyclic having two or more rings, containing one or more N. The non-aromatic heterocyclyl, which is polycyclic having two or more rings, includes a fused ring group wherein a non-aromatic nitrogen-containing heterocyclyl, which is monocyclic or polycyclic having two or more rings, is fused with a ring of the above “aromatic carbocyclyl”, “non-aromatic carbocyclyl” and/or “aromatic heterocyclyl”, the bond may be held in any ring.

Examples include rings as follows.

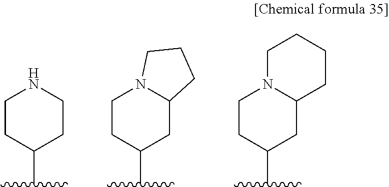 [SMILES: *C1CCNCC1 ; *C1CCN2CCCC2C1 ; *C1CCN2CCCCC2C1]

Furthermore, “non-aromatic nitrogen-containing heterocyclyl” also include a group having a bridge or a group to form a spiro ring as follows:

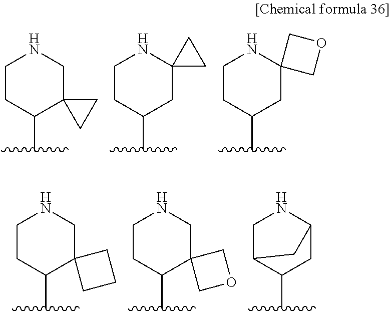

The term “non-aromatic heterocycle” means a ring derived from the above “non-aromatic heterocyclyl”.

The term “non-aromatic carbocycle that R² and R^{2′}, R³ and R^{3′}, R³² and R³³, or R³⁴ and R³⁵ are taken together with the carbon atom to which they are bonded to form” means rings as follows as examples.

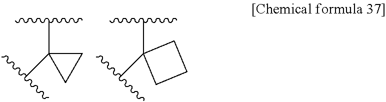

The term “trialkylsilyl” means a group in which the above three “alkyls” are bound to a silicon atom. The three alkyls may be the same or different. Examples include trimethylsilyl, triethylsilyl and tert-butyldimethylsilyl.

In the present description, the phrase “may be substituted with substituent group α” means that “may be substituted with one or more group(s) selected from substituent group α”. The same also applies to substituent groups β, γ, and γ′.

Substituents for “substituted alkyl”, “substituted alkenyl”, “substituted alkynyl”, “substituted alkyloxy”, “substituted alkenyloxy”, “substituted alkynyloxy”, “substituted alkylcarbonyloxy”, “substituted alkenylcarbonyloxy”, “substituted alkynylcarbonyloxy”, “substituted alkylcarbonyl”, “substituted alkenylcarbonyl”, “substituted alkynylcarbonyl”, “substituted alkyloxycarbonyl”, “substituted alkenyloxycarbonyl”, “substituted alkynyloxycarbonyl”, “substituted alkylsulfanyl”, “substituted alkenylsulfanyl”, “substituted alkynylsulfanyl”, “substituted alkylsulfinyl”, “substituted alkenylsulfinyl”, “substituted alkynylsulfinyl”, “substituted alkylsulfonyl”, “substituted alkenylsulfonyl”, “substituted alkynylsulfonyl”, and the like include the following substituent group A. A carbon atom at any position may be bonded to one or more group(s) selected from the following substituent group A.

Substituent group A: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureide, amidino, guanidino, pentafluorothio, trialkylsilyl, alkyloxy which may be substituted with substituent group α, alkenyloxy which may be substituted with substituent group α, alkynyloxy which may be substituted with substituent group α, alkylcarbonyloxy which may be substituted with substituent group α, alkenylcarbonyloxy which may be substituted with substituent group α, alkynylcarbonyloxy which may be substituted with substituent group α, alkylcarbonyl which may be substituted with substituent group α, alkenylcarbonyl which may be substituted with substituent group α, alkynylcarbonyl which may be substituted with substituent group α, alkyloxycarbonyl which may be substituted with substituent group α, alkenyloxycarbonyl which may be substituted with substituent group α, alkynyloxycarbonyl which may be substituted with substituent group α, alkylsulfanyl which may be substituted with substituent group α, alkenylsulfanyl which may be substituted with substituent group α, alkynylsulfanyl which may be substituted with substituent group α, alkylsulfinyl which may be substituted with substituent group α, alkenylsulfinyl which may be substituted with substituent group α, alkynylsulfinyl which may be substituted with substituent group α, alkylsulfonyl which may be substituted with substituent group α, alkenylsulfonyl which may be substituted with substituent group α, alkynylsulfonyl which may be substituted with substituent group α,

amino which may be substituted with substituent group β, imino which may be substituted with substituent group β, carbamoyl which may be substituted with substituent group β, sulfamoyl which may be substituted with substituent group 3, aromatic carbocyclyl which may be substituted with substituent group γ, non-aromatic carbocyclyl which may be substituted with substituent group γ′, aromatic heterocyclyl which may be substituted with substituent group γ, non-aromatic heterocyclyl which may be substituted with substituent group γ′, aromatic carbocyclyloxy which may be substituted with substituent group γ, non-aromatic carbocyclyloxy which may be substituted with substituent group γ′, aromatic heterocyclyloxy which may be substituted with substituent group γ, non-aromatic heterocyclyloxy which may be substituted with substituent group γ′, aromatic carbocyclylcarbonyloxy which may be substituted with substituent group γ, non-aromatic carbocyclylcarbonyloxy which may be substituted with substituent group γ′, aromatic heterocyclylcarbonyloxy which may be substituted with substituent group γ, non-aromatic heterocyclylcarbonyloxy which may be substituted with substituent group γ′, aromatic carbocyclylcarbonyl which may be substituted with substituent group γ, non-aromatic carbocyclylcarbonyl which may be substituted with substituent group γ′, aromatic heterocyclylcarbonyl which may be substituted with substituent group γ, non-aromatic heterocyclylcarbonyl which may be substituted with substituent group γ′, aromatic carbocyclyloxycarbonyl which may be substituted with substituent group γ, non-aromatic carbocyclyloxycarbonyl which may be substituted with substituent group γ′, aromatic heterocyclyloxycarbonyl which may be substituted with substituent group γ, non-aromatic heterocyclyloxycarbonyl which may be substituted with substituent group γ′, aromatic carbocyclylalkyloxy which may be substituted with substituent group γ, non-aromatic carbocyclylalkyloxy which may be substituted with substituent group γ′, aromatic heterocyclylalkyloxy which may be substituted with substituent group γ, non-aromatic heterocyclylalkyloxy which may be substituted with substituent group γ′, aromatic carbocyclylalkyloxycarbonyl which may be substituted with substituent group γ, non-aromatic carbocyclylalkyloxycarbonyl which may be substituted with substituent group γ′, aromatic heterocyclylalkyloxycarbonyl which may be substituted with substituent group γ, non-aromatic heterocyclylalkyloxycarbonyl which may be substituted with substituent group γ′, aromatic carbocyclylsulfanyl which may be substituted with substituent group γ, non-aromatic carbocyclylsulfanyl which may be substituted with substituent group γ′, aromatic heterocyclylsulfanyl which may be substituted with substituent group γ, non-aromatic heterocyclylsulfanyl which may be substituted with substituent group γ′, aromatic carbocyclylsulfinyl which may be substituted with substituent group γ, non-aromatic carbocyclylsulfinyl which may be substituted with substituent group γ′, aromatic heterocyclylsulfinyl which may be substituted with substituent group γ, non-aromatic heterocyclylsulfinyl which may be substituted with substituent group γ′, aromatic carbocyclylsulfonyl which may be substituted with substituent group γ, non-aromatic carbocyclylsulfonyl which may be substituted with substituent group γ′, aromatic heterocyclylsulfonyl which may be substituted with substituent group γ, and non-aromatic heterocyclylsulfonyl which may be substituted with substituent group γ′.

Substituent group α: halogen, hydroxy, carboxy, alkyloxy, haloalkyloxy, alkenyloxy, alkynyloxy, sulfanyl, and cyano.

Substituent group β: halogen, hydroxy, carboxy, cyano, alkyl which may be substituted with substituent group α, alkenyl which may be substituted with substituent group α, alkynyl which may be substituted with substituent group α, alkylcarbonyl which may be substituted with substituent group α, alkenylcarbonyl which may be substituted with substituent group α, alkynylcarbonyl which may be substituted with substituent group α, alkylsulfanyl which may be substituted with substituent group α, alkenylsulfanyl which may be substituted with substituent group α, alkynylsulfanyl which may be substituted with substituent group α, alkylsulfinyl which may be substituted with substituent group α, alkenylsulfinyl which may be substituted with substituent group α, alkynylsulfinyl which may be substituted with substituent group α, alkylsulfonyl which may be substituted with substituent group α, alkenylsulfonyl which may be substituted with substituent group α, alkynylsulfonyl which may be substituted with substituent group α, aromatic carbocyclyl which may be substituted with substituent group γ, non-aromatic carbocyclyl which may be substituted with substituent group γ′, aromatic heterocyclyl which may be substituted with substituent group γ, non-aromatic heterocyclyl which may be substituted with substituent group γ′, aromatic carbocyclylalkyl which may be substituted with substituent group γ, non-aromatic carbocyclylalkyl which may be substituted with substituent group γ′, aromatic heterocyclylalkyl which may be substituted with substituent group γ, non-aromatic heterocyclylalkyl which may be substituted with substituent group γ′, aromatic carbocyclylcarbonyl which may be substituted with substituent group γ, non-aromatic carbocyclylcarbonyl which may be substituted with substituent group γ′, aromatic heterocyclylcarbonyl which may be substituted with substituent group γ, non-aromatic heterocyclylcarbonyl which may be substituted with substituent group γ′, aromatic carbocyclyloxycarbonyl which may be substituted with substituent group γ, non-aromatic carbocyclyloxycarbonyl which may be substituted with substituent group γ′, aromatic heterocyclyloxycarbonyl which may be substituted with substituent group γ, non-aromatic heterocyclyloxycarbonyl which may be substituted with substituent group γ′, aromatic carbocyclylsulfanyl which may be substituted with substituent group γ, non-aromatic carbocyclylsulfanyl which may be substituted with substituent group γ′, aromatic heterocyclylsulfanyl which may be substituted with substituent group γ, non-aromatic heterocyclylsulfanyl which may be substituted with substituent group γ′, aromatic carbocyclylsulfinyl which may be substituted with substituent group γ, non-aromatic carbocyclylsulfinyl which may be substituted with substituent group γ′, aromatic heterocyclylsulfinyl which may be substituted with substituent group γ, non-aromatic heterocyclylsulfinyl which may be substituted with substituent group γ′, aromatic carbocyclylsulfonyl which may be substituted with substituent group γ, non-aromatic carbocyclylsulfonyl which may be substituted with substituent group γ′, aromatic heterocyclylsulfonyl which may be substituted with substituent group γ, and non-aromatic heterocyclylsulfonyl which may be substituted with substituent group γ′.

Substituent group γ: substituent group α, alkyl, haloalkyl, hydroxyalkyl, alkenyl, alkynyl, alkylcarbonyl, haloalkylcarbonyl, alkenylcarbonyl, and alkynylcarbonyl.

Substituent group γ′: substituent group γ and oxo.

The substituents on the rings of “aromatic carbocycle” and “aromatic heterocycle”, such as “substituted aromatic carbocyclyl”, “substituted aromatic heterocyclyl”, “substituted aromatic nitrogen-containing heterocyclyl”, “substituted aromatic carbocyclyloxy”, “substituted aromatic heterocyclyloxy”, “substituted aromatic carbocyclylcarbonyloxy”, “substituted aromatic heterocyclylcarbonyloxy”, “substituted aromatic carbocyclylcarbonyl”, “substituted aromatic heterocyclylcarbonyl”, “substituted aromatic carbocyclyloxycarbonyl”, “substituted aromatic heterocyclyloxycarbonyl”, “substituted aromatic carbocyclylsulfanyl”, “substituted aromatic heterocyclylsulfanyl”, “substituted aromatic carbocyclylsulfinyl”, “substituted aromatic heterocyclylsulfinyl”, “substituted aromatic carbocyclylsulfonyl”, and “substituted aromatic heterocyclylsulfonyl” include the following substituent group B. An atom at any position on the ring may be bonded to one or more group(s) selected from the following substituent group B.

Substituent group B: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureide, amidino, guanidino, pentafluorothio, trialkylsilyl, alkyl which may be substituted with substituent group α, alkenyl which may be substituted with substituent group α, alkynyl which may be substituted with substituent group α, alkyloxy which may be substituted with substituent group α, alkenyloxy which may be substituted with substituent group α, alkynyloxy which may be substituted with substituent group α, alkylcarbonyloxy which may be substituted with substituent group α, alkenylcarbonyloxy which may be substituted with substituent group α, alkynylcarbonyloxy which may be substituted with substituent group α, alkylcarbonyl which may be substituted with substituent group α, alkenylcarbonyl which may be substituted with substituent group α, alkynylcarbonyl which may be substituted with substituent group α, alkyloxycarbonyl which may be substituted with substituent group α, alkenyloxycarbonyl which may be substituted with substituent group α, alkynyloxycarbonyl which may be substituted with substituent group α, alkylsulfanyl which may be substituted with substituent group α, alkenylsulfanyl which may be substituted with substituent group α, alkynylsulfanyl which may be substituted with substituent group α, alkylsulfinyl which may be substituted with substituent group α, alkenylsulfinyl which may be substituted with substituent group α, alkynylsulfinyl which may be substituted with substituent group α, alkylsulfonyl which may be substituted with substituent group α, alkenylsulfonyl which may be substituted with substituent group α, alkynylsulfonyl which may be substituted with substituent group α, amino which may be substituted with substituent group B, imino which may be substituted with substituent group β, carbamoyl which may be substituted with substituent group B, sulfamoyl which may be substituted with substituent group β, aromatic carbocyclyl which may be substituted with substituent group γ, non-aromatic carbocyclyl which may be substituted with substituent group γ′, aromatic heterocyclyl which may be substituted with substituent group γ, non-aromatic heterocyclyl which may be substituted with substituent group γ′, aromatic carbocyclyloxy which may be substituted with substituent group γ, non-aromatic carbocyclyloxy which may be substituted with substituent group γ′, aromatic heterocyclyloxy which may be substituted with substituent group γ, non-aromatic heterocyclyloxy which may be substituted with substituent group γ′, aromatic carbocyclylcarbonyloxy which may be substituted with substituent group γ, non-aromatic carbocyclylcarbonyloxy which may be substituted with substituent group γ′, aromatic heterocyclylcarbonyloxy which may be substituted with substituent group γ, and non-aromatic heterocyclylcarbonyloxy which may be substituted with substituent group γ′, aromatic carbocyclylcarbonyl which may be substituted with substituent group γ, non-aromatic carbocyclylcarbonyl which may be substituted with substituent group γ′, aromatic heterocyclylcarbonyl which may be substituted with substituent group γ, non-aromatic heterocyclylcarbonyl which may be substituted with substituent group γ′, aromatic carbocyclyloxycarbonyl which may be substituted with substituent group γ, non-aromatic carbocyclyloxycarbonyl which may be substituted with substituent group γ′, aromatic heterocyclyloxycarbonyl which may be substituted with substituent group γ, non-aromatic heterocyclyloxycarbonyl which may be substituted with substituent group γ′, aromatic carbocyclylalkyl which may be substituted with substituent group γ, non-aromatic carbocyclylalkyl which may be substituted with substituent group γ′, aromatic heterocyclylalkyl which may be substituted with substituent group γ, non-aromatic heterocyclylalkyl which may be substituted with substituent group γ′, aromatic carbocyclylalkyloxy which may be substituted with substituent group γ, non-aromatic carbocyclylalkyloxy which may be substituted with substituent group γ′, aromatic heterocyclylalkyloxy which may be substituted with substituent group γ, non-aromatic heterocyclylalkyloxy which may be substituted with substituent group γ′, aromatic carbocyclylalkyloxycarbonyl which may be substituted with substituent group γ, non-aromatic carbocyclylalkyloxycarbonyl which may be substituted with substituent group γ′, aromatic heterocyclylalkyloxycarbonyl which may be substituted with substituent group γ, non-aromatic heterocyclylalkyloxycarbonyl which may be substituted with substituent group γ′, aromatic carbocyclylalkyloxyalkyl which may be substituted with substituent group γ, non-aromatic carbocyclylalkyloxyalkyl which may be substituted with substituent group γ′, aromatic heterocyclylalkyloxyalkyl which may be substituted with substituent group γ, non-aromatic heterocyclylalkyloxyalkyl which may be substituted with substituent group γ′, aromatic carbocyclylsulfanyl which may be substituted with substituent group γ, non-aromatic carbocyclylsulfanyl which may be substituted with substituent group γ′, aromatic heterocyclylsulfanyl which may be substituted with substituent group γ, non-aromatic heterocyclylsulfanyl which may be substituted with substituent group γ′, aromatic carbocyclylsulfinyl which may be substituted with substituent group γ, non-aromatic carbocyclylsulfinyl which may be substituted with substituent group γ′, aromatic heterocyclylsulfinyl which may be substituted with substituent group γ, non-aromatic heterocyclylsulfinyl which may be substituted with substituent group γ′, aromatic carbocyclylsulfonyl which may be substituted with substituent group γ, non-aromatic carbocyclylsulfonyl which may be substituted with substituent group γ′, aromatic heterocyclylsulfonyl which may be substituted with substituent group γ, and non-aromatic heterocyclylsulfonyl which may be substituted with substituent group γ′.

The substituents on the ring of “non-aromatic carbocycle” and “non-aromatic heterocycle” of “substituted non-aromatic carbocyclyl”, “substituted non-aromatic heterocyclyl”, “substituted non-aromatic nitrogen-containing heterocyclyl”, “substituted non-aromatic carbocycle that R² and R^{2′} are taken together with the carbon atom to which they are bonded to form”, “substituted non-aromatic heterocycle that R² and R^{2′} are taken together with the carbon atom to which they are bonded to form”, “substituted non-aromatic carbocycle that R³ and R^{3′} are taken together with the carbon atom to which they are bonded to form”, “substituted non-aromatic heterocycle that R³ and R^{3′} are taken together with the carbon atom to which they are bonded to form”, “substituted non-aromatic carbocycle that R³² and R³³ are taken together to form”, “substituted non-aromatic heterocycle that R³² and R³³ are taken together to form”, “substituted non-aromatic carbocycle that R³⁴ and R³⁵ are taken together with the carbon atom to which they are bonded to form”, “substituted non-aromatic heterocycle that R³⁴ and R³⁵ are taken together with the carbon atom to which they are bonded to form”, “substituted non-aromatic carbocyclyloxy”, “substituted non-aromatic heterocyclyloxy”, “substituted non-aromatic carbocyclylcarbonyloxy”, “substituted non-aromatic heterocyclylcarbonyloxy”, “substituted non-aromatic carbocyclylcarbonyl”, “substituted non-aromatic heterocyclylcarbonyl”, “substituted non-aromatic carbocyclyloxycarbonyl”, “substituted non-aromatic heterocyclyloxycarbonyl”, “substituted non-aromatic carbocyclylsulfanyl”, “substituted non-aromatic heterocyclylsulfanyl”, “substituted non-aromatic carbocyclylsulfinyl”, “substituted non-aromatic heterocyclylsulfinyl”, “substituted non-aromatic carbocyclylsulfonyl”, and “substituted non-aromatic heterocyclylsulfonyl” include the following substituent group C. An atom at any position on the ring may be bonded to one or more group(s) selected from the following substituent group C.

Substituent group C: substituent group B and oxo.

When the “non-aromatic carbocycle”, the “non-aromatic heterocycle” and the “non-aromatic nitrogen-containing heterocycle” are substituted with “oxo”, this means a ring in which two hydrogen atoms on a carbon atom are substituted as follows.

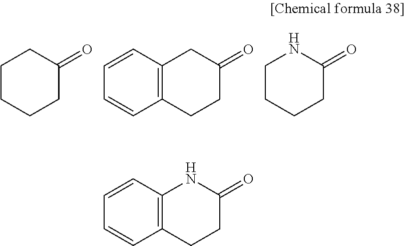

The substituents for “substituted amino”, “substituted imino”, “substituted carbamoyl”, and “substituted sulfamoyl” include the following substituent group D. These moieties may be substituted with one or two group(s) selected from substituent group D.

Substituent group D: halogen, hydroxy, carboxy, cyano, alkyl which may be substituted with substituent group α, alkenyl which may be substituted with substituent group α, alkynyl which may be substituted with substituent group α, alkylcarbonyl which may be substituted with substituent group α, alkenylcarbonyl which may be substituted with substituent group α, alkynylcarbonyl which may be substituted with substituent group α, alkylsulfanyl which may be substituted with substituent group α, alkenylsulfanyl which may be substituted with substituent group α, alkynylsulfanyl which may be substituted with substituent group α, alkylsulfinyl which may be substituted with substituent group α, alkenylsulfinyl which may be substituted with substituent group α, alkynylsulfinyl which may be substituted with substituent group α, alkylsulfonyl which may be substituted with substituent group α, alkenylsulfonyl which may be substituted with substituent group α, alkynylsulfonyl which may be substituted with substituent group α, amino which may be substituted with substituent group β, imino which may be substituted with substituent group β, carbamoyl which may be substituted with substituent group β, sulfamoyl which may be substituted with substituent group β, aromatic carbocyclyl which may be substituted with substituent group γ, non-aromatic carbocyclyl which may be substituted with substituent group γ′, aromatic heterocyclyl which may be substituted with substituent group γ, non-aromatic heterocyclyl which may be substituted with substituent group γ′, aromatic carbocyclylalkyl which may be substituted with substituent group γ, non-aromatic carbocyclylalkyl which may be substituted with substituent group γ′, aromatic heterocyclylalkyl which may be substituted with substituent group γ, non-aromatic heterocyclylalkyl which may be substituted with substituent group γ′, aromatic carbocyclylcarbonyl which may be substituted with substituent group γ, non-aromatic carbocyclylcarbonyl which may be substituted with substituent group γ′, aromatic heterocyclylcarbonyl which may be substituted with substituent group γ, non-aromatic heterocyclylcarbonyl which may be substituted with substituent group γ′, aromatic carbocyclyloxycarbonyl which may be substituted with substituent group γ, non-aromatic carbocyclyloxycarbonyl which may be substituted with substituent group γ′, aromatic heterocyclyloxycarbonyl which may be substituted with substituent group γ, non-aromatic heterocyclyloxycarbonyl which may be substituted with substituent group γ′, aromatic carbocyclylsulfanyl which may be substituted with substituent group γ, non-aromatic carbocyclylsulfanyl which may be substituted with substituent group γ′, aromatic heterocyclylsulfanyl which may be substituted with substituent group γ, non-aromatic heterocyclylsulfanyl which may be substituted with substituent group γ′, aromatic carbocyclylsulfinyl which may be substituted with substituent group γ, non-aromatic carbocyclylsulfinyl which may be substituted with substituent group γ′, aromatic heterocyclylsulfinyl which may be substituted with substituent group γ, non-aromatic heterocyclylsulfinyl which may be substituted with substituent group γ′, aromatic carbocyclylsulfonyl which may be substituted with substituent group γ, non-aromatic carbocyclylsulfonyl which may be substituted with substituent group γ′, aromatic heterocyclylsulfonyl which may be substituted with substituent group γ, and non-aromatic heterocyclylsulfonyl which may be substituted with substituent group γ′.

Preferred embodiments of R¹, A¹, A², m, n, and ring B in the compound represented by Formula (I) are described below. Regarding the compound represented by Formula (I), embodiments of all the combinations of specific examples shown below are mentioned as examples.

R¹ may be a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as A-1).

R¹ may be a hydrogen atom or substituted or unsubstituted alkyl (hereinafter, referred to as A-2).

R¹ may be substituted or unsubstituted alkyl (hereinafter, referred to as A-3).

R¹ may be a hydrogen atom or alkyl (hereinafter, referred to as A-4).

R¹ may be alkyl (hereinafter, referred to as A-5).

A¹ may be CR²R^{2′} wherein R² is each independently a hydrogen atom, halogen, substituted or unsubstituted alkyl, or substituted or unsubstituted alkyloxy; R^{2′} is each independently a hydrogen atom, halogen, substituted or unsubstituted alkyl, or substituted or unsubstituted alkyloxy; R² and R^{2′} may be taken together with the identical carbon atom to which they are bonded to form a substituted or unsubstituted non-aromatic carbocycle or a substituted or unsubstituted non-aromatic heterocycle (hereinafter, referred to as B-1).

A¹ may be CR²R^{2′} wherein R² is each independently a hydrogen atom, halogen, or substituted or unsubstituted alkyl; R^{2′} is each independently a hydrogen atom, halogen, or substituted or unsubstituted alkyl; R² and R^{2′} may be taken together with the identical carbon atom to which they are bonded to form a substituted or unsubstituted non-aromatic carbocycle or a substituted or unsubstituted non-aromatic heterocycle (hereinafter, referred to as B-2).

A¹ may be CR²R^{2′} wherein R² is each independently a hydrogen atom or halogen; R^{2′} is each independently a hydrogen atom or halogen; R² and R^{2′} may be taken together with the identical carbon atom to which they are bonded to form a substituted or unsubstituted non-aromatic carbocycle or a substituted or unsubstituted non-aromatic heterocycle (hereinafter, referred to as B-3).

A¹ may be CR²R^{2′} wherein R² is each independently a hydrogen atom or halogen; R^{2′} is each independently a hydrogen atom or halogen; R² and R^{2′} may be taken together with the identical carbon atom to which they are bonded to form a substituted or unsubstituted non-aromatic carbocycle (hereinafter, referred to as B-4).

A¹ may be CR²R^{2′} wherein R² is a hydrogen atom; R^{2′} is a hydrogen atom; R² and R^{2′} may be taken together with the identical carbon atom to which they are bonded to form a substituted or unsubstituted non-aromatic carbocycle (hereinafter, referred to as B-5).

A¹ may be CR²R^{2′} wherein R² is each independently a hydrogen atom or halogen; R^{2′} is each independently a hydrogen atom or halogen (hereinafter, referred to as B-6).

A¹ may be CR²R^{2′} wherein R² is a hydrogen atom; R^{2′} is a hydrogen atom (hereinafter, referred to as B-7).

A² may be CR³R^{3′} wherein R³ is each independently a hydrogen atom, halogen, substituted or unsubstituted alkyl, or substituted or unsubstituted alkyloxy; R^{3′} is each independently a hydrogen atom, halogen, substituted or unsubstituted alkyl, or substituted or unsubstituted alkyloxy; R³ and R^{3′} may be taken together with the identical carbon atom to which they are bonded to form a substituted or unsubstituted non-aromatic carbocycle or a substituted or unsubstituted non-aromatic heterocycle (hereinafter, referred to as C-1).

A² may be CR³R^{3′} wherein R³ is each independently a hydrogen atom, halogen, or substituted or unsubstituted alkyl; R^{3′} is each independently a hydrogen atom, halogen, or substituted or unsubstituted alkyl; R³ and R^{3′} may be taken together with the identical carbon atom to which they are bonded to form a substituted or unsubstituted non-aromatic carbocycle or a substituted or unsubstituted non-aromatic heterocycle (hereinafter, referred to as C-2).

A² may be CR³R^{3′} wherein R³ is each independently a hydrogen atom or halogen; R^{3′} is each independently a hydrogen atom or halogen; R³ and R^{3′} may be taken together with the identical carbon atom to which they are bonded to form a substituted or unsubstituted non-aromatic carbocycle or a substituted or unsubstituted non-aromatic heterocycle (hereinafter, referred to as C-3).

A² may be CR³R^{3′} wherein R³ is each independently a hydrogen atom or halogen; R^{3′} is each independently a hydrogen atom or halogen; R³ and R^{3′} may be taken together with the identical carbon atom to which they are bonded to form a substituted or unsubstituted non-aromatic carbocycle (hereinafter, referred to as C-4).

A² may be CR³R^{3′} wherein R³ is a hydrogen atom; R^{3′} is a hydrogen atom; R³ and R^{3′} may be taken together with the identical carbon atom to which they are bonded to form a substituted or unsubstituted non-aromatic carbocycle (hereinafter, referred to as C-5).

A² may be CR³R^{3′} wherein R³ is each independently a hydrogen atom or halogen; R^{3′} is each independently a hydrogen atom or halogen (hereinafter, referred to as C-6).

A² may be CR³R^{3′} wherein R³ is a hydrogen atom; R^{3′} is a hydrogen atom (hereinafter, referred to as C-7).

m may be 1, 2, or 3 (hereinafter, referred to as D-1).

m may be 1 or 2 (hereinafter, referred to as D-2).

m may be 1 (hereinafter, referred to as D-3).

m may be 2 (hereinafter, referred to as D-4).

m may be 3 (hereinafter, referred to as D-5).

n may be 1, 2, or 3 (hereinafter, referred to as E-1).

n may be 1 or 2 (hereinafter, referred to as E-2).

n may be 1 (hereinafter, referred to as E-3).

n may be 2 (hereinafter, referred to as E-4).

n may be 3 (hereinafter, referred to as E-5).

Ring B may be a ring represented by the following group (hereinafter, referred to as F-1).

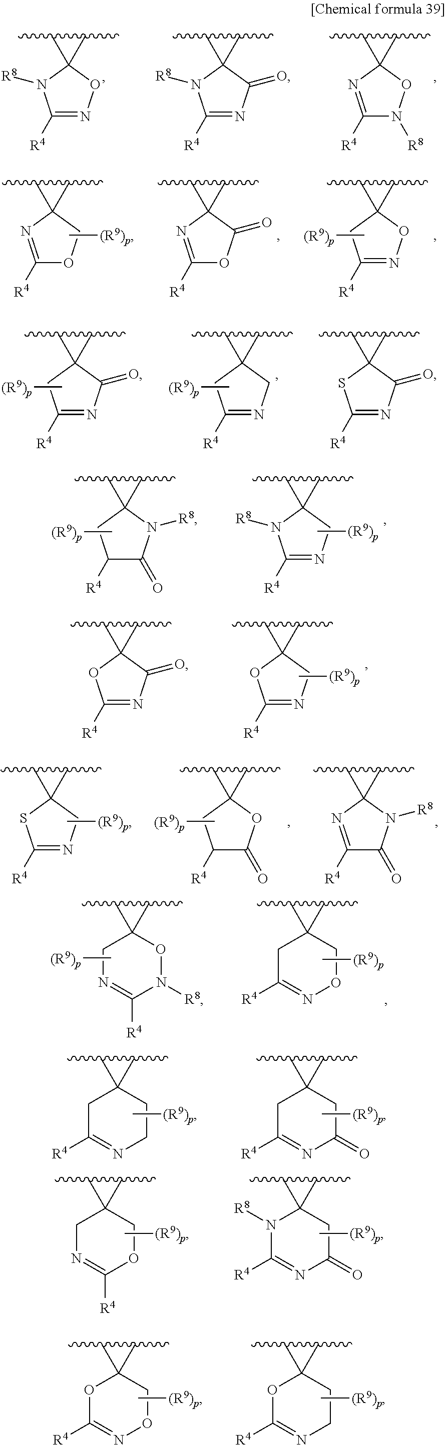

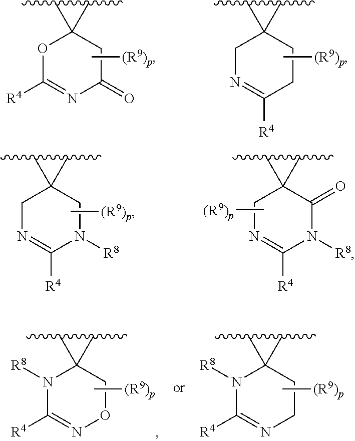

Ring B may be a ring represented by the following group (hereinafter, referred to as F-2).

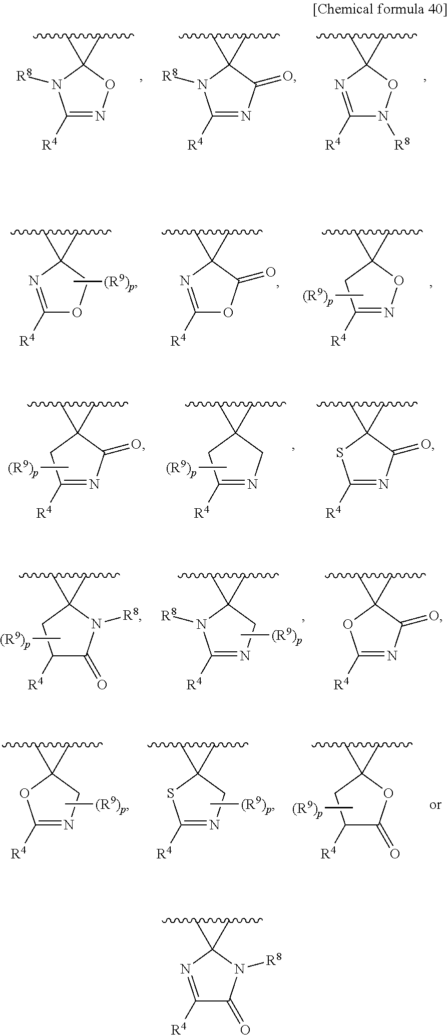

Ring B may be a ring represented by the following group (hereinafter, referred to as F-3).

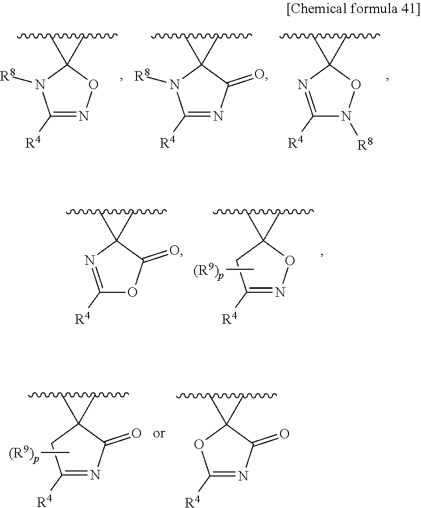

Ring B may be a ring represented by the following group (hereinafter, referred to as F-4).

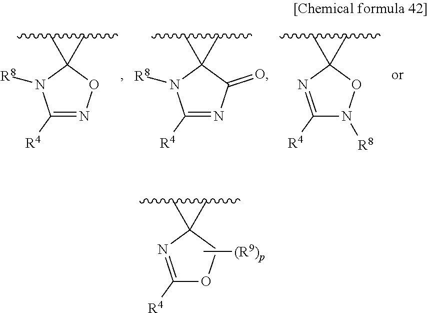

Ring B may be a ring represented by the following group (hereinafter, referred to as F-5).

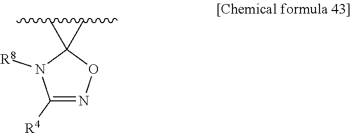

Ring B may be a ring represented by the following group (hereinafter, referred to as F-6).

[Chemical Formula 44] [symbol]
Ring B may be a ring represented by the following group (hereinafter, referred to as F-7).

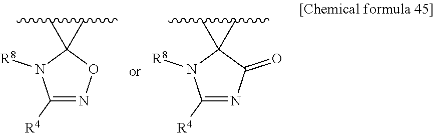

R⁴ may be the following group (hereinafter, referred to as G-1).

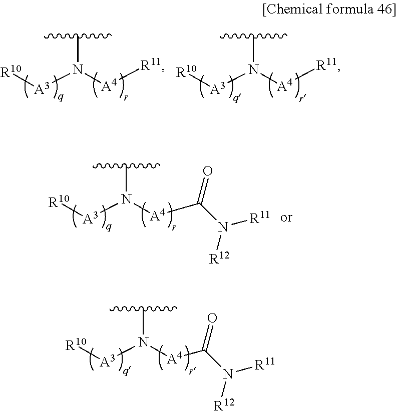

R⁴ may be the following group (hereinafter, referred to as G-2).

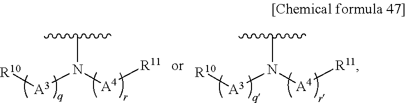

R⁴ may be the following group (hereinafter, referred to as G-3).

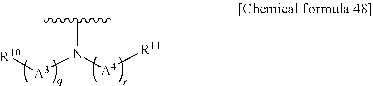

R⁴ may be the following group (hereinafter, referred to as G-4).

[Chemical Formula 49]

[symbol]

A³ may be CR¹³R^{13′} wherein R¹³ is each independently a hydrogen atom, substituted or unsubstituted alkyl, or substituted or unsubstituted alkyloxy; R^{13′} is each independently a hydrogen atom, substituted or unsubstituted alkyl, or substituted or unsubstituted alkyloxy (hereinafter, referred to as H-1).

A³ may be CR¹³R^{13′} wherein R¹³ is each independently a hydrogen atom or substituted or unsubstituted alkyl; R^{13′} is each independently a hydrogen atom or substituted or unsubstituted alkyl (hereinafter, referred to as H-2).

A³ may be CR¹³R^{13′} wherein R¹³ is a hydrogen atom; R^{13′} is a hydrogen atom (hereinafter, referred to as H-3).

A⁴ may be CR¹⁴R^{14′} wherein R¹⁴ is each independently a hydrogen atom, substituted or unsubstituted alkyl, or substituted or unsubstituted alkyloxy; R^{14′} is each independently a hydrogen atom, substituted or unsubstituted alkyl, or substituted or unsubstituted alkyloxy (hereinafter, referred to as I-1).

A⁴ may be CR¹⁴R^{14′} wherein R¹⁴ is each independently a hydrogen atom or substituted or unsubstituted alkyl; R^{14′} is each independently a hydrogen atom or substituted or unsubstituted alkyl (hereinafter, referred to as I-2).

A⁴ may be CR¹⁴R^{14′} wherein R¹⁴ is a hydrogen atom; R^{14′} is a hydrogen atom (hereinafter, referred to as I-3).

q may be 0, 1, or 2 (hereinafter, referred to as J-1).

q may be 1 or 2 (hereinafter, referred to as J-2).

q may be 1 (hereinafter, referred to as J-3).

q may be 2 (hereinafter, referred to as J-4).

q′ may be 1 or 2 (hereinafter, referred to as K-1).

q′ may be 1 (hereinafter, referred to as K-2).

q′ may be 2 (hereinafter, referred to as K-3).

r may be 0, 1, or 2 (hereinafter, referred to as L-1).

r may be 1 or 2 (hereinafter, referred to as L-2).

r may be 1 (hereinafter, referred to as L-3).

r may be 2 (hereinafter, referred to as L-4).

r′ may be 1 or 2 (hereinafter, referred to as M-1).

r′ may be 1 (hereinafter, referred to as M-2).

r′ may be 2 (hereinafter, referred to as M-3).

R¹⁰ may be substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as O-1).

R¹⁰ may be substituted or unsubstituted aromatic carbocyclyl, or substituted or unsubstituted aromatic heterocyclyl (hereinafter, referred to as O-2).

R¹⁰ may be substituted or unsubstituted aromatic heterocyclyl (hereinafter, referred to as O-3).

R¹⁰ may be substituted or unsubstituted 5-membered aromatic heterocyclyl (hereinafter, referred to as O-4).

R¹⁰ may be substituted or unsubstituted oxazolyl (hereinafter, referred to as O-5).

R¹⁰ may be substituted or unsubstituted pyrazolyl (hereinafter, referred to as O-6).

R¹⁰ may be substituted or unsubstituted isoxazolyl (hereinafter, referred to as O-7).

R¹⁰ may be substituted or unsubstituted furyl (hereinafter, referred to as O-8).

R¹⁰ may be substituted or unsubstituted triazolyl (hereinafter, referred to as O-9).

R¹⁰ may be phenyl substituted with halogen, phenyl, 5-membered aromatic heterocyclyl substituted with one or more substituents selected from substituent group ω (substituent group ω: alkyl, haloalkyl, and non-aromatic carbocyclyl), or 6-membered aromatic heterocyclyl substituted with one or more substituents selected from substituent group ω′ (substituent group ω′: alkyl and halogen) (hereinafter, referred to as O-10).

R¹⁰ may be phenyl substituted with halogen, or unsubstituted phenyl (hereinafter, referred to as O-11).

R¹⁰ may be 5-membered aromatic heterocyclyl substituted with one or more substituents selected from substituent group ω (substituent group ω: alkyl, haloalkyl, and non-aromatic carbocyclyl), or 6-membered aromatic heterocyclyl substituted with one or more substituents selected from substituent group ω′ (substituent group ω′: alkyl and halogen) (hereinafter, referred to as O-12).

R¹⁰ may be 5-membered aromatic heterocyclyl substituted with one or more substituents selected from substituent group ω (substituent group ω: alkyl, haloalkyl, and non-aromatic carbocyclyl) (hereinafter, referred to as O-13).

R¹⁰ may be oxazolyl substituted with one or more substituents selected from substituent group ω or triazolyl substituted with one or more substituents selected from substituent group co (substituent group ω: alkyl, haloalkyl, and non-aromatic carbocyclyl) (hereinafter, referred to as O-14).

R¹¹ may be substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as P-1).

R¹¹ may be substituted or unsubstituted aromatic carbocyclyl, or substituted or unsubstituted aromatic heterocyclyl (hereinafter, referred to as P-2).

R¹¹ may be substituted or unsubstituted aromatic carbocyclyl (hereinafter, referred to as P-3).

R¹¹ may be substituted or unsubstituted phenyl (hereinafter, referred to as P-4).

R¹¹ may be phenyl substituted with substituent group ψ′ (substituent group ψ′: alkyl, halogen, haloalkyl, alkyl substituted with aromatic carbocyclyl, alkyloxy, alkyloxy substituted with non-aromatic carbocyclyl, alkyloxy substituted with non-aromatic carbocyclyl substituted with halogen, and haloalkyloxy), phenyl, 9-membered aromatic heterocyclyl, which is bicyclic, or 9-membered aromatic heterocyclyl, which is bicyclic, substituted with one or more substituents selected from substituent group ψ (substituent group ψ: halogen, alkyl, and alkyloxy) (hereinafter, referred to as P-5).

R¹¹ may be a group represented by Formula:

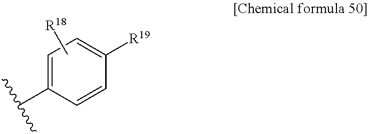

wherein R¹⁸ is a hydrogen atom or halogen;
R¹⁹ is alkyl, haloalkyl, alkyl substituted with aromatic carbocyclyl, alkyloxy, alkyloxy substituted with non-aromatic carbocyclyl, alkyloxy substituted with non-aromatic carbocyclyl substituted with halogen, or haloalkyloxy, 9-membered aromatic heterocyclyl, which is bicyclic, or 9-membered aromatic heterocyclyl, which is bicyclic, substituted with one or more substituents selected from substituent group ψ (substituent group ψ: halogen, alkyl, and alkyloxy) (hereinafter, referred to as P-6).

R¹¹ may be a group represented by Formula:

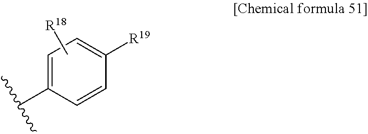

wherein R¹⁸ is a hydrogen atom or halogen;
R¹⁹ is C1-C6 alkyloxy or C1-C6 haloalkyloxy (hereinafter, referred to as P-7).

R¹² may be a hydrogen atom or substituted or unsubstituted alkyl (hereinafter, referred to as Q-1).

R¹² may be a hydrogen atom (hereinafter, referred to as Q-2).

R⁸ may be a hydrogen atom or substituted or unsubstituted alkyl (hereinafter, referred to as R-1).

R⁸ may be substituted or unsubstituted alkyl (hereinafter, referred to as R-2).

R⁸ may be a hydrogen atom (hereinafter, referred to as R-3).

R⁹ may be each independently halogen or substituted or unsubstituted alkyl (hereinafter, referred to as S-1).

R⁹ may be each independently substituted or unsubstituted alkyl (hereinafter, referred to as S-2).

R⁹ may be each independently halogen (hereinafter, referred to as S-3).

p may be an integer of any of 0 to 6 (hereinafter, referred to as T-1).

p may be 0, 1, or 2 (hereinafter, referred to as T-2).

p may be 1 (hereinafter, referred to as T-3).

p may be 0 (hereinafter, referred to as T-4).

Preferred embodiments of R¹, R², R³, R^{2′}, R^{3′}, and ring B in the compound represented by Formula (II) are described below. Regarding the compound represented by Formula (II), embodiments of all the combinations of specific examples shown below are mentioned as examples.

R¹ may be a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as AA-1).

R¹ may be a hydrogen atom or substituted or unsubstituted alkyl (hereinafter, referred to as AA-2).

R¹ may be substituted or unsubstituted alkyl (hereinafter, referred to as AA-3).

R¹ may be a hydrogen atom or alkyl (hereinafter, referred to as AA-4).

R¹ may be alkyl (hereinafter, referred to as AA-5).

R² may be a hydrogen atom, halogen, or substituted or unsubstituted alkyl (hereinafter, referred to as BA-1).

R² may be a hydrogen atom (hereinafter, referred to as BA-2).

R² may be halogen (hereinafter, referred to as BA-3).

R² may be substituted or unsubstituted alkyl (hereinafter, referred to as BA-4).

R² may be a hydrogen atom, halogen, or substituted or unsubstituted alkyl (hereinafter, referred to as CA-1).

R² may be a hydrogen atom (hereinafter, referred to as CA-2).

R² may be halogen (hereinafter, referred to as CA-3).

R² may be substituted or unsubstituted alkyl (hereinafter, referred to as CA-4).

R³ may be a hydrogen atom, halogen, or substituted or unsubstituted alkyl (hereinafter, referred to as DA-1).

R³ may be a hydrogen atom (hereinafter, referred to as DA-2).

R³ may be halogen (hereinafter, referred to as DA-3).

R³ may be substituted or unsubstituted alkyl (hereinafter, referred to as DA-4).

R^{3′} may be a hydrogen atom, halogen, or substituted or unsubstituted alkyl (hereinafter, referred to as EA-1).

R^{3′} may be a hydrogen atom (hereinafter, referred to as EA-2).

R^{3′} may be halogen (hereinafter, referred to as EA-3).

R^{3′} may be substituted or unsubstituted alkyl (hereinafter, referred to as EA-4).

Ring B may be a ring represented by the following group (hereinafter, referred to as FA-1).

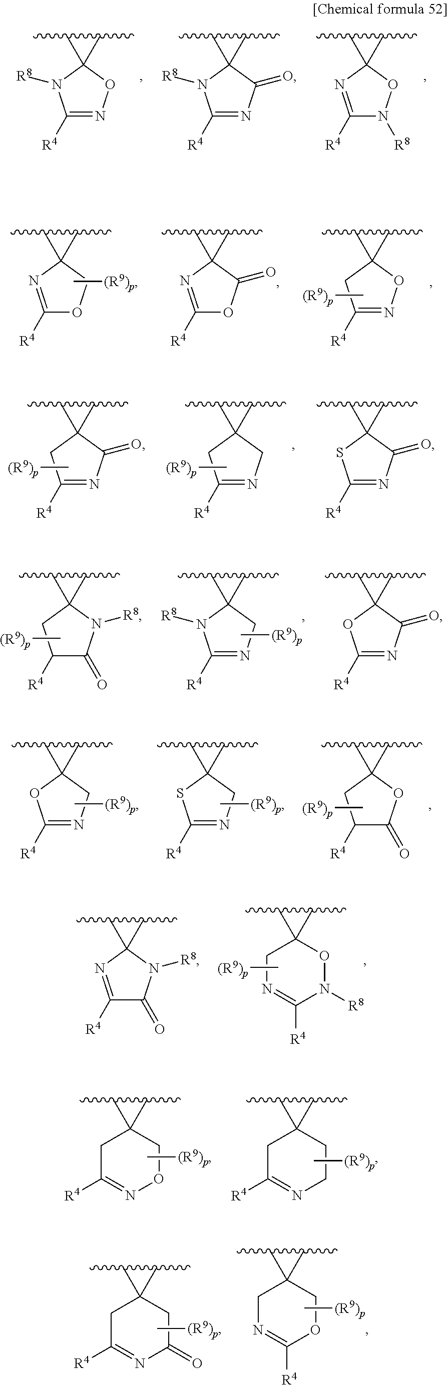

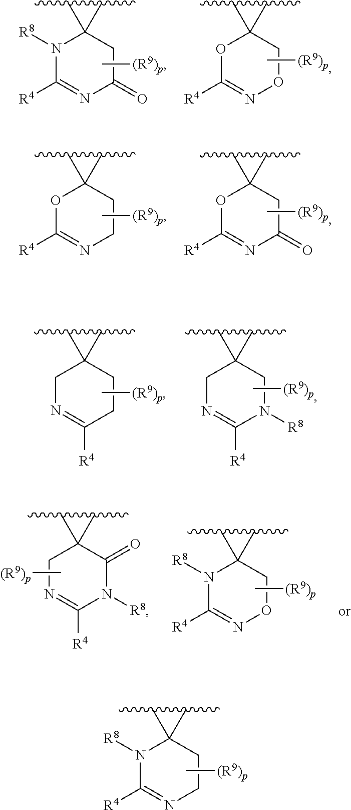

Ring B may be a ring represented by the following group (hereinafter, referred to as FA-2).

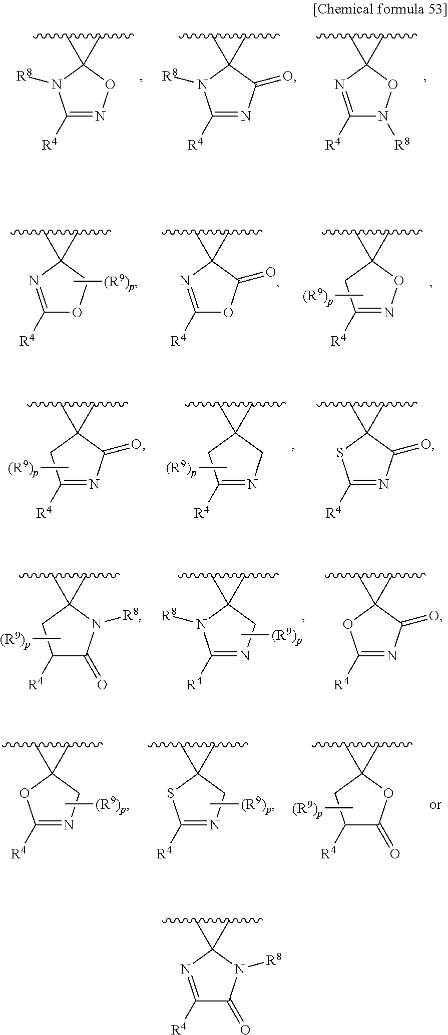

Ring B may be a ring represented by the following group (hereinafter, referred to as FA-3).

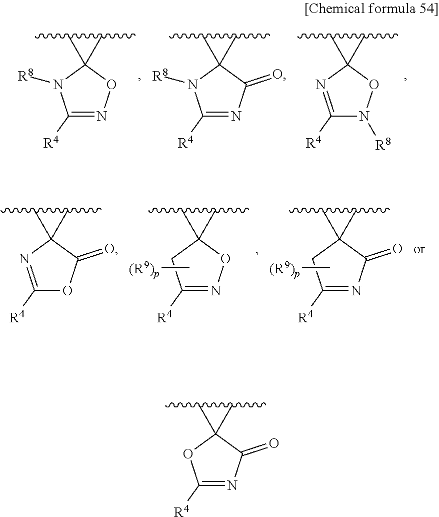

Ring B may be a ring represented by the following group (hereinafter, referred to as FA-4).

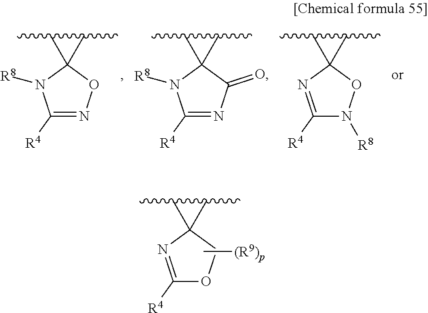

Ring B may be a ring represented by the following group (hereinafter, referred to as FA-5).

### [Chemical Formula 56]

Ring B may be a ring represented by the following group (hereinafter, referred to as FA-6).

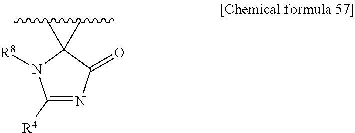

Ring B may be a ring represented by the following group (hereinafter, referred to as FA-7).

### [Chemical Formula 58]

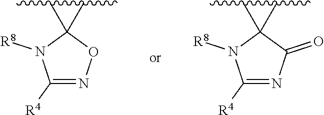

R⁴ may be the following group (hereinafter, referred to as GA-1)

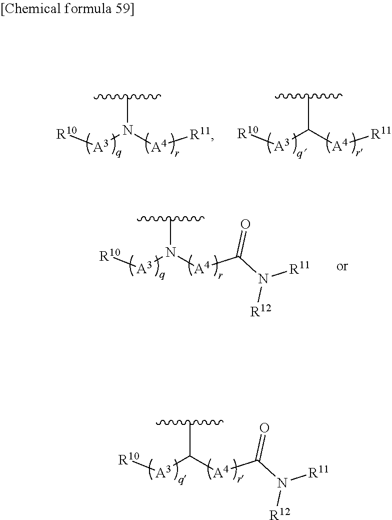

R⁴ may be the following group (hereinafter, referred to as GA-2).

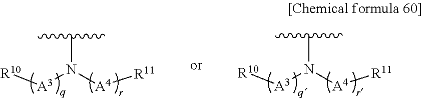

R⁴ may be the following group (hereinafter, referred to as GA-3).

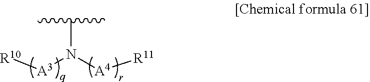

R⁴ may be the following group (hereinafter, referred to as GA-4).

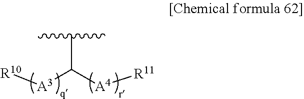

A³ may be CR¹³R^{13′} wherein R¹³ is each independently a hydrogen atom, substituted or unsubstituted alkyl, or substituted or unsubstituted alkyloxy; R^{13′} is each independently a hydrogen atom, substituted or unsubstituted alkyl, or substituted or unsubstituted alkyloxy (hereinafter, referred to as HA-1).

A³ may be CR¹³R^{13′} wherein R¹³ is each independently a hydrogen atom or substituted or unsubstituted alkyl; R^{13′} is each independently a hydrogen atom or substituted or unsubstituted alkyl (hereinafter, referred to as HA-2).

A³ may be CR¹³R^{13′} wherein R¹³ is a hydrogen atom; R^{13′} is a hydrogen atom (hereinafter, referred to as HA-3).

A⁴ may be CR¹⁴R^{14′} wherein R¹⁴ is each independently a hydrogen atom, substituted or unsubstituted alkyl, or substituted or unsubstituted alkyloxy; R^{14′} is each independently a hydrogen atom, substituted or unsubstituted alkyl, or substituted or unsubstituted alkyloxy (hereinafter, referred to as IA-1).

A⁴ may be CR¹⁴R^{14′} wherein R¹⁴ is each independently a hydrogen atom or substituted or unsubstituted alkyl; R^{14′} is each independently a hydrogen atom or substituted or unsubstituted alkyl (hereinafter, referred to as IA-2).

A⁴ may be CR¹⁴R^{14′} wherein R¹⁴ is a hydrogen atom; R^{14′} is a hydrogen atom (hereinafter, referred to as IA-3).

q may be 0, 1, or 2 (hereinafter, referred to as JA-1).

q may be 1 or 2 (hereinafter, referred to as JA-2).

q may be 1 (hereinafter, referred to as JA-3).

q may be 2 (hereinafter, referred to as JA-4).

q′ may be 1 or 2 (hereinafter, referred to as KA-1).

q′ may be 1 (hereinafter, referred to as KA-2).

q′ may be 2 (hereinafter, referred to as KA-3).

r may be 0, 1, or 2 (hereinafter, referred to as LA-1).

r may be 1 or 2 (hereinafter, referred to as LA-2).

r may be 1 (hereinafter, referred to as LA-3).

r may be 2 (hereinafter, referred to as LA-4).

r′ may be 1 or 2 (hereinafter, referred to as MA-1).

r′ may be 1 (hereinafter, referred to as MA-2).

r′ may be 2 (hereinafter, referred to as MA-3).

R¹⁰ may be substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as NA-1).

R¹⁰ may be substituted or unsubstituted aromatic carbocyclyl, or substituted or unsubstituted aromatic heterocyclyl (hereinafter, referred to as NA-2).

R¹⁰ may be substituted or unsubstituted aromatic heterocyclyl (hereinafter, referred to as NA-3).

R¹⁰ may be substituted or unsubstituted 5-membered aromatic heterocyclyl (hereinafter, referred to as NA-4).

R¹⁰ may be substituted or unsubstituted oxazolyl (hereinafter, referred to as NA-5).

R¹⁰ may be substituted or unsubstituted pyrazolyl (hereinafter, referred to as NA-6).

R¹⁰ may be substituted or unsubstituted isoxazolyl (hereinafter, referred to as NA-7).

R¹⁰ may be substituted or unsubstituted furyl (hereinafter, referred to as NA-8).

R¹⁰ may be substituted or unsubstituted triazolyl (hereinafter, referred to as NA-9).

R¹⁰ may be phenyl substituted with halogen, phenyl, 5-membered aromatic heterocyclyl substituted with one or more substituents selected from substituent group ω (substituent group ω: alkyl, haloalkyl, and non-aromatic carbocyclyl), or 6-membered aromatic heterocyclyl substituted with one or more substituents selected from substituent group ω′ (substituent group ω′: alkyl and halogen) (hereinafter, referred to as NA-10).

R¹⁰ may be phenyl substituted with halogen, or unsubstituted phenyl (hereinafter, referred to as NA-11).

R¹⁰ may be 5-membered aromatic heterocyclyl substituted with one or more substituents selected from substituent group ω (substituent group ω: alkyl, haloalkyl, and non-aromatic carbocyclyl), or 6-membered aromatic heterocyclyl substituted with one or more substituents selected from substituent group ω′ (substituent group ω′: alkyl and halogen) (hereinafter, referred to as NA-12).

R¹⁰ may be 5-membered aromatic heterocyclyl substituted with one or more substituents selected from substituent group ω (substituent group ω: alkyl, haloalkyl, and non-aromatic carbocyclyl) (hereinafter, referred to as NA-13).

R¹⁰ may be oxazolyl substituted with one or more substituents selected from substituent group ω or triazolyl substituted with one or more substituents selected from substituent group ω (substituent group ω: alkyl, haloalkyl, and non-aromatic carbocyclyl) (hereinafter, referred to as NA-14).

R¹¹ may be substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as OA-1).

R¹¹ may be substituted or unsubstituted aromatic carbocyclyl, or substituted or unsubstituted aromatic heterocyclyl (hereinafter, referred to as OA-2).

R¹¹ may be substituted or unsubstituted aromatic carbocyclyl (hereinafter, referred to as OA-3).

R¹¹ may be substituted or unsubstituted phenyl (hereinafter, referred to as OA-4).

R¹¹ may be phenyl substituted with substituent group ψ′ (substituent group ψ′: alkyl, halogen, haloalkyl, alkyl substituted with aromatic carbocyclyl, alkyloxy, alkyloxy substituted with non-aromatic carbocyclyl, alkyloxy substituted with non-aromatic carbocyclyl substituted with halogen, and haloalkyloxy), phenyl, 9-membered aromatic heterocyclyl, which is bicyclic, or 9-membered aromatic heterocyclyl, which is bicyclic, substituted with one or more substituents selected from substituent group ψ (substituent group ψ: halogen, alkyl, and alkyloxy) (hereinafter, referred to as OA-5).

R¹¹ may be a group represented by Formula:

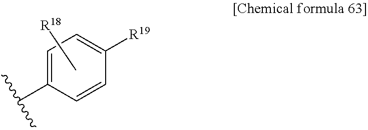

wherein R¹⁸ is a hydrogen atom or halogen;
R¹⁹ is alkyl, haloalkyl, alkyl substituted with aromatic carbocyclyl, alkyloxy, alkyloxy substituted with non-aromatic carbocyclyl, alkyloxy substituted with non-aromatic carbocyclyl substituted with halogen, or haloalkyloxy, 9-membered aromatic heterocyclyl, which is bicyclic, or 9-membered aromatic heterocyclyl, which is bicyclic, substituted with one or more substituents selected from substituent group ψ (substituent group ψ: halogen, alkyl, and alkyloxy) (hereinafter, referred to as OA-6).

R¹¹ may be a group represented by Formula:

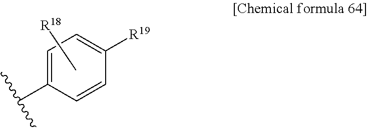

wherein R¹⁸ is a hydrogen atom or halogen;
R¹⁹ is C1-C6 alkyloxy or C1-C6 haloalkyloxy (hereinafter, referred to as OA-7).

R¹² may be a hydrogen atom or substituted or unsubstituted alkyl (hereinafter, referred to as PA-1).

R¹² may be a hydrogen atom (hereinafter, referred to as PA-2).

R⁸ may be a hydrogen atom or substituted or unsubstituted alkyl (hereinafter, referred to as QA-1).

R⁸ may be substituted or unsubstituted alkyl (hereinafter, referred to as QA-2).

R⁸ may be a hydrogen atom (hereinafter, referred to as QA-3).

R⁹ may be each independently halogen or substituted or unsubstituted alkyl (hereinafter, referred to as RA-1).

R⁹ may be each independently substituted or unsubstituted alkyl (hereinafter, referred to as RA-2).

R⁹ may be each independently halogen (hereinafter, referred to as RA-3).

p may be an integer of any of 0 to 6 (hereinafter, referred to as SA-1).

p may be 0, 1, or 2 (hereinafter, referred to as SA-2).

p may be 1 (hereinafter, referred to as SA-3).

p may be 0 (hereinafter, referred to as SA-4).

Preferred embodiments of R³¹, R³², R³³, R³⁴, R³⁵, and ring B′ in the compound represented by Formula (III) are described below. Regarding the compound represented by Formula (III), embodiments of all the combinations of specific examples shown below are mentioned as examples.

R³¹ may be a hydrogen atom or C1-C3 alkyl (hereinafter, referred to as AB-1).

R³¹ may be C1-C3 alkyl (hereinafter, referred to as AB-2).

R³² may be each independently a hydrogen atom or substituted or unsubstituted alkyl; R³³ may be each independently a hydrogen atom or substituted or unsubstituted alkyl; R³² and R³³ may be taken together with the identical carbon atom to which they are bonded to form a substituted or unsubstituted non-aromatic carbocycle or a substituted or unsubstituted non-aromatic heterocycle (hereinafter, referred to as BB-1).

R³² may be each independently a hydrogen atom or substituted or unsubstituted alkyl; R³³ may be each independently a hydrogen atom or substituted or unsubstituted alkyl; R³² and R³³ may be taken together with the identical carbon atom to which they are bonded to form a substituted or unsubstituted non-aromatic carbocycle (hereinafter, referred to as BB-2).

R³² may be a hydrogen atom; R³³ may be a hydrogen atom; R³² and R³³ may be taken together with the identical carbon atom to which they are bonded to form a substituted or unsubstituted non-aromatic carbocycle (hereinafter, referred to as BB-3).

R³² may be a hydrogen atom; R³³ may be a hydrogen atom; R³² and R³³ may be taken together with the identical carbon atom to which they are bonded to form a non-aromatic carbocycle (hereinafter, referred to as BB-4).

R³² may be a hydrogen atom; R³³ may be a hydrogen atom (hereinafter, referred to as BB-5).

R³⁴ may be each independently a hydrogen atom, halogen, or substituted or unsubstituted alkyl; R³⁵ may be each independently a hydrogen atom, halogen, or substituted or unsubstituted alkyl; R³⁴ and R³⁵ may be taken together with the identical carbon atom to which they are bonded to form a substituted or unsubstituted non-aromatic carbocycle or a substituted or unsubstituted non-aromatic heterocycle (hereinafter, referred to as CB-1).

R³⁴ may be each independently a hydrogen atom or halogen; R³⁵ may be each independently a hydrogen atom or halogen; R³⁴ and R³⁵ may be taken together with the identical carbon atom to which they are bonded to form a substituted or unsubstituted non-aromatic carbocycle or a substituted or unsubstituted non-aromatic heterocycle (hereinafter, referred to as CB-2).

R³⁴ may be each independently a hydrogen atom or halogen; R³⁵ may be each independently a hydrogen atom or halogen; R³⁴ and R³⁵ may be taken together with the identical carbon atom to which they are bonded to form a substituted or unsubstituted non-aromatic carbocycle (hereinafter, referred to as CB-3).

R³⁴ may be a hydrogen atom; R³⁵ may be a hydrogen atom; R³⁴ and R³⁵ may be taken together with the identical carbon atom to which they are bonded to form a substituted or unsubstituted non-aromatic carbocycle (hereinafter, referred to as CB-4).

R³⁴ may be a hydrogen atom; R³⁵ may be a hydrogen atom; R³⁴ and R³⁵ may be taken together with the identical carbon atom to which they are bonded to form a non-aromatic carbocycle (hereinafter, referred to as CB-5).

R³⁴ may be a hydrogen atom; R³⁵ may be a hydrogen atom (hereinafter, referred to as CB-6).

Ring B′ may be a ring represented by the following group (hereinafter, referred to as DB-1).

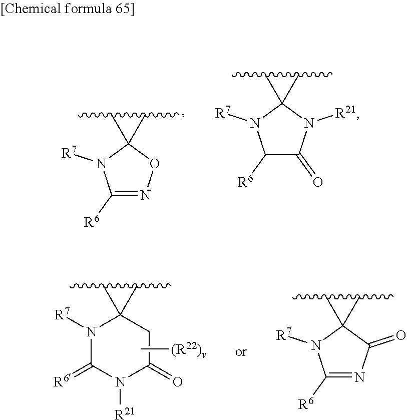

Ring B′ may be a ring represented by the following group (hereinafter, referred to as DB-2).

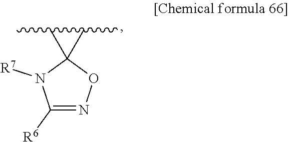

Ring B′ may be a ring represented by the following group (hereinafter, referred to as DB-3).

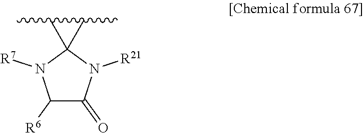

Ring B′ may be a ring represented by the following group (hereinafter, referred to as DB-4).

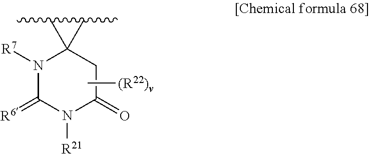

R⁶ may be the following group (hereinafter, referred to as EB-1).

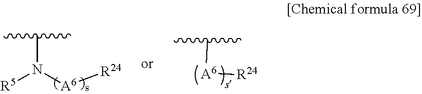

R⁶ may be the following group (hereinafter, referred to as EB-2).

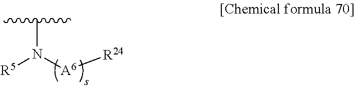

R⁶ may be the following group (hereinafter, referred to as EB-3).

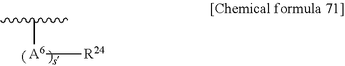

A⁶ may be CR²⁵R^{25′} wherein R²⁵ is each independently a hydrogen atom, halogen, substituted or unsubstituted alkyl, or substituted or unsubstituted alkyloxy; R^{25′} is each independently a hydrogen atom, halogen, substituted or unsubstituted alkyl, or substituted or unsubstituted alkyloxy (hereinafter, referred to as FB-1).

A⁶ may be CR²⁵R^{25′} wherein R²⁵ is each independently a hydrogen atom, halogen, or substituted or unsubstituted alkyl; R^{25′} is each independently a hydrogen atom, halogen, or substituted or unsubstituted alkyl (hereinafter, referred to as FB-2).

A⁶ may be CR²⁵R^{25′} wherein R²⁵ is each independently a hydrogen atom or substituted or unsubstituted alkyl; R^{25′} is each independently a hydrogen atom or substituted or unsubstituted alkyl (hereinafter, referred to as FB-3).

A⁶ may be CR²⁵R^{25′} wherein R²⁵ is a hydrogen atom; R^{25′} is a hydrogen atom (hereinafter, referred to as FB-4).

s may be 0 or 1 (hereinafter, referred to as GB-1).

s may be 0 (hereinafter, referred to as GB-2).

s may be 1 (hereinafter, referred to as GB-3).

s′ may be 0, 1, or 2 (hereinafter, referred to as HB-1).

s′ may be 1 (hereinafter, referred to as HB-2).

s′ may be 2 (hereinafter, referred to as HB-3).

R²⁴ may be substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as IB-1).

R²⁴ may be substituted or unsubstituted aromatic carbocyclyl, or substituted or unsubstituted aromatic heterocyclyl (hereinafter, referred to as IB-2).

R²⁴ may be substituted or unsubstituted aromatic carbocyclyl (hereinafter, referred to as IB-3).

R²⁴ may be substituted or unsubstituted phenyl (hereinafter, referred to as IB-4).

R²⁴ may be phenyl which is substituted with alkyl, halogen, haloalkyl, alkyloxy, non-aromatic carbocyclyloxy, or haloalkyloxy, or unsubstituted (hereinafter, referred to as IB-5).

R²⁴ may be phenyl which is substituted with alkyloxy, non-aromatic carbocyclyloxy, or haloalkyloxy, or unsubstituted (hereinafter, referred to as IB-6).

R⁵ may be a hydrogen atom or substituted or unsubstituted alkyl (hereinafter, referred to as JB-1).

R⁵ may be a hydrogen atom (hereinafter, referred to as JB-2).

R^{6′} may be a group represented by Formula:

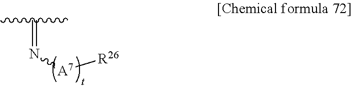

wherein A⁷ is R²⁷R^{27′} wherein R²⁷ is a hydrogen atom, halogen, substituted or unsubstituted alkyl, or substituted or unsubstituted alkyloxy; R^{27′} is a hydrogen atom, halogen, substituted or unsubstituted alkyl, or substituted or unsubstituted alkyloxy (hereinafter, referred to as KB-1).

R^{6′} may be a group represented by Formula:

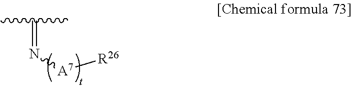

wherein A⁷ is CR²⁷R^{27′} wherein R²⁷ is a hydrogen atom or substituted or unsubstituted alkyl; R^{27′} is a hydrogen atom or substituted or unsubstituted alkyl (hereinafter, referred to as KB-2).

R^{6′} may be a group represented by Formula:

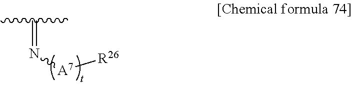

wherein A⁷ is CR²⁷R^{27′} wherein R²⁷ is a hydrogen atom; R^{27′} is a hydrogen atom (hereinafter, referred to as KB-3).

t may be 0 or 1 (hereinafter, referred to as LB-1).

t may be 1 (hereinafter, referred to as LB-2).

R²⁶ may be substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as MB-1).

R²⁶ may be substituted or unsubstituted aromatic carbocyclyl, or substituted or unsubstituted aromatic heterocyclyl (hereinafter, referred to as MB-2).

R²⁶ may be substituted or unsubstituted aromatic carbocyclyl (hereinafter, referred to as MB-3).

R²⁶ may be substituted or unsubstituted phenyl (hereinafter, referred to as MB-4).

R²⁶ may be phenyl which is substituted with alkyl, halogen, haloalkyl, alkyloxy, non-aromatic carbocyclyloxy, or haloalkyloxy, or unsubstituted (hereinafter, referred to as MB-5).

R²⁶ may be phenyl which is substituted with alkyloxy, non-aromatic carbocyclyloxy, or haloalkyloxy, or unsubstituted (hereinafter, referred to as MB-6).

R⁷ may be a group represented by Formula:

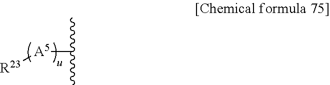

wherein A⁵ is CR²⁸R^{28′} wherein R²⁸ is each independently a hydrogen atom, halogen, substituted or unsubstituted alkyl, or substituted or unsubstituted alkyloxy; R^{28′} is each independently a hydrogen atom, halogen, substituted or unsubstituted alkyl, or substituted or unsubstituted alkyloxy (hereinafter, referred to as NB-1).

R⁷ may be a group represented by Formula:

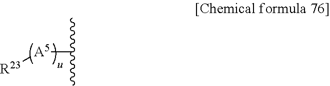

wherein A⁵ is CR²⁸R^{28′} wherein R²⁸ is each independently a hydrogen atom or substituted or unsubstituted alkyl; R^{28′} is each independently a hydrogen atom or substituted or unsubstituted alkyl (hereinafter, referred to as NB-2).

R⁷ may be a group represented by Formula:

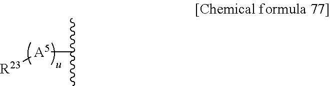

wherein A⁵ is CR²⁸R^{28′} wherein R²⁸ is a hydrogen atom; R^{28′} is a hydrogen atom (hereinafter, referred to as NB-3).

u may be 0, 1, or 2 (hereinafter, referred to as OB-1).

u may be 1 or 2 (hereinafter, referred to as OB-2).

u may be 2 (hereinafter, referred to as OB-3).

u may be 1 (hereinafter, referred to as OB-4).

R²³ may be substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl (hereinafter, referred to as PB-1).

R²³ may be substituted or unsubstituted aromatic carbocyclyl, or substituted or unsubstituted aromatic heterocyclyl (hereinafter, referred to as PB-2).

R²³ may be substituted or unsubstituted aromatic heterocyclyl (hereinafter, referred to as PB-3).

R²³ may be substituted or unsubstituted pyrazolyl (hereinafter, referred to as PB-4).

R²³ may be substituted or unsubstituted pyridyl (hereinafter referred to as PB-5).

R²³ may be substituted or unsubstituted aromatic carbocyclyl (hereinafter, referred to as PB-6).

R²³ may be pyrazolyl which is substituted with alkyl, or unsubstituted (hereinafter, referred to as PB-7).

R²³ may be pyridyl which is substituted with halogen, or unsubstituted (hereinafter referred to as PB-8).

R²³ may be phenyl which is substituted with halogen, alkoxy, or hydroxy, or unsubstituted (hereinafter referred to as PB-9).

R²¹ may be a hydrogen atom or substituted or unsubstituted alkyl (hereinafter, referred to as QB-1).

R²¹ may be a hydrogen atom (hereinafter, referred to as QB-2).

R²¹ may be substituted or unsubstituted alkyl (hereinafter, referred to as QB-3).

R²² may be each independently halogen or substituted or unsubstituted alkyl (hereinafter, referred to as RB-1).

R²² may be each independently substituted or unsubstituted alkyl (hereinafter, referred to as RB-2).

R²² may be each independently halogen (hereinafter, referred to as RB-3).

v may be 0, 1, or 2 (hereinafter, referred to as RB-1).

v may be 1 (hereinafter, referred to as RB-2).

v may be 0 (hereinafter, referred to as RB-3).

In particular, the following embodiments are preferable.

(i) A compound represented by Formula (I):

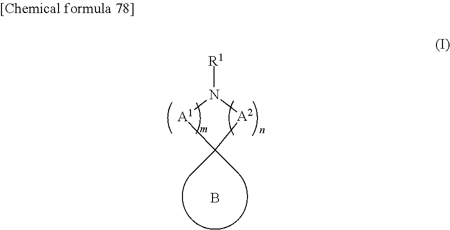

wherein R¹ is a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
A¹ is each independently CR²R^{2′};
A² is each independently CR³R^{3′};
R² is each independently a hydrogen atom, halogen, substituted or unsubstituted alkyl, or substituted or unsubstituted alkyloxy;
R^{2′} is each independently a hydrogen atom, halogen, substituted or unsubstituted alkyl, or substituted or unsubstituted alkyloxy;
R³ is each independently a hydrogen atom, halogen, substituted or unsubstituted alkyl, or substituted or unsubstituted alkyloxy;
R^{3′} is each independently a hydrogen atom, halogen, substituted or unsubstituted alkyl, or substituted or unsubstituted alkyloxy;
R² and R^{2′} and R³ and R^{3′} may be taken together with an identical carbon atom to which they are bonded to form a substituted or unsubstituted non-aromatic carbocycle or a substituted or unsubstituted non-aromatic heterocycle;
m and n are each independently 1, 2, or 3;
ring B is a ring represented by Formula:

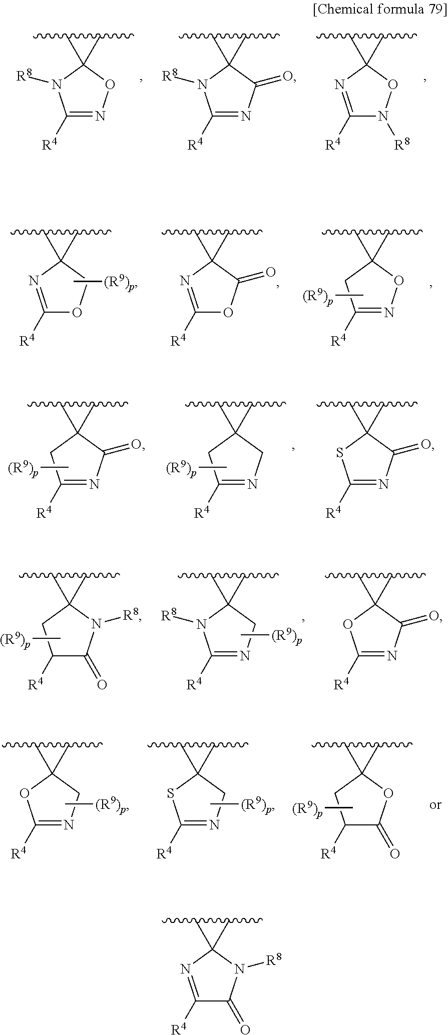

wherein R⁴ is a group represented by Formula:

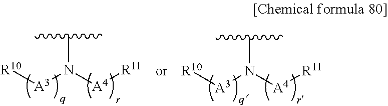

wherein A³ is each independently CR¹³R^{13′};
A⁴ is each independently CR¹⁴R^{14′};
R¹³ is each independently a hydrogen atom, substituted or unsubstituted alkyl, or substituted or unsubstituted alkyloxy;
R^{13′} is each independently a hydrogen atom, halogen, substituted or unsubstituted alkyl, or substituted or unsubstituted alkyloxy;
R¹⁴ is each independently a hydrogen atom, halogen, substituted or unsubstituted alkyl, or substituted or unsubstituted alkyloxy;
R^{14′} is each independently a hydrogen atom, halogen, substituted or unsubstituted alkyl, or substituted or unsubstituted alkyloxy;
q and r are each independently 0, 1, or 2;
q′ and r′ are each independently 1 or 2;
R¹⁰ and R¹¹ are each independently substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁸ is a hydrogen atom or substituted or unsubstituted alkyl;
R⁹ is each independently halogen or substituted or unsubstituted alkyl;
p is an integer of any of 0 to 6,
   or a pharmaceutically acceptable salt thereof.
   (ii) A compound represented by Formula (II):

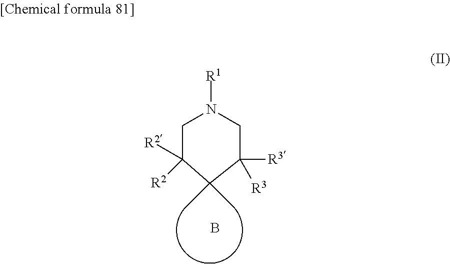

wherein R¹ is a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R² is a hydrogen atom, halogen, substituted or unsubstituted alkyl, or substituted or unsubstituted alkyloxy;
R^{2′} is a hydrogen atom, halogen, substituted or unsubstituted alkyl, or substituted or unsubstituted alkyloxy;
R³ is a hydrogen atom, halogen, substituted or unsubstituted alkyl, or substituted or unsubstituted alkyloxy;
R^{3′} is a hydrogen atom, halogen, substituted or unsubstituted alkyl, or substituted or unsubstituted alkyloxy;
R² and R^{2′} and R³ and R^{3′} may be taken together with the identical carbon atom to which they are bonded to form a substituted or unsubstituted non-aromatic carbocycle or a substituted or unsubstituted non-aromatic heterocycle;
ring B is a ring represented by Formula:

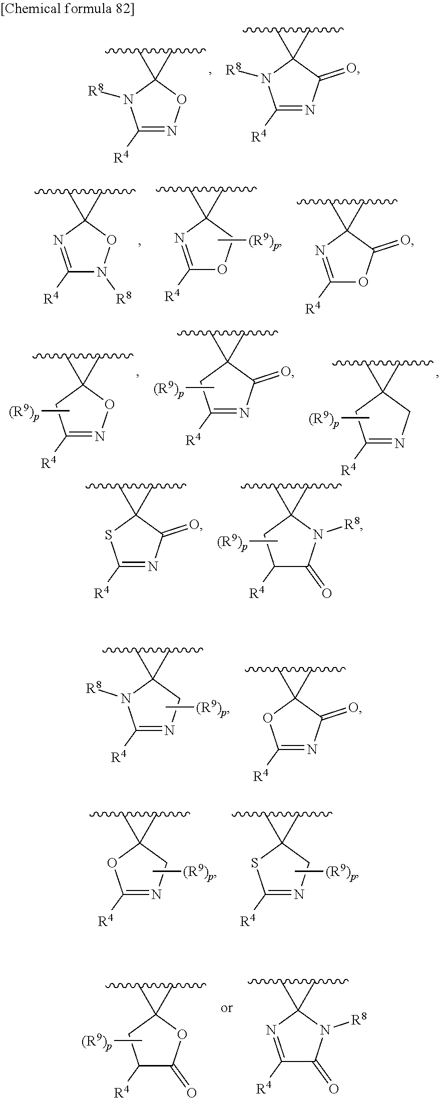

wherein R⁴ is a group represented by Formula:

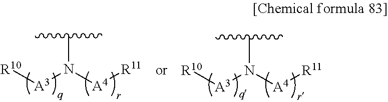

wherein A³ is each independently CR¹³R^{13′};
A⁴ is each independently CR¹⁴R¹⁴;
R¹³ is each independently a hydrogen atom, substituted or unsubstituted alkyl, or substituted or unsubstituted alkyloxy;
R^{13′} is each independently a hydrogen atom, halogen, substituted or unsubstituted alkyl, or substituted or unsubstituted alkyloxy;
R¹⁴ is each independently a hydrogen atom, halogen, substituted or unsubstituted alkyl, or substituted or unsubstituted alkyloxy;
R^{14′} is each independently a hydrogen atom, halogen, substituted or unsubstituted alkyl, or substituted or unsubstituted alkyloxy;
q and r are each independently 0, 1, or 2;
q′ and r′ are each independently 1 or 2;
R¹⁰ and R¹¹ are each independently substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁸ is a hydrogen atom or substituted or unsubstituted alkyl;
R⁹ is each independently halogen or substituted or unsubstituted alkyl;
p is an integer of any of 0 to 6,
   or a pharmaceutically acceptable salt thereof.
   (iii) A compound represented by Formula (II):
   [Chemical Formula 84] [symbol]
wherein R¹ is a hydrogen atom or substituted or unsubstituted alkyl;
R² is a hydrogen atom, halogen, substituted or unsubstituted alkyl, or substituted or unsubstituted alkyloxy;
R^{2′} is a hydrogen atom, halogen, substituted or unsubstituted alkyl, or substituted or unsubstituted alkyloxy;
R³ is a hydrogen atom, halogen, substituted or unsubstituted alkyl, or substituted or unsubstituted alkyloxy;
R^{3′} is a hydrogen atom, halogen, substituted or unsubstituted alkyl, or substituted or unsubstituted alkyloxy;
R² and R^{2′} and R³ and R^{3′} may be taken together with the identical carbon atom to which they are bonded to form a substituted or unsubstituted non-aromatic carbocycle or a substituted or unsubstituted non-aromatic heterocycle;
ring B is a ring represented by Formula:

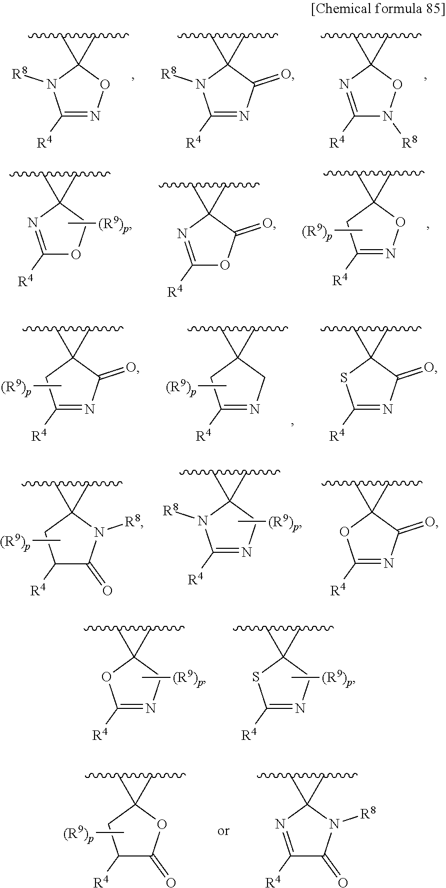

wherein R⁴ is a group represented by Formula:

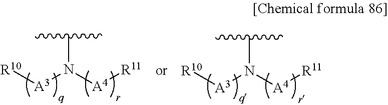

wherein A³ is CR¹³R^{13′};
A⁴ is CR¹⁴R^{14′};
R¹³ is a hydrogen atom, substituted or unsubstituted alkyl, or substituted or unsubstituted alkyloxy;
R^{13′} is a hydrogen atom, halogen, substituted or unsubstituted alkyl, or substituted or unsubstituted alkyloxy;
R¹⁴ is a hydrogen atom, halogen, substituted or unsubstituted alkyl, or substituted or unsubstituted alkyloxy;
R^{14′} is a hydrogen atom, halogen, substituted or unsubstituted alkyl, or substituted or unsubstituted alkyloxy;
q and r are each 1;
q′ and r′ are each 1;
R¹⁰ and R¹¹ are each independently substituted or unsubstituted aromatic carbocyclyl or substituted or unsubstituted aromatic heterocyclyl;
R⁸ is a hydrogen atom or substituted or unsubstituted alkyl;
R⁹ is each independently halogen or substituted or unsubstituted alkyl;
p is an integer of any of 0 to 2,
   or a pharmaceutically acceptable salt thereof.
   (iv) A compound represented by Formula (II):
   [Chemical Formula 87] [symbol]
wherein R¹ is a hydrogen atom or substituted or unsubstituted alkyl;
R² is a hydrogen atom, halogen, substituted or unsubstituted alkyl, or substituted or unsubstituted alkyloxy;
R^{2′} is a hydrogen atom, halogen, substituted or unsubstituted alkyl, or substituted or unsubstituted alkyloxy;
R³ is a hydrogen atom, halogen, substituted or unsubstituted alkyl, or substituted or unsubstituted alkyloxy;
R^{3′} is a hydrogen atom, halogen, substituted or unsubstituted alkyl, or substituted or unsubstituted alkyloxy;
R² and R^{2′} and R³ and R^{3′} may be taken together with the identical carbon atom to which they are bonded to form a substituted or unsubstituted non-aromatic carbocycle or a substituted or unsubstituted non-aromatic heterocycle;
ring B is a ring represented by Formula:

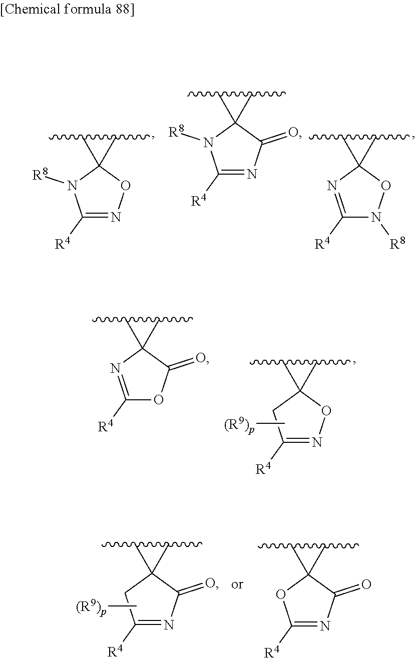

wherein R⁴ is a group represented by Formula:

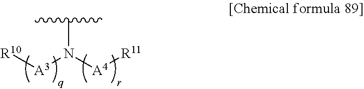

wherein A³ is CR¹³R^{13′};
A⁴ is CR¹⁴R¹⁴;
R¹³ is a hydrogen atom, substituted or unsubstituted alkyl, or substituted or unsubstituted alkyloxy;
R^{13′} is a hydrogen atom, halogen, substituted or unsubstituted alkyl, or substituted or unsubstituted alkyloxy;
R¹⁴ is a hydrogen atom, halogen, substituted or unsubstituted alkyl, or substituted or unsubstituted alkyloxy;
R^{14′} is a hydrogen atom, halogen, substituted or unsubstituted alkyl, or substituted or unsubstituted alkyloxy;
q and r are each 1;
R¹⁰ and R¹¹ are each independently substituted or unsubstituted aromatic carbocyclyl or substituted or unsubstituted aromatic heterocyclyl;
R⁸ is a hydrogen atom or substituted or unsubstituted alkyl;
R⁹ is each independently halogen or substituted or unsubstituted alkyl;
p is an integer of any of 0 to 2,
   or a pharmaceutically acceptable salt thereof.
   (v) A compound represented by Formula (III):

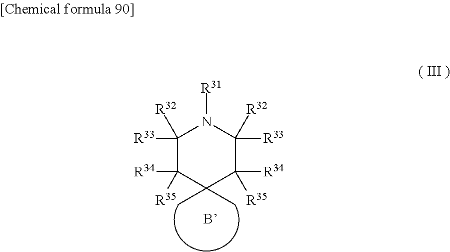

wherein R³¹ is a hydrogen atom or C1-C3 alkyl;
R³² is each independently a hydrogen atom or substituted or unsubstituted alkyl;
R³³ is each independently a hydrogen atom or substituted or unsubstituted alkyl;
R³⁴ is each independently a hydrogen atom, halogen, or substituted or unsubstituted alkyl;
R³⁵ is each independently a hydrogen atom, halogen, or substituted or unsubstituted alkyl;
R³² and R³³ and R³⁴ and R³⁵ may be taken together with an identical carbon atom to which they are bonded to form a substituted or unsubstituted non-aromatic carbocycle or a substituted or unsubstituted non-aromatic heterocycle;
ring B′ is a group represented by Formula:

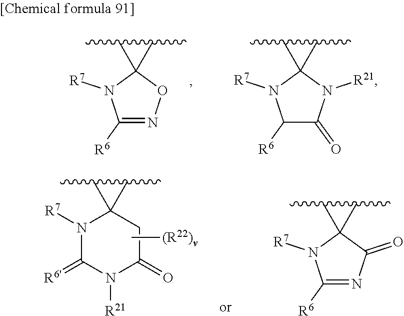

wherein R⁶ is a group represented by Formula:

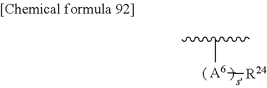

wherein A⁶ is CR²⁵R^{25′};
R²⁵ is a hydrogen atom, halogen, substituted or unsubstituted alkyl, or substituted or unsubstituted alkyloxy;
R^{25′} is a hydrogen atom, halogen, substituted or unsubstituted alkyl, or substituted or unsubstituted alkyloxy;
s′ is 1;
R²⁴ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R^{6′} is a group represented by Formula:

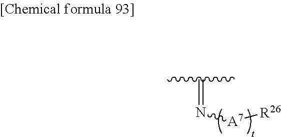

wherein A⁷ is CR²⁷R^{27′};
R²⁷ is a hydrogen atom, halogen, substituted or unsubstituted alkyl, or substituted or unsubstituted alkyloxy;
R^{27′} is a hydrogen atom, halogen, substituted or unsubstituted alkyl, or substituted or unsubstituted alkyloxy;
t is 1;
R²⁶ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁷ is a group represented by Formula:

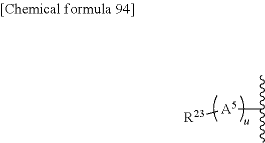

wherein A⁵ is CR²⁸R^{28′};
R²⁸ is a hydrogen atom, halogen, substituted or unsubstituted alkyl, or substituted or unsubstituted alkyloxy;
R^{28′} is a hydrogen atom, halogen, substituted or unsubstituted alkyl, or substituted or unsubstituted alkyloxy;
u is 1;
R²³ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl,
R²¹ is a hydrogen atom or substituted or unsubstituted alkyl;
R²² is each independently halogen or substituted or unsubstituted alkyl;
v is 0, 1, or 2,
   or a pharmaceutically acceptable salt thereof.
   (vi) A compound represented by Formula (III):

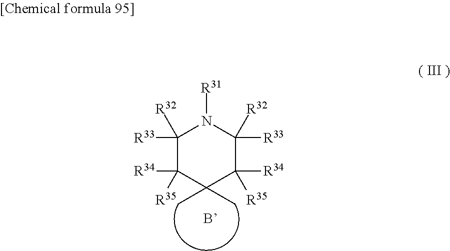

wherein R³¹ is a hydrogen atom or C1-C3 alkyl;
R³² is each independently a hydrogen atom or substituted or unsubstituted alkyl;
R³³ is each independently a hydrogen atom or substituted or unsubstituted alkyl;
R³⁴ is each independently a hydrogen atom, halogen, or substituted or unsubstituted alkyl;
R³⁵ is each independently a hydrogen atom, halogen, or substituted or unsubstituted alkyl;
R³² and R³³ and R³⁴ and R³⁵ may be taken together with an identical carbon atom to which they are bonded to form a substituted or unsubstituted non-aromatic carbocycle or a substituted or unsubstituted non-aromatic heterocycle;
ring B′ is a group represented by Formula:

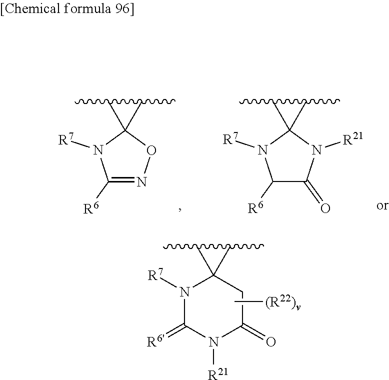

wherein R⁶ is a group represented by Formula:

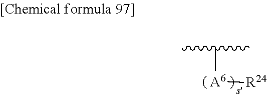

wherein A⁶ is CR²⁵R^{25′};
R²⁵ is a hydrogen atom, halogen, substituted or unsubstituted alkyl, or substituted or unsubstituted alkyloxy;
R^{25′} is a hydrogen atom, halogen, substituted or unsubstituted alkyl, or substituted or unsubstituted alkyloxy;
s′ is 1;
R²⁴ is substituted or unsubstituted aromatic carbocyclyl or substituted or unsubstituted aromatic heterocyclyl;
R^{6′} is a group represented by Formula:

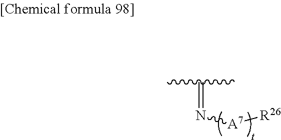

wherein A⁷ is CR²⁷R^{27′};
R²⁷ is a hydrogen atom, halogen, substituted or unsubstituted alkyl, or substituted or unsubstituted alkyloxy;
R^{27′} is a hydrogen atom, halogen, substituted or unsubstituted alkyl, or substituted or unsubstituted alkyloxy;
t is 1;
R²⁶ is substituted or unsubstituted aromatic carbocyclyl or substituted or unsubstituted aromatic heterocyclyl;
R⁷ is a group represented by Formula:

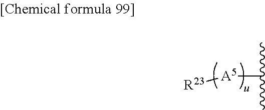

wherein A⁵ is CR²⁸R^{28′};
R²⁸ is a hydrogen atom, halogen, substituted or unsubstituted alkyl, or substituted or unsubstituted alkyloxy;
R^{28′} is a hydrogen atom, halogen, substituted or unsubstituted alkyl, or substituted or unsubstituted alkyloxy;
u is 1;
R²³ is substituted or unsubstituted aromatic carbocyclyl or substituted or unsubstituted aromatic heterocyclyl,
R²¹ is a hydrogen atom or substituted or unsubstituted alkyl;
R²² is each independently halogen or substituted or unsubstituted alkyl;
v is 0, 1, or 2,
   or a pharmaceutically acceptable salt thereof.
   (vii) A compound represented by Formula (II):

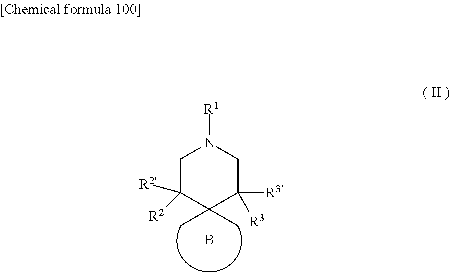

wherein R¹ is a hydrogen atom or alkyl;
R² is a hydrogen atom;
R^{2′} is a hydrogen atom;
R³ is a hydrogen atom;
R^{3′} is a hydrogen atom;
ring B is a ring represented by Formula:

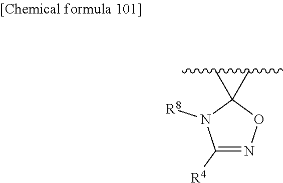

wherein R⁴ is a group represented by Formula:

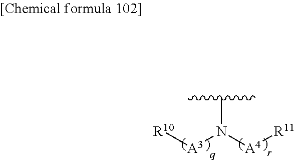

wherein A³ is CR¹³R^{13′};
A⁴ is CR¹⁴R¹⁴;
R¹³ is a hydrogen atom;
R^{13′} is a hydrogen atom;
R¹⁴ is a hydrogen atom;
R^{14′} is a hydrogen atom;
q and r are each 1;
R¹⁰ is 5-membered aromatic heterocyclyl substituted with substituent group ω (substituent group ω: alkyl, haloalkyl, and non-aromatic carbocyclyl);
R¹¹ is a group represented by Formula:

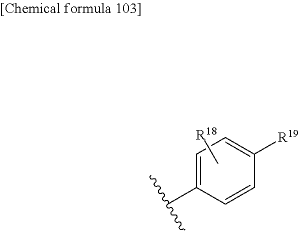

wherein R¹⁸ is a hydrogen atom or halogen;
R¹⁹ is alkyloxy or haloalkyloxy;
R⁸ is a hydrogen atom,
   or a pharmaceutically acceptable salt thereof.

The compounds represented by Formula (I), Formula (II), or Formula (III) are not limited to specific isomers, but include all possible isomers (e.g., keto-enol isomers, imine-enamine isomers, diastereoisomers, optical isomers, rotational isomers, tautomers as shown below, etc.), racemates, or mixtures thereof.

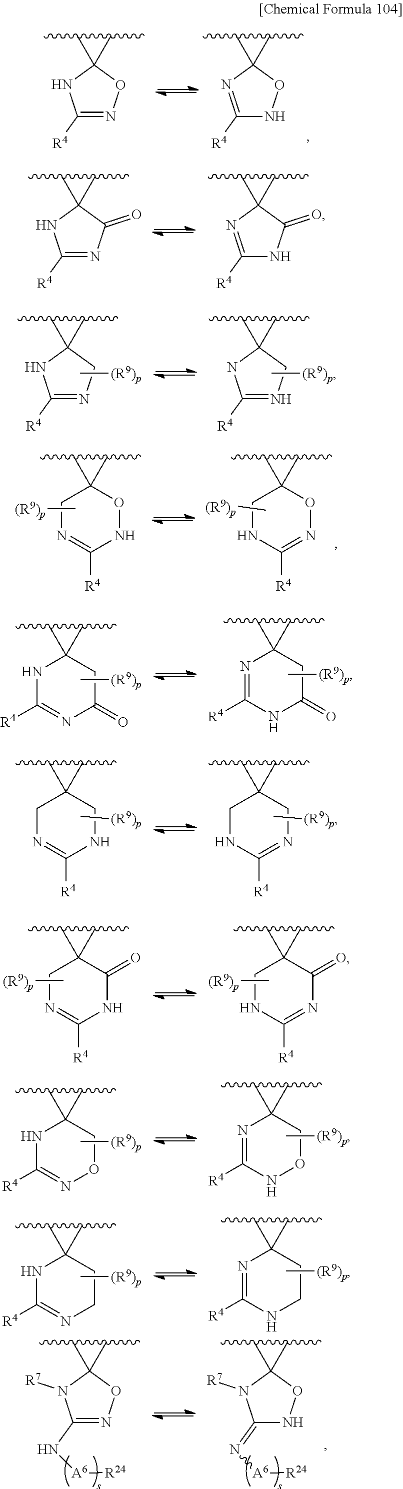

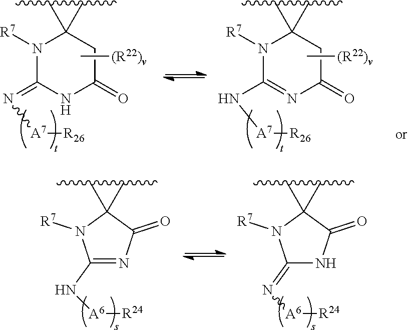

One or more hydrogen, carbon, and/or other atom(s) of the compounds represented by Formula (I), Formula (II), or Formula (III) may be substituted with isotope(s) of hydrogen, carbon, and/or other atom(s), respectively. Examples of such isotopes include hydrogen, carbon, nitrogen, oxygen, phosphorus, sulfur, fluorine, iodine, and chlorine, as in the cases of ²H, ³H, ¹¹C, ¹³C, ¹⁴C, ¹⁵N, ¹⁸O, ¹⁷O, ³¹P, ³²P ³⁵S, ¹⁸F, ¹²³I, and ³⁶Cl, respectively. The compounds represented by Formula (I), Formula (II), or Formula (III) also include compounds substituted with such isotopes. The compounds substituted with the isotopes are also useful as pharmaceutical products and include all radiolabeled forms of the compounds represented by Formula (I), Formula (II), or Formula (III). Furthermore, a “radiolabeling method” for producing the “radiolabeled forms” is also included in the present invention, and the “radiolabeled forms” are useful as tools for metabolic pharmacokinetics studies, studies on binding assay, and/or diagnostics.

Radiolabeled forms of the compounds represented by Formula (I), Formula (II), or Formula (III) can be prepared by methods well known in the pertinent art. For example, a tritium-labeled compound represented by Formula (I), Formula (II), or Formula (III) can be prepared by introducing tritium into a specific compound represented by Formula (I), Formula (II), or Formula (III), by a catalytic dehalogenation reaction using tritium. This method comprises reacting an appropriately-halogenated precursor of the compound represented by Formula (I), Formula (II), or Formula (III) with tritium gas in the presence of an appropriate catalyst, such as Pd/C, and in the presence or absence of a base. The other appropriate method of preparing a tritium-labeled compound can be referred to “Isotopes in the Physical and Biomedical Sciences, Vol. 1, Labeled Compounds (Part A), Chapter 6 (1987)”. A ¹⁴C-labeled compound can be prepared by using a raw material having ¹⁴C carbon.

The pharmaceutically acceptable salts of the compounds represented by Formula (I), Formula (II), or Formula (III) include, for example, salts of compounds represented by Formula (I), Formula (II), or Formula (III) with alkaline metal (e.g., lithium, sodium, or potassium), alkaline earth metal (e.g., calcium or barium), magnesium, transition metal (e.g., zinc or iron), ammonia, organic bases (e.g., trimethylamine, triethylamine, dicyclohexylamine, ethanolamine, diethanolamine, triethanolamine, meglumine, ethylenediamine, pyridine, picoline, or quinoline), or amino acids, or salts with inorganic acids (e.g., hydrochloric acid, sulfuric acid, nitric acid, carbonic acid, hydrobromic acid, phosphoric acid, or hydroiodic acid) or organic acids (e.g., formic acid, acetic acid, propionic acid, trifluoroacetic acid, citric acid, lactic acid, tartaric acid, oxalic acid, maleic acid, fumaric acid, succinic acid, mandelic acid, glutaric acid, malic acid, benzoic acid, phthalic acid, ascorbic acid, benzenesulfonic acid, p-toluenesulfonic acid, methanesulfonic acid, ethanesulfonic acid, or trifluoroacetic acid). These salts can be formed by the usual methods.

The compounds represented by Formula (I), Formula (II), or Formula (III) of the present invention or pharmaceutically acceptable salts thereof may form solvates (e.g., hydrates), co-crystals, and/or crystal polymorphs. The present invention encompasses those various solvates, co-crystals, and crystal polymorphs. The “solvates” may have the compounds represented by Formula (I), Formula (II), or Formula (III) coordinated with any number of solvent molecules (e.g., water molecules). When the compounds represented by Formula (I), Formula (II), or Formula (III), or pharmaceutically acceptable salts thereof are allowed to stand in the atmosphere, the compounds may absorb water, resulting in attachment of adsorbed water or formation of hydrates. Recrystallization of the compounds represented by Formula (I), Formula (II), or Formula (III), or pharmaceutically acceptable salts thereof may produce crystal polymorphs. The “co-crystal” means that a compound represented by Formula (I), Formula (II), or Formula (III), or a salt thereof and a counter molecule exist in the same crystal lattice, and it can include any number of counter molecules.

The compounds represented by Formula (I), Formula (II), or Formula (III) of the present invention or pharmaceutically acceptable salts thereof may form prodrugs. The present invention also encompasses such various prodrugs. Prodrugs are derivatives of the compounds according to the present invention that have chemically or metabolically degradable groups, and compounds that are converted to the pharmaceutically active compounds according to the present invention through solvolysis or under physiological conditions in vivo. Prodrugs include compounds that are converted to the compounds represented by Formula (I), Formula (II), or Formula (III) through enzymatic oxidation, reduction, hydrolysis, or the like under physiological conditions in vivo, compounds that are converted to the compounds represented by Formula (I), Formula (II), or Formula (III) through hydrolysis by gastric acid etc., and the like. Methods for selecting and preparing suitable prodrug derivatives are described in, for example, “Design of Prodrugs, Elsevier, Amsterdam, 1985”. Prodrugs themselves may have some activity.

When the compounds represented by Formula (I), Formula (II), or Formula (III) or pharmaceutically acceptable salts thereof have hydroxyl group(s), prodrugs include acyloxy derivatives and sulfonyloxy derivatives that are prepared by, for example, reacting compounds having hydroxyl group(s) with suitable acyl halide, suitable acid anhydride, suitable sulfonyl chloride, suitable sulfonyl anhydride, and mixed anhydride, or with a condensing agent. For example, they include CH₃COO—, C₂H₅COO—, tert-BuCOO—, C₁₅H₃₁COO—, PhCOO—, (m-NaOOCPh)COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃, CF₃CH₂SO₃—, p-CH₃₀-PhSO₃—, PhSO₃— and p-CH₃PhSO₃—.

Since the compound according to the present invention has serotonin 5-HT2A receptor antagonism and/or inverse agonism, the compound is useful as a therapeutic and/or prophylactic agent for a disease associated with a serotonin 5-HT2A receptor. Diseases associated with serotonin 5-HT2A receptor include serotonin-mediated diseases such as Parkinson's disease-related hallucinations and delusions, dementia-related hallucinations and delusions, schizophrenia-related hallucinations and delusions, depression-related hallucinations and delusions, neurodegenerative diseases-related hallucinations and delusions, depression, schizophrenia, autism, dependence, dyskinesia, sleep disorder, Parkinson's disease-related irritability, dementia-related irritability, schizophrenia-related irritability, sexual dysfunction and the like. Preferable examples include Parkinson's disease-related hallucinations and delusions, dementia-related hallucinations and delusions, schizophrenia-related hallucinations and delusions, depression-related hallucinations and delusions, Parkinson's disease-related irritability, dementia-related irritability, and schizophrenia-related irritability. More preferable examples include Parkinson's disease-related hallucinations and delusions, and dementia-related hallucinations and delusions.

The “serotonin 5-HT2A receptor antagonist and/or inverse agonist” means a pharmaceutical product having serotonin 5-HT2A receptor antagonism and/or inverse agonism.

The “composition for serotonin 5-HT2A receptor antagonism and/or inverse agonism” means a composition having serotonin 5-HT2A receptor antagonism and/or inverse agonism, and it is not limited to pharmaceutical use.

### (Method for Producing Compounds of the Present Invention)

The compounds represented by Formula (I), Formula (II), or Formula (III) according to the present invention can be produced by, for example, the general synthetic method described below. Regarding extraction, purification, and the like, the treatments carried out in ordinary experiments of organic chemistry may be carried out.

The compounds of the present invention can be synthesized with reference to methods known in the art.

### General Synthetic Method 1

### (Method A)

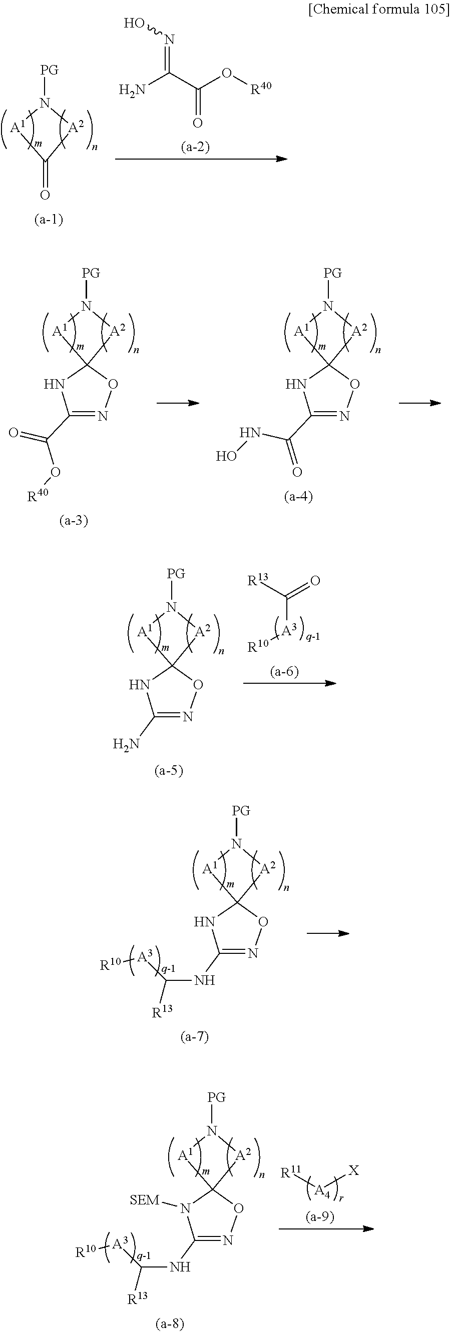

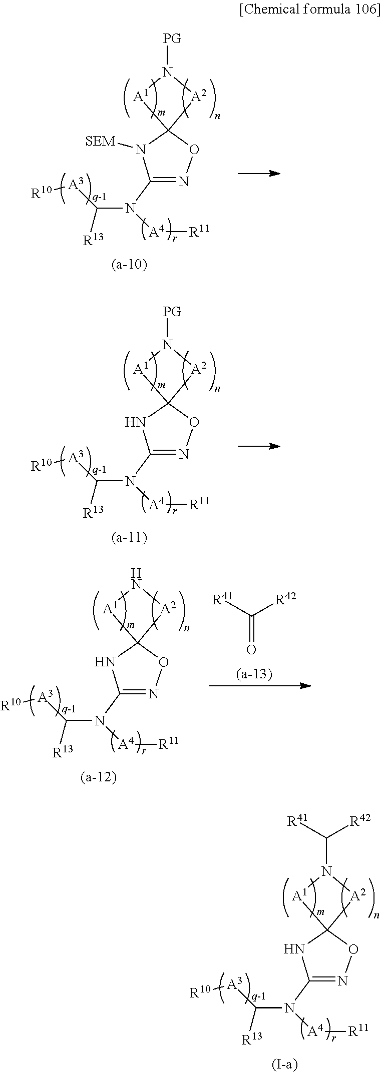

wherein PG is an appropriate protecting group for an amino group such as Boc or Z; R⁴⁰ is alkyl; X is a leaving group such as halogen; R⁴¹ and R⁴² are each independently a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl; R⁴¹ and R⁴² may be taken together with the identical carbon atom to which they are bonded to form a substituted or unsubstituted non-aromatic carbocycle or a substituted or unsubstituted non-aromatic heterocycle; the other symbols have the same meanings as those in the above item (1).

### Step 1

Compound (a-3) can be obtained by reacting Compounds (a-1) and (a-2) in the presence of an acid in the absence of a solvent or in an appropriate solvent.

Examples of the acid include hydrochloric acid, sulfuric acid, TFA, formic acid, trifluoroborane, toluenesulfonic acid, and pyridinium toluenesulfonate, and the acid can be used in an amount of 0.1 molar equivalents or more, preferably 0.1 to 10 molar equivalents relative to Compound (a-1).

Examples of the reaction solvent include methanol, ethanol, tert-butanol, isopropanol, toluene, benzene, xylene, cyclohexane, hexane, tetrahydrofuran, diethyl ether, dioxane, dimethoxyethane, chloroform, dichloromethane, DMF, DMSO, NMP, acetonitrile, and pyridine, and each solvent can be used alone or mixed with the others.

The reaction temperature is 0 to 200° C., preferably 20 to 120° C.

The reaction time is 0.1 to 24 hours, preferably 0.5 to 6 hours.

### Step 2

Compound (a-4) can be obtained by allowing hydroxylamine to act on Compound (a-3).

Hydroxylamine can be used in an amount of 1 to 30 molar equivalents.

The reaction temperature is 0° C. to the reflux temperature of the solvent, preferably 40 to 80° C.

The reaction time is 0.5 to 48 hours, preferably 1 hour to 6 hours.

Examples of the reaction solvent include methanol, ethanol, 2-propanol, tetrahydrofuran, toluene, chloroform, DMF, and DMA, and each solvent can be used alone or mixed with the others.

### Step 3

Compound (a-5) can be obtained by allowing a condensing agent and 2-(trimethylsilyl)ethanol to act on Compound (a-4) in the presence or absence of a base, and then allowing a fluoride to act.

Examples of the base include NMM and triethylamine, and the base can be used in an amount of 1 to 10 molar equivalents relative to Compound (a-4).

Examples of the condensing agent include T3P, CDI, MsCl, and TsCl, and the condensing agent can be used in an amount of 1 to 10 molar equivalents relative to Compound (a-4).

2-(Trimethylsilyl)ethanol can be used in an amount of 1 to 10 molar equivalents relative to Compound (a-4).

Examples of the fluoride include TBAF, KF, and pyridinium fluoride, and the fluoride can be used in an amount of 1 to 10 molar equivalents relative to Compound (a-4).

The reaction temperature is 0° C. to the reflux temperature of the solvent, preferably 40 to 80° C.

The reaction time is 0.5 to 48 hours, preferably 1 hour to 6 hours.

Examples of the reaction solvent include tetrahydrofuran, toluene, chloroform, DMF, and DMA, and each solvent can be used alone or mixed with the others.

### Step 4

Compound (a-7) can be obtained by reacting Compound (a-5) and Compound (a-6) in the presence or absence of a condensing agent and reducing with a reducing agent.

Examples of the condensing agent include 4-toluenesulfonic acid, methanesulfonic acid, acetic acid, anhydrous magnesium sulfate, tetraisopropyl orthotitanate, titanium tetrachloride, and molecular sieve, and the condensing agent can be used in an amount of 1 to 10 molar equivalents relative to Compound (a-5).

Compound (a-6) can be used in an amount of 1 to 10 molar equivalents relative to Compound (a-5).

Examples of the base include sodium hydroxide, sodium carbonate, sodium hydrogen carbonate, potassium carbonate, calcium carbonate, cesium carbonate, pyridine, triethylamine, and DMAP, and the base can be used in an amount of 1 to 5 molar equivalents relative to Compound (a-5).

Examples of the reducing agent include sodium borohydride, sodium cyanoborohydride, sodium triacetoxyborohydride, borane and complexes thereof, lithium borohydride, potassium borohydride, and diisobutylaluminum hydride, and the reducing agent can be used in an amount of 1 to 10 molar equivalents relative to Compound (a-5).

The reaction temperature is −78° C. to the reflux temperature of the solvent, preferably 25 to 100° C.

The reaction time is 0.5 to 48 hours, preferably 1 hour to 6 hours.

Examples of the reaction solvent include tetrahydrofuran, toluene, dichloromethane, chloroform, methanol, and ethanol, and each solvent can be used alone or mixed with the others.

### Step 5

Compound (a-8) can be obtained by allowing 2-(chloromethoxy)ethyltrimethylsilane to act on Compound (a-7) in the presence of a base.

2-(Chloromethoxy)ethyltrimethylsilane can be used in an amount of 1 to 10 molar equivalents relative to Compound (a-7).

Examples of the base include sodium hydroxide, sodium carbonate, sodium hydrogen carbonate, potassium carbonate, calcium carbonate, cesium carbonate, pyridine, triethylamine, and DMAP, and the base can be used in an amount of 1 to 5 molar equivalents relative to Compound (a-7).

The reaction temperature is −10° C. to 80° C., preferably 0° C. to 25° C.

The reaction time is 0.5 hours to 24 hours, preferably 0.5 to 6 hours.

Examples of the reaction solvent include DMF, DMA, DMSO, tetrahydrofuran, dioxane, and acetonitrile, and each solvent can be used alone or mixed with the others.

### Step 6

Compound (a-10) can be obtained by reacting Compound (a-9) with Compound (a-8) in the presence of a base.

The reaction temperature is 0° C. to 40° C., preferably 0° C. to 20° C.

The reaction time is 0.5 hours to 12 hours, preferably 1 hour to 6 hours.

As the base, sodium carbonate, potassium carbonate, cesium carbonate, lithium hydroxide, sodium hydroxide, potassium hydroxide, sodium hydride, or the like can be used.

Examples of the reaction solvent include DMF, DMA, DMSO, tetrahydrofuran, dioxane, and acetonitrile, and each solvent can be used alone or mixed with the others.

### Step 7

Compound (a-11) can be obtained by allowing a fluoride to act on Compound (a-10).

Examples of the fluoride include TBAF, KF, and pyridinium fluoride, and the fluoride can be used in an amount of 1 to 10 molar equivalents relative to Compound (a-10).

The reaction temperature is 0° C. to the reflux temperature of the solvent, preferably 0 to 25° C.

The reaction time is 0.5 to 48 hours, preferably 1 hour to 6 hours.

Examples of the reaction solvent include tetrahydrofuran, toluene, chloroform, DMF, and DMA, and each solvent can be used alone or mixed with the others.

### Step 8

Compound (a-12) can be obtained by reacting Compound (a-11) in the presence of an acid in the absence of a solvent or in an appropriate solvent, or reacting Compound (a-11) with hydrogen gas in the presence of a metal catalyst.

Examples of the acid include hydrochloric acid, sulfuric acid, TFA, formic acid, and trifluoroborane, and the acid can be used in an amount of 1.0 molar equivalent or more, preferably 1.0 to 30 molar equivalents relative to Compound (a-11).

Examples of the metal catalyst include palladium-carbon, platinum oxide, rhodium-aluminum oxide, and chlorotris(triphenylphosphine)rhodium(I), and the metal catalyst can be used at 0.01 to 100 weight percent relative to Compound (a-11).

The hydrogen pressure can be 1 to 50 atm. As the hydrogen source, cyclohexene, 1,4-cyclohexadiene, formic acid, ammonium formate, or the like can also be used.

Examples of the reaction solvent include methanol, ethanol, tert-butanol, isopropanol, toluene, benzene, xylene, cyclohexane, hexane, tetrahydrofuran, diethyl ether, dioxane, dimethoxyethane, and the like), chloroform, dichloromethane, DMF, DMSO, NMP, acetonitrile, and pyridine, and each solvent can be used alone or mixed with the others.

The reaction temperature is 0 to 80° C., preferably 0 to 20° C.

The reaction time is 0.1 to 24 hours, preferably 0.5 to 6 hours.

### Step 9

Compound (I-a) can be obtained by reacting Compound (a-12) and Compound (a-13) with an appropriate reducing agent and, if necessary, acetic acid in an appropriate solvent.

Examples of the reducing agent include sodium triacetoxyborohydride and sodium cyanoborohydride, and the reducing agent can be used in an amount of 1.0 molar equivalent or more, preferably 1.0 to 2.0 molar equivalents relative to Compound (a-12).

Acetic acid can be used in an amount of 1.0 molar equivalent or more, preferably 1.0 to 2.0 molar equivalents relative to Compound (a-12).

Examples of the reaction solvent include methanol, ethanol, tert-butanol, isopropanol, and the like), toluene, benzene, xylene, cyclohexane, hexane, tetrahydrofuran, diethyl ether, dioxane, dimethoxyethane, chloroform, dichloromethane, DMF, DMSO, NMP, acetonitrile, and pyridine, and each solvent can be used alone or mixed with the others.

The reaction temperature is 0 to 80° C., preferably 0 to 20° C.

The reaction time is 0.1 to 24 hours, preferably 0.5 to 6 hours.

### General Synthetic Method 2

### (Method B)

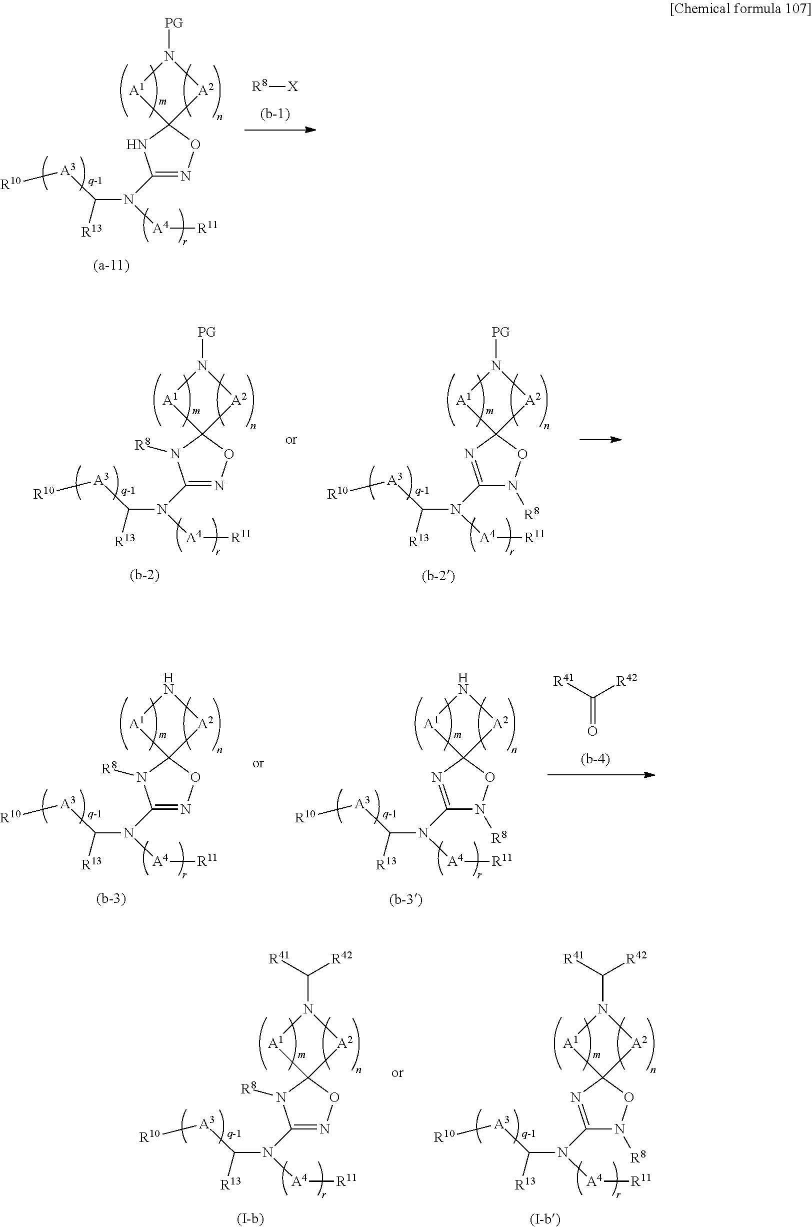

wherein symbols have the same meanings as those in the above Method A or the above item (1).

### Step 1

Compound (b-2) or (b-2′) can be obtained by reacting Compound (b-1) with Compound (a-11) in the presence of a base.

The reaction temperature is 0° C. to 40° C., preferably 0° C. to 20° C.

The reaction time is 0.5 hours to 12 hours, preferably 1 hour to 6 hours.

As the base, sodium carbonate, potassium carbonate, cesium carbonate, lithium hydroxide, sodium hydroxide, potassium hydroxide, sodium hydride, or the like can be used.

Examples of the reaction solvent include methanol, ethanol, water, acetone, acetonitrile, and tetrahydrofuran, and each solvent can be used alone or mixed with the others.

Examples of the reaction solvent include DMF, DMA, DMSO, tetrahydrofuran, dioxane, and acetonitrile, and each solvent can be used alone or mixed with the others.

### Step 2

Compound (b-3) or (b-3′) can be obtained by using Compound (b-2) or (b-2′) as a raw material and using the same method as in Step 8 of Method A described above.

### Step 3

Compound (I-b) or (I-b′) can be obtained by using Compound (b-3) or (b-3′) as a starting raw material and using the same method as in Step 9 of Method A described above.

### General Synthetic Method 3

### (Method C)

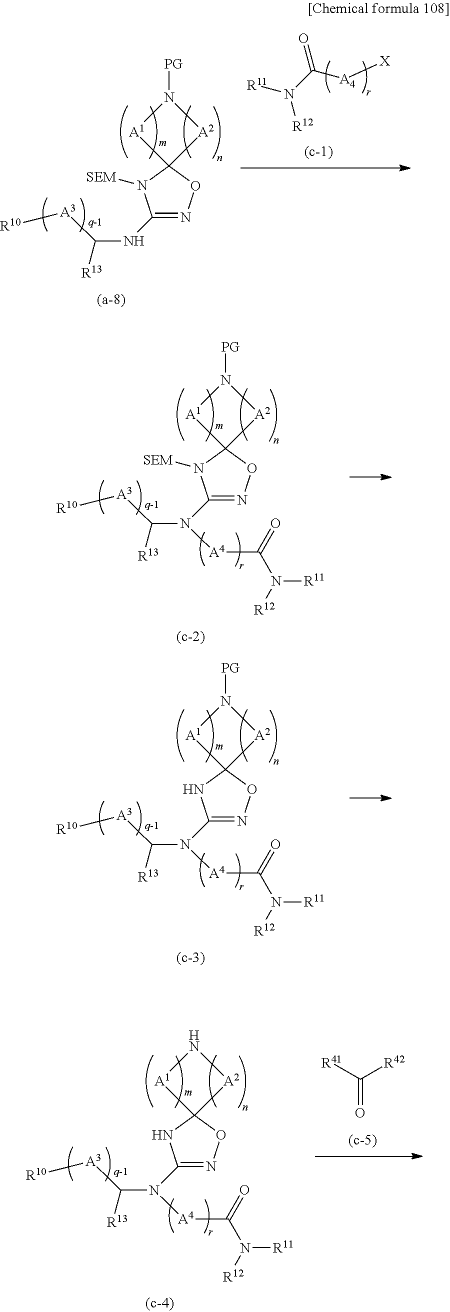

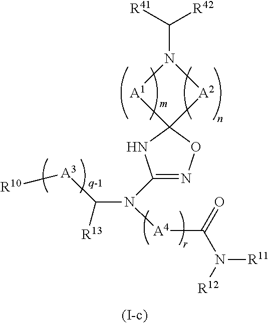

wherein symbols have the same meanings as those in the above Method A or the above item (1).

### Step 1

Compound (c-2) can be obtained by reacting Compound (c-1) with Compound (a-8) in the presence of a base.

The reaction temperature is 0° C. to 40° C., preferably 0° C. to 20° C.

The reaction time is 0.5 hours to 12 hours, preferably 1 hour to 6 hours.

As the base, sodium carbonate, potassium carbonate, cesium carbonate, lithium hydroxide, sodium hydroxide, potassium hydroxide, sodium hydride, or the like can be used.

Examples of the reaction solvent include DMF, DMA, DMSO, tetrahydrofuran, dioxane, and acetonitrile, and each solvent can be used alone or mixed with the others.

### Step 2

Compound (c-3) can be obtained by using Compound (c-2) as a raw material and using the same method as in Step 7 of Method A described above.

### Step 3

Compound (c-4) can be obtained by using Compound (c-3) as a raw material and using the same method as in Step 8 of Method A described above.

### Step 4

Compound (I-c) can be obtained by using Compound (c-4) as a raw material and using the same method as in Step 9 of Method A described above.

### General Synthetic Method 4

### (Method D)

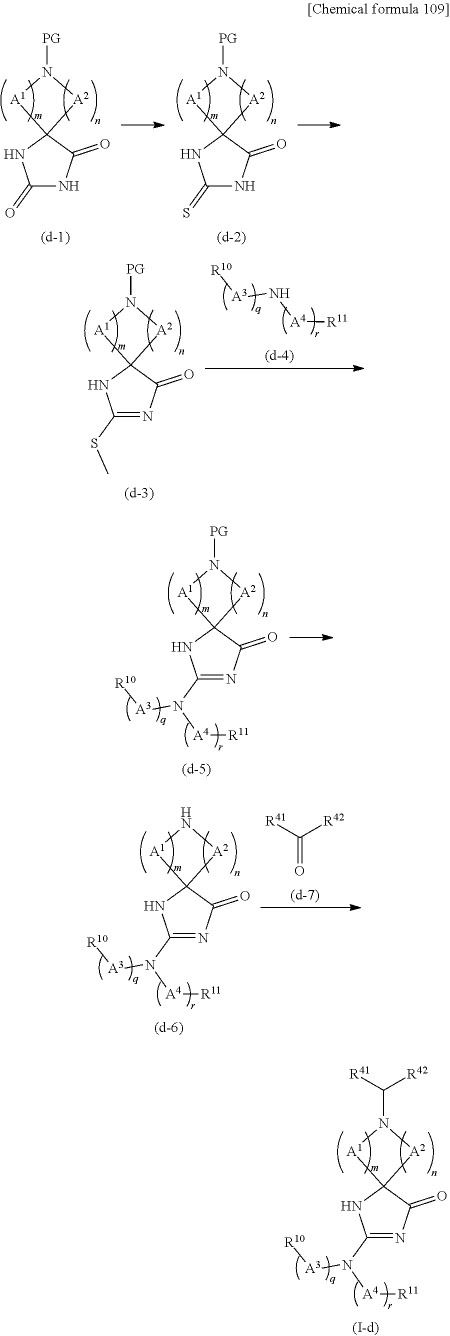

wherein symbols have the same meanings as those in the above Method A or the above (1).

### Step 1

Compound (d-2) can be obtained by reacting Compound (d-1) with a Lawesson's reagent, followed by ethanolamine.

The reaction temperature is 0 to 200° C., preferably 60 to 140° C.

The reaction time is 0.5 to 48 hours, preferably 1 hour to 6 hours.

Examples of the reaction solvent include tetrahydrofuran, DMF, DMA, DMSO, and toluene, and each solvent can be used alone or mixed with the others.

### Step 2

Compound (d-3) can be obtained by adding methyl iodide to Compound (d-2) in the presence of a base.

Methyl iodide can be used in an amount of 1 to 10 molar equivalents relative to Compound (d-2).

Examples of the base include DIEA and triethylamine, and the base can be used in an amount of 1 to 5 molar equivalents relative to Compound (d-2).

The reaction temperature is −78° C. to the reflux temperature of the solvent, preferably 0 to 25° C.

The reaction time is 0.5 to 48 hours, preferably 1 hour to 6 hours.

Examples of the reaction solvent include methanol, ethanol, tetrahydrofuran, DMF, DMA, toluene, dichloromethane, and chloroform, and each solvent can be used alone or mixed with the others.

### Step 3

Compound (d-5) can be obtained by reacting Compound (d-3) with Compound (d-4).

The reaction temperature is 0° C. to the reflux temperature of the solvent, preferably 80 to 130° C.

The reaction time is 0.5 to 48 hours, preferably 1 hour to 12 hours.

Examples of the reaction solvent include acetic acid, DMF, DMA, DMSO, tetrahydrofuran, toluene, t-BuOH, and t-amyl alcohol, and each solvent can be used alone or mixed with the others.

### Step 4

Compound (d-6) can be obtained by using Compound (d-5) as a raw material and using the same method as in Step 8 of Method A described above.

### Step 5

Compound (I-d) can be obtained by using Compound (d-6) as a raw material and using the same method as in Step 9 of Method A described above.

### General Synthetic Method 5

### (Method E)

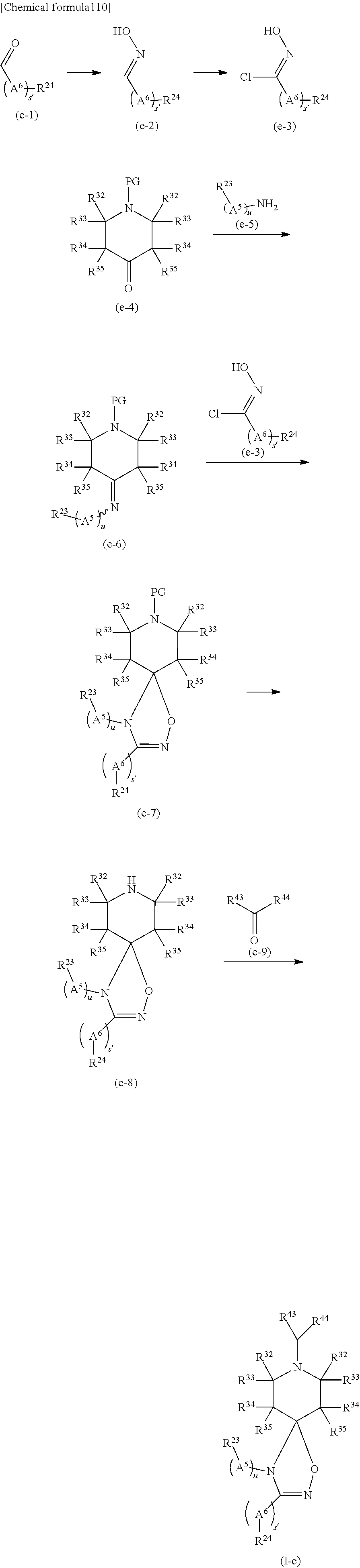

wherein PG is an appropriate protecting group for an amino group such as Boc or Z; R⁴³ and R⁴⁴ are each independently a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl; R⁴³ and R⁴⁴ may be taken together with the identical carbon atom to which they are bonded to form a substituted or unsubstituted non-aromatic carbocycle or a substituted or unsubstituted non-aromatic heterocycle; the other symbols have the same meanings as those in the above item (14).

### Step 1

Compound (e-2) can be obtained by reacting Compound (e-1) with hydroxylamine or hydroxylamine chloride in the presence or absence of a base.

Examples of the base include sodium hydroxide, sodium carbonate, sodium hydrogen carbonate, potassium carbonate, calcium carbonate, cesium carbonate, pyridine, triethylamine, and DMAP, and the base can be used in an amount of 1 to 5 molar equivalents relative to Compound (e-1).

The reaction temperature is −78° C. to the reflux temperature of the solvent, preferably 0 to 25° C.

The reaction time is 0.5 to 48 hours, preferably 1 hour to 6 hours.

Examples of the reaction solvent include water, tetrahydrofuran, DMF, DMA, DMSO, toluene, dichloromethane, chloroform, methanol, and ethanol, and each solvent can be used alone or mixed with the others.

### Step 2

Compound (e-3) can be obtained by adding N-chlorosuccinimide to Compound (e-2).

N-Chlorosuccinimide can be used in an amount of 1 to 10 molar equivalents relative to Compound (e-2).

The reaction temperature is −78° C. to the reflux temperature of the solvent, preferably 0 to 25° C.

The reaction time is 0.5 to 48 hours, preferably 1 hour to 6 hours.

Examples of the reaction solvent include tetrahydrofuran, DMF, DMA, toluene, dichloromethane, and chloroform, and each solvent can be used alone or mixed with the others.

### Step 3

Compound (e-6) can be obtained by condensing Compound (e-4) with Compound (e-5) or a salt thereof in the presence or absence of a condensing agent.

Examples of the condensing agent include anhydrous magnesium sulfate, anhydrous sodium sulfate, titanium tetrachloride, and molecular sieve, and the condensing agent can be used in an amount of 1 to 10 molar equivalents relative to Compound (e-4).

The reaction temperature is −78° C. to the reflux temperature of the solvent, preferably 25 to 120° C.

The reaction time is 0.5 to 48 hours, preferably 1 hour to 6 hours.

Examples of the reaction solvent include tetrahydrofuran, DMF, DMA, DMSO, toluene, dichloromethane, chloroform, methanol, and ethanol, and each solvent can be used alone or mixed with the others.

### Step 4

Compound (e-7) can be obtained by reacting Compound (e-3) with Compound (e-6) in the presence of a base.

Examples of the base include sodium hydroxide, sodium carbonate, sodium hydrogen carbonate, potassium carbonate, calcium carbonate, cesium carbonate, pyridine, triethylamine, and DMAP, and the base can be used in an amount of 1 to 5 molar equivalents relative to Compound (e-6).

The reaction temperature is −78° C. to the reflux temperature of the solvent, preferably 0 to 25° C.

The reaction time is 0.5 to 48 hours, preferably 1 hour to 6 hours.

Examples of the reaction solvent include tetrahydrofuran, DMF, DMA, DMSO, toluene, dichloromethane, chloroform, and water, and each solvent can be used alone or mixed with the others.

### Step 5

Compound (e-8) can be obtained by using Compound (e-7) as a raw material and using the same method as in Step 8 of Method A described above.

### Step 6

Compound (I-e) can be obtained by using Compound (e-8) as a raw material and using the same method as in Step 9 of Method A described above.

### General Synthetic Method 6

### (Method F)

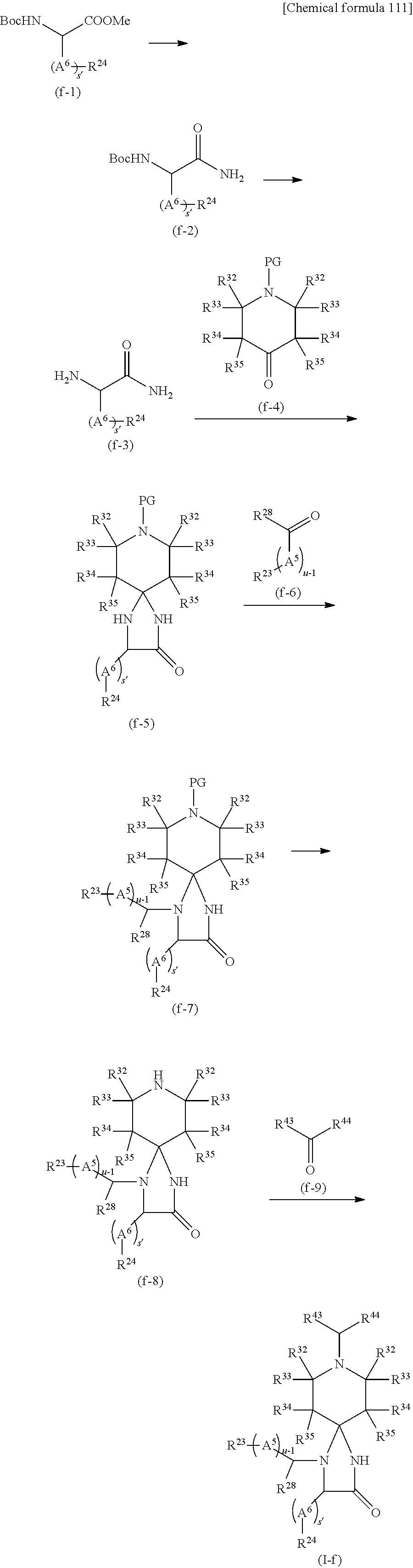

wherein symbols have the same meanings as those in the above Method E.

### Step 1

Compound (f-2) can be obtained by reacting Compound (f-1) with an aqueous ammonia solution.

Ammonia can be used in an amount of 1 to 100 molar equivalents or more relative to Compound (f-1).

Examples of the reaction solvent include methanol, ethanol, DMF, and DMA, and each solvent can be used alone or mixed with the others.

The reaction temperature is −78 to 100° C., preferably 0 to 25° C.

The reaction time is 0.1 to 24 hours, preferably 0.5 to 6 hours.

### Step 2

Compound (f-3) can be obtained by reacting Compound (f-2) in the presence of an acid in the absence of a solvent or in an appropriate solvent.

Examples of the acid include hydrochloric acid, sulfuric acid, TFA, formic acid, and trifluoroborane, and the acid can be used in an amount of 1.0 molar equivalent or more, preferably 1.0 to 30 molar equivalents relative to Compound (f-2).

Examples of the reaction solvent include tetrahydrofuran, diethyl ether, dioxane, dimethoxyethane, chloroform, and dichloromethane, and each solvent can be used alone or mixed with the others.

The reaction temperature is 0 to 80° C., preferably 0 to 20° C.

The reaction time is 0.1 to 24 hours, preferably 0.5 to 6 hours.

### Step 3

Compound (f-5) can be obtained by reacting Compound (f-4) with Compound (f-3) in the presence of a condensing agent.

Examples of the condensing agent include acetic acid, anhydrous magnesium sulfate, and molecular sieve, and the condensing agent can be used in an amount of 0.1 to 10 molar equivalents relative to Compound (f-3).

The reaction temperature is 0 to 150° C., preferably 80 to 120° C.

The reaction time is 0.5 to 48 hours, preferably 1 hour to 6 hours.

Examples of the reaction solvent include 2-propanol, tetrahydrofuran, toluene, DMF, and DMA, and each solvent can be used alone or mixed with the others.

### Step 4

Compound (f-7) can be obtained by condensing Compound (f-5) and Compound (f-6) in the presence or absence of a condensing agent and reducing with a reducing agent.

Examples of the condensing agent include 4-toluenesulfonic acid, methanesulfonic acid, acetic acid, anhydrous magnesium sulfate, tetraisopropyl orthotitanate, titanium tetrachloride, and molecular sieve, and the condensing agent can be used in an amount of 1 to 10 molar equivalents relative to Compound (f-5).

Examples of the reducing agent include sodium borohydride, sodium cyanoborohydride, sodium triacetoxyborohydride, borane and complexes thereof, lithium borohydride, potassium borohydride, and diisobutylaluminum hydride, and the reducing agent can be used in an amount of 1 to 10 molar equivalents relative to Compound (f-5).

The reaction temperature is −78° C. to the reflux temperature of the solvent, preferably 0 to 25° C.

The reaction time is 0.5 to 48 hours, preferably 1 hour to 6 hours.

Examples of the reaction solvent include acetic acid, methanol, ethanol, tetrahydrofuran, dichloromethane, and chloroform, and each solvent can be used alone or mixed with the others.

### Step 5

Compound (f-8) can be obtained by using Compound (f-7) as a raw material and using the same method as in Step 8 of Method A described above.

### Step 6

Compound (I-f) can be obtained by using Compound (f-8) as a raw material and using the same method as in Step 9 of Method A described above.

### General Synthetic Method 7

### (Method G)

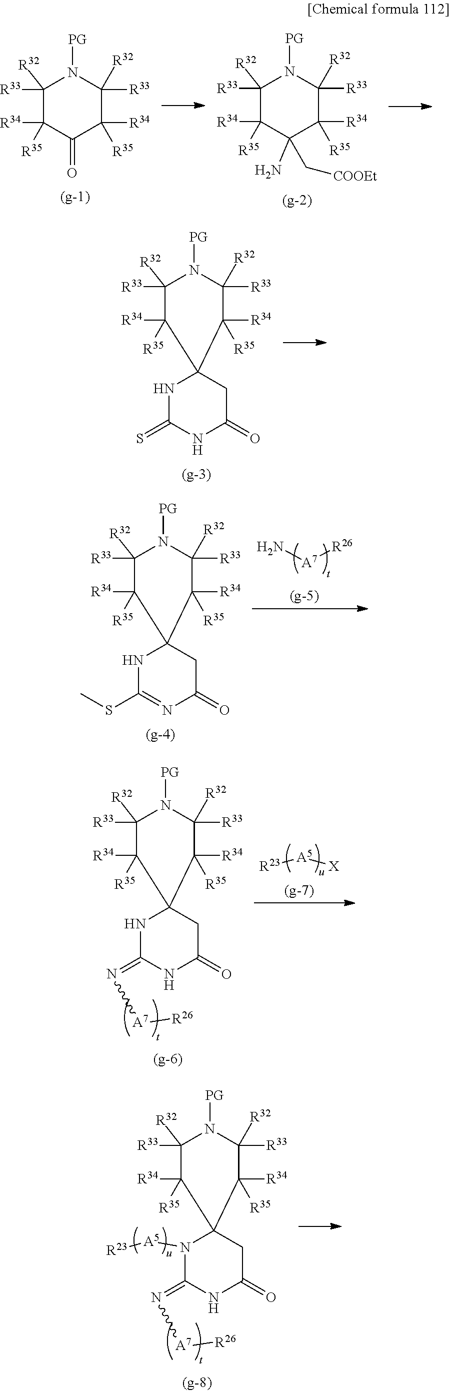

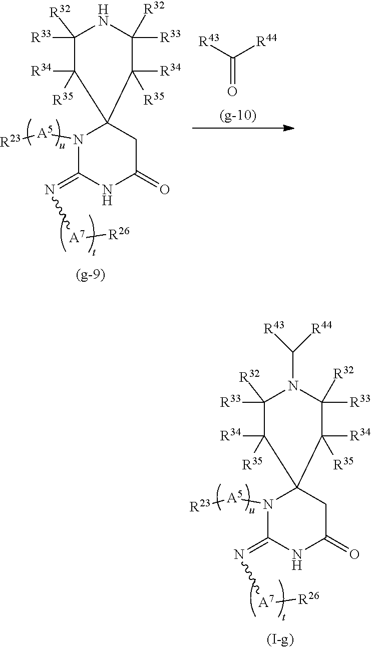

wherein X is a leaving group such as halogen; the other symbols have the same meanings as those in the above Method E.

### Step 1

Compound (g-2) can be obtained by reacting Compound (g-1) with monoethyl malonate and ammonium acetate.

Monoethyl malonate and ammonium acetate can be used in an amount of 1 to 10 molar equivalents or more relative to Compound (g-1).

Examples of the reaction solvent include methanol, ethanol, DMF, and DMA, and each solvent can be used alone or mixed with the others.

The reaction temperature is −78 to 100° C., preferably 60 to 80° C.

The reaction time is 0.1 to 24 hours, preferably 0.5 to 6 hours.

### Step 2

Compound (g-3) can be obtained by reacting Compound (g-2) with benzoyl isothiocyanate, followed by a base.

Benzoyl isothiocyanate can be used in an amount of 1 to 10 molar equivalents relative to Compound (g-2).

Examples of the base include sodium hydroxide, sodium carbonate, sodium hydrogen carbonate, potassium carbonate, calcium carbonate, and cesium carbonate, and the base can be used in an amount of 1 to 5 molar equivalents relative to Compound (g-2).

The reaction temperature is 0 to 150° C., preferably 0 to 80° C.

The reaction time is 0.5 to 48 hours, preferably 1 hour to 6 hours.

Examples of the reaction solvent include dichloromethane, ethanol, 2-propanol, tetrahydrofuran, and toluene, and each solvent can be used alone or mixed with the others.

### Step 3

Compound (g-4) can be obtained by adding methyl iodide to Compound (g-3) in the presence of a base.

Methyl iodide can be used in an amount of 1 to 10 molar equivalents relative to Compound (g-3).

Examples of the base include DIEA and triethylamine, and the base can be used in an amount of 1 to 5 molar equivalents relative to Compound (g-3).

The reaction temperature is −78° C. to the reflux temperature of the solvent, preferably 0 to 25° C.

The reaction time is 0.5 to 48 hours, preferably 1 hour to 6 hours.

Examples of the reaction solvent include methanol, ethanol, tetrahydrofuran, DMF, DMA, toluene, dichloromethane, and chloroform, and each solvent can be used alone or mixed with the others.

### Step 4

Compound (g-6) can be obtained by reacting Compound (g-5) with Compound (g-4).

The reaction temperature is 0 to the reflux temperature of the solvent, preferably 80 to 130° C.

The reaction time is 0.5 to 48 hours, preferably 1 hour to 6 hours.

Examples of the reaction solvent include acetic acid, DMF, DMA, DMSO, tetrahydrofuran, toluene, t-BuOH, and t-amyl alcohol, and each solvent can be used alone or mixed with the others.

### Step 5

Compound (g-8) can be obtained by reacting Compound (g-7) with Compound (g-6) in the presence of a base.

The reaction temperature is 0° C. to 40° C., preferably 0° C. to 20° C.

The reaction time is 0.5 hours to 12 hours, preferably 1 hour to 6 hours.

As the base, sodium carbonate, potassium carbonate, cesium carbonate, lithium hydroxide, sodium hydroxide, potassium hydroxide, sodium hydride, or the like can be used.

Examples of the reaction solvent include methanol, ethanol, water, acetone, acetonitrile, and tetrahydrofuran, and each solvent can be used alone or mixed with the others.

Examples of the reaction solvent include DMF, DMA, DMSO, tetrahydrofuran, dioxane, and acetonitrile, and each solvent can be used alone or mixed with the others.

### Step 5

Compound (g-9) can be obtained by using Compound (g-8) as a raw material and using the same method as in Step 8 of Method A described above.

### Step 6

Compound (I-g) can be obtained by using Compound (g-9) as a raw material and using the same method as in Step 9 of Method A described above.

### General Synthetic Method 8

### (Method H)

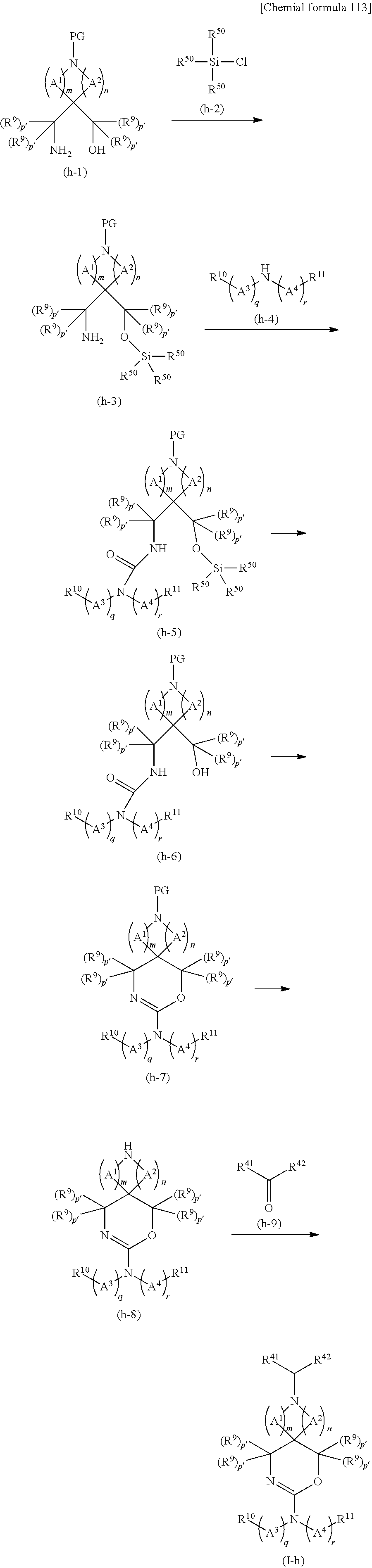

wherein R⁵⁰ is each independently phenyl, tert-butyl, isopropyl, or methyl; p′ is 0 or 1; R⁹ is each independently substituted or unsubstituted alkyl; the other symbols have the same meanings as those in the above Method A.

### Step 1

Compound (h-3) can be obtained by reacting Compound (h-2) with a silylating agent (h-1) in the presence of a base.

Examples of the silylating agent include tert-butyldimethylchlorosilane, triisopropylsilyl chloride, and tert-butyldiphenylchlorosilane, and the silylating agent can be used in an amount of 1 to 10 molar equivalents or more relative to Compound (h-1).

Examples of the base include triethylamine, imidazole, pyridine, and DMAP, and the base can be used in an amount of 1 to 5 molar equivalents relative to Compound (h-1).

Examples of the reaction solvent include dichloromethane, chloroform, DMF, DMA, toluene, and tetrahydrofuran, and each solvent can be used alone or mixed with the others.

The reaction temperature is −78 to 100° C., preferably 0 to 25° C.

The reaction time is 0.1 to 24 hours, preferably 0.5 to 6 hours.

### Step 2

Compound (h-5) can be obtained by reacting Compound (h-3) and Compound (h-4) with an acylating agent in the presence or absence of a base.

Examples of the acylating agent include diphosgene, triphosgene, and CDI, and the acylating agent can be used in an amount of 1 to 10 molar equivalents or more relative to Compound (h-3).

Examples of the base include sodium hydroxide, sodium carbonate, sodium hydrogen carbonate, potassium carbonate, calcium carbonate, cesium carbonate, pyridine, triethylamine, and DMAP, and the base can be used in an amount of 1 to 5 molar equivalents relative to Compound (h-3).

Examples of the reaction solvent include water, ethyl acetate, dichloromethane, and tetrahydrofuran, and each solvent can be used alone or mixed with the others.

The reaction temperature is −78 to 100° C., preferably 0 to 25° C.

The reaction time is 0.1 to 24 hours, preferably 0.5 to 6 hours.

### Step 3

Compound (h-6) can be obtained by allowing a fluoride to act on Compound (h-5).

Examples of the fluoride include TBAF, KF, and pyridinium fluoride, and the fluoride can be used in an amount of 1 to 10 molar equivalents relative to Compound (h-5).

The reaction temperature is 0° C. to the reflux temperature of the solvent, preferably 0 to 25° C.

The reaction time is 0.5 to 48 hours, preferably 1 hour to 6 hours.

Examples of the reaction solvent include tetrahydrofuran, toluene, chloroform, DMF, and DMA, and each solvent can be used alone or mixed with the others.

### Step 4

Compound (h-7) can be obtained by reacting Compound (h-6) with a condensing agent.

The reaction temperature is −78 to 150° C., preferably −78 to 80° C.

Examples of the condensing agent include DAST, dicyclohexylcarbodiimide, carbonyldiimidazole, dicyclohexylcarbodiimide-N-hydroxybenzotriazole, EDC, 4-(4,6-dimethoxy-1,3,5-triazin-2-yl)-4-methylmorpholinium chloride, and HATU, and the condensing agent can be used in an amount of 1 to 5 molar equivalents relative to Compound (h-6). The reaction time is 0.5 to 48 hours, preferably 1 hour to 6 hours.

Examples of the reaction solvent include dichloromethane, ethanol, 2-propanol, tetrahydrofuran, and toluene, and each solvent can be used alone or mixed with the others.

### Step 5

Compound (h-8) can be obtained by using Compound (h-7) as a raw material and using the same method as in Step 8 of Method A described above.

### Step 6

Compound (I-h) can be obtained by using Compound (h-8) as a raw material and using the same method as in Step 9 of Method A described above.

### General Synthetic Method 9

### [Method I]

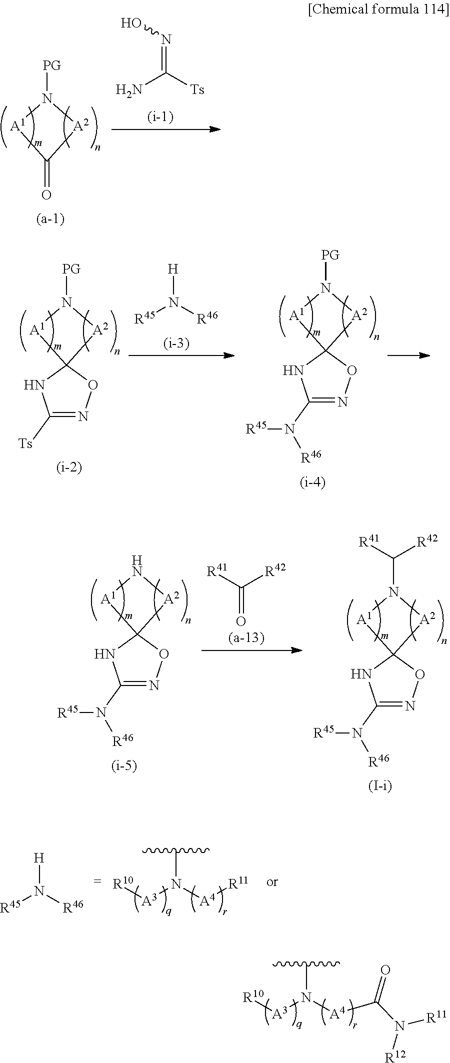

wherein PG is an appropriate protecting group for an amino group such as Boc or Z; R⁴¹ and R⁴² are each independently a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl; R⁴¹ and R⁴² may be taken together with the identical carbon atom to which they are bonded to form a substituted or unsubstituted non-aromatic carbocycle or a substituted or unsubstituted non-aromatic heterocycle; the other symbols have the same meanings as those in the above item (1).

### Step 1

Compound (i-2) can be obtained by reacting Compound (i-1) with Compound (a-1) in the presence of an acid.

The reaction temperature is 30° C. to 150° C., preferably 100° C. to 130° C.

The reaction time is 0.5 hours to 12 hours, preferably 1 hour to 6 hours.

Examples of the acid include hydrochloric acid, sulfuric acid, TFA, formic acid, trifluoroborane, p-TsOH, and PPTS, and the acid can be used in an amount of 0.1 molar equivalents or more, preferably 0.1 to 1 molar equivalents relative to Compound (a-1).

Examples of the reaction solvent include methanol, ethanol, 2-propanol, t-butyl alcohol, water, acetone, acetonitrile, tetrahydrofuran, and dioxane, and each solvent can be used alone or mixed with the others.

### Step 2

Compound (i-4) can be obtained by reacting Compound (i-3) with Compound (i-2) in the presence of a base.

The reaction temperature is 30° C. to 150° C., preferably 100° C. to 130° C.

The reaction time is 1 hour to 24 hours, preferably 3 hours to 9 hours.

Examples of the base include pyridine, triethylamine, DIPEA, and DMAP, and the base can be used in an amount of 1 to 5 molar equivalents relative to Compound (i-2).

Examples of the reaction solvent include DMF, DMA, DMSO, tetrahydrofuran, dioxane, and acetonitrile, and each solvent can be used alone or mixed with the others.

### Step 3

Compound (i-5) can be obtained by using Compound (i-4) as a raw material and using the same method as in Step 8 of Method A described above.

### Step 4

Compound (I-i) can be obtained by using Compound (i-5) as a raw material and using the same method as in Step 9 of Method A described above.

Since the compound according to the present invention has serotonin 5-HT2A receptor antagonism and/or inverse agonism, the compound is useful as a therapeutic and/or prophylactic agent for Parkinson's disease- and/or dementia-related hallucinations and delusions.

Furthermore, the compound according to the present invention has utility as a medicine, and preferably, the compound has any one or a plurality of the following excellent features.
a) Inhibitory activity against CYP enzymes (for example, CYP1A2, CYP2C9, CYP2C19, CYP2D6, and CYP3A4) is weak.
b) Satisfactory pharmacokinetics such as high bioavailability and adequate clearance are exhibited.
c) Metabolic stability is high.
d) Irreversible inhibitory activity is not exhibited against CYP enzymes (for example, CYP3A4) within the concentration range of the measurement conditions described in the present description.
e) Mutagenicity is not exhibited.
f) The cardiovascular risk is low.
g) High solubility is exhibited.
h) High binding ability for a serotonin 5-HT2A receptor is exhibited.
i) High binding ability for a serotonin 5-HT2C receptor is exhibited.
j) Brain distribution ability is high.
k) P-gp substrate property is low.

A pharmaceutical composition of the present invention can be administered orally or parenterally. Methods for parenteral administration include dermal, subcutaneous, intravenous, intraarterial, intramuscular, intraperitoneal, transmucosal, inhalation, transnasal, ophthalmic, inner ear or vaginal administration.

In the case of oral administration of pharmaceutical composition of the present invention, any forms, which are usually used, such as oral solid formulations (e.g., tablets, powders, granules, capsules, pills, or films), and oral liquid formulations (e.g., suspension, emulsion, elixir, syrup, lemonade, spirit, aromatic water, extract, decoction, or tincture) may be prepared according to the usual method and administered. The tablets can be sugar-coated tablets, film-coated tablets, enteric-coating tablets, sustained-release tablets, troche tablets, sublingual tablets, buccal tablets, chewable tablets or orally disintegrating tablets. Powders and granules can be dry syrups. Capsules can be soft capsules, micro capsules or sustained-release capsules.

In the case of parenteral administration of pharmaceutical composition of the present invention, any forms, which are usually used, such as injections, drips, and external preparations (e.g., ophthalmic drops, nasal drops, ear drops, aerosols, inhalations, lotion, infusion, liniment, mouthwash, enema, ointment, plaster, jelly, cream, patch, cataplasm, external powder, or suppository) can be preferably administered. Injections can be emulsions whose type is O/W, W/O, O/W/O, W/O/W or the like.

A pharmaceutical composition can be obtained by mixing an effective amount of the compound according to the present invention with various pharmaceutical additives appropriate for the dosage form, such as an excipient, a binder, a disintegrating agent, and a lubricating agent, as necessary. Furthermore, the pharmaceutical composition can be prepared into a pharmaceutical composition for use for a child, an elderly, a patient with a serious case, or a surgical operation, by appropriately changing the effective amount of the compound according to the present invention, the dosage form, and/or various pharmaceutical additives. For example, a pharmaceutical composition for use for a child may be administered to a neonate (less than 4 weeks after birth), an infant (from 4 weeks after birth to less than 1 year), a preschool child (from 1 year to less than 7 years), a child (from 7 years to less than 15 years), or a patient 15 years to 18 years of age. For example, a pharmaceutical composition for an elderly may be administered to a patient 65 years of age or older.

It is desirable to set the amount of administration of the pharmaceutical composition of the present invention, after considering the age and body weight of the patient, the type and degree of the disease, the route of administration, and the like; however, in the case of oral administration, the amount of administration is usually 0.05 to 100 mg/kg/day and is preferably in the range of 0.1 to 10 mg/kg/day. In the case of parenteral administration, the amount of administration may vary greatly depending on the route of administration; however, the amount of administration is usually 0.005 to 10 mg/kg/day and is preferably in the range of 0.01 to 1 mg/kg/day. This may be administered once a day or several times a day.

The compound according to the present invention can be used in combination with another therapeutic agent for Parkinson's disease, Alzheimer's disease, psychosis or depression (hereinafter, referred to as concomitant drug), for the purpose of enhancing the action of the compound, reducing the amount of administration of the compound, or the like. At this time, the timing of administration for the compound according to the present invention and the concomitant drug is not limited, and these may be administered simultaneously to the target of administration or may be administered with a time difference. Furthermore, the compound according to the present invention and the concomitant drug may be administered as two or more kinds of preparations each including active ingredients, or may be administered as a single preparation including those active ingredients.

The amount of administration of the concomitant drug can be appropriately selected based on the clinically used dosage. Furthermore, the blending ratio of the compound according to the present invention and the concomitant drug can be appropriately selected according to the target of administration, the route of administration, the target disease, symptoms, combination, and the like. For example, when the target of administration is a human being, 0.01 to 100 parts by weight of the concomitant drug may be used with respect to 1 part by weight of the compound according to the present invention.

Examples of the therapeutic agent for Parkinson's disease include levodopa preparations.

Examples of the therapeutic agent for Alzheimer's disease include donepezil.

Examples of the therapeutic agent for psychosis include quetiapine.

Examples of the therapeutic agent for depression include escitalopram.

### EXAMPLES

Hereinafter, the present invention will be described in more detail by way of Examples, Reference Examples, and Test Examples; however, the present invention is not intended to be limited by these.

Furthermore, abbreviations used in the present description denote the following meanings.
CDCl₃: Deuterated chloroform
DMSO-D6: Deuterated dimethyl sulfoxide
Boc: tert-Butoxycarbonyl
Z: Benzyloxycarbonyl
Cbz: Benzyloxycarbonyl
SEM: 2-(Trimethylsilyl)ethoxymethyl
DMF: N,N-Dimethylformamide
DMSO: Dimethyl sulfoxide
NMP: N-Methylpyrrolidone
DMA: N,N-Dimethylacetamide
NMM: N-Methylmorpholine
T₃P: 2,4,6-Tripropyl-1,3,5,2,4,6-trioxatriphosphorinane-2,4,6-trioxide
CDI: Carbonyldiimidazole
MsCl: Methanesulfonyl chloride
TsCl: p-Toluenesulfonyl chloride
TBAF: Tetrabutylammonium fluoride
KF: Potassium fluoride
DMAP: 4-Dimethylaminopyridine
TFA: Trifluoroacetic acid
DIEA: N,N-Diisopropylethylamine
CDI: Carbonyldiimidazole
EDC: 1-Ethyl-3-(3-dimethylaminopropyl)carbodiimide
HATU: O-(7-Azabenzotriazol-1-yl)-1,1,3,3-tetramethyluronium hexafluorophosphate
DAST: N,N-Diethylaminosulfur trifluoride
THF: Tetrahydrofuran
DIAD: Diisopropyl azodicarboxylate
DIPEA: N,N-Diisopropylethylamine
TBS: tert-Butyldimethylsilyl
PPTS: Pyridinium p-toluenesulfonate

### (Method for Identifying Compound)

The NMR analysis obtained in each Example was performed at 400 MHz, and measurement was made using DMSO-d₆, CDCl₃. Furthermore, when NMR data are shown, there are occasions in which all the measured peaks are not described. The term RT in the description indicates retention time in an LC/MS: liquid chromatography/mass analysis, and the retention time was measured under the following conditions.

### (Measurement Condition 1)

Column: Shim-pack XR-ODS (2.2 μm, i.d. 3.0×50 mm) (Shimadzu)

Flow rate: 1.6 mL/min

UV detection wavelength: 254 nm

Mobile phase: [A] was 0.1% formic acid-containing aqueous solution, and [B] was 0.1% formic acid-containing acetonitrile solution.

Gradient: A linear gradient of 10% to 100% solvent [B] was carried out for 3 minutes, and then 100% solvent [B] was maintained for 0.5 minutes.

### (Measurement Condition 2)

Column: ACQUITY UPLC (registered trademark) BEH C18 (1.7 μm i.d. 2.1×50 mm) (Waters)

Flow rate: 0.8 mL/min

UV detection wavelength: 254 nm

Mobile phase: [A] was 0.1% formic acid-containing aqueous solution, and [B] was 0.1% formic acid-containing acetonitrile solution.

Gradient: A linear gradient of 5% to 100% solvent [B] was carried out for 3.5 minutes, and then 100% solvent [B] was maintained for 0.5 minutes.

### (Measurement Condition 3)

Column: ACQUITY UPLC (registered trademark) BEH C18 (1.7 μm i.d. 2.1×50 mm)

### (Waters)

Flow rate: 0.8 mL/min

UV detection wavelength: 254 nm

Mobile phase: [A] was 10 mM ammonium carbonate-containing aqueous solution, and [B] was acetonitrile.

Gradient: A linear gradient of 5% to 100% solvent [B] was carried out for 3.5 minutes, and then 100% solvent [B] was maintained for 0.5 minutes.

Incidentally, in the description, the description of MS(m/z) indicates a value observed by mass analysis.

### Example 1

### Synthesis of Compound (I-009)

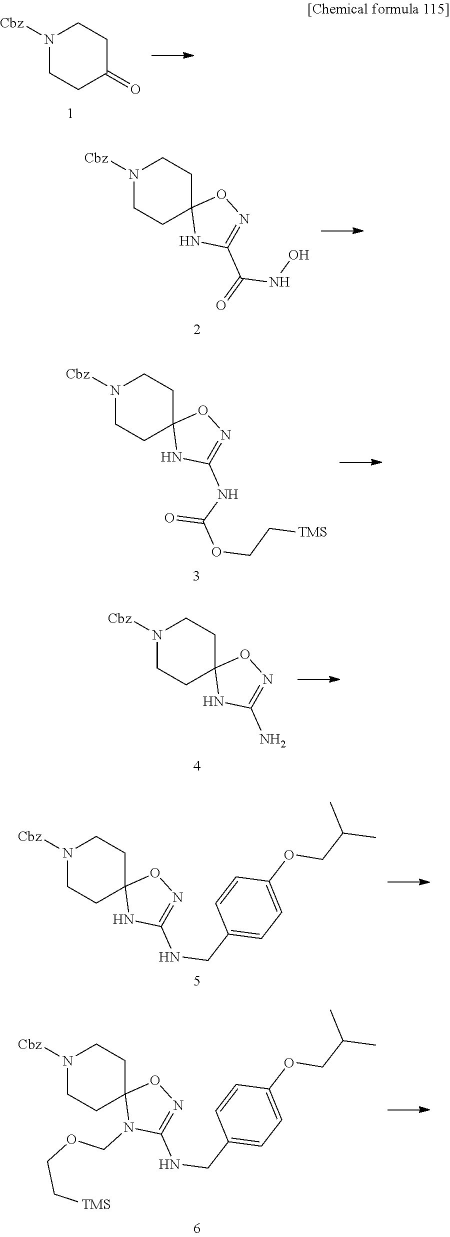 [SMILES: O=C1CCN(C(=O)OCc2ccccc2)CC1 ; O=C(NO)C1=NOC2(CCN(C(=O)OCc3ccccc3)CC2)N1 ; C[Si](C)(C)CCOC(=O)NC1=NOC2(CCN(C(=O)OCc3ccccc3)CC2)N1 ; NC1=NOC2(CCN(C(=O)OCc3ccccc3)CC2)N1 ; CC(C)COc1ccc(CNC2=NOC3(CCN(C(=O)OCc4ccccc4)CC3)N2)cc1 ; CC(C)COc1ccc(CNC2=NOC3(CCN(C(=O)OCc4ccccc4)CC3)N2COCC[Si](C)(C)C)cc1]

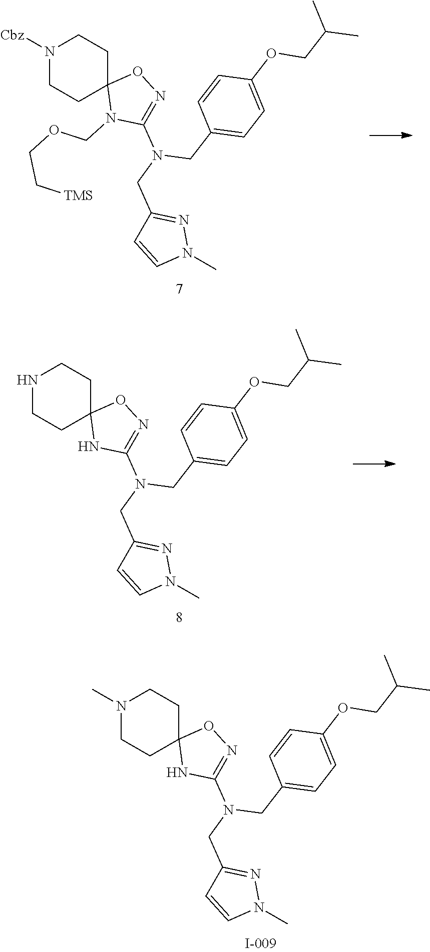

### Step 1 Synthesis of Compound 2

Ethyl aminohydroxyiminoacetate (25.0 g, 189 mmol) and Compound 1 (40.1 g, 172 mmol) were dissolved in 2-propanol (250 mL), and pyridinium p-toluenesulfonate (8.65 g, 34.4 mmol) was added, then the mixture was stirred at 100° C. for 5 hours. The mixture was allowed to cool to room temperature under stirring, and water (750 mL) was added to the obtained suspension, then the mixture was stirred for 30 minutes. The precipitated solid was collected by filtration, washed three times with 2-propanol/water (1:3) (50 mL), and then air-dried overnight to afford 137 g of a white solid. The obtained solid was suspended in 2-propanol (200 mL), a 50% aqueous solution of hydroxylamine (114 g, 1720 mmol) was added thereto, and the mixture was stirred at 75° C. for 10 minutes. 2-Propanol (100 mL) was added, and the mixture was stirred at room temperature for 30 minutes. The precipitated solid was collected by filtration and washed with 2-propanol (150 mL). The obtained solid was dried under reduced pressure under heating to afford Compound 2 (29.6 g, yield 52%) as a white solid.

1H-NMR (DMSO-D6) δ: 1.59-1.79 (m, 4H), 3.17-3.46 (m, 2H), 3.61-3.81 (m, 2H), 5.07 (s, 2H), 5.60 (s, 1H), 6.41 (s, 1H), 7.25-7.64 (m, 6H).

### Step 2 Synthesis of Compound 3

Compound 2 (8.61 g, 25.8 mmol) was suspended in THF (86 mL), N-methylmorpholine (7.08 mL, 64.4 mmol) was added to the suspension, then a 50% 2,4,6-tripropyl-1,3,5,2,4,6-trioxatriphosphorinane-2,4,6-trioxide/ethyl acetate solution (38.3 mL, 64.4 mmol) was added to the suspension over about 7 minutes under ice cooling, and the mixture was stirred at room temperature for 1 hour. 2-(Trimethylsilyl)ethanol (18.4 mL, 129 mmol) was added, and the mixture was stirred at 60° C. for 100 minutes. A 20% aqueous solution of potassium carbonate (170 mL) was added, then the mixture was extracted with ethyl acetate, the organic layer was washed with water, and the solvent was distilled off under reduced pressure. The obtained solid was suspended in methanol, then collected by filtration, and dried under reduced pressure under heating to afford Compound 3 (6.54 g, yield 58%) as a white solid.

1H-NMR (DMSO-D6) δ: 0.00 (s, 9H), 0.95 (t, J=8.4 Hz, 2H), 1.64-1.79 (m, 4H), 2.87-3.15 (m, 3H), 3.75-3.89 (m, 2H), 4.13 (t, J=8.4 Hz, 2H), 5.05 (s, 2H), 7.23-7.41 (m, 5H), 10.29 (s, 1H).

### Step 3 Synthesis of Compound 4

Compound 3 (5.51 g, 12.9 mmol) was suspended in THF (12.7 mL), and a 1 mol/L tetrabutylammonium fluoride/THF solution (15.2 mL, 15.2 mmol) was added, then the mixture was heated and refluxed for 2 hours. A 1 mol/L tetrabutylammonium fluoride/THF solution (3.80 mL, 3.80 mmol) was added again, and the mixture was heated and refluxed for 1.5 hours. 2-Propanol (50 mL) was added, and the solvent was distilled off under reduced pressure until reaching 16.2 g. 2-Propanol (20 mL) was added again, and the solvent was distilled off under reduced pressure until reaching 18.7 g. 2-Propanol (10 mL) was added, and the precipitated solid was collected by filtration, and dried under reduced pressure under heating to afford Compound 4 (3.34 g, yield 91%) as a white solid.

### Step 4 Synthesis of Compound 5

Compound 4 (3.00 g, 10.3 mmol) was suspended in THF (30 mL), and 4-isobutoxybenzaldehyde (2.39 g, 13.4 mmol) and tetraisobutoxytitanium (7.57 mL, 25.8 mmol) were added, then the mixture was stirred under heat-reflux for 6 hours. After the temperature was adjusted to 40° C., THF (30 mL) and sodium triacetoxyborohydride (8.76 g, 41.3 mmol) were added, and the mixture was stirred at that temperature for 2 hours. A 20% aqueous solution of citric acid (60 mL) was added to the reaction solution, and then the mixture was stirred for 10 minutes. Chloroform (50 mL) was added, then the mixture was made weakly basic (pH 9) with a 20% aqueous solution of potassium carbonate (240 mL), and the organic solvent was distilled off under reduced pressure. The residue was extracted with chloroform/methanol (3:1, 600 mL) and chloroform (150 mL). The organic layers were combined, and the solvent was distilled off under reduced pressure to afford a yellow solid. This solid was suspended in methanol (100 mL), water (20 mL) was added, and the mixture was collected by filtration. The solid was washed with a 90% aqueous solution of methanol and then dried under reduced pressure under heating to afford Compound 5 (2.42 g, yield 52%) as a yellow solid.

1H-NMR (DMSO-D6) δ: 0.96 (d, J=6.5 Hz, 6H), 1.51-1.75 (m, 4H), 1.92-2.07 (m, 1H), 3.20-3.48 (m, 2H), 3.58-3.68 (m, 2H), 3.71 (d, J=6.5 Hz, 2H), 4.01 (d, J=6.0 Hz, 2H), 5.07 (s, 2H), 6.07 (t, J=6.1 Hz, 1H), 6.34 (s, 1H), 6.86 (d, J=8.5 Hz, 2H), 7.20 (d, J=8.5 Hz, 2H), 7.27-7.47 (m, 5H).

### Step 5 Synthesis of Compound 6

Compound 5 (2.42 g, 5.36 mmol) was dissolved in DMF (24 mL), and 2-(chloromethoxy)ethyltrimethylsilane (1.05 mL, 5.89 mmol) and cesium carbonate (2.62 g, 8.03 mmol) were added, then the mixture was stirred at room temperature for 3 hours. N-methylpiperazine (1.79 mL, 16.1 mmol) was added, and the mixture was stirred at room temperature for 15 minutes. Water (100 mL) was added to the reaction solution, and the mixture was extracted with ethyl acetate (100 mL). The organic layer was washed with water, and the solvent was distilled off under reduced pressure. The obtained residue was purified by silica gel column chromatography (hexane-ethyl acetate) to afford Compound 6 (3.03 g, yield 97%) as a colorless oil.

1H-NMR (CDCl3) δ: 0.00 (s, 9H), 0.92 (t, J=8.4 Hz, 2H), 1.01 (d, J=6.8 Hz, 6H), 1.69-1.91 (m, 4H), 2.02-2.12 (m, 1H), 3.38-3.51 (m, 2H), 3.63-3.75 (m, 4H), 3.75-3.86 (m, 2H), 4.12 (t, J=5.3 Hz, 1H), 4.34 (d, J=5.3 Hz, 2H), 4.55 (s, 2H), 5.13 (s, 2H), 6.85 (d, J=8.5 Hz, 2H), 7.23 (d, J=8.5 Hz, 2H), 7.28-7.38 (m, 5H).

### Step 6 Synthesis of Compound 7

Compound 6 (3.03 g, 5.20 mmol) was dissolved in DMF (15 mL) and THF (15 mL), and sodium hydride (0.624 g, 15.6 mmol) was added under ice cooling, then the mixture was stirred at room temperature for 10 minutes. 3-(Chloromethyl)-1-methyl-1H-pyrazole hydrochloride (1.04 g, 6.24 mmol) was added, and the mixture was stirred at room temperature for 17 hours. Sodium hydride (0.416 g, 10.4 mmol) was added again, and then the mixture was stirred at 50° C. for 2 hours and at 70° C. for 4 hours. Water (100 mL) was added to the reaction solution, and the mixture was extracted with ethyl acetate (200 mL). The organic layer was washed with water, and the solvent was distilled off under reduced pressure. The obtained residue was purified by silica gel column chromatography (hexane-ethyl acetate) to afford Compound 7 (2.60 g, yield 74%) as a yellow oil.

1H-NMR (CDCl3) δ: −0.01 (s, 9H), 0.93 (t, J=8.4 Hz, 2H), 1.02 (d, J=6.8 Hz, 6H), 1.72-1.91 (m, 4H), 1.99-2.15 (m, 1H), 3.53-3.76 (m, 8H), 3.86 (s, 3H), 4.31 (s, 2H), 4.32 (s, 2H), 4.67 (s, 2H), 5.14 (s, 2H), 6.19 (d, J=2.3 Hz, 1H), 6.83 (d, J=8.5 Hz, 2H), 7.20 (d, J=8.5 Hz, 2H), 7.27-7.39 (m, 6H).

### Step 7 Synthesis of Compound 8

Compound 7 (2.60 g, 3.83 mmol) was dissolved in methylene chloride (26 mL), and a boron trifluoride diethyl ether complex (4.86 mL, 38.3 mmol) and dimethyl sulfide (8.51 mL, 115 mmol) were added, then the mixture was stirred at 40° C. for 2 hours. A 20% aqueous solution of potassium carbonate (100 mL) was added, the mixture was extracted with ethyl acetate (100 mL), the organic layer was washed with water, and the solvent was distilled off under reduced pressure. The obtained residue was purified by aminosilica gel column chromatography (chloroform-methanol) to afford Compound 8 (765 mg, yield 48%) as a white foam.

1H-NMR (CDCl3) δ: 1.02 (d, J=6.8 Hz, 6H), 1.71-1.80 (m, 2H), 1.88-1.98 (m, 2H), 2.00-2.15 (m, 1H), 2.85-2.94 (m, 2H), 2.98-3.10 (m, 2H), 3.71 (d, J=6.5 Hz, 2H), 3.85 (s, 3H), 4.15 (s, 2H), 4.30 (s, 2H), 5.42 (br s, 1H), 6.01 (d, J=2.3 Hz, 1H), 6.86 (d, J=8.8 Hz, 2H), 7.24 (d, J=8.5 Hz, 2H), 7.25-7.28 (m, 1H).

### Step 8 Synthesis of Compound (I-009)

Compound 9 (765 mg, 1.85 mmol) was dissolved in ethanol (7.65 mL), a 37% aqueous solution of formaldehyde (0.690 mL) and sodium triacetoxyborohydride (1.18 g, 5.56 mmol) were added, and the mixture was stirred at room temperature for 3 hours. A 20% aqueous solution of potassium carbonate (20 mL) was added, then the mixture was extracted with ethyl acetate (40 mL), the organic layer was washed with water, and the solvent was distilled off under reduced pressure. The obtained residue was purified by diol silica gel column chromatography (chloroform-methanol) to afford Compound (I-009) (320 mg, yield 41%) as a colorless oil.

1H-NMR (CDCl3) δ: 1.02 (d, J=6.8 Hz, 6H), 1.78-1.91 (m, 2H), 1.96-2.14 (m, 3H), 2.32 (s, 3H), 2.44-2.70 (m, 4H), 3.71 (d, J=6.5 Hz, 2H), 3.85 (s, 3H), 4.14 (s, 2H), 4.30 (s, 2H), 5.41 (br s, 1H), 6.00 (d, J=1.8 Hz, 1H), 6.86 (d, J=8.5 Hz, 2H), 7.23-7.29 (m, 1H), 7.24 (d, J=8.5 Hz, 2H).

### Example 2

### Synthesis of Compound (I-002)

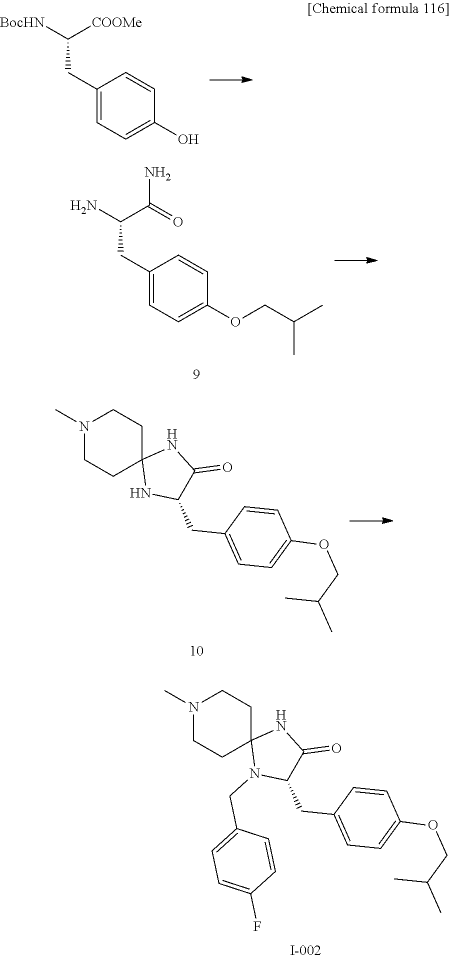

### Step 1 Synthesis of Compound 9

N-(tert-butoxycarbonyl)-L-tyrosine methyl (15.0 g, 50.8 mmol) was dissolved in methanol (75 mL), and a 30% aqueous solution of ammonia (75 mL) was added dropwise over about 2 minutes under ice cooling. The mixture was allowed to stand at room temperature for 10 days, and then methanol was distilled off under reduced pressure. The obtained suspension was extracted with ethyl acetate, then the organic layer was washed with water, the solvent was distilled off under reduced pressure, and dehydration azeotropy with ethyl acetate was performed twice to afford about 37 g of a white solid. This white solid was dissolved in tetrahydrofuran (225 mL), triphenylphosphine (4.00 g, 15.2 mmol) and isobutanol (9.41 mL, 102 mmol) were added, and then DIAD (11.9 mL, 60.9 mmol) was added dropwise over about 3 minutes under ice cooling. Thereafter, the temperature was raised to 50° C. over 30 minutes, and the mixture was stirred at that temperature for 2 hours. Triphenylphosphine (4.00 g, 15.2 mmol), isobutanol (4.71 mL, 50.8 mmol), and DIAD (2.96 mL, 15.2 mmol) were added again, and the mixture was stirred at 50° C. for 30 minutes. The solvent was distilled off from the reaction solution under reduced pressure to 104 g, then water (7.5 mL) and ethanol (150 mL) were added, and the solvent was distilled off under reduced pressure again to afford about 87 g of a residue. Ethanol (75 mL) was added, and the solvent was distilled off under reduced pressure twice to afford about 83 g of a residue. Ethanol (225 mL) and water (225 mL) were added to this residue, and the obtained suspension was filtered. The mud collected by filtration was washed with a 50% aqueous solution of ethanol (30 mL) four times to afford about 60 g of white mud. This mud was dissolved in 1,4-dioxane (120 mL) and ethanol (60 mL), concentrated hydrochloric acid (31.7 mL) was added, and the mixture was stirred at room temperature for 14 hours and at 50° C. for 30 minutes. Under ice cooling, an 8 mol/L aqueous solution of sodium hydroxide (45 mL) was added for neutralization, and the solvent was distilled off under reduced pressure to about 100 g. Methanol (50 mL) was added to the obtained suspension, and a solid was separated by filtration. A 20% aqueous solution of potassium carbonate (5 mL) was added to the filtrate, and the mixture was extracted with ethyl acetate. The organic layer was washed with water, and the solvent was distilled off under reduced pressure. The obtained residue was purified by aminosilica gel column chromatography (ethyl acetate-methanol) to afford Compound 9 (5.22 g, yield 43%) as a white solid.

¹H-NMR (CDCl₃) δ: 0.96 (d, J=6.7 Hz, 6H), 1.92-2.04 (m, 1H), 2.52 (dd, J=11.5, 9.8 Hz, 1H), 2.82 (dd, J=13.4, 5.1 Hz, 1H), 3.26 (dd, J=8.1, 5.2 Hz, 1H), 3.69 (d, J=6.5 Hz, 2H), 6.82 (d, J=8.5 Hz, 2H), 6.92 (s, 1H), 7.10 (d, J=8.5 Hz, 2H), 7.27 (s, 1H).

### Step 2 Synthesis of Compound 10

Compound 9 (500 mg, 2.12 mmol), acetic acid (0.242 mL, 0.423 mmol), and 1-methylpiperidin-4-one (479 mg, 4.23 mmol) were dissolved in 2-propanol (2.5 mL), and the mixture was stirred at 100° C. for 2 hours. A 20% aqueous solution of potassium carbonate was added to the reaction solution, and then the mixture was extracted with ethyl acetate. The organic layer was washed with water, and the solvent was distilled off under reduced pressure. The obtained residue was purified by aminosilica gel column chromatography (ethyl acetate-methanol) to afford Compound 10 (515 mg, yield 73%).

¹H-NMR (CDCl₃) δ: 1.02 (d, J=6.7 Hz, 6H), 1.34-1.82 (m, 4H), 2.03-2.12 (m, 1H), 2.17-2.56 (m, 2H), 2.27 (s, 3H), 2.47 (t, J=6.2 Hz, 1H), 2.72 (t, J=6.1 Hz, 1H), 2.95-3.07 (m, 2H), 3.70 (d, J=6.5 Hz, 2H), 3.80 (t, J=5.3 Hz, 1H), 5.95 (s, 1H), 6.84 (d, J=8.7 Hz, 2H), 7.15 (d, J=8.7 Hz, 2H).

### Step 3 Synthesis of Compound (I-002)

Compound 10 (200 mg, 0.603 mmol) and 4-fluorobenzaldehyde (0.127 mL, 1.21 mmol) were dissolved in acetic acid (1 mL), and the mixture was stirred at room temperature for 15 minutes. Then, sodium triacetoxyborohydride (192 mg, 0.905 mmol) was added, and the mixture was stirred at room temperature for 8 hours. Sodium triacetoxyborohydride (192 mg, 0.905 mmol) was added again, and the mixture was stirred at room temperature for 8 hours. A 20% aqueous solution of potassium carbonate was added, and then the mixture was extracted with ethyl acetate. The organic layer was washed with water, and the solvent was distilled off under reduced pressure. The obtained residue was purified by aminosilica gel column chromatography (hexane-ethyl acetate) to afford 113 mg of a white solid. This solid was suspended in 20% ethyl acetate/hexane and collected by filtration to afford Compound (I-002) (75.3 mg, 28%) as a white solid.

¹H-NMR (CDCl₃) δ: 1.01 (d, J=6.8 Hz, 6H), 1.08-1.16 (m, 1H), 1.52-1.69 (m, 3H), 1.76-1.86 (m, 1H), 1.90-1.99 (m, 1H), 2.00-2.13 (m, 2H), 2.22 (s, 3H), 2.68-2.78 (m, 2H), 2.85-2.93 (m, 2H), 3.65-3.71 (m, 3H), 3.96 (d, J=14.4 Hz, 1H), 6.50 (s, 1H), 6.73 (d, J=8.7 Hz, 2H), 6.94-7.03 (m, 4H), 7.24 (dd, J=8.5, 5.6 Hz, 2H).

### Example 3

### Synthesis of Compound (I-005)

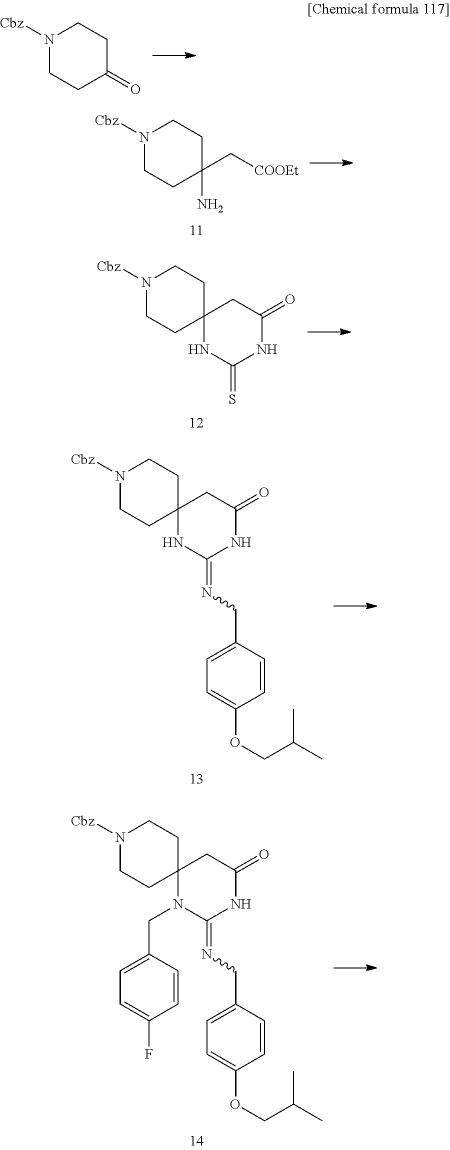

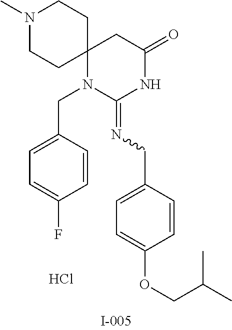 [SMILES: CC(C)COc1ccc(CN=C2NC(=O)CC3(CCN(C)CC3)N2Cc2ccc(F)cc2)cc1 ; Cl]

### Step 1 Synthesis of Compound 11

Monoethyl malonate (6.80 g, 51.4 mmol), benzyl 4-oxo-1-piperidinecarboxylate (10.0 g, 42.9 mmol), and ammonium acetate (4.96 g, 64.3 mmol) were dissolved in ethanol (50 mL), and the mixture was stirred under heat-reflux for 3 hours. Ethyl acetate was added, the reaction solution was washed with a 20% aqueous solution of potassium carbonate and water, and the solvent was distilled off under reduced pressure. Compound 11 (13.8 g, 100%) was obtained as a yellow oil.

1H-NMR (CDCl3) δ: 1.26 (t, J=7.2 Hz, 3H), 1.47-1.80 (m, 4H), 2.40 (s, 2H), 3.34-3.46 (m, 2H), 3.66-3.79 (m, 2H), 4.15 (q, J=7.2 Hz, 2H), 5.12 (s, 2H), 7.29-7.42 (m, 5H).

### Step 2 Synthesis of Compound 12

Compound 11 (7.00 g, 17.5 mmol) was dissolved in methylene chloride (35 mL), and benzoyl isothiocyanate (2.82 mL, 21.0 mmol) was added dropwise under ice cooling. After stirring at room temperature for 20 minutes, the solvent was distilled off under reduced pressure. This residue was dissolved in ethanol (35 mL), and potassium carbonate (4.83 g, 35.0 mmol) was added, then the mixture was stirred at 70° C. for 20 minutes and at 90° C. for 60 minutes. The pH was adjusted to 4 with a 20% aqueous solution of sodium dihydrogen phosphate, and then the mixture was extracted with ethyl acetate. The organic layer was washed with water, and the solvent was distilled off under reduced pressure. The obtained residue was purified by silica gel column chromatography (hexane-ethyl acetate) to afford Compound 12 (5.37 g, yield 61%) as a white solid.

¹H-NMR (CDCl₃) δ: 1.58-1.90 (m, 4H), 2.65 (s, 2H), 3.50-3.71 (m, 4H), 5.14 (s, 2H), 7.31-7.42 (m, 5H), 7.46 (br s, 1H), 8.68 (br s, 1H).

### Step 3 Synthesis of Compound 13

Compound 12 (100 mg, 0.300 mmol) was dissolved in DMF (1 mL), and methyl iodide (0.0563 mL, 0.900 mmol) was added, then the mixture was stirred at room temperature for 1 hour. A 5% aqueous solution of sodium hydrogen carbonate was added to the reaction solution, and then the mixture was extracted with ethyl acetate. The organic layer was washed with water and brine, and dried over sodium sulfate. Thereafter, the solid was filtered off, and the solvent was distilled off under reduced pressure. The obtained residue was dissolved in t-amyl alcohol (1 mL), and 4-isobutoxybenzylamine (108 mg, 0.600 mmol) and DIPEA (0.262 mL, 1.50 mmol) were added, then the mixture was stirred at 100° C. for 4 hours. Ethyl acetate was added to the reaction solution, the mixture was washed with a 20% aqueous solution of citric acid and water, and the solvent was distilled off under reduced pressure. The obtained residue was purified by diol silica gel column chromatography (chloroform-methanol) and aminosilica gel column chromatography (chloroform-methanol) to afford Compound 13 (65.9 mg, yield 46%).

1H-NMR (DMSO-D6) δ: 0.93-0.98 (m, 6H), 1.27-1.42 (m, 0.72H), 1.46-1.60 (m, 3.28H), 1.91-2.06 (m, 1H), 2.19 (s, 0.36H), 2.27 (s, 1.64H), 3.27-3.74 (m, 6H), 4.20 (d, J=6.3 Hz, 0.36H), 4.31 (d, J=5.8 Hz, 1.64H), 5.05 (s, 0.36H), 5.07 (s, 1.64H), 5.62-5.74 (br m, 0.18H), 6.59 (br s, 0.82H), 6.85 (d, J=8.8 Hz, 0.36H), 6.89 (d, J=8.5 Hz, 1.64H), 7.10-7.26 (m, 2.82H), 7.29-7.44 (m, 5H), 9.45 (s, 0.18H).

### Step 4 Synthesis of Compound 14

Compound 13 (65.0 mg, 0.136 mmol) was dissolved in DMF (0.65 mL) and THF (0.65 mL), and sodium hydride (6.0 mg, 0.149 mmol) was added, then the mixture was stirred at room temperature for 1 hour. 4-Fluorobenzyl bromide (0.0201 mL, 0.163 mmol) was added, and the mixture was stirred at room temperature for 1.5 hours. An aqueous solution of ammonium chloride was added, then the mixture was extracted with ethyl acetate, the organic layer was washed with water, and the solvent was distilled off under reduced pressure. The obtained residue was purified by aminosilica gel column chromatography (chloroform-methanol) to afford Compound 14 (67.8 mg, yield 85%) as a colorless oil.

1H-NMR (CDCl3) δ: 1.02 (d, J=6.8 Hz, 6H), 1.35-1.69 (m, 4H), 1.96-2.16 (m, 1H), 2.46 (s, 2H), 3.14-3.37 (m, 2H), 3.69 (d, J=6.5 Hz, 2H), 3.72-3.98 (m, 3H), 4.21 (s, 2H), 4.73-5.07 (m, 2H), 5.12 (s, 2H), 6.80 (d, J=8.3 Hz, 2H), 6.93-7.09 (m, 4H), 7.10-7.21 (m, 2H), 7.29-7.40 (m, 5H).

### Step 5 Synthesis of Compound (I-005)

Compound 14 (67.5 mg, 0.115 mmol) was dissolved in THF (0.625 mL) and methanol (0.625 mL), 10 w/w % palladium on carbon (15 mg) was added, and the mixture was stirred under a 1 atm hydrogen atmosphere for 8 hours. The reaction solution was filtered through Celite, and the solvent of the filtrate was distilled off under reduced pressure. The obtained residue was dissolved in THF (0.625 mL) and methanol (0.625 mL), and a 37% aqueous solution of formaldehyde (0.026 mL) and sodium triacetoxyborohydride (48.8 mg, 0.230 mmol) were added, then the mixture was stirred at room temperature for 2 hours. A 20% aqueous solution of potassium carbonate was added, then the mixture was extracted with ethyl acetate, the organic layer was washed with water, and the solvent was distilled off under reduced pressure. The obtained residue was purified by aminosilica gel column chromatography (chloroform-methanol) to afford a colorless oil. This oil was dissolved in ethyl acetate, and a 4 mol/L hydrochloric acid/ethyl acetate solution (0.022 mL) was added, and the solvent was distilled off under reduced pressure to afford Compound (I-005) (40.1 mg, 69%) as a white powder.

1H-NMR (DMSO-D6) δ: 0.96 (d, J=6.5 Hz, 6H), 1.26-1.38 (m, 2H), 1.59-1.72 (m, 2H), 1.92-2.11 (m, 2H), 2.33-2.45 (m, 2H), 2.58-2.73 (m, 1H), 2.92-3.06 (m, 2H), 3.33 (s, 3H), 3.62-3.78 (m, 2H), 4.11-4.29 (m, 2H), 4.99 (s, 2H), 6.77 (d, J=8.3 Hz, 2H), 6.97 (d, J=8.0 Hz, 2H), 7.13-7.22 (m, 2H), 7.25-7.33 (m, 2H), 9.81 (br s, 1H).

### Reference Example 1

### Synthesis of Compound 18

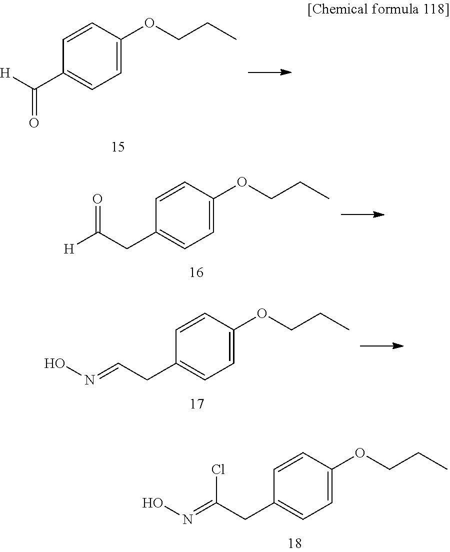

### Step 1 Synthesis of Compound 16

Methoxymethyltriphenylphosphonium chloride (12.5 g, 36.5 mmol) was dissolved in tetrahydrofuran (50 mL), and potassium tert-butoxide (4.10 g, 36.5 mmol) was added, then the mixture was stirred at room temperature for 1 hour. Compound 15 (5.0 g, 30.4 mmol) was added, and the mixture was stirred at room temperature for 18 hours. A saturated aqueous solution of ammonium chloride was added, and then the mixture was extracted with ethyl acetate. The organic layer was washed with brine, and dried over anhydrous magnesium sulfate. The solvent was distilled off under reduced pressure, the obtained residue was dissolved in acetone (200 mL), and a 2 mol/L aqueous solution of hydrochloric acid (22.8 mL, 45.7 mmol) was added, then the mixture was stirred at 45° C. for 4 hours. The reaction solvent was distilled off under reduced pressure, saturated aqueous sodium bicarbonate was added, and the mixture was extracted with diethyl ether. After drying over magnesium sulfate, the solvent was distilled off under reduced pressure. The obtained residue was purified by silica gel column chromatography (hexane-ethyl acetate) to afford Compound 16 (1.96 g, yield 36%) as a colorless oil.

¹H-NMR (CDCl₃) δ: 1.04 (t, J=7.4 Hz, 3H), 1.76-1.87 (m, 2H), 3.62 (s, 2H), 3.92 (t, J=7.4 Hz, 3H), 6.90 (d, J=8.5 Hz, 2H), 7.12 (d, J=8.5 Hz, 2H), 9.72 (t, J=2.3 Hz, 1H).

### Step 2 Synthesis of Compound 17

Compound 16 (1.94 g, 10.9 mmol) was dissolved in methanol (20 mL) and water (10 mL), and hydroxylamine chloride (2.27 g, 32.7 mmol) and sodium carbonate (3.46 g, 32.7 mmol) were added, then the mixture was stirred at room temperature for 24 hours. Brine was added, and the mixture was extracted with ethyl acetate, and the resultant was dried over anhydrous magnesium sulfate. The solvent was distilled off under reduced pressure, and hexane was added to the obtained residue to precipitate a solid, which was then collected by filtration to afford Compound 17 (1.55 g, yield 74%) as a white solid.

¹H-NMR (CDCl₃) δ: 1.03 (t, J=7.4 Hz, 3H), 1.75-1.85 (m, 2H), 3.69 (d, J=6.0 Hz, 2H), 3.91 (t, J=7.4 Hz, 3H), 6.83-6.93 (m, 3H), 7.13 (d, J=8.5 Hz, 2H), 7.64 (s, 1H).

### Step 3 Synthesis of Compound 18

Compound 17 (300 mg, 1.55 mmol) was dissolved in DMF (3 mL), and N-chlorosuccinimide (207 mg, 1.55 mmol) was added, then the mixture was stirred at room temperature for 1 hour. Saturated aqueous sodium bicarbonate was added, and the mixture was extracted with ethyl acetate. The organic layer was washed with water and brine, then dried over anhydrous magnesium sulfate, and the solvent was distilled off under reduced pressure to afford Compound 18 (366 mg, yield 104%) as a crude product.

¹H-NMR (CDCl₃) δ: 1.03 (t, J=7.4 Hz, 3H), 1.76-1.85 (m, 2H), 3.74 (s, 2H), 3.91 (t, J=7.4 Hz, 3H), 6.88 (d, J=8.5 Hz, 2H), 7.17 (d, J=8.5 Hz, 2H).

### Example 4

### Synthesis of Compound (I-027)

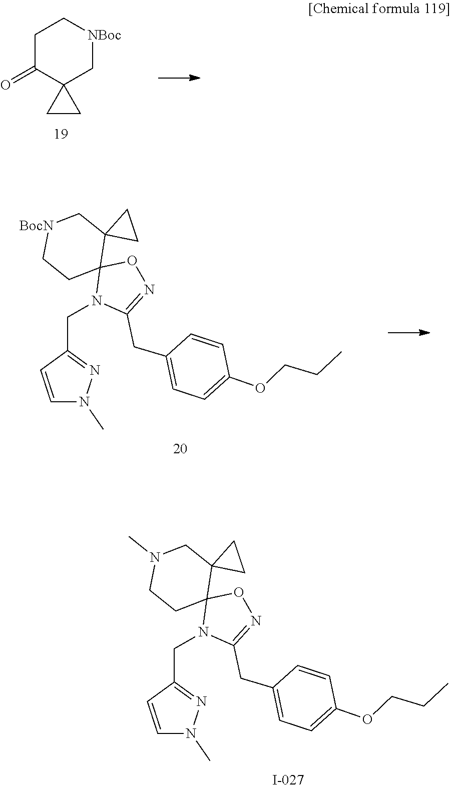

### Step 1 Synthesis of Compound 20

Compound 19 (200 mg, 0.89 mmol) was dissolved in toluene (2 mL), and (1-methyl-1H-pyrazol-3-yl)methanamine (99 mg, 0.89 mmol) and magnesium sulfate (321 mg, 3.66 mmol) were added, then the mixture was stirred at 100° C. for 3 hours. The solid was filtered off, and the solvent was distilled off under reduced pressure. The obtained residue was dissolved in tetrahydrofuran (2 mL), and Compound 18 (202 mg, 0.89 mmol) and triethylamine (0.185 mL, 1.33 mmol) were added, then the mixture was stirred at room temperature for 16 hours. Water was added to the reaction solution, and the mixture was extracted with ethyl acetate. The organic layer was washed with brine, then dried over anhydrous magnesium sulfate, and the solvent was distilled off under reduced pressure. The obtained residue was purified by silica gel column chromatography (hexane-ethyl acetate) to afford Compound 20 (105 mg, yield 23%) as a yellow oil.

1H-NMR (CDCl3) δ: 0.14-0.30 (m, 2H), 0.33-0.49 (m, 1H), 0.58-0.65 (m, 1H), 1.04 (t, J=7.4 Hz, 3H), 1.42 (m, 10H), 1.75-2.00 (m, 4H), 3.06-3.24 (m, 1H), 3.37-3.56 (m, 2H), 3.95-4.10 (m, 2H), 3.86 (s, 3H), 3.90 (t, J=6.7 Hz, 2H), 4.00 (d, J=17.2 Hz, 1H), 4.27 (d, J=17.2 Hz, 1H), 6.00 (d, J=1.8 Hz, 1H), 6.83 (d, J=8.5 Hz, 2H), 7.16 (d, J=8.4 Hz, 2H), 7.25 (br s, 1H).

### Step 2 Synthesis of Compound (I-027)

Compound 20 (100 mg, 0.20 mmol) was dissolved in dichloromethane (1 mL), and 2,6-lutidine (0.14 mL, 1.18 mmol) and trimethylsilyl triflate (0.18 mL, 0.98 mmol) were added under ice cooling, then the mixture was stirred for 1 hour. Saturated aqueous sodium bicarbonate was added, and the mixture was extracted with ethyl acetate. After drying over magnesium sulfate, the solvent was distilled off under reduced pressure. The obtained residue was dissolved in methanol (1 mL), and a 37% formaldehyde solution (0.15 mL, 1.96 mmol) and sodium triacetoxyborohydride (125 mg, 0.59 mmol) were added, then the mixture was stirred at room temperature for 1 hour. Saturated aqueous sodium bicarbonate was added, and the mixture was extracted with ethyl acetate. The organic layer was washed with brine, then dried over magnesium sulfate, and the solvent was distilled off under reduced pressure. The obtained residue was purified by silica gel column chromatography (chloroform-methanol) to afford Compound (I-027) (28 mg, yield 34%) as a yellow oil.

1H-NMR (CDCl3) δ: 0.05-0.12 (m, 1H), 0.37-0.40 (m, 2H), 0.54-0.59 (m, 1H), 1.03 (t, J=7.4 Hz, 3H), 1.76-1.94 (m, 2H), 1.88-1.94 (m, 2H), 2.11 (td, J=12.7 Hz, 4.7 Hz, 1H), 2.26 (s, 3H), 2.47 (t, J=11.0 Hz, 1H), 2.78-2.84 (m, 2H), 3.41 (d, J=15.6 Hz, 1H), 3.62 (d, J=15.6 Hz, 1H), 3.86 (s, 3H), 3.90 (t, J=6.7 Hz, 2H), 4.03 (d, J=17.1 Hz, 1H), 4.26 (d, J=17.1 Hz, 1H), 6.02 (d, J=2.3 Hz, 1H), 6.82 (d, J=8.5 Hz, 2H), 7.15 (d, J=8.5 Hz, 2H), 7.25 (d, J=2.3 Hz, 1H).

### Example 5

### Synthesis of Compound (I-040)

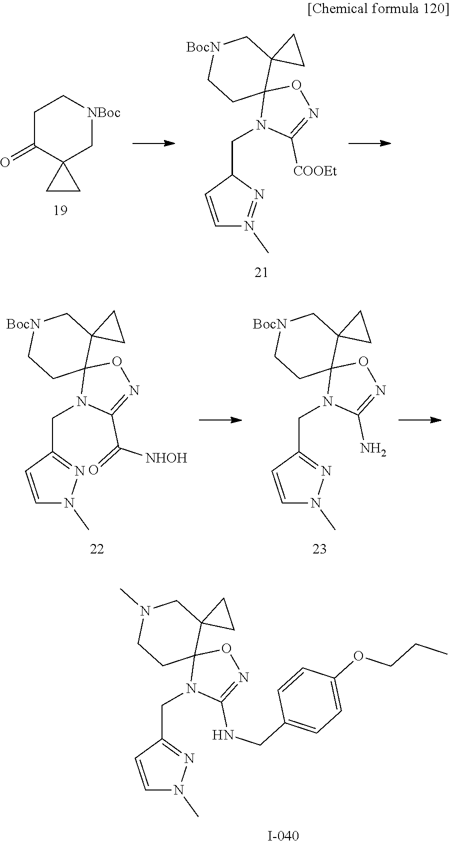

### Step 1 Synthesis of Compound 21

Compound 19 (2.00 g, 8.88 mmol) was dissolved in toluene (20 mL), and (1-methyl-1H-pyrazol-3-yl)methanamine (0.99 g, 8.88 mmol) and magnesium sulfate (1.60 g, 13.32 mmol) were added, then the mixture was stirred at 100° C. for 4 hours. The solid was filtered off, and the solvent was distilled off under reduced pressure. The obtained residue was dissolved in tetrahydrofuran (20 mL), ethyl 2-chloro-2-(hydroxyimino)acetate (2.02 g, 13.32 mmol) and triethylamine (2.46 mL, 17.76 mmol) were added, and the mixture was stirred at room temperature for 24 hours. Water was added to the reaction solution, and the mixture was extracted with ethyl acetate. The organic layer was washed with brine, then dried over anhydrous magnesium sulfate, and the solvent was distilled off under reduced pressure. The obtained residue was purified by silica gel column chromatography (hexane-ethyl acetate) to afford Compound 21 (872 mg, yield 23%) as a colorless oil.

1H-NMR (CDCl3) δ: 0.27-0.35 (m, 1H), 0.39-0.62 (m, 2H), 0.77-0.86 (m, 1H), 1.35 (t, J=7.2 Hz, 3H), 1.45 (m, 10H), 1.86-1.96 (m, 1H), 2.07-2.16 (m, 1H), 3.05-3.39 (m, 2H), 3.42-3.64 (m, 1H), 3.82 (s, 3H), 3.98-4.17 (m, 2H), 4.26-4.41 (m, 2H), 4.87 (d, J=16.7 Hz, 1H), 6.04 (br s, 1H), 7.23 (br s, 1H).

### Step 2 Synthesis of Compound 22

Compound 21 (800 mg, 1.85 mmol) was dissolved in ethanol (8 mL), and hydroxylamine chloride (1.22 mL, 18.45 mmol) was added, then the mixture was heated and refluxed for 3 hours. The reaction solvent was distilled off under reduced pressure, and the obtained residue was purified by silica gel column chromatography (chloroform-methanol) to afford Compound 22 (655 mg, yield 84%) as a colorless oil.

1H-NMR (CDCl3) δ: 0.27-0.36 (m, 1H), 0.37-0.60 (m, 2H), 0.78-0.86 (m, 1H), 1.45 (m, 10H), 1.80-1.96 (m, 1H), 2.07-2.18 (m, 1H), 3.05-3.28 (m, 1H), 3.40-3.66 (m, 2H), 3.83 (s, 3H), 4.00-4.20 (m, 2H), 4.90 (d, J=16.7 Hz, 1H), 6.06 (br s, 1H), 7.25 (br s, 1H).

### Step 3 Synthesis of Compound 23

Compound 22 (400 mg, 1.85 mmol) was dissolved in tetrahydrofuran (2 mL), and a 50% tetrahydrofuran solution of propylphosphonic anhydride (cyclic trimer) (1.42 mL, 2.38 mmol) and N-methylmorpholine (0.26 mL, 2.38 mmol) were added, then the mixture was stirred at room temperature for 1 hour. 2-(Trimethylsilyl)ethanol (0.68 mL, 4.76 mmol) was added, and the mixture was heated and refluxed for 6 hours. Saturated aqueous sodium bicarbonate was added to the reaction solution, and the mixture was extracted with ethyl acetate. The organic layer was washed with brine, then dried over magnesium sulfate, and the solvent was distilled off under reduced pressure. The obtained residue was dissolved in tetrahydrofuran (5.3 mL), and a 1 mol/L tetrahydrofuran solution of tetrabutylammonium fluoride (1.54 mL, 1.54 mmol) was added, then the mixture was heated and refluxed for 4 hours. The reaction solvent was distilled off under reduced pressure, and the obtained residue was purified by aminosilica gel column chromatography (chloroform-methanol) to afford Compound 23 (288 mg, yield 75%) as a white solid.

1H-NMR (CDCl3) δ: 0.26-0.54 (m, 3H), 0.81-0.89 (m, 1H), 1.45 (m, 9H), 1.88-1.95 (m, 1H), 2.00-2.12 (m, 1H), 3.12-3.41 (m, 2H), 3.44-3.64 (m, 1H), 3.86 (s, 3H), 4.03 (d, J=15.9 Hz, 1H), 4.07-4.24 (m, 1H), 4.27 (d, J=15.9 Hz, 1H), 4.53 (br s, 2H), 6.12 (br s, 1H), 7.29 (br s, 1H).

### Step 4 Synthesis of Compound (I-040)

Compound 23 (50 mg, 0.20 mmol) was dissolved in 2-propanol (0.5 mL), and Compound 15 (26 mg, 0.16 mmol) and isopropyl orthotitanate (0.06 mL, 0.20 mmol) were added, then the mixture was heated and refluxed for 2 hours. After the mixture was allowed to cool, sodium borohydride (15 mg, 0.39 mmol) was added, and the mixture was stirred at room temperature for 1 hour. Saturated aqueous sodium bicarbonate was added, and the mixture was extracted with ethyl acetate. After drying over magnesium sulfate, the solvent was distilled off under reduced pressure. The obtained residue was dissolved in dichloromethane (1.2 mL), and 2,6-lutidine (0.016 mL, 0.137 mmol) and trimethylsilyl triflate (0.021 mL, 0.114 mmol) were added under ice cooling, then the mixture was stirred for 1 hour. Saturated aqueous sodium bicarbonate was added, and the mixture was extracted with ethyl acetate. After drying over magnesium sulfate, the solvent was distilled off under reduced pressure. The obtained residue was dissolved in methanol (1.2 mL), and a 37% formaldehyde solution (0.017 mL, 1.96 mmol) and sodium triacetoxyborohydride (15 mg, 0.069 mmol) were added, then the mixture was stirred at room temperature for 1 hour. Saturated aqueous sodium bicarbonate was added, and the mixture was extracted with ethyl acetate. The organic layer was washed with brine, then dried over magnesium sulfate, and the solvent was distilled off under reduced pressure. The obtained residue was purified by silica gel column chromatography (chloroform-methanol) to afford Compound (I-040) (5 mg, yield 8%).

1H-NMR (CDCl3) δ: 0.14-0.23 (m, 1H), 0.40-0.54 (m, 2H), 0.76-0.83 (m, 1H), 1.03 (t, J=7.4 Hz, 3H), 1.75-1.84 (m, 2H), 1.92-2.04 (m, 2H), 2.15-2.26 (m, 1H), 2.30 (s, 3H), 2.53-2.67 (m, 1H), 2.83-2.91 (m, 2H), 3.73 (s, 3H), 3.90 (t, J=6.7 Hz, 2H), 4.02 (d, J=16.1 Hz, 1H), 4.19-4.26 (m, 3H), 5.63 (br s, 1H), 6.07 (d, J=2.3 Hz, 1H), 6.84 (d, J=8.8 Hz, 2H), 7.21-7.25 (m, 3H).

### Example 6

### Synthesis of Compound (I-022)

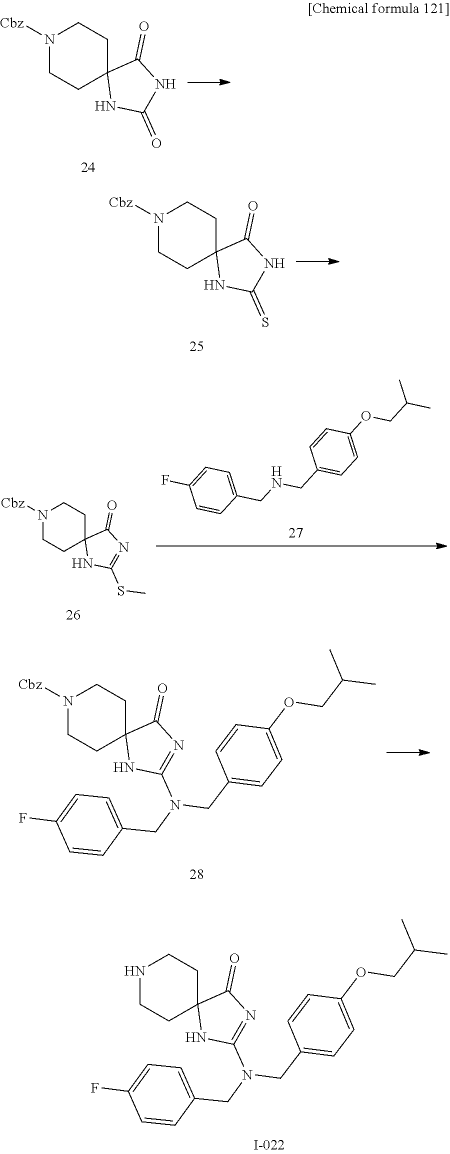 [SMILES: O=C1NC(=O)C2(CCN(C(=O)OCc3ccccc3)CC2)N1 ; O=C(OCc1ccccc1)N1CCC2(CC1)NC(=S)NC2=O ; CSC1=NC(=O)C2(CCN(C(=O)OCc3ccccc3)CC2)N1 ; CC(C)COc1ccc(CNCc2ccc(F)cc2)cc1 ; CC(C)COc1ccc(CN(Cc2ccc(F)cc2)C2=NC(=O)C3(CCN(C(=O)OCc4ccccc4)CC3)N2)cc1 ; CC(C)COc1ccc(CN(Cc2ccc(F)cc2)C2=NC(=O)C3(CCNCC3)N2)cc1]

### Step 1 Synthesis of Compound 25

Compound 24 (the synthetic method is described in WO2008014311 A2) (1 g, 3.30 mmol) was dissolved in tetrahydrofuran (10 mL), and a Lawesson's reagent (1.33 g, 3.30 mmol) was added, then the mixture was stirred at 140° C. for 30 minutes under microwave irradiation. An aqueous solution (5 mL) of ethanolamine (1.99 mL, 33.0 mmol) was added, and the mixture was stirred at 80° C. for 1 hour. 2 mol/L hydrochloric acid (33.0 mL, 65.9 mmol) was added, and the mixture was stirred at 80° C. for 4 hours. After the mixture was allowed to cool, a 20% aqueous solution of potassium carbonate (2 mL) was added, and the mixture was extracted with ethyl acetate. The organic layer was washed with brine, and then dried over anhydrous sodium sulfate. The solvent was distilled off under reduced pressure, and the obtained residue was purified by silica gel column chromatography (hexane-ethyl acetate) to afford Compound 25 (731 mg, yield 70%).

¹H-NMR (CDCl₃) δ: 1.69-1.81 (2H, m), 1.98-2.09 (2H, m), 3.38-3.47 (2H, m), 3.94-4.05 (2H, m), 5.15 (2H, s), 7.31-7.42 (6H, m).

### Step 2 Synthesis of Compound 26

Compound 25 (720 mg, 2.25 mmol) was dissolved in ethanol (14 mL), and DIPEA (0.47 mL, 2.70 mmol) and methyl iodide (0.17 mL, 2.70 mmol) were added, then the mixture was stirred at room temperature for 20 hours. DIPEA (0.47 mL, 2.70 mmol) and methyl iodide (0.17 mL, 2.70 mmol) were added, and the mixture was stirred at room temperature for 2 hours. Water was added and the mixture was extracted with ethyl acetate. The organic layer was washed with brine, and then dried over anhydrous sodium sulfate. The solvent was distilled off under reduced pressure, and the obtained residue was purified by silica gel column chromatography (hexane-ethyl acetate) to afford Compound 26 (468 mg, yield 62%).

¹H-NMR (CDCl₃) δ: 1.43-1.55 (2H, m), 1.81-1.93 (2H, m), 2.55 (3H, s), 3.40-3.54 (2H, m), 3.98-4.15 (2H, m), 5.15 (2H, s), 7.29-7.41 (5H, m), 7.65 (1H, s).

### Step 3 Synthesis of Compound 28

Acetic acid (1.5 mL) and Compound 27 (the synthetic method is described in WO2019040105 A²) (132 mg, 0.460 mmol) were added to Compound 26 (153 mg, 0.459 mmol), and the mixture was stirred at 130° C. for 9 hours. The solvent was distilled off under reduced pressure, a saturated aqueous solution of sodium hydrogen carbonate was added to the obtained residue, and the mixture was extracted with chloroform. The organic layer was dried over anhydrous sodium sulfate, and the solvent was distilled off under reduced pressure. The obtained residue was purified by silica gel column chromatography (hexane-ethyl acetate, followed by chloroform-methanol) to afford Compound 28 (127 mg, yield 49%).

¹H-NMR (CDCl₃) δ: 1.02 (6H, d, J=6.6 Hz), 1.39-1.53 (2H, m), 1.89-2.01 (2H, m), 2.03-2.14 (1H, m), 3.03-3.19 (2H, m), 3.70 (2H, d, J=6.6 Hz), 3.95-4.07 (2H, m), 4.35 (2H, br s), 4.85 (2H, br s), 5.09 (2H, s), 5.22 (1H, s), 6.87 (2H, d, J=8.3 Hz), 7.00-7.10 (4H, m), 7.28-7.39 (7H, m).

### Step 4 Synthesis of Compound (I-022)

Under a hydrogen atmosphere, Compound 28 (126 mg, 0.221 mmol) was dissolved in tetrahydrofuran 2.5 mL), and 10 w/w % palladium on carbon (47 mg) was added, then the mixture was stirred under a 1 atm hydrogen atmosphere for 5 hours. The reaction solution was filtered through Celite, and the solvent of the filtrate was distilled off under reduced pressure. The obtained residue was purified by aminosilica gel column chromatography (chloroform-methanol) to afford Compound (I-022) (77 mg, yield 79%).

1H-NMR (CDCl3) δ: 1.02 (6H, d, J=6.5 Hz), 1.36-1.44 (2H, m), 1.90-2.00 (2H, m), 2.03-2.13 (1H, m), 2.38-2.49 (2H, m), 3.11-3.19 (2H, m), 3.71 (2H, d, J=6.5 Hz), 4.38 (2H, br s), 4.87 (2H, br s), 5.29 (1H, s), 6.88 (2H, d, J=8.5 Hz), 7.00-7.40 (6H, m).

### Example 7

### Synthesis of Compound (I-021)

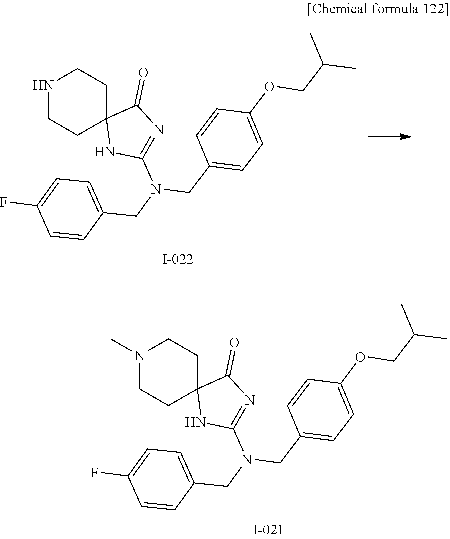 [SMILES: CC(C)COc1ccc(CN(Cc2ccc(F)cc2)C2=NC(=O)C3(CCNCC3)N2)cc1 ; CC(C)COc1ccc(CN(Cc2ccc(F)cc2)C2=NC(=O)C3(CCN(C)CC3)N2)cc1]

### Step 1 Synthesis of Compound (I-021)

Compound (I-022) (31.3 mg, 0.071 mmol) was dissolved in THF (0.470 mL) and methanol (0.470 mL), and a 37% aqueous solution of formaldehyde (0.017 mL) and sodium triacetoxyborohydride (30.3 mg, 0.143 mmol) were added, then the mixture was stirred at room temperature for 1 hour. A saturated aqueous solution of sodium hydrogen carbonate was added, then the mixture was extracted with ethyl acetate, the organic layer was washed with brine, and the solvent was distilled off under reduced pressure. The obtained residue was purified by silica gel column chromatography (chloroform-methanol) to afford a colorless oil. This oil was solidified with diisopropyl ether to afford Compound (I-021) (18.8 mg, 58%) as a white powder.

1H-NMR (CDCl3) δ: 1.03 (6H, d, J=6.8 Hz), 1.44-1.54 (2H, m), 1.75-1.88 (2H, m), 2.02-2.16 (3H, m), 2.25 (3H, s), 2.82-2.93 (2H, m), 3.71 (2H, d, J=6.5 Hz), 4.36 (2H, br s), 4.84 (2H, br s), 5.23 (1H, s), 6.88 (2H, d, J=8.3 Hz), 6.98-7.39 (6H, m).

### Example 8

### Synthesis of Compound (I-020)

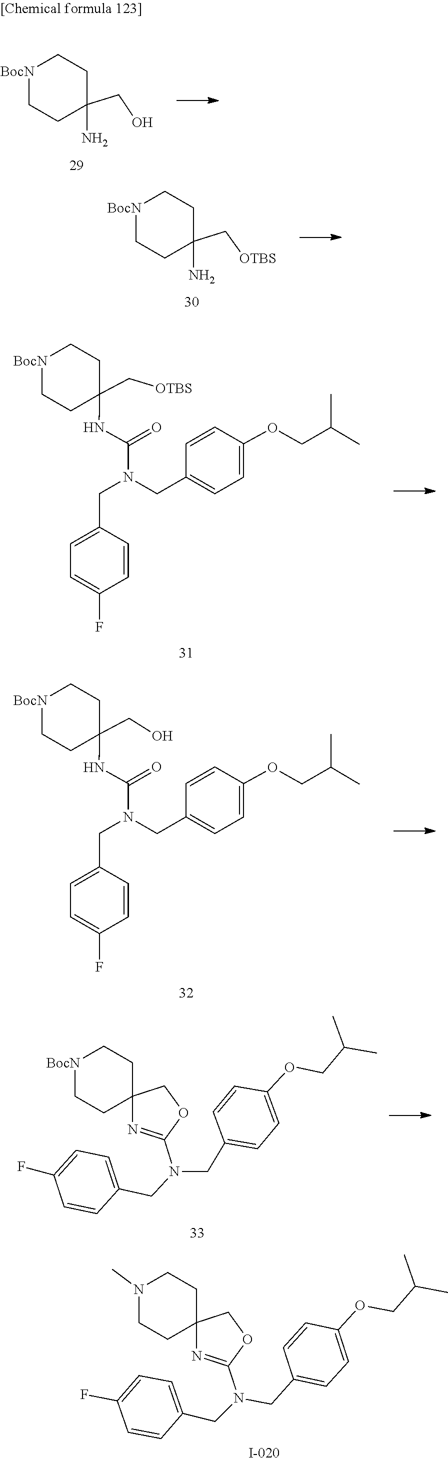 [SMILES: CC(C)(C)OC(=O)N1CCC(N)(CO)CC1 ; CC(C)(C)OC(=O)N1CCC(N)(CO[Si](C)(C)C(C)(C)C)CC1 ; CC(C)COc1ccc(CN(Cc2ccc(F)cc2)C(=O)NC2(CO[Si](C)(C)C(C)(C)C)CCN(C(=O)OC(C)(C)C)CC2)cc1 ; CC(C)COc1ccc(CN(Cc2ccc(F)cc2)C(=O)NC2(CO)CCN(C(=O)OC(C)(C)C)CC2)cc1 ; CC(C)COc1ccc(CN(Cc2ccc(F)cc2)C2=NC3(CCN(C(=O)OC(C)(C)C)CC3)CO2)cc1 ; CC(C)COc1ccc(CN(Cc2ccc(F)cc2)C2=NC3(CCN(C)CC3)CO2)cc1]

### Step 1 Synthesis of Compound 30

Compound 29 (250 mg, 1.09 mmol) was dissolved in dichloromethane (2.5 mL), and triethylamine (0.451 mL, 3.26 mmol) and tert-butyldimethylsilyl chloride (196 mg, 1.30 mmol) were added, then the mixture was stirred at room temperature for 5 hours. tert-Butyldimethylsilyl chloride (94 mg, 0.625 mmol) was added, and the mixture was stirred at room temperature for 18 hours. Water was added and the mixture was extracted with ethyl acetate. The organic layer was washed with brine, and then dried over anhydrous sodium sulfate. The solvent was distilled off under reduced pressure, and the obtained residue was purified by silica gel column chromatography (hexane-ethyl acetate) to afford Compound 30 (296 mg, yield 79%).

¹H-NMR (CDCl₃) δ: 0.05 (6H, s), 0.90 (9H, s), 1.29-1.38 (2H, m), 1.40-1.52 (2H, m), 3.18-3.28 (2H, m), 3.33 (2H, s), 3.64-3.78 (2H, m).

### Step 2 Synthesis of Compound 31

Compound 30 (278 mg, 0.807 mmol) was dissolved in ethyl acetate (2.8 mL), and an aqueous solution (1.7 mL) of potassium carbonate (558 mg, 4.04 mmol) was added. Under ice cooling, an ethyl acetate solution (1.4 mL) of triphosgene (240 mg, 0.807 mmol) was added dropwise over about 5 minutes. The mixture was stirred at room temperature for 30 minutes, then water was added, and the mixture was extracted with ethyl acetate. The organic layer was washed with brine, and then dried over anhydrous sodium sulfate. The solvent was distilled off under reduced pressure, the obtained residue was dissolved in THF (3.4 mL), a THF solution (2.8 mL) of Compound 27 (240 mg, 0.834 mmol) was added, and the mixture was stirred at room temperature for 2.5 hours. Water was added and the mixture was extracted with ethyl acetate. The organic layer was washed with brine, and the solvent was distilled off under reduced pressure. The obtained residue was purified by silica gel column chromatography (hexane-ethyl acetate) to afford Compound 31 (389 mg, 77%).

¹H-NMR (CDCl₃) δ: 0.00 (6H, s), 0.84 (9H, s), 1.02 (6H, d, J=6.7 Hz), 1.35-1.47 (2H, m), 1.44 (9H, s), 1.93-2.13 (3H, m), 2.63-2.78 (2H, m), 3.56-3.80 (6H, m), 4.11 (1H, s), 4.35 (2H, s), 4.46 (2H, s), 6.85 (2H, d, J=8.2 Hz), 6.97-7.05 (2H, m), 7.11 (2H, d, J=8.2 Hz), 7.17-7.24 (2H, m).

### Step 3 Synthesis of Compound 32

Compound 31 (198 mg, 0.301 mmol) was dissolved in THF (2.0 mL), and an aqueous solution (1.7 mL) of a 1 mol/L TBAF-THF solution (0.904 mL, 0.904 mmol) was added, then the mixture was stirred at room temperature for 16.5 hours. Water was added and the mixture was extracted with ethyl acetate. The organic layer was washed with brine, and then dried over anhydrous sodium sulfate. The solvent was distilled off under reduced pressure, and the obtained residue was purified by silica gel column chromatography (hexane-ethyl acetate) to afford Compound 32 (144 mg, 88%).

¹H-NMR (CDCl₃) δ: 1.02 (6H, d, J=6.5 Hz), 1.44 (10H, s), 1.49-1.62 (2H, m), 1.66-1.79 (2H, m), 2.01-2.13 (1H, m), 2.70 (2H, t, J=11.5 Hz), 3.53-3.66 (4H, m), 3.70 (2H, d, J=6.5 Hz), 4.32 (1H, s), 4.39 (2H, s), 4.52 (2H, s), 5.14 (1H, s), 6.87 (2H, d, J=8.5 Hz), 7.01-7.08 (2H, m), 7.11 (2H, d, J=8.5 Hz), 7.19-7.25 (2H, m).

### Step 4 Synthesis of Compound 33

Compound 32 (139 mg, 0.256 mmol) was dissolved in dichloromethane (4.2 mL) and cooled to −78° C. with dry ice-acetone. N,N-Diethylaminosulfur trifluoride (0.034 mL, 0.256 mmol) was added, and the mixture was stirred at −78° C. for 30 minutes. N,N-Diethylaminosulfur trifluoride (0.044 mL, 0.333 mmol) was added, and the mixture was stirred at −78° C. for 30 minutes. A saturated aqueous solution of sodium hydrogen carbonate was added, and the temperature was raised to room temperature. The mixture was extracted with ethyl acetate, and the organic layer was washed with brine, then dried over anhydrous sodium sulfate. The solvent was distilled off under reduced pressure, and the obtained residue was purified by silica gel column chromatography (hexane-ethyl acetate) to afford Compound 33 (119 mg, yield 89%).

¹H-NMR (CDCl3) δ: 1.03 (6H, d, J=6.6 Hz), 1.46 (9H, s), 1.58-1.65 (2H, m), 1.66-1.77 (2H, m), 2.02-2.14 (1H, m), 3.35-3.47 (2H, m), 3.58-3.67 (2H, m), 3.71 (2H, d, J=6.6 Hz), 4.05 (2H, s), 4.30 (2H, s), 4.32 (2H, s), 6.84 (2H, d, J=8.2 Hz), 6.95-7.04 (2H, m), 7.11 (2H, d, J=8.2 Hz), 7.14-7.22 (2H, m).

### Step 5 Synthesis of Compound (I-020)

Compound 33 (40.5 mg, 0.077 mmol) was dissolved in THF (0.81 mL), and lithium aluminum hydride (8.8 mg, 0.231 mmol) was added, then the mixture was refluxed for 1.5 hours. Water was added and the mixture was extracted with ethyl acetate. The organic layer was dried over anhydrous sodium sulfate. The solvent was distilled off under reduced pressure, and the obtained residue was purified by silica gel column chromatography (chloroform-methanol-water) to afford Compound (I-020) (23.5 mg, 69%).

1H-NMR (CDCl3) δ: 1.02 (6H, d, J=6.6 Hz), 1.64-1.73 (2H, m), 1.82-1.94 (2H, m), 2.01-2.26 (3H, m), 2.29 (3H, s), 2.67-2.79 (2H, m), 3.71 (2H, d, J=6.6 Hz), 4.05 (2H, s), 4.29 (2H, s), 4.32 (2H, s), 6.84 (2H, d, J=8.5 Hz), 6.99 (2H, t, J=8.5 Hz), 7.10 (2H, d, J=8.5 Hz), 7.17 (2H, t, J=6.8 Hz).

### Example 9

### Synthesis of Compound (I-080)

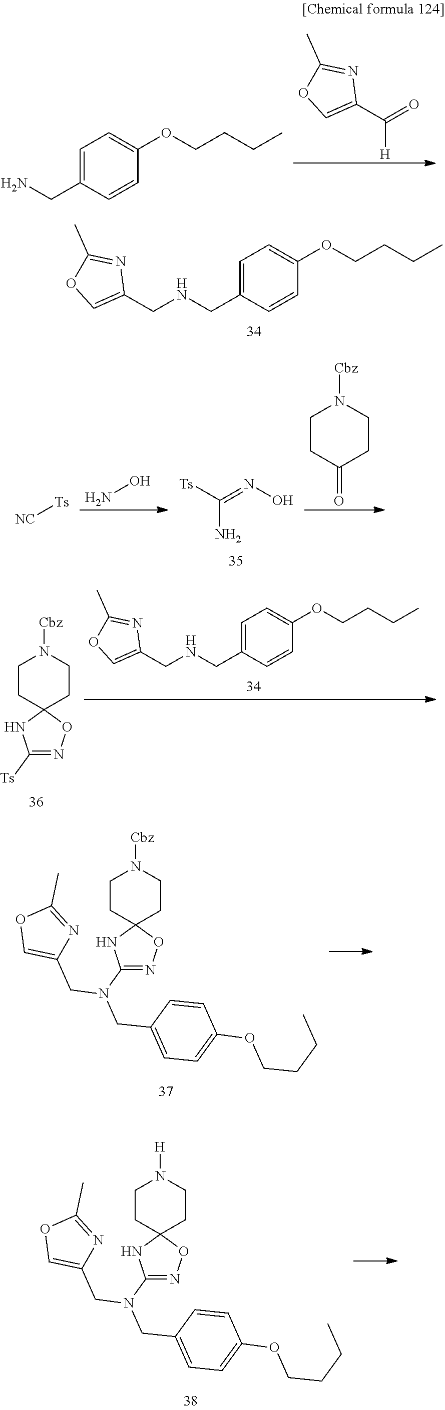

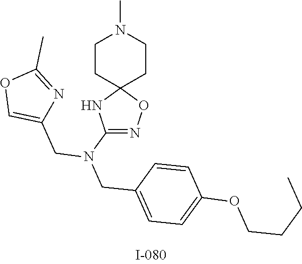 [SMILES: CCCCOc1ccc(CN(Cc2coc(C)n2)C2=NOC3(CCN(C)CC3)N2)cc1]

### Step 1 Synthesis of Compound 34

4-Butoxybenzenemethanamine (4.84 g, 27.0 mmol) was dissolved in ethanol (15 mL), 2-methyloxazole-4-carboxaldehyde (3.0 g, 27.0 mmol) was added, and the mixture was stirred at 80° C. for 1 hour. The mixture was ice-cooled, sodium borohydride (1.02 g, 27.0 mmol) was added, and the mixture was stirred at room temperature for 1 hour. Saturated aqueous sodium bicarbonate was added, and the mixture was extracted with chloroform. The organic layer was dried over anhydrous sodium sulfate, and the solvent was distilled off under reduced pressure. The obtained residue was purified by amino column chromatography (hexane-ethyl acetate) to afford Compound 34 (6.16 g, yield 83%).

1H-NMR (CDCl3) δ: 0.97 (3H, t, J=7.5 Hz), 1.49 (2H, dt, J=22.7, 7.5 Hz), 1.72-1.79 (2H, m), 2.44 (3H, s), 3.65 (2H, s), 3.74 (2H, s), 3.95 (2H, t, J=6.6 Hz), 6.85 (2H, d, J=8.7 Hz), 7.23 (2H, d, J=8.7 Hz), 7.40 (1H, s).

### Step 2 Synthesis of Compound 35

4-Methylbenzenesulfonyl cyanide (25.1 g, 138 mmol) was dissolved in 2-propanol (125 mL), a 50% aqueous solution of hydroxylamine (25.4 mL, 415 mmol) was added under ice cooling, and the mixture was stirred at room temperature for 3 hours. Water was added and the mixture was extracted with ethyl acetate. The organic layer was washed with water and then dried over anhydrous sodium sulfate. The solvent was distilled off under reduced pressure, and the obtained solid was washed with ethyl acetate-hexane to afford Compound 35 (25.2 g, yield 85%).

1H-NMR (DMSO-D6) δ: 2.42 (3H, s), 6.44 (2H, s), 7.49 (2H, d, J=8.1 Hz), 7.80 (2H, d, J=8.1 Hz), 10.71 (1H, br s).

### Step 3 Synthesis of Compound 36

Compound 35 (25.2 g, 117 mmol) was dissolved in 2-propanol (126 mL), benzyl 4-oxopiperidine-1-carboxylate (30.1 g, 129 mmol) and PPTS (5.91 g, 23.5 mmol) were added, and the mixture was stirred at 130° C. for 3 hours. Benzyl 4-oxopiperidine-1-carboxylate (4.48 g, 19.2 mmol) and PPTS (1.20 g, 4.78 mmol) were added, and the mixture was stirred at 130° C. for 3 hours. After the mixture was allowed to cool, water was added, and the mixture was extracted with ethyl acetate. The organic layer was washed with water and then dried over anhydrous sodium sulfate. The solvent was distilled off under reduced pressure, and the obtained residue was purified by silica gel column chromatography (hexane-ethyl acetate) to afford Compound 36 (11.8 g, yield 16%).

1H-NMR (CDCl3) δ: 1.66-1.78 (2H, m), 1.92-2.01 (2H, m), 2.48 (3H, s), 3.26-3.37 (2H, m), 3.82-3.93 (2H, m), 5.12 (2H, s), 7.30-7.38 (5H, m), 7.40 (2H, d, J=8.3 Hz), 7.89 (2H, d, J=8.3 Hz).

### Step 4 Synthesis of Compound 37

Compound 36 (4.51 g, 10.5 mmol) was dissolved in 1,4-dioxane (18 mL), and Compound 34 (2.88 g, 10.5 mmol) and DIPEA (18 mL, 103 mmol) were added, then the mixture was stirred at 130° C. for 9 hours. The solvent was distilled off under reduced pressure, and the obtained residue was purified by column chromatography (hexane-ethyl acetate) to afford Compound 37 (2.99 g, yield 52%).

1H-NMR (CDCl3) δ: 0.98 (3H, t, J=7.3 Hz), 1.45-1.54 (3H, m), 1.71-1.84 (4H, m), 2.41 (3H, s), 3.49 (2H, dt, J=16.9, 5.9 Hz), 3.77-3.90 (2H, m), 3.95 (2H, t, J=6.5 Hz), 4.02 (2H, s), 4.28 (2H, s), 5.14 (2H, s), 5.88 (1H, s), 6.84 (2H, d, J=8.5 Hz), 7.20 (2H, d, J=8.5 Hz), 7.24 (1H, s), 7.30-7.39 (5H, m).

### Step 5 Synthesis of Compound 38

Compound 37 (2.40 g, 4.38 mmol) was dissolved in dichloromethane (36 mL), and dimethyl sulfide (6.48 mL, 88.0 mmol) and a boron trifluoride diethyl ether complex (5.55 mL, 43.8 mmol) were added, then the mixture was stirred at room temperature for 5 hours. A 10% aqueous solution of potassium carbonate was added, and the mixture was extracted with chloroform. The organic layer was washed with water and then dried over anhydrous sodium sulfate. The solvent was distilled off under reduced pressure, and the obtained residue was purified by amino column chromatography (chloroform-methanol) to afford Compound 38 (1.45 g, yield 80%).

1H-NMR (CDCl3) δ: 0.98 (3H, t, J=7.4 Hz), 1.49 (3H, td, J=14.9, 7.4 Hz), 1.72-1.85 (4H, m), 1.93-1.99 (1H, m), 2.42 (3H, s), 2.87-2.93 (2H, m), 3.03-3.09 (2H, m), 3.95 (2H, t, J=6.5 Hz), 4.04 (2H, s), 4.29 (2H, s), 5.71 (1H, s), 6.85 (2H, d, J=8.7 Hz), 7.21 (2H, d, J=8.7 Hz), 7.26 (1H, s).

### Step 6 Synthesis of Compound (I-080)

Compound 38 (1.43 g, 3.45 mmol) was dissolved in methanol (14 mL) and THF (14 mL), and a 37% aqueous solution of formaldehyde (0.77 mL, 10.4 mmol) and sodium triacetoxyborohydride (2.19 g, 10.4 mmol) were added, then the mixture was stirred at room temperature for 2 hours. Saturated aqueous sodium bicarbonate was added, then the mixture was extracted with chloroform, and the organic layer was dried over anhydrous sodium sulfate. The solvent was distilled off under reduced pressure, and the obtained residue was purified by silica gel column chromatography (chloroform-methanol) to afford Compound (I-080) (1.16 g, yield 79%).

1H-NMR (CDCl3) δ: 0.98 (3H, t, J=7.3 Hz), 1.44-1.55 (2H, m), 1.72-1.81 (2H, m), 1.83-1.94 (2H, m), 2.00-2.09 (2H, m), 2.31 (3H, s), 2.42 (3H, s), 2.48-2.60 (4H, m), 3.95 (2H, t, J=6.5 Hz), 4.03 (2H, s), 4.29 (2H, s), 5.67 (1H, s), 6.84 (2H, d, J=8.6 Hz), 7.20 (2H, d, J=8.6 Hz), 7.25 (1H, s).

### Example 10

### Synthesis of Compound (I-114)

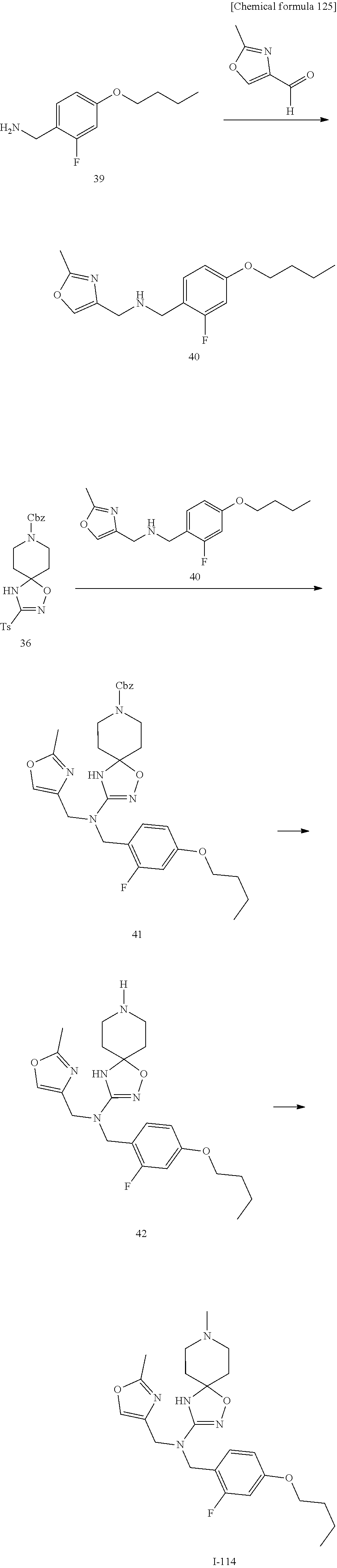

### Step 1 Synthesis of Compound 40

Compound 39 (0.8 g, 7.06 mmol) was dissolved in ethanol (12 mL), and 2-methyloxazole-4-carboxaldehyde (1.53 g, 7.76 mmol) was added, then the mixture was stirred at 80° C. for 1 hour. The mixture was ice-cooled, sodium borohydride (0.294 g, 7.76 mmol) was added, and the mixture was stirred at room temperature for 1 hour. Saturated aqueous sodium bicarbonate was added, and the mixture was extracted with chloroform. The organic layer was dried over anhydrous sodium sulfate, and the solvent was distilled off under reduced pressure. The obtained residue was purified by amino column chromatography (hexane-ethyl acetate) to afford Compound 40 (2.18 g, yield 100%).

1H-NMR (CDCl3) δ: 0.98 (3H, t, J=7.4 Hz), 1.43-1.53 (2H, m), 1.72-1.79 (2H, m), 2.43 (3H, s), 3.65 (2H, s), 3.79 (2H, s), 3.93 (2H, t, J=7.4 Hz), 6.59 (1H, dd, J=12.0, 2.4 Hz), 6.64 (1H, dd, J=8.5, 2.4 Hz), 7.21 (1H, t, J=8.7 Hz), 7.41 (1H, s).

### Step 2 Synthesis of Compound 41

Compound 36 (0.705 g, 1.64 mmol) was dissolved in 1,4-dioxane (6 mL), and Compound 40 (0.480 g, 1.64 mmol) and DIPEA (6 mL, 34.4 mmol) were added, then the mixture was stirred at 130° C. for 6 hours. The solvent was distilled off under reduced pressure, and the obtained residue was purified by column chromatography (hexane-ethyl acetate) to afford Compound 41 (394 mg, yield 42%).

1H-NMR (CDCl3) δ: 0.97 (3H, t, J=7.4 Hz), 1.43-1.53 (2H, m), 1.71-1.92 (4H, m), 1.98-2.05 (2H, m), 2.41 (3H, s), 3.44-3.54 (2H, m), 3.77-3.90 (2H, m), 3.93 (2H, t, J=6.5 Hz), 4.05 (2H, s), 4.31 (2H, s), 5.14 (2H, s), 5.95 (1H, s), 6.58 (1H, dd, J=12.0, 2.3 Hz), 6.66 (1H, dd, J=8.9, 2.3 Hz), 7.29-7.40 (7H, m).

### Step 3 Synthesis of Compound 42

Compound 41 (394 mg, 0.697 mmol) was dissolved in dichloromethane (6 mL), and dimethyl sulfide (1.03 mL, 13.9 mmol) and boron trifluoride diethyl ether complex (0.883 mL, 6.97 mmol) were added, then the mixture was stirred at room temperature for 17 hours. A 10% aqueous solution of potassium carbonate was added, and the mixture was extracted with chloroform. The organic layer was washed with water and then dried over anhydrous sodium sulfate. The solvent was distilled off under reduced pressure, and the obtained residue was purified by amino column chromatography (chloroform-methanol) to afford Compound 42 (224 mg, yield 73%).

### Step 4 Synthesis of Compound (I-114)

Compound 42 (224 mg, 0.520 mmol) was dissolved in methanol (2.2 mL) and THF (2.2 mL), and a 37% aqueous solution of formaldehyde (0.116 mL, 1.56 mmol) and sodium triacetoxyborohydride (330 mg, 1.56 mmol) were added, then the mixture was stirred at room temperature for 1 hour. Saturated aqueous sodium bicarbonate was added, then the mixture was extracted with chloroform, and the organic layer was dried over anhydrous sodium sulfate. The solvent was distilled off under reduced pressure, and the obtained residue was purified by amino column chromatography (chloroform-methanol) to afford Compound (I-114) (217 mg, yield 94%).

1H-NMR (CDCl3) δ: 0.97 (3H, t, J=7.4 Hz), 1.48 (2H, dd, J=15.1, 7.4 Hz), 1.72-1.79 (2H, m), 1.84-1.93 (2H, m), 2.00-2.08 (2H, m), 2.32 (3H, s), 2.41 (3H, s), 2.49-2.60 (4H, m), 3.93 (2H, t, J=6.5 Hz), 4.06 (2H, s), 4.32 (2H, s), 5.77 (1H, s), 6.58 (1H, dd, J=12.0, 2.4 Hz), 6.66 (1H, dd, J=8.6, 2.4 Hz), 7.36 (1H, s), 7.36 (1H, dd, J=8.6, 8.6 Hz).

### Example 11

### Synthesis of Compound (I-113)

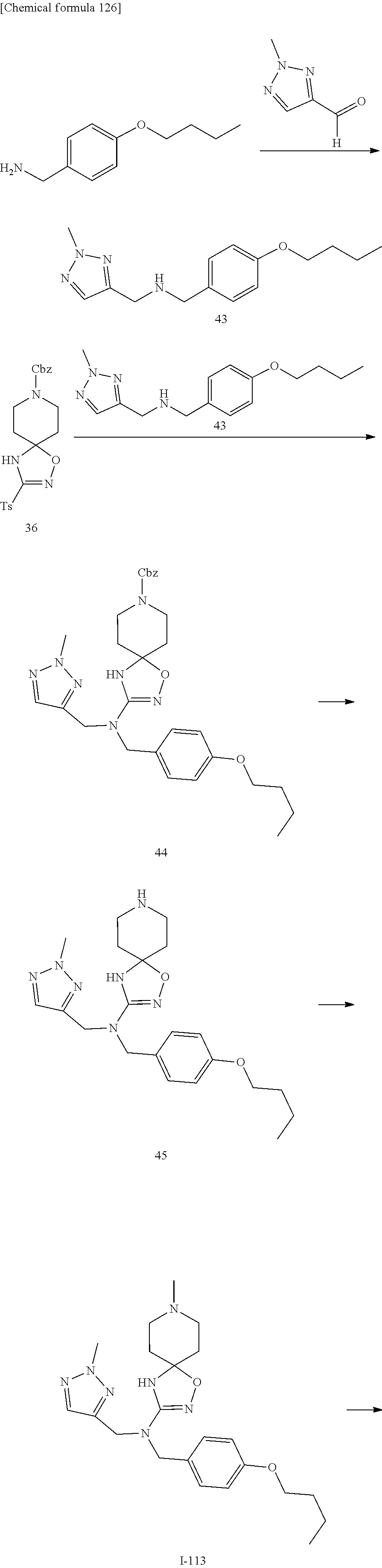

### Step 1 Synthesis of Compound 43

4-Butoxybenzenemethanamine (1.05 g, 5.85 mmol) was dissolved in ethanol (9.8 mL), 2-methyl-2H-1,2,3-triazole-4-carbaldehyde (650 mg, 5.85 mmol) was added, and the mixture was stirred at 80° C. for 1 hour. The mixture was ice-cooled, sodium borohydride (0.221 g, 5.85 mmol) was added, and the mixture was stirred at room temperature for 1 hour. Saturated aqueous sodium bicarbonate was added, and the mixture was extracted with chloroform. The organic layer was dried over-anhydrous sodium sulfate, and the solvent was distilled off under reduced pressure. The obtained residue was purified by amino column chromatography (hexane-ethyl acetate) to afford Compound 43 (1.38 g, yield 86%).

1H-NMR (CDCl3) δ: 0.97 (3H, t, J=7.4 Hz), 1.43-1.54 (2H, m), 1.72-1.79 (2H, m), 3.76 (2H, s), 3.85 (2H, s), 3.95 (2H, t, J=6.5 Hz), 4.16 (3H, s), 6.86 (2H, d, J=8.7 Hz), 7.23 (2H, d, J=8.7 Hz), 7.48 (1H, s).

### Step 2 Synthesis of Compound 44

Compound 36 (0.910 g, 2.11 mmol) was dissolved in 1,4-dioxane (6 mL), and Compound 43 (580 mg, 2.11 mmol) and DIPEA (6 mL, 34.4 mmol) were added, then the mixture was stirred at 130° C. for 8 hours. The solvent was distilled off under reduced pressure, and the obtained residue was purified by column chromatography (hexane-ethyl acetate) to afford Compound 44 (716 mg, yield 62%).

1H-NMR (CDCl3) δ: 0.98 (3H, t, J=7.4 Hz), 1.44-1.55 (2H, m), 1.66-1.80 (4H, m), 1.95-2.06 (2H, m), 3.43 (2H, t, J=10.5 Hz), 3.77-3.91 (2H, m), 3.95 (2H, t, J=6.7 Hz), 4.15 (3H, s), 4.27 (4H, s), 4.66 (1H, s), 5.13 (2H, s), 6.86 (2H, d, J=8.6 Hz), 7.19 (2H, d, J=8.6 Hz), 7.29-7.40 (6H, m).

### Step 3 Synthesis of Compound 45

Compound 44 (716 mg, 1.31 mmol) was dissolved in dichloromethane (11 mL), and dimethyl sulfide (1.93 mL, 26.1 mmol) and boron trifluoride diethyl ether complex (1.66 mL, 13.1 mmol) were added, then the mixture was stirred at room temperature overnight. A 10% aqueous solution of potassium carbonate was added, and the mixture was extracted with chloroform. The organic layer was washed with water and then dried over anhydrous sodium sulfate. The solvent was distilled off under reduced pressure, and the obtained residue was purified by amino column chromatography (chloroform-methanol) to afford Compound 45 (502 mg, yield 93%).

### Step 4 Synthesis of Compound (I-113)

Compound 45 (460 mg, 1.1 mmol) was dissolved in methanol (4.6 mL) and THF (4.6 mL), and a 37% aqueous solution of formaldehyde (0.249 mL, 3.34 mol) and sodium triacetoxyborohydride (708 mg, 3.34 mmol) were added, then the mixture was stirred at room temperature for 2 hours. Saturated aqueous sodium bicarbonate was added, then the mixture was extracted with chloroform, and the organic layer was dried over anhydrous sodium sulfate. The solvent was distilled off under reduced pressure, and the obtained residue was purified by amino column chromatography (chloroform-methanol) to afford Compound (I-113) (415 mg, yield 87%).

1H-NMR (CDCl3) δ: 0.98 (3H, t, J=7.4 Hz), 1.50 (2H, td, J=14.9, 7.4 Hz), 1.72-1.88 (4H, m), 1.99-2.07 (2H, m), 2.31 (3H, s), 2.44-2.59 (4H, m), 3.95 (2H, t, J=6.5 Hz), 4.15 (3H, s), 4.27 (4H, s), 4.57 (1H, s), 6.86 (2H, d, J=8.7 Hz), 7.20 (2H, d, J=8.7 Hz), 7.36 (1H, s).

### Example 12

### Synthesis of Compound (I-105)

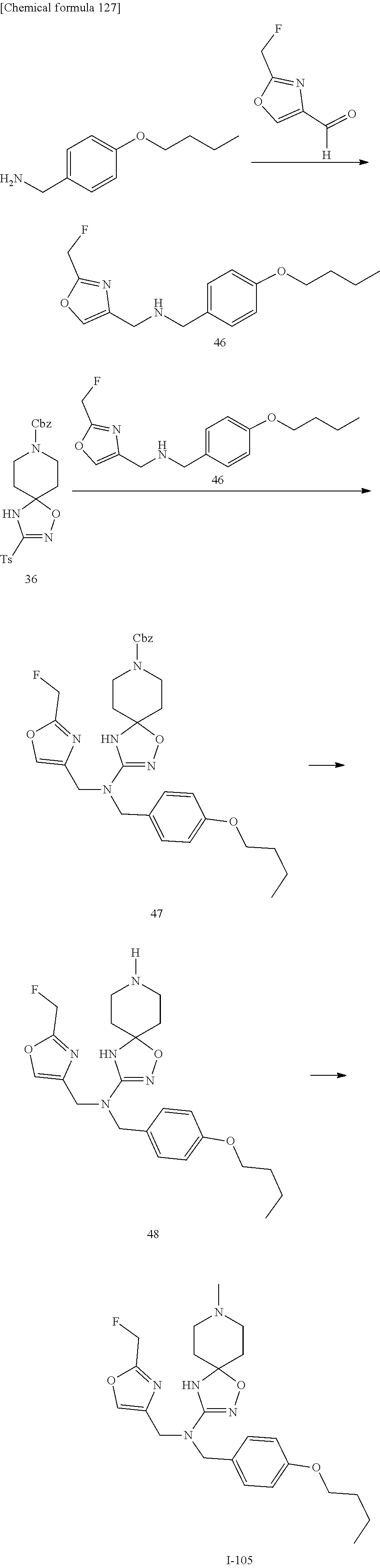

### Step 1 Synthesis of Compound 46

4-Butoxybenzenemethanamine (540 mg, 3.01 mmol) was dissolved in ethanol (9 mL), 2-fluoromethyl-4-oxazolecarboxaldehyde (389 mg, 3.01 mmol) was added, and the mixture was stirred at 80° C. for 1 hour. The mixture was ice-cooled, sodium borohydride (0.114 g, 3.01 mmol) was added, and the mixture was stirred at room temperature for 1 hour. Saturated aqueous sodium bicarbonate was added, and the mixture was extracted with chloroform. The organic layer was dried over anhydrous sodium sulfate, and the solvent was distilled off under reduced pressure. The obtained residue was purified by amino column chromatography (hexane-ethyl acetate) to afford Compound 46 (0.588 g, yield 67%).

1H-NMR (CDCl3) δ: 0.97 (3H, t, J=7.5 Hz), 1.49 (2H, td, J=15.0, 7.5 Hz), 1.73-1.80 (2H, m), 3.73 (2H, d, J=0.9 Hz), 3.75 (2H, s), 3.95 (2H, t, J=6.5 Hz), 5.36 (2H, d, J=47.4 Hz), 6.86 (2H, d, J=8.7 Hz), 7.23 (2H, d, J=8.7 Hz), 7.58 (1H, s).

### Step 2 Synthesis of Compound 47

Compound 36 (0.556 g, 1.30 mmol) was dissolved in 1,4-dioxane (2 mL), and Compound 46 (379 mg, 1.30 mmol) and DIPEA (2 mL, 11.5 mmol) were added, then the mixture was stirred at 130° C. for 7 hours. The solvent was distilled off under reduced pressure, and the obtained residue was purified by column chromatography (hexane-ethyl acetate) to afford Compound 47 (234 mg, yield 32%).

1H-NMR (CDCl3) δ: 0.98 (3H, t, J=7.6 Hz), 1.49 (2H, td, J=15.2, 7.6 Hz), 1.73-1.80 (4H, m), 1.97-2.05 (2H, m), 3.46 (2H, t, J=11.1 Hz), 3.77-3.91 (2H, m), 3.95 (2H, t, J=6.5 Hz), 4.12 (2H, s), 4.31 (2H, s), 5.14 (2H, s), 5.27 (2H, s), 5.39 (1H, s), 6.85 (2H, d, J=8.6 Hz), 7.19 (2H, d, J=8.6 Hz), 7.29-7.39 (5H, m), 7.44 (1H, s).

### Step 3 Synthesis of Compound 48

Compound 47 (234 mg, 0.414 mmol) was dissolved in dichloromethane (3.5 mL), and dimethyl sulfide (0.613 mL, 8.28 mmol) and boron trifluoride diethyl ether complex (0.525 mL, 4.14 mmol) were added, then the mixture was stirred at room temperature overnight. A 10% aqueous solution of potassium carbonate was added, and the mixture was extracted with chloroform. The organic layer was washed with water and then dried over anhydrous sodium sulfate. The solvent was distilled off under reduced pressure, and the obtained residue was purified by amino column chromatography (chloroform-methanol) to afford Compound 48 (127 mg, yield 71%).

### Step 4 Synthesis of Compound (I-105)

Compound 48 (127 mg, 0.294 mmol) was dissolved in methanol (1.3 mL) and THF (1.3 mL), and a 37% aqueous solution of formaldehyde (0.066 mL, 0.883 mol) and sodium triacetoxyborohydride (187 mg, 0.883 mmol) were added, then the mixture was stirred at room temperature for 1 hour. Saturated aqueous sodium bicarbonate was added, then the mixture was extracted with chloroform, and the organic layer was dried over anhydrous sodium sulfate. The solvent was distilled off under reduced pressure, and the obtained residue was purified by amino column chromatography (chloroform-methanol) to afford Compound (I-105) (122 mg, yield 93%).

1H-NMR (CDCl3) δ: 0.98 (3H, t, J=7.4 Hz), 1.49 (2H, td, J=15.0, 7.4 Hz), 1.73-1.80 (2H, m), 1.83-1.89 (2H, m), 2.00-2.09 (2H, m), 2.31 (3H, s), 2.45-2.62 (4H, m), 3.95 (2H, t, J=6.5 Hz), 4.14 (2H, s), 4.31 (2H, s), 5.10 (1H, s), 5.34 (2H, d, J=47.4 Hz), 6.85 (2H, d, J=8.5 Hz), 7.20 (2H, d, J=8.5 Hz), 7.46 (1H, s).

The following compounds were synthesized according to the above general synthetic method and the method described in Examples. The structure and physical properties (LC/MS data) are shown in the table below.

Incidentally, in the structural formula, “wedge shape” and “dashed line” indicate the steric configuration. Particularly, with regard to compounds whose steric configurations are described, a compound described as “racemate” in the item of “Configuration” is a racemic compound whose relative configuration has been specified.

In addition, with regard to compounds in which the bonds forming an asymmetric carbon are described by solid lines, a compound described as “racemate” in the item of “Configuration” is a racemic compound.

A compound described as “single isomer” in the item of “Configuration” is a single compound whose configuration has not been determined.

TABLE 1

| | | | Retention | LC/MS | |
|---|---|---|---|---|---|
| Compound | | | time | measurement | |
| No. | Structure | MS (m/z) | (min) | condition | Configuration |
| I-001 | 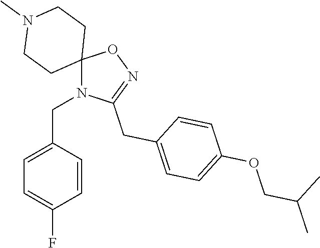 [SMILES: CC(C)COc1ccc(CC2=NOC3(CCN(C)CC3)N2Cc2ccc(F)cc2)cc1] | 467 | 0.95 | 1 | |
| I-002 | 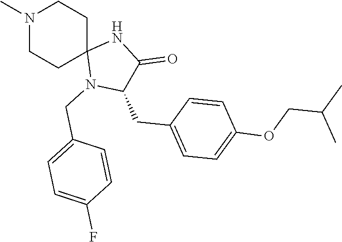 [SMILES: CC(C)COc1ccc(C[C@H]2C(=O)NC3(CCN(C)CC3)N2Cc2ccc(F)cc2)cc1] | 440 | 1.82 | 1 | |
| I-003 | 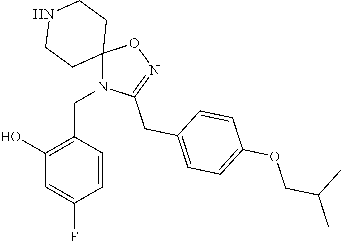 [SMILES: CC(C)COc1ccc(CC2=NOC3(CCNCC3)N2Cc2ccc(F)cc2O)cc1] | 428 | 1.54 | 1 | |
| I-004 | 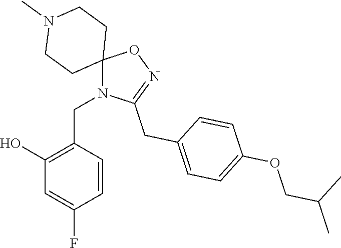 [SMILES: CC(C)COc1ccc(CC2=NOC3(CCN(C)CC3)N2Cc2ccc(F)cc2O)cc1] | 442 | 1.56 | 1 | |

TABLE 2

| | | | | |
|---|---|---|---|---|
| I-005 | 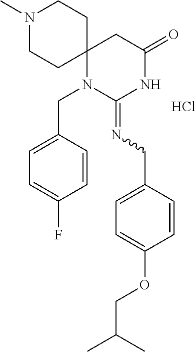 [SMILES: CC(C)COc1ccc(CN=C2NC(=O)CC3(CCN(C)CC3)N2Cc2ccc(F)cc2)cc1 ; Cl] | 467 | 0.95 | 1 |
| I-006 | 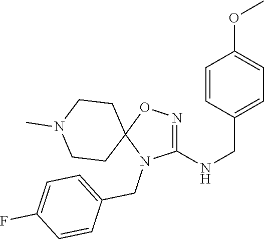 [SMILES: COc1ccc(CNC2=NOC3(CCN(C)CC3)N2Cc2ccc(F)cc2)cc1] | 399 | 0.89 | 1 |
| I-007 | 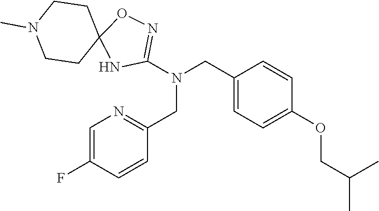 [SMILES: CC(C)COc1ccc(CN(Cc2ccc(F)cn2)C2=NOC3(CCN(C)CC3)N2)cc1] | 442 | 1.32 | 1 |
| I-008 | 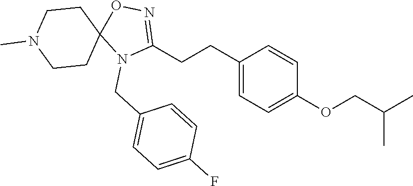 [SMILES: CC(C)COc1ccc(CCC2=NOC3(CCN(C)CC3)N2Cc2ccc(F)cc2)cc1] | 440 | 1.57 | 1 |
| I-009 | 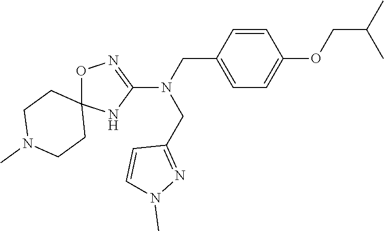 [SMILES: CC(C)COc1ccc(CN(Cc2ccn(C)n2)C2=NOC3(CCN(C)CC3)N2)cc1] | 427 | 1.34 | 1 |
| I-010 | 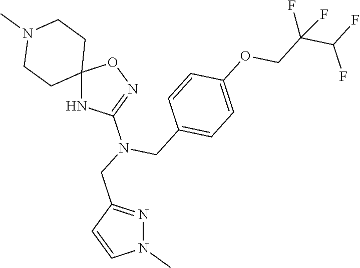 [SMILES: CN1CCC2(CC1)NC(N(Cc1ccc(OCC(F)(F)C(F)F)cc1)Cc1ccn(C)n1)=NO2] | 485 | 1.1 | 1 |

TABLE 3

| | | | |
|---|---|---|---|
| I- 011 | 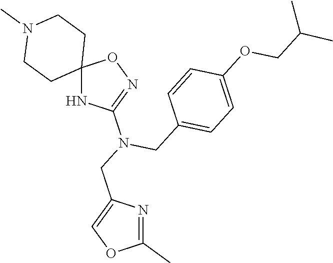 [SMILES: Cc1nc(CN(Cc2ccc(OCC(C)C)cc2)C2=NOC3(CCN(C)CC3)N2)co1] | 428 | 1.25 |

| | | | | |
|---|---|---|---|---|
| I- 012 | 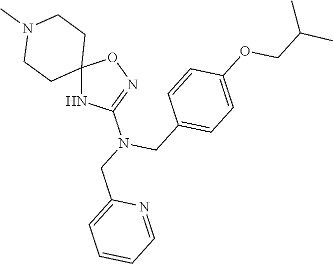 [SMILES: CC(C)COc1ccc(CN(Cc2ccccn2)C2=NOC3(CCN(C)CC3)N2)cc1] | 424 | 1.08 | 1 |
| I- 013 | 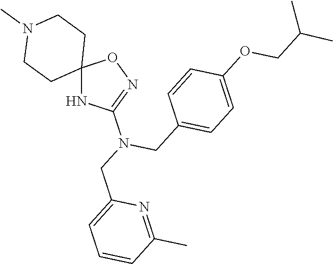 [SMILES: Cc1cccc(CN(Cc2ccc(OCC(C)C)cc2)C2=NOC3(CCN(C)CC3)N2)n1] | 438 | 0.98 | 1 |
| I- 014 | 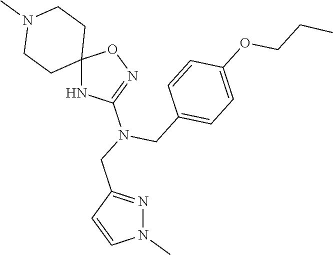 [SMILES: CCCOc1ccc(CN(Cc2ccn(C)n2)C2=NOC3(CCN(C)CC3)N2)cc1] | 413 | 1.09 | 1 |
| I- 015 | 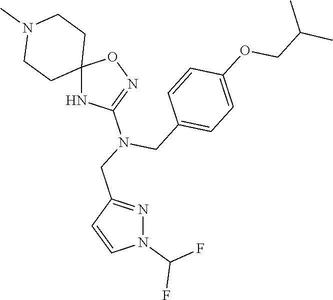 [SMILES: CC(C)COc1ccc(CN(Cc2ccn(C(F)F)n2)C2=NOC3(CCN(C)CC3)N2)cc1] | 463 | 1.32 | 1 |
| I- 016 | 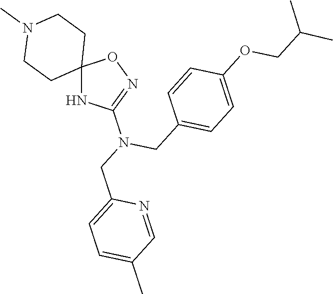 [SMILES: Cc1ccc(CN(Cc2ccc(OCC(C)C)cc2)C2=NOC3(CCN(C)CC3)N2)nc1] | 438 | 1.12 | 1 |
| I- 017 | 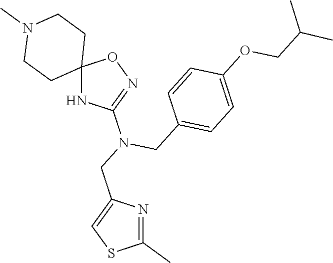 [SMILES: Cc1nc(CN(Cc2ccc(OCC(C)C)cc2)C2=NOC3(CCN(C)CC3)N2)cs1] | 444 | 1.67 | 1 |

TABLE 4

| | | | | |
|---|---|---|---|---|
| I-018 | 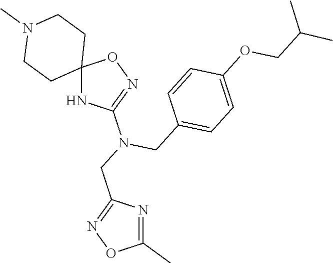 [SMILES: Cc1nc(CN(Cc2ccc(OCC(C)C)cc2)C2=NOC3(CCN(C)CC3)N2)no1] | 429 | 1.24 | 1 |
| I-019 | 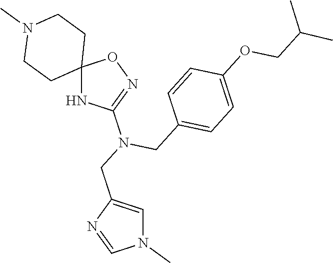 [SMILES: CC(C)COc1ccc(CN(Cc2cn(C)cn2)C2=NOC3(CCN(C)CC3)N2)cc1] | 427 | 0.75 | 1 |
| I-020 | 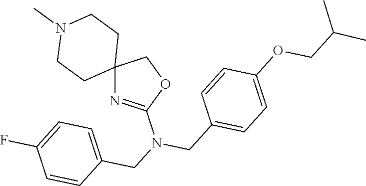 [SMILES: CC(C)COc1ccc(CN(Cc2ccc(F)cc2)C2=NC3(CCN(C)CC3)CO2)cc1] | 440 | 2.15 | 3 |
| I-021 | 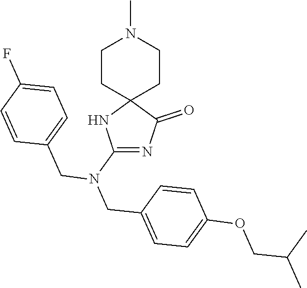 [SMILES: CC(C)COc1ccc(CN(Cc2ccc(F)cc2)C2=NC(=O)C3(CCN(C)CC3)N2)cc1] | 453 | 1.45 | 1 |
| I-022 | 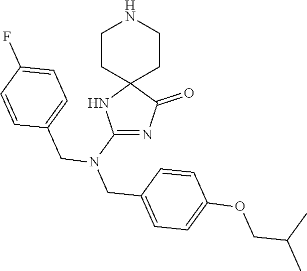 [SMILES: CC(C)COc1ccc(CN(Cc2ccc(F)cc2)C2=NC(=O)C3(CCNCC3)N2)cc1] | 439 | 1.42 | 1 |
| I-023 | 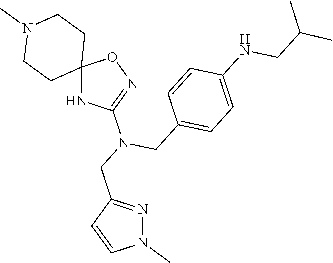 [SMILES: CC(C)CNc1ccc(CN(Cc2ccn(C)n2)C2=NOC3(CCN(C)CC3)N2)cc1] | 426 | 0.88 | 1 |

TABLE 5

| | | | | | |
|---|---|---|---|---|---|
| I-024 | 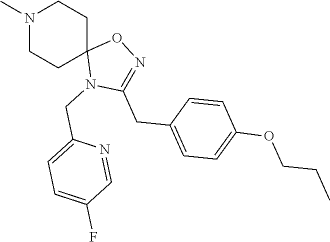 [SMILES: CCCOc1ccc(CC2=NOC3(CCN(C)CC3)N2Cc2ccc(F)cn2)cc1] | 413 | 1.24 | 1 | |
| I-025 | 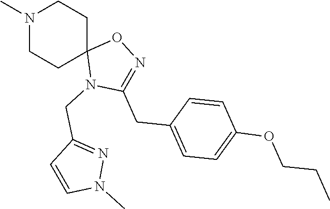 [SMILES: CCCOc1ccc(CC2=NOC3(CCN(C)CC3)N2Cc2ccn(C)n2)cc1] | 398 | 1.16 | 1 | |
| I-026 | 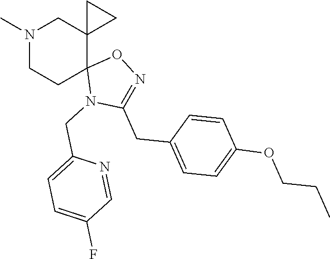 [SMILES: CCCOc1ccc(CC2=NOC3(CCN(C)CC34CC4)N2Cc2ccc(F)cn2)cc1] | 439 | 1.35 | 1 | |
| I-027 | 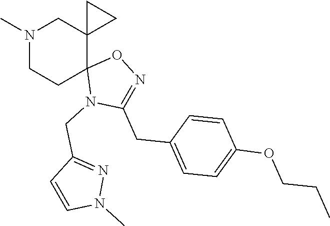 [SMILES: CCCOc1ccc(CC2=NOC3(CCN(C)CC34CC4)N2Cc2ccn(C)n2)cc1] | 424 | 1.23 | 1 | |
| I-028 | 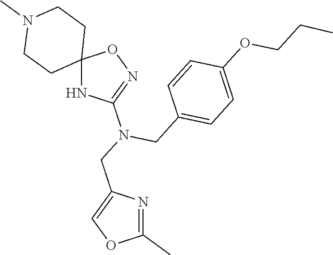 [SMILES: CCCOc1ccc(CN(Cc2coc(C)n2)C2=NOC3(CCN(C)CC3)N2)cc1] | 414 | 1.11 | 1 | |
| I-029 | 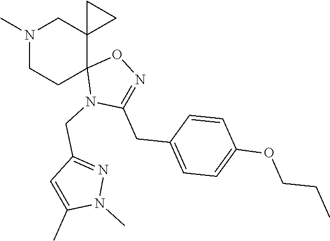 [SMILES: CCCOc1ccc(CC2=NOC3(CCN(C)CC34CC4)N2Cc2cc(C)n(C)n2)cc1] | 438 | 1.27 | 1 | racemate |

TABLE 6

| | | | | | |
|---|---|---|---|---|---|
| I-030 | 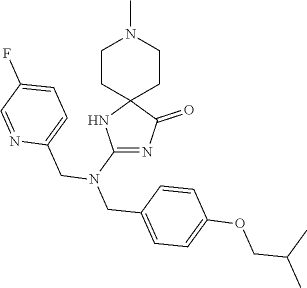 [SMILES: CC(C)COc1ccc(CN(Cc2ccc(F)cn2)C2=NC(=O)C3(CCN(C)CC3)N2)cc1] | 454 | 1.25 | 1 | |
| I-031 | 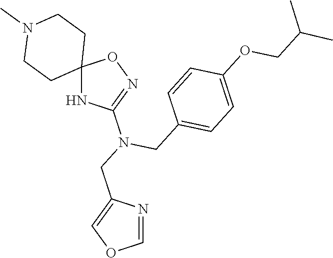 [SMILES: CC(C)COc1ccc(CN(Cc2cocn2)C2=NOC3(CCN(C)CC3)N2)cc1] | 414 | 1.21 | 1 | |
| I-032 | 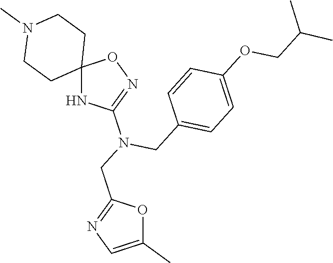 [SMILES: Cc1cnc(CN(Cc2ccc(OCC(C)C)cc2)C2=NOC3(CCN(C)CC3)N2)o1] | 428 | 1.3 | 1 | |
| I-033 | 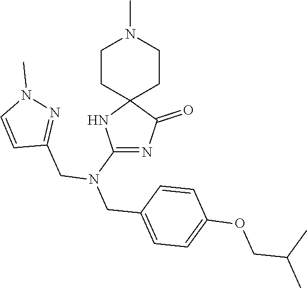 [SMILES: CC(C)COc1ccc(CN(Cc2ccn(C)n2)C2=NC(=O)C3(CCN(C)CC3)N2)cc1] | 439 | 1.66 | 3 | |
| I-034 | 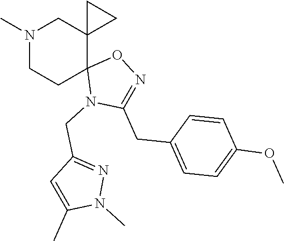 | 410 | 1.04 | 1 | racemate |
| I-035 | 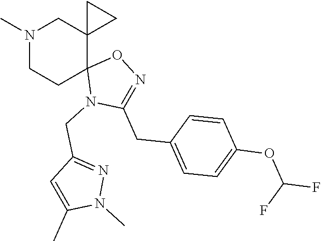 [SMILES: Cc1cc(CN2C(Cc3ccc(OC(F)F)cc3)=NOC23CCN(C)CC32CC2)nn1C] | 446 | 1.16 | 1 | racemate |
| I-036 | 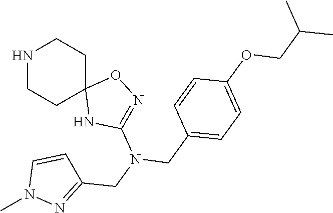 [SMILES: CC(C)COc1ccc(CN(Cc2ccn(C)n2)C2=NOC3(CCNCC3)N2)cc1] | 413 | 1.2 | 1 | |

TABLE 7

| | | | | | |
|---|---|---|---|---|---|
| I-037 | 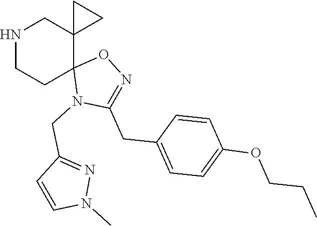 [SMILES: CCCOc1ccc(CC2=NOC3(CCNCC34CC4)N2Cc2ccn(C)n2)cc1] | 410 | 1.24 | 1 | racemate |
| I-038 | 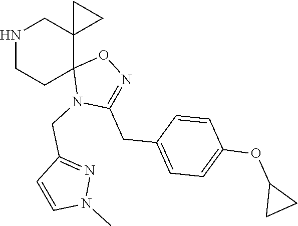 [SMILES: Cn1ccc(CN2C(Cc3ccc(OC4CC4)cc3)=NOC23CCNCC32CC2)n1] | 408 | 1.15 | 1 | racemate |
| I-039 | 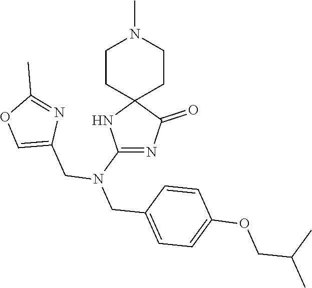 [SMILES: Cc1nc(CN(Cc2ccc(OCC(C)C)cc2)C2=NC(=O)C3(CCN(C)CC3)N2)co1] | 440 | 1.21 | 1 | |
| I-040 | 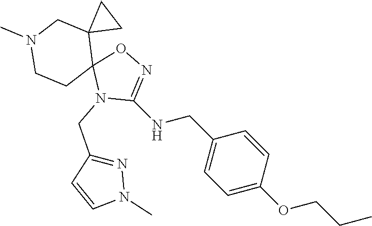 [SMILES: CCCOc1ccc(CNC2=NOC3(CCN(C)CC34CC4)N2Cc2ccn(C)n2)cc1] | 439 | 1.2 | 1 | racemate |
| I-041 | 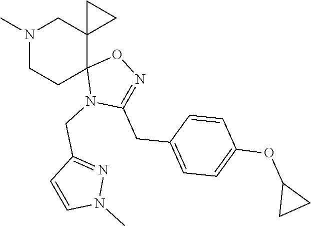 [SMILES: CN1CCC2(ON=C(Cc3ccc(OC4CC4)cc3)N2Cc2ccn(C)n2)C2(CC2)C1] | 422 | 1.18 | 1 | racemate |
| I-042 | 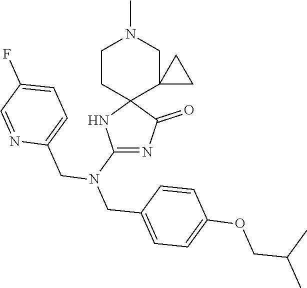 [SMILES: CC(C)COc1ccc(CN(Cc2ccc(F)cn2)C2=NC(=O)C3(CCN(C)CC34CC4)N2)cc1] | 480 | 1.36 | 1 | racemate |

TABLE 8

| | | | | | |
|---|---|---|---|---|---|
| I-043 | 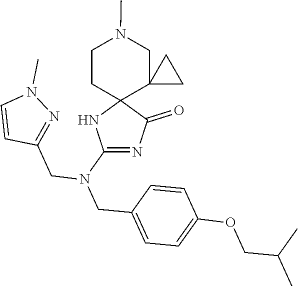 [SMILES: CC(C)COc1ccc(CN(Cc2ccn(C)n2)C2=NC(=O)C3(CCN(C)CC34CC4)N2)cc1] | 465 | 1.26 | 1 | racemate |
| I-044 | 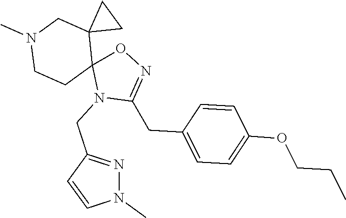 [SMILES: CCCOc1ccc(CC2=NOC3(CCN(C)CC34CC4)N2Cc2ccn(C)n2)cc1] | 424 | 1.3 | 1 | single isomer |
| I-045 | 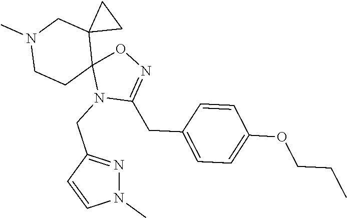 [SMILES: CCCOc1ccc(CC2=NOC3(CCN(C)CC34CC4)N2Cc2ccn(C)n2)cc1] | 424 | 1.29 | 1 | single isomer |
| I-046 | 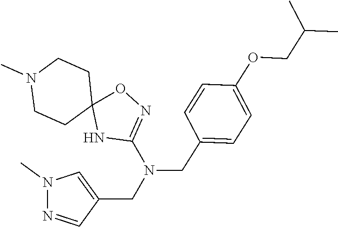 [SMILES: CC(C)COc1ccc(CN(Cc2cnn(C)c2)C2=NOC3(CCN(C)CC3)N2)cc1] | 427 | 1.5 | 1 | |
| I-047 | 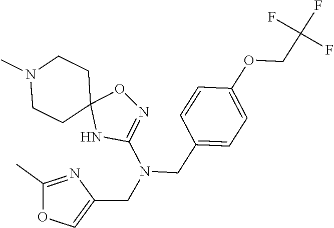 [SMILES: Cc1nc(CN(Cc2ccc(OCC(F)(F)F)cc2)C2=NOC3(CCN(C)CC3)N2)co1] | 454 | 1.25 | 1 | 1 |
| I-048 | 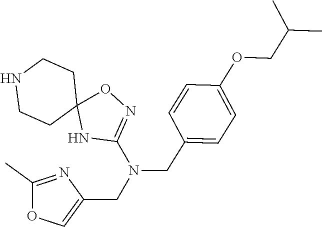 [SMILES: Cc1nc(CN(Cc2ccc(OCC(C)C)cc2)C2=NOC3(CCNCC3)N2)co1] | 414 | 1.42 | 1 | |

TABLE 9

| | | | | |
|---|---|---|---|---|
| I- 049 | 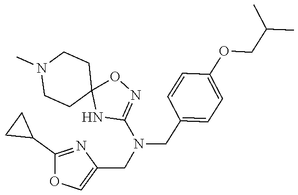 [SMILES: CC(C)COc1ccc(CN(Cc2coc(C3CC3)n2)C2=NOC3(CCN(C)CC3)N2)cc1] | 454 | 1.44 | 1 |
| I- 050 | 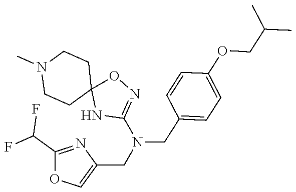 [SMILES: CC(C)COc1ccc(CN(Cc2coc(C(F)F)n2)C2=NOC3(CCN(C)CC3)N2)cc1] | 464 | 1.4 | 1 |
| I- 051 | 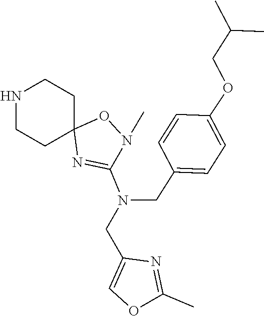 [SMILES: Cc1nc(CN(Cc2ccc(OCC(C)C)cc2)C2=NC3(CCNCC3)ON2C)co1] | 428 | 1.46 | 1 |
| I- 052 | 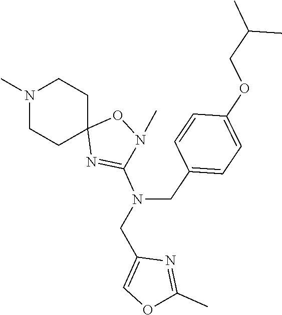 [SMILES: Cc1nc(CN(Cc2ccc(OCC(C)C)cc2)C2=NC3(CCN(C)CC3)ON2C)co1] | 442 | 1.48 | 1 |
| I- 053 | 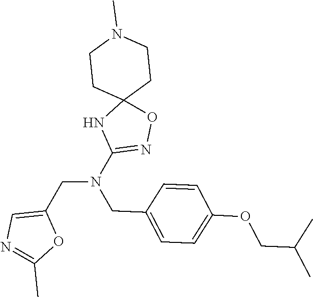 [SMILES: Cc1ncc(CN(Cc2ccc(OCC(C)C)cc2)C2=NOC3(CCN(C)CC3)N2)o1] | 428 | 1.35 | 1 |
| I- 054 | 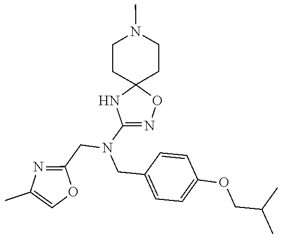 [SMILES: Cc1coc(CN(Cc2ccc(OCC(C)C)cc2)C2=NOC3(CCN(C)CC3)N2)n1] | 428 | 1.45 | 1 |

TABLE 10

| | | | | |
|---|---|---|---|---|
| I-055 | 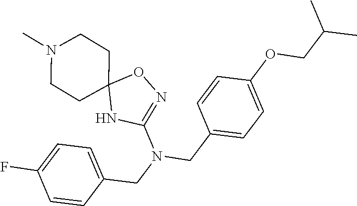 [SMILES: CC(C)COc1ccc(CN(Cc2ccc(F)cc2)C2=NOC3(CCN(C)CC3)N2)cc1] | 441 | 1.9 | 2 |
| I-056 | 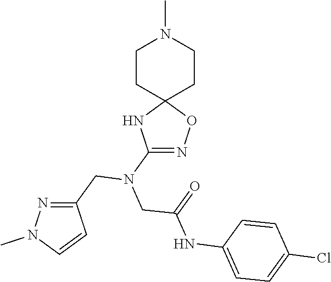 [SMILES: CN1CCC2(CC1)NC(N(CC(=O)Nc1ccc(Cl)cc1)Cc1ccn(C)n1)=NO2] | 432 | 1.03 | 1 |
| I-057 | 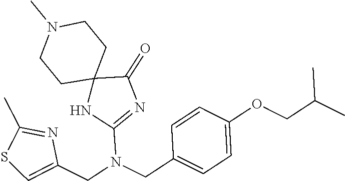 [SMILES: Cc1nc(CN(Cc2ccc(OCC(C)C)cc2)C2=NC(=O)C3(CCN(C)CC3)N2)cs1] | 456 | 1.4 | 1 |
| I-058 | 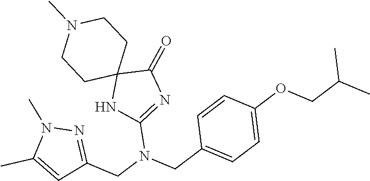 [SMILES: Cc1cc(CN(Cc2ccc(OCC(C)C)cc2)C2=NC(=O)C3(CCN(C)CC3)N2)nn1C] | 453 | 1.67 | 1 |
| I-059 | 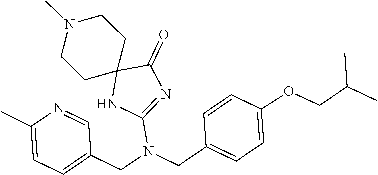 [SMILES: Cc1ccc(CN(Cc2ccc(OCC(C)C)cc2)C2=NC(=O)C3(CCN(C)CC3)N2)cn1] | 450 | 1.16 | 1 |
| I-060 | 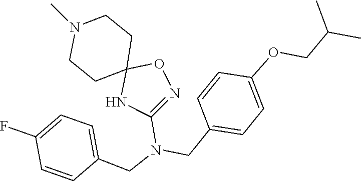 [SMILES: CC(C)COc1ccc(CN(Cc2ccc(F)cc2)C2=NOC3(CCN(C)CC3)N2)cc1] | 469 | 1.9 | 1 |

TABLE 11

| | | | | |
|---|---|---|---|---|
| I-061 | 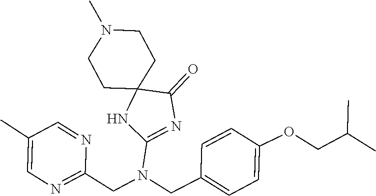 [SMILES: Cc1cnc(CN(Cc2ccc(OCC(C)C)cc2)C2=NC(=O)C3(CCN(C)CC3)N2)nc1] | 451 | 1.49 | 1 |
| I-062 | 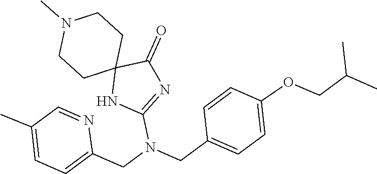 [SMILES: Cc1ccc(CN(Cc2ccc(OCC(C)C)cc2)C2=NC(=O)C3(CCN(C)CC3)N2)nc1] | 450 | 1.41 | 1 |
| I-063 | 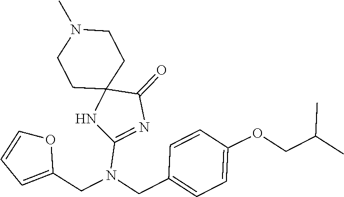 [SMILES: CC(C)COc1ccc(CN(Cc2ccco2)C2=NC(=O)C3(CCN(C)CC3)N2)cc1] | 425 | 1.48 | 1 |
| I-064 | 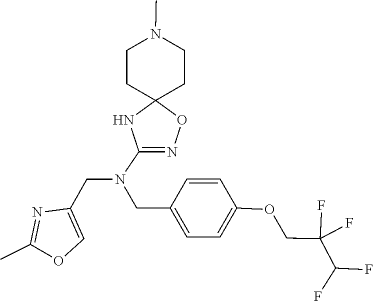 [SMILES: Cc1nc(CN(Cc2ccc(OCC(F)(F)C(F)F)cc2)C2=NOC3(CCN(C)CC3)N2)co1] | 486 | 1.31 | 1 |
| I-065 | 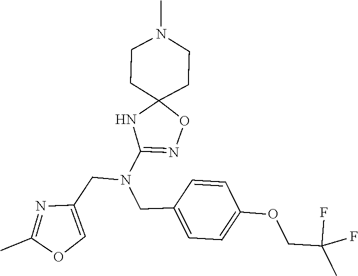 [SMILES: Cc1nc(CN(Cc2ccc(OCC(C)(F)F)cc2)C2=NOC3(CCN(C)CC3)N2)co1] | 450 | 1.25 | 1 |
| I-066 | 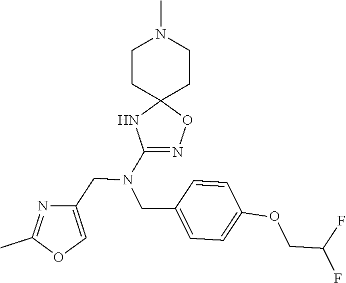 [SMILES: Cc1nc(CN(Cc2ccc(OCC(F)F)cc2)C2=NOC3(CCN(C)CC3)N2)co1] | 436 | 1.12 | 1 |

TABLE 12

| | | | | |
|---|---|---|---|---|
| I-067 | 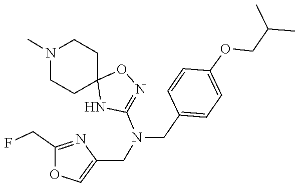 [SMILES: CC(C)COc1ccc(CN(Cc2coc(CF)n2)C2=NOC3(CCN(C)CC3)N2)cc1] | 446 | 1.94 | 2 |
| I-068 | 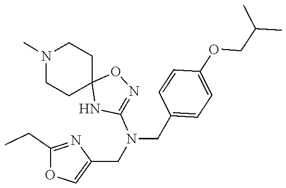 [SMILES: CCc1nc(CN(Cc2ccc(OCC(C)C)cc2)C2=NOC3(CCN(C)CC3)N2)co1] | 442 | 1.65 | 2 |
| I-069 | 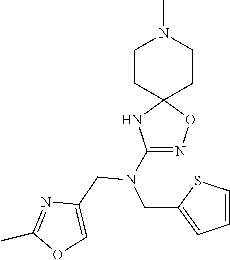 [SMILES: Cc1nc(CN(Cc2cccs2)C2=NOC3(CCN(C)CC3)N2)co1] | 362 | 0.91 | 1 |
| I-070 | 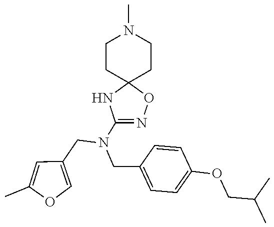 [SMILES: Cc1cc(CN(Cc2ccc(OCC(C)C)cc2)C2=NOC3(CCN(C)CC3)N2)co1] | 427 | 1.7 | 1 |
| I-071 | 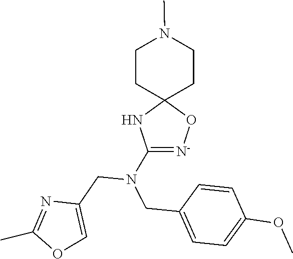 | 386 | 0.98 | 1 |
| I-072 | 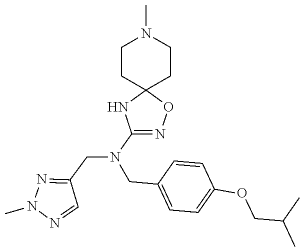 [SMILES: CC(C)COc1ccc(CN(Cc2cnn(C)n2)C2=NOC3(CCN(C)CC3)N2)cc1] | 428 | 1.39 | 1 |

TABLE 13

| | | | | | |
|---|---|---|---|---|---|
| I-073 | 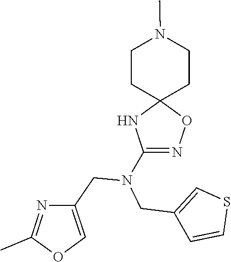 [SMILES: Cc1nc(CN(Cc2ccsc2)C2=NOC3(CCN(C)CC3)N2)co1] | 362 | 0.89 | 1 | |
| I-074 | 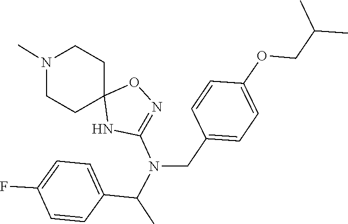 [SMILES: CC(C)COc1ccc(CN(C2=NOC3(CCN(C)CC3)N2)C(C)c2ccc(F)cc2)cc1] | 455 | 1.78 | 1 | single isomer |
| I-075 | 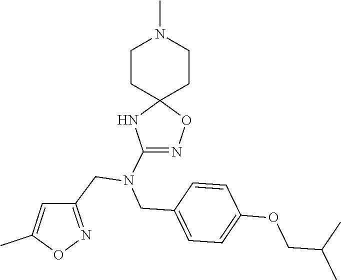 [SMILES: Cc1cc(CN(Cc2ccc(OCC(C)C)cc2)C2=NOC3(CCN(C)CC3)N2)no1] | 428 | 1.48 | 1 | |
| I-076 | 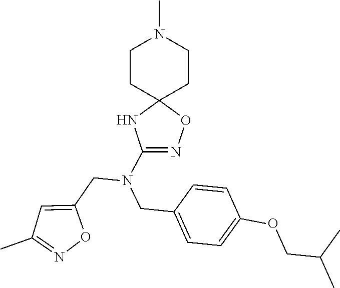 [SMILES: Cc1cc(CN(Cc2ccc(OCC(C)C)cc2)C2=NOC3(CCN(C)CC3)N2)on1] | 428 | 1.44 | 1 | |
| I-077 | 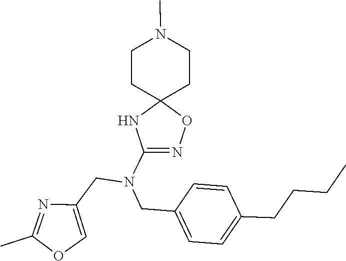 [SMILES: CCCCc1ccc(CN(Cc2coc(C)n2)C2=NOC3(CCN(C)CC3)N2)cc1] | 412 | 1.52 | 1 | |
| I-078 | 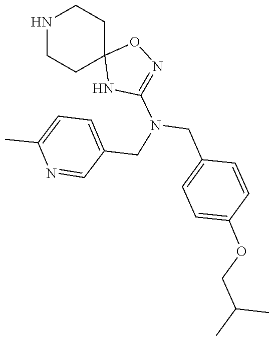 [SMILES: Cc1ccc(CN(Cc2ccc(OCC(C)C)cc2)C2=NOC3(CCNCC3)N2)cn1] | 424 | 1.19 | 1 | |

TABLE 14

| | | | | |
|---|---|---|---|---|
| I-079 | 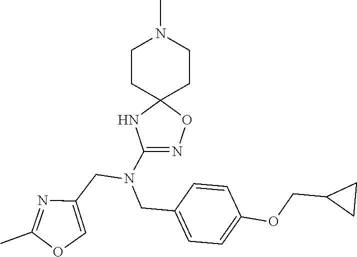 [SMILES: Cc1nc(CN(Cc2ccc(OCC3CC3)cc2)C2=NOC3(CCN(C)CC3)N2)co1] | 426 | 1.27 | 1 |
| I-080 | 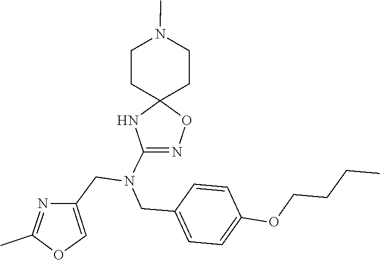 [SMILES: CCCCOc1ccc(CN(Cc2coc(C)n2)C2=NOC3(CCN(C)CC3)N2)cc1] | 428 | 1.45 | 1 |
| I-081 | 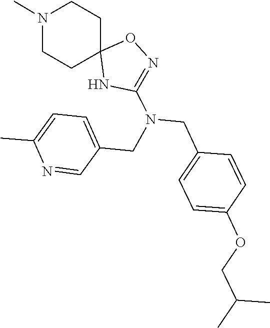 [SMILES: Cc1ccc(CN(Cc2ccc(OCC(C)C)cc2)C2=NOC3(CCN(C)CC3)N2)cn1] | 438 | 1.11 | 1 |
| I-082 | 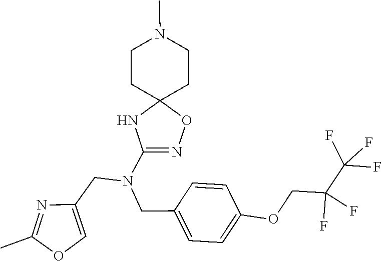 [SMILES: Cc1nc(CN(Cc2ccc(OCC(F)(F)C(F)(F)F)cc2)C2=NOC3(CCN(C)CC3)N2)co1] | 504 | 1.54 | 1 |
| I-083 | 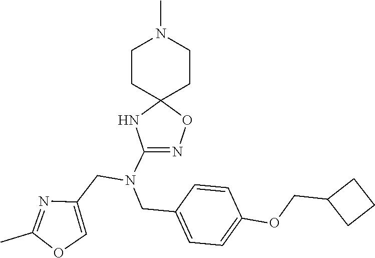 [SMILES: Cc1nc(CN(Cc2ccc(OCC3CCC3)cc2)C2=NOC3(CCN(C)CC3)N2)co1] | 440 | 1.49 | 1 |
| I-084 | 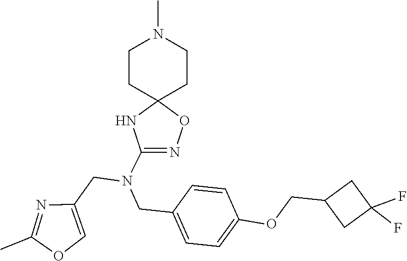 [SMILES: Cc1nc(CN(Cc2ccc(OCC3CC(F)(F)C3)cc2)C2=NOC3(CCN(C)CC3)N2)co1] | 476 | 1.36 | 1 |

TABLE 15

| | | | | | |
|---|---|---|---|---|---|
| I-085 | 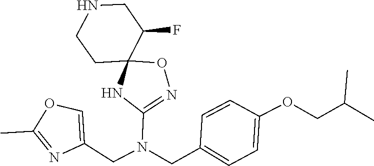 [SMILES: Cc1nc(CN(Cc2ccc(OCC(C)C)cc2)C2=NO[C@@]3(CCNC[C@H]3F)N2)co1] | 432 | 1.39 | 1 | racemate |
| I-086 | 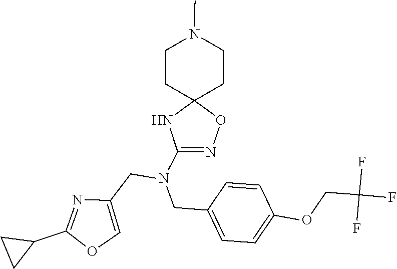 [SMILES: CN1CCC2(CC1)NC(N(Cc1ccc(OCC(F)(F)F)cc1)Cc1coc(C3CC3)n1)=NO2] | 480 | 1.42 | 1 | |
| I-087 | 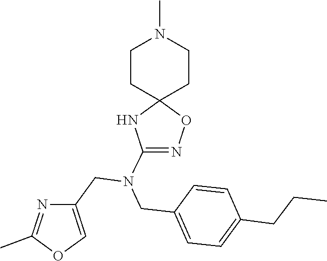 [SMILES: CCCc1ccc(CN(Cc2coc(C)n2)C2=NOC3(CCN(C)CC3)N2)cc1] | 398 | 1.34 | 1 | |

TABLE 16

| | | | | |
|---|---|---|---|---|
| I-088 | 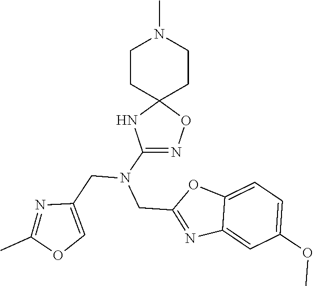 [SMILES: COc1ccc2oc(CN(Cc3coc(C)n3)C3=NOC4(CCN(C)CC4)N3)nc2c1] | 427 | 0.87 | 1 |
| I-089 | 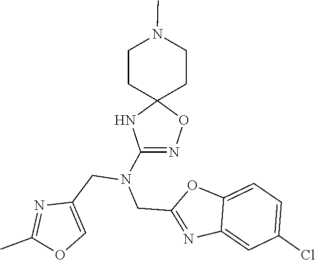 [SMILES: Cc1nc(CN(Cc2nc3cc(Cl)ccc3o2)C2=NOC3(CCN(C)CC3)N2)co1] | 431 | 0.95 | 1 |
| I-090 | 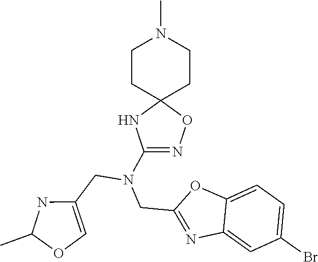 | 475 | 1.03 | 1 |
| I-091 | 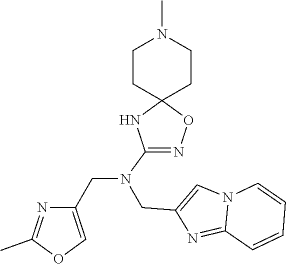 [SMILES: Cc1nc(CN(Cc2cn3ccccc3n2)C2=NOC3(CCN(C)CC3)N2)co1] | 396 | 1.07 | 3 |
| I-092 | 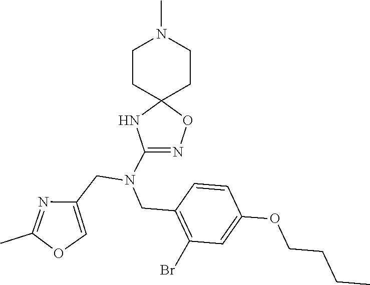 [SMILES: CCCCOc1ccc(CN(Cc2coc(C)n2)C2=NOC3(CCN(C)CC3)N2)c(Br)c1] | 506 | 1.84 | 1 |
| I-093 | 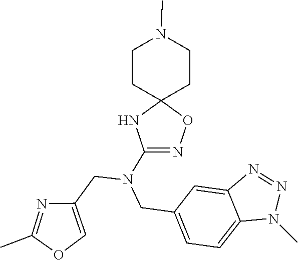 | 411 | 0.66 | 1 |
| I-094 | 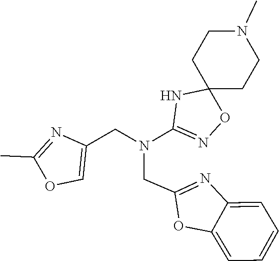 [SMILES: Cc1nc(CN(Cc2nc3ccccc3o2)C2=NOC3(CCN(C)CC3)N2)co1] | 397 | 0.81 | 1 |

TABLE 17

| | | | | |
|---|---|---|---|---|
| I-095 | 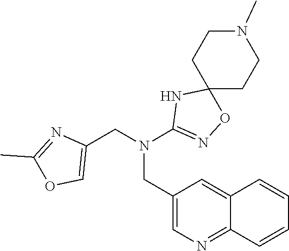 [SMILES: Cc1nc(CN(Cc2cnc3ccccc3c2)C2=NOC3(CCN(C)CC3)N2)co1] | 407 | 0.62 | 1 |
| I-096 | 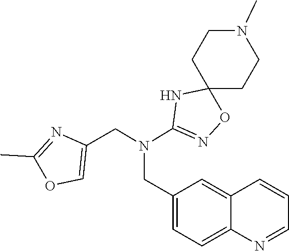 [SMILES: Cc1nc(CN(Cc2ccc3ncccc3c2)C2=NOC3(CCN(C)CC3)N2)co1] | 407 | 1.34 | 3 |
| I-097 | 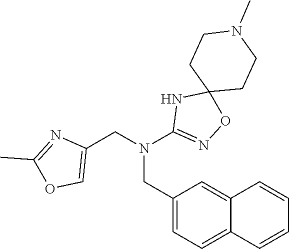 [SMILES: Cc1nc(CN(Cc2ccc3ccccc3c2)C2=NOC3(CCN(C)CC3)N2)co1] | 406 | 1.09 | 1 |
| I-098 | 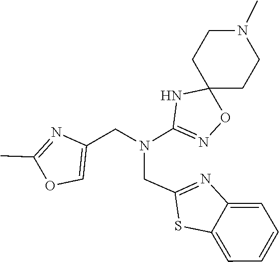 [SMILES: Cc1nc(CN(Cc2nc3ccccc3s2)C2=NOC3(CCN(C)CC3)N2)co1] | 413 | 0.99 | 1 |
| I-099 | 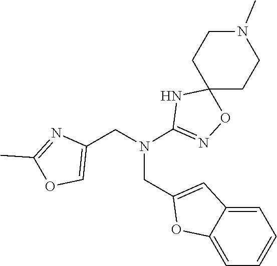 [SMILES: Cc1nc(CN(Cc2cc3ccccc3o2)C2=NOC3(CCN(C)CC3)N2)co1] | 396 | 1.09 | 1 |
| I-100 | 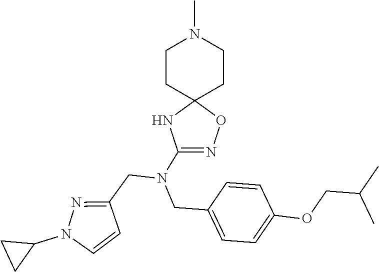 [SMILES: CC(C)COc1ccc(CN(Cc2ccn(C3CC3)n2)C2=NOC3(CCN(C)CC3)N2)cc1] | 396 | 1.09 | 1 |
| I-101 | 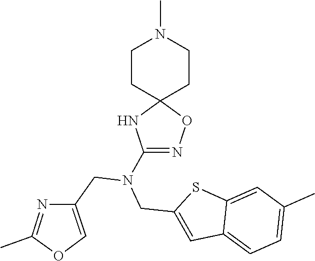 | 426 | 1.31 | 1 |

TABLE 18

| | | | | |
|---|---|---|---|---|
| I-102 | 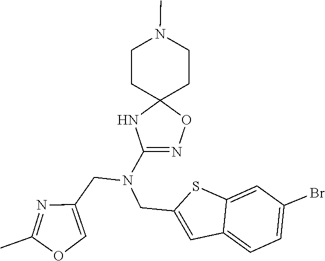 [SMILES: Cc1nc(CN(Cc2cc3ccc(Br)cc3s2)C2=NOC3(CCN(C)CC3)N2)co1] | 490 | 1.38 | 1 |
| I-103 | 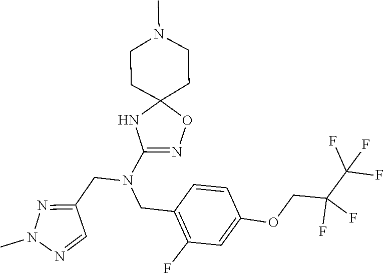 [SMILES: CN1CCC2(CC1)NC(N(Cc1cnn(C)n1)Cc1ccc(OCC(F)(F)C(F)(F)F)cc1F)=NO2] | 522 | 1.7 | 1 |
| I-104 | 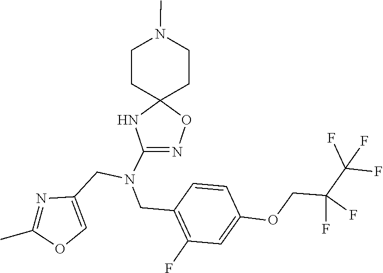 [SMILES: Cc1nc(CN(Cc2ccc(OCC(F)(F)C(F)(F)F)cc2F)C2=NOC3(CCN(C)CC3)N2)co1] | 522 | 1.7 | 1 |
| I-105 | 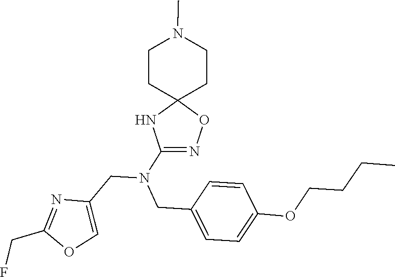 [SMILES: CCCCOc1ccc(CN(Cc2coc(CF)n2)C2=NOC3(CCN(C)CC3)N2)cc1] | 446 | 1.42 | 1 |
| I-106 | 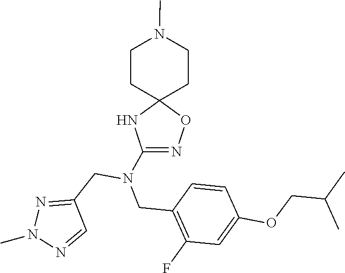 [SMILES: CC(C)COc1ccc(CN(Cc2cnn(C)n2)C2=NOC3(CCN(C)CC3)N2)c(F)c1] | 446 | 1.9 | 2 |
| I-107 | 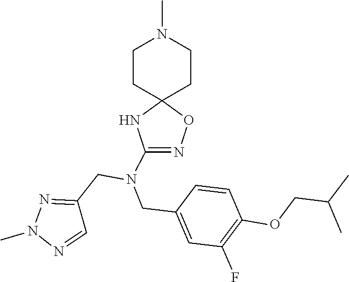 [SMILES: CC(C)COc1ccc(CN(Cc2cnn(C)n2)C2=NOC3(CCN(C)CC3)N2)cc1F] | 446 | 1.87 | 2 |
| I-108 | 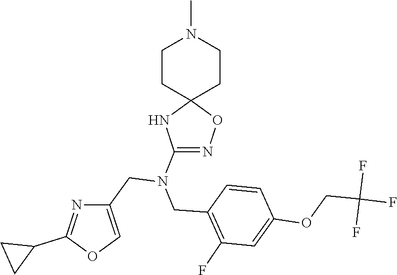 [SMILES: CN1CCC2(CC1)NC(N(Cc1coc(C3CC3)n1)Cc1ccc(OCC(F)(F)F)cc1F)=NO2] | 498 | 1.41 | 1 |

TABLE 19

| | | | | |
|---|---|---|---|---|
| I-109 | 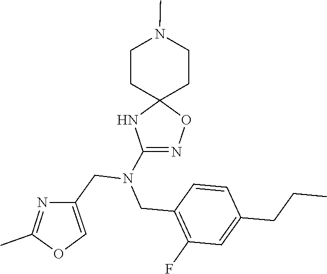 [SMILES: CCCc1ccc(CN(Cc2coc(C)n2)C2=NOC3(CCN(C)CC3)N2)c(F)c1] | 416 | 1.58 | 1 |
| I-110 | 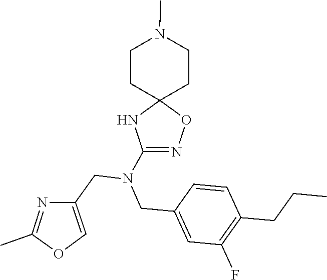 [SMILES: CCCc1ccc(CN(Cc2coc(C)n2)C2=NOC3(CCN(C)CC3)N2)cc1F] | 416 | 1.58 | 2 |
| I-111 | 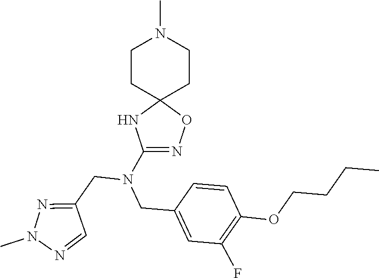 [SMILES: CCCCOc1ccc(CN(Cc2cnn(C)n2)C2=NOC3(CCN(C)CC3)N2)cc1F] | 446 | 1.35 | 1 |
| I-112 | 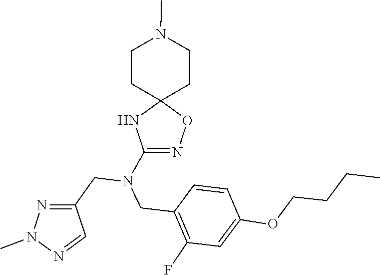 [SMILES: CCCCOc1ccc(CN(Cc2cnn(C)n2)C2=NOC3(CCN(C)CC3)N2)c(F)c1] | 446 | 1.37 | 1 |
| I-113 | 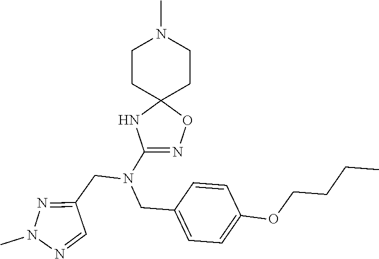 [SMILES: CCCCOc1ccc(CN(Cc2cnn(C)n2)C2=NOC3(CCN(C)CC3)N2)cc1] | 428 | 1.33 | 1 |
| I-114 | 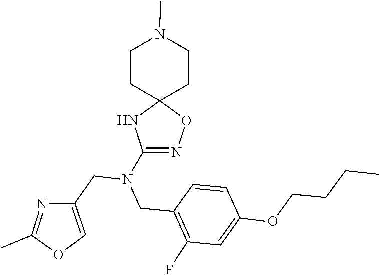 [SMILES: CCCCOc1ccc(CN(Cc2coc(C)n2)C2=NOC3(CCN(C)CC3)N2)c(F)c1] | 446 | 1.45 | 1 |
| I-115 | 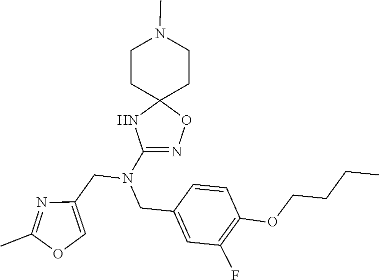 [SMILES: CCCCOc1ccc(CN(Cc2coc(C)n2)C2=NOC3(CCN(C)CC3)N2)cc1F] | 446 | 1.42 | 1 |

TABLE 20

| | | | | |
|---|---|---|---|---|
| I-116 | 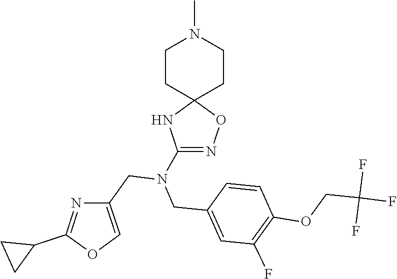 [SMILES: CN1CCC2(CC1)NC(N(Cc1ccc(OCC(F)(F)F)c(F)c1)Cc1coc(C3CC3)n1)=NO2] | 498 | 1.39 | 1 |
| I-117 | 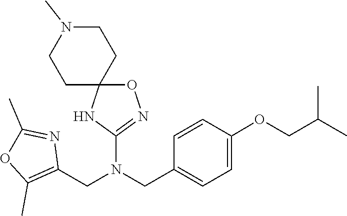 [SMILES: Cc1nc(CN(Cc2ccc(OCC(C)C)cc2)C2=NOC3(CCN(C)CC3)N2)c(C)o1] | 442 | 1.66 | 2 |
| I-118 | 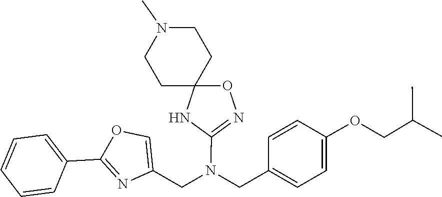 [SMILES: CC(C)COc1ccc(CN(Cc2coc(-c3ccccc3)n2)C2=NOC3(CCN(C)CC3)N2)cc1] | 490 | 1.91 | 2 |
| I-119 | 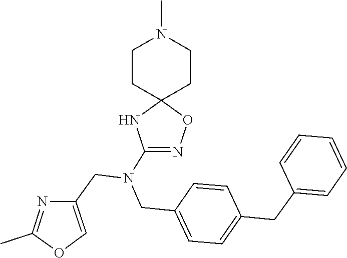 [SMILES: Cc1nc(CN(Cc2ccc(Cc3ccccc3)cc2)C2=NOC3(CCN(C)CC3)N2)co1] | 446 | 1.43 | 1 |
| I-120 | 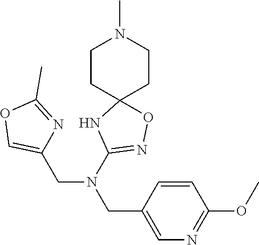 [SMILES: COc1ccc(CN(Cc2coc(C)n2)C2=NOC3(CCN(C)CC3)N2)cn1] | 387 | 0.79 | 1 |
| I-121 | 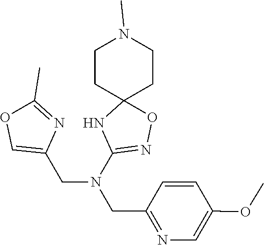 [SMILES: COc1ccc(CN(Cc2coc(C)n2)C2=NOC3(CCN(C)CC3)N2)nc1] | 387 | 0.66 | 1 |
| I-122 | 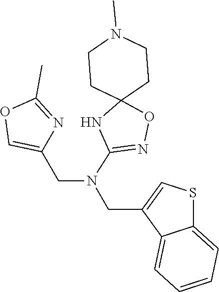 [SMILES: Cc1nc(CN(Cc2csc3ccccc23)C2=NOC3(CCN(C)CC3)N2)co1] | 412 | 1.2 | 1 |

TABLE 21

| | | | | |
|---|---|---|---|---|
| I-123 | 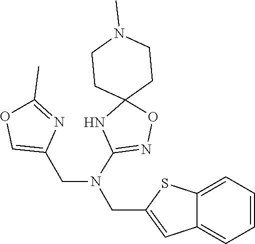 [SMILES: Cc1nc(CN(Cc2cc3ccccc3s2)C2=NOC3(CCN(C)CC3)N2)co1] | 412 | 1.2 | 1 |
| I-124 | 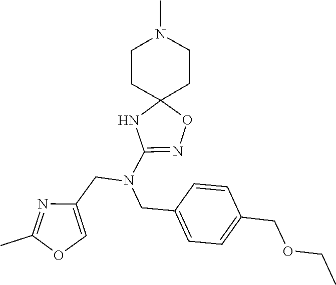 [SMILES: CCOCc1ccc(CN(Cc2coc(C)n2)C2=NOC3(CCN(C)CC3)N2)cc1] | 414 | 1.09 | 1 |
| I-125 | 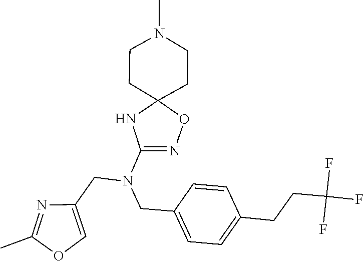 [SMILES: Cc1nc(CN(Cc2ccc(CCC(F)(F)F)cc2)C2=NOC3(CCN(C)CC3)N2)co1] | 452 | 1.81 | 1 |
| I-126 | 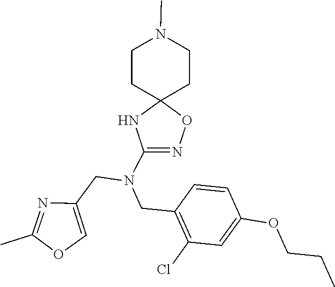 [SMILES: CCCOc1ccc(CN(Cc2coc(C)n2)C2=NOC3(CCN(C)CC3)N2)c(Cl)c1] | 448 | 1.61 | 2 |
| I-127 | 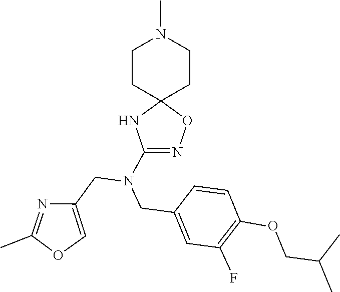 [SMILES: Cc1nc(CN(Cc2ccc(OCC(C)C)c(F)c2)C2=NOC3(CCN(C)CC3)N2)co1] | 446 | 1.65 | 2 |
| I-128 | 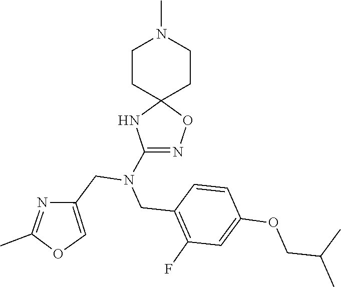 [SMILES: Cc1nc(CN(Cc2ccc(OCC(C)C)cc2F)C2=NOC3(CCN(C)CC3)N2)co1] | 446 | 1.66 | 2 |
| I-129 | 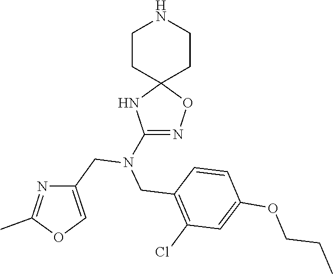 [SMILES: CCCOc1ccc(CN(Cc2coc(C)n2)C2=NOC3(CCNCC3)N2)c(Cl)c1] | 434 | 1.57 | 2 |

TABLE 22

| | | | | |
|---|---|---|---|---|
| I-130 | 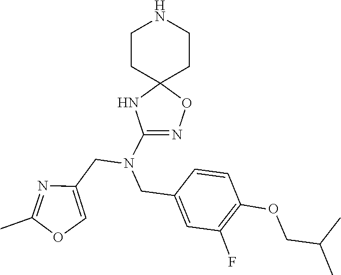 [SMILES: Cc1nc(CN(Cc2ccc(OCC(C)C)c(F)c2)C2=NOC3(CCNCC3)N2)co1] | 432 | 1.56 | 2 |
| I-131 | 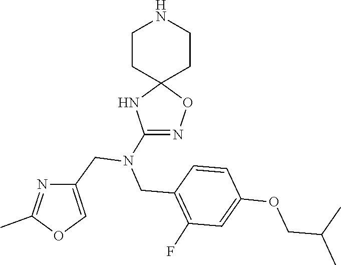 [SMILES: Cc1nc(CN(Cc2ccc(OCC(C)C)cc2F)C2=NOC3(CCNCC3)N2)co1] | 432 | 1.57 | 2 |
| I-132 | 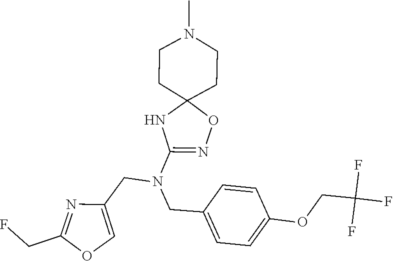 [SMILES: CN1CCC2(CC1)NC(N(Cc1ccc(OCC(F)(F)F)cc1)Cc1coc(CF)n1)=NO2] | 472 | 1.29 | 1 |
| I-133 | 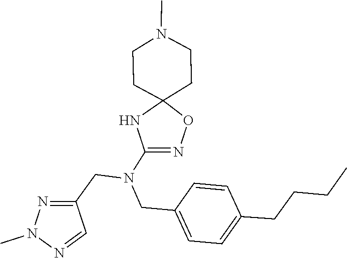 [SMILES: CCCCc1ccc(CN(Cc2cnn(C)n2)C2=NOC3(CCN(C)CC3)N2)cc1] | 412 | 1.47 | 1 |
| I-134 | 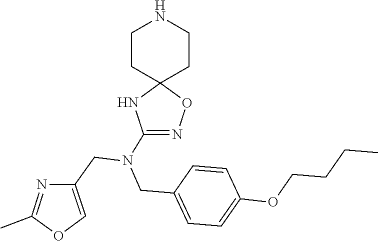 [SMILES: CCCCOc1ccc(CN(Cc2coc(C)n2)C2=NOC3(CCNCC3)N2)cc1] | 414 | 1.59 | 1 |

TABLE 23

| Compound | |
|---|---|
| No. | NMR |
| I - 001 | 1H-NMR (CDCl3) δ: 1.02 (d, J = 6.8 Hz, 6H), 1.60-1.72 (m, 2H), 1.81- |
| | 1.92 (m, 2H), 2.02-2.14 (m, 1H), 2.29 (s, 3H), 2.31-2.41 (m, 2H), 2.57- |
| | 2.66 (m, 2H), 3.65 (s, 2H), 3.70 (d, J = 6.5 Hz, 2H), 4.28 (s, 2H), 6.86 |
| | (d, J = 8.5 Hz, 2H), 6.99 (t, J = 8.5 Hz, 2H), 7.14-7.22 (m, 4H). |
| I - 003 | 1H-NMR (DMSO-D6) δ: 0.97 (d, J = 6.5 Hz, 6H), 1.26-1.38 (m, 2H), |
| | 1.42-1.51 (m, 2H), 1.93-2.04 (m, 1H), 2.59-2.77 (m, 4H), 3.67 (s, 2H), |
| | 3.71 (d, J = 6.5 Hz, 2H), 4.33 (s, 2H), 6.54-6.62 (m, 2H), 6.87 (d, J = |
| | 8.3 Hz, 2H), 7.15-7.24 (m, 3H). |
| I - 004 | 1H-NMR (CDCl3) δ: 1.02 (d, J = 6.5 Hz, 6H), 1.76-1.86 (m, 2H), 1.89- |
| | 2.01 (m, 2H), 2.01-2.13 (m, 1H), 2.34 (s, 3H), 2.38-2.53 (m, 2H), 2.65- |
| | 2.76 (m, 2H), 3.66 (s, 2H), 3.70 (d, J = 6.5 Hz, 2H), 4.34 (s, 2H), 6.51- |
| | 6.62 (m, 2H), 6.86 (d, J = 8.5 Hz, 2H), 6.94 (t, J = 7.4 Hz, 1H), 7.16 (d, |
| | J = 8.5 Hz, 2H). |
| I - 006 | 1H-NMR (CDCl3) δ: 1.51-1.65 (m, 2H), 1.73-1.93 (m, 2H), 2.23-2.38 |
| | (m, 2H), 2.28 (s, 3H), 2.55-2.66 (m, 2H), 3.69-3.77 (m, 1H), 3.82 (s, |
| | 3H), 4.27 (s, 2H), 4.34 (d, J = 5.0 Hz, 2H), 6.88 (d, J = 8.4 Hz, 2H), 7.00 |
| | (t, J = 8.5 Hz, 2H), 7.20-7.36 (m, 4H). |

TABLE 24

| | |
|---|---|
| I - 007 | 1H-NMR (CDCl3) δ: 1.02 (d, J = 6.8 Hz, 6H), 1.81-1.95 (m, 2H), 2.02- |
| | 2.13 (m, 3H), 2.33 (s, 3H), 2.47-2.63 (m, 4H), 3.70 (d, J = 6.4 Hz, 2H), |
| | 4.28 (s, 2H), 4.33 (s, 2H), 5.68 (br s, 1H), 6.82 (d, J = 8.4 Hz, 2H), 6.99 |
| | (dd, J = 8.3, 4.1 Hz, 1H), 7.16 (d, J = 8.3 Hz, 2H), 7.31 (td, J = 8.3, 4.1 |
| | Hz, 1H), 8.37 (d, J = 1.9 Hz, 1H). |
| I - 008 | 1H-NMR (CDCl3) δ: 1.02 (6H, d, J = 6.6 Hz), 1.53-1.63 (2H, m), 1.74- |
| | 1.85 (2H, m), 2.01-2.13 (2H, m), 2.28 (3H, s), 2.29-2.40 (2H, m), 2.51- |
| | 2.64 (4H, m), 2.92 (2H, t, J = 7.8 Hz), 3.69 (2H, d, J = 6.6 Hz), 4.28 (2H, |
| | s), 6.83 (2H, d, J = 8.5 Hz), 7.00 (2H, dd, J = 8.7, 8.7 Hz), 7.12 (2H, d, |
| | J = 8.5 Hz), 7.21-7.26 (2H, m). |
| I - 010 | 1H-NMR (CDCl3) δ: 1.78-1.92 (m, 2H), 1.97-2.13 (m, 2H), 2.31 (s, 3H), |
| | 2.42-2.62 (m, 4H), 3.85 (s, 3H), 4.14 (s, 2H), 4.33 (s, 2H), 4.34 (t, J = |
| | 11.8 Hz, 2H), 5.45 (br s, 1H), 5.99 (d, J = 1.3 Hz, 1H), 6.07 (tt, J = 53.3, |
| | 4.9 Hz, 1H), 6.89 (d, J = 8.3 Hz, 2H), 7.24-7.32 (m, 3H). |
| I - 011 | 1H-NMR (CDCl3) δ: 1.02 (d, J = 6.7 Hz, 6H), 1.82-1.95 (m, 2H), 2.02- |
| | 2.13 (m, 3H), 2.32 (s, 3H), 2.42 (s, 3H), 2.52-2.68 (m, 4H), 3.71 (d, J = |
| | 6.5 Hz, 2H), 4.03 (s, 2H), 4.29 (s, 2H), 5.69 (br s, 1H), 6.85 (d, J = 8.5 |
| | Hz, 2H), 7.20 (d, J = 8.5 Hz, 2H), 7.26 (s, 1H). |
| I - 012 | 1H-NMR (CDCl3) δ: 1.02 (d, J = 6.7 Hz, 6H), 1.83-1.96 (m, 2H), 2.00- |
| | 2.13 (m, 3H), 2.32 (s, 3H), 2.45-2.65 (m, 4H), 3.70 (d, J = 6.5 Hz, 2H), |
| | 4.26 (s, 2H), 4.33 (s, 2H), 6.23 (br s, 1H), 6.83 (d, J = 8.5 Hz, 2H), 6.97 |
| | (d, J = 7.8 Hz, 1H), 7.16-7.24 (m, 3H), 7.61 (t, J = 7.5 Hz, 1H), 8.51 (d, |
| | J = 3.9 Hz, 1H). |
| I - 013 | 1H-NMR (CDCl3) δ: 1.02 (d, J = 6.7 Hz, 6H), 1.87-1.98 (m, 2H), 2.01- |
| | 2.13 (m, 3H), 2.32 (s, 3H), 2.47-2.68 (m, 4H), 2.51 (s, 3H), 3.71 (d, J = |
| | 6.5 Hz, 2H), 4.18 (s, 2H), 4.30 (s, 2H), 6.72-6.77 (m, 2H), 6.83 (d, J = |
| | 8.5 Hz, 2H), 7.06 (d, J = 7.8 Hz, 1H), 7.20 (d, J = 8.4 Hz, 2H), 7.49 (t, |
| | J = 7.7 Hz, 1H). |
| I - 014 | 1H-NMR (CDCl3) δ: 1.04 (t, J = 7.4 Hz, 3H), 1.73-1.91 (m, 4H), 1.98- |
| | 2.07 (m, 2H), 2.31 (s, 3H), 2.46-2.58 (m, 4H), 3.85 (s, 3H), 3.91 (t, J = |
| | 6.6 Hz, 2H), 4.14 (s, 2H), 4.30 (s, 2H), 5.41 (br s, 1H), 5.99 (d, J = 1.9 |
| | Hz, 1H), 6.86 (d, J = 8.5 Hz, 2H), 7.19-7.29 (m, 3H). |

TABLE 25

| | |
|---|---|
| I - 015 | 1H-NMR (CDCl3) δ: 1.02 (d, J = 6.4 Hz, 6H), 1.73-1.90 (m, 2H), 1.97- |
| | 2.14 (m, 3H), 2.30 (s, 3H), 2.41-2.61 (m, 4H), 3.71 (d, J = 6.3 Hz, 2H), |
| | 4.26 (s, 2H), 4.30 (s, 2H), 4.60 (s, 1H), 6.28 (br s, 1H), 6.86 (d, J = 7.7 |
| | Hz, 2H), 7.11 (t, J = 60.8 Hz, 1H), 7.20 (d, J = 7.8 Hz, 2H), 7.74 (s, 1H). |
| I - 016 | 1H-NMR (CDCl3) δ: 1.02 (d, J = 6.8 Hz, 6H), 1.81-1.96 (m, 2H), 2.01- |
| | 2.14 (m, 3H), 2.33 (s, 3H), 2.33 (s, 3H), 2.47-2.65 (m, 4H), 3.71 (d, J = |
| | 6.5 Hz, 2H), 4.21 (s, 2H), 4.31 (s, 2H), 6.37 (br s, 1H), 6.83 (d, J = 8.3 |
| | Hz, 2H), 6.87 (d, J = 7.8 Hz, 1H), 7.19 (d, J = 8.0 Hz, 2H), 7.41 (d, J = |
| | 7.8 Hz, 1H), 8.33 (s, 1H). |
| I - 017 | 1H-NMR (CDCl3) δ: 1.02 (d, J = 6.8 Hz, 6H), 1.84-1.96 (m, 2H), 2.02- |
| | 2.14 (m, 3H), 2.32 (s, 3H), 2.48-2.61 (m, 4H), 2.67 (s, 3H), 3.71 (d, J = |
| | 6.8 Hz, 2H), 4.18 (s, 2H), 4.30 (s, 2H), 6.10 (br s, 1H), 6.74 (s, 1H), |
| | 6.84 (d, J = 8.5 Hz, 2H), 7.21 (d, J = 8.5 Hz, 2H). |
| I - 018 | 1H-NMR (CDCl3) δ: 1.02 (d, J = 6.8 Hz, 6H), 1.82-1.93 (m, 2H), 2.00- |
| | 2.11 (m, 3H), 2.31 (s, 3H), 2.47-2.64 (m, 4H), 2.59 (s, 3H), 3.70 (d, J = |
| | 6.5 Hz, 2H), 4.29 (s, 2H), 4.33 (s, 2H), 4.89 (s, 1H), 6.85 (d, J = 8.8 Hz, |
| | 2H), 7.25 (d, J = 8.5 Hz, 2H). |
| I - 019 | 1H-NMR (CDCl3) δ: 1.02 (d, J = 6.8 Hz, 6H), 1.81-1.95 (m, 2H), 1.98- |
| | 2.13 (m, 3H), 2.31 (s, 3H), 2.48-2.62 (m, 4H), 3.63 (s, 3H), 3.71 (d, J = |
| | 6.5 Hz, 2H), 4.06 (s, 2H), 4.30 (s, 2H), 6.35 (br s, 1H), 6.58 (s, 1H), |
| | 6.85 (d, J = 8.5 Hz, 2H), 7.22 (d, J = 8.5 Hz, 2H), 7.32 (s, 1H). |
| I - 023 | 1H-NMR (CDCl3) δ: 0.98 (d, J = 6.5 Hz, 6H), 1.76-1.97 (m, 2H), 1.97- |
| | 2.08 (m, 3H), 2.31 (s, 3H), 2.40-2.61 (m, 4H), 2.93 (d, J = 6.8 Hz, 2H), |
| | 3.55-3.89 (m, 1H), 3.84 (s, 3H), 4.14 (s, 2H), 4.24 (s, 2H), 5.37 (br s, |
| | 1H), 6.01 (d, J = 2.0 Hz, 1H), 6.56 (d, J = 8.5 Hz, 2H), 7.13 (d, J = 8.5 |
| | Hz, 2H), 7.26 (t, J = 2.5 Hz, 1H). |
| I - 024 | 1H-NMR (CDCl3) δ: 1.03 (t, J = 7.4 Hz, 3H), 1.63-1.84 (m, 6H), 2.27 (s, |
| | 3H), 2.36 (t, J = 10.0 Hz, 2H), 2.68-2.83 (m, 2H), 3.58 (s, 2H), 3.87 (t, |
| | J = 6.6 Hz, 2H), 4.19 (s, 2H), 6.78 (d, J = 8.5 Hz, 2H), 6.93-7.00 (m, 1H), |
| | 7.10 (d, J = 8.5 Hz, 2H), 7.20-7.26 (m, 1H), 8.35 (d, J = 2.5 Hz, 1H). |

TABLE 26

| | |
|---|---|
| I - 025 | 1H-NMR (CDCl3) δ: 1.03 (t, J = 7.4 Hz, 3H), 1.73-1.86 (m, 6H), 2.28 (s, |
| | 3H), 2.31-2.41 (m, 2H), 2.69-2.77 (m, 2H), 3.61 (s, 2H), 3.84 (s, 3H), |
| | 3.90 (t, J = 6.5 Hz, 2H), 4.07 (s, 2H), 5.85 (d, J = 1.9 Hz, 1H), 6.84 (d, |
| | J = 8.5 Hz, 2H), 7.18-7.23 (m, 3H). |
| I - 026 | 1H-NMR (CDCl3) δ: 0.12-0.17 (m, 1H), 0.36-0.48 (m, 2H), 0.62-0.69 |
| | (m, 1H), 1.03 (t, J = 7.4 Hz, 3H), 1.75-1.84 (m, 3H), 1.89-2.06 (m, 2H), |
| | 2.27 (s, 3H), 2.50 (t, J = 11.0 Hz, 1H), 2.73-2.86 (m, 2H), 3.31 (d, J = |
| | 15.7 Hz, 1H), 3.65 (d, J = 15.6 Hz, 1H), 3.89 (t, J = 6.6 Hz, 2H), 4.14 (d, |
| | J = 18.1 Hz, 1H), 4.39 (d, J = 18.1 Hz, 1H), 6.79 (d, J = 8.5 Hz, 2H), |
| | 6.99-7.07 (m, 3H), 7.26-7.32 (m, 1H), 8.41 (d, J = 2.5 Hz, 1H). |
| I - 028 | 1H-NMR (CDCl3) δ: 1.04 (t, J = 7.5 Hz, 3H), 1.73-1.95 (m, 4H), 2.00- |
| | 2.09 (m, 2H), 2.32 (s, 3H), 2.42 (s, 3H), 2.47-2.62 (m, 4H), 3.91 (t, J = |
| | 6.5 Hz, 2H), 4.03 (s, 2H), 4.29 (s, 2H), 5.68 (br s, 1H), 6.85 (d, J = 8.5 |
| | Hz, 2H), 7.21 (d, J = 8.5 Hz, 2H), 7.25 (s, 1H). |
| I - 029 | 1H-NMR (CDCl3) δ: 0.04-0.10 (m, 1H), 0.35-0.42 (m, 2H), 0.51-0.58 |
| | (m, 1H), 1.03 (t, J = 7.4 Hz, 3H), 1.75-1.84 (m, 2H), 1.87-1.98 (m, 2H), |
| | 2.07-2.16 (m, 1H), 2.20 (s, 3H), 2.26 (s, 3H), 2.47 (t, J = 10.9 Hz, 1H), |
| | 2.78-2.85 (m, 2H), 3.38 (d, J = 15.0 Hz, 1H), 3.62 (d, J = 15.0 Hz, 1H), |
| | 3.72 (s, 3H), 3.89 (t, J = 6.5 Hz, 2H), 3.98 (d, J = 17.1 Hz, 1H), 4.21 (d, |
| | J = 17.1 Hz, 1H), 5.78 (s, 1H), 6.81 (d, J = 8.5 Hz, 2H), 7.14 (d, J = 8.5 |
| | Hz, 2H). |
| I - 030 | 1H-NMR (CDCl3) δ: 1.03 (7H, d, J = 6.6 Hz), 2.01-2.13 (2H, m), 2.13- |
| | 2.28 (6H, m), 2.36 (2H, s), 2.93-3.07 (2H, m), 3.71 (2H, d, J = 6.6 Hz), |
| | 4.40 (2H, s), 4.80 (2H, s), 6.77-6.89 (3H, m), 7.20 (2H, d, J = 10.0 Hz), |
| | 7.33 (1H, t, J = 10.0 Hz), 8.27 (1H, s), 8.36 (1H, s). |
| I - 031 | 1H-NMR (CDCl3) δ: 1.02 (d, J = 6.8 Hz, 7H), 1.79-1.96 (m, 2H), 1.99- |
| | 2.13 (m, 3H), 2.31 (s, 3H), 2.43-2.64 (m, 3H), 3.71 (d, J = 6.5 Hz, 2H), |
| | 4.14 (s, 2H), 4.31 (s, 2H), 5.30 (br s, 1H), 6.85 (d, J = 8.5 Hz, 2H), 7.20 |
| | (d, J = 8.5 Hz, 2H), 7.42 (s, 1H), 7.84 (s, 1H). |
| I - 032 | 1H-NMR (CDCl3) δ: 1.02 (d, J = 6.8 Hz, 6H), 1.80-1.92 (m, 2H), 2.00- |
| | 2.13 (m, 3H), 2.27 (d, J = 1.3 Hz, 3H), 2.31 (s, 3H), 2.46-2.59 (m, 4H), |
| | 3.70 (d, J = 6.5 Hz, 2H), 4.23 (s, 2H), 4.36 (s, 2H), 4.93 (br s, 1H), 6.65 |
| | (s, 1H), 6.85 (d, J = 8.8 Hz, 2H), 7.23 (d, J = 8.5 Hz, 2H). |

TABLE 27

| | |
|---|---|
| I - 033 | 1H-NMR (CDCl3) δ: 1.03 (6H, d, J = 6.6 Hz), 2.01-2.24 (7H, m), 2.36 |
| | (3H, s), 2.90-3.03 (2H, m), 3.72 (2H, d, J = 6.6 Hz), 3.85 (3H, s), 4.26 |
| | (2H, s), 4.78 (2H, s), 5.76 (1H, s), 6.87 (2H, d, J = 8.4 Hz), 7.21-7.34 |
| | (2H, m), 7.52 (1H, s). |
| I - 034 | 1H-NMR (CDCl3) δ: 0.05-0.10 (m, 1H), 0.35-0.42 (m, 2H), 0.51-0.58 |
| | (m, 1H), 1.86-1.98 (m, 2H), 2.06-2.15 (m, 1H), 2.20 (s, 3H), 2.26 (s, |
| | 3H), 2.47 (t, J = 10.8 Hz, 1H), 2.77-2.85 (m, 2H), 3.38 (d, J = 15.4 Hz, |
| | 1H), 3.61 (d, J = 15.4 Hz, 1H), 3.72 (s, 3H), 3.79 (s, 3H), 3.98 (d, J = |
| | 17.2 Hz, 1H), 4.21 (d, J = 17.2 Hz, 1H), 5.79 (s, 1H), 6.82 (d, J = 8.5 |
| | Hz, 2H), 7.14 (d, J = 8.5 Hz, 2H). |
| I - 035 | 1H-NMR (CDCl3) δ: 0.07-0.16 (m, 1H), 0.40-0.47 (m, 2H), 0.52-0.57 |
| | (m, 1H), 1.89-1.99 (m, 2H), 2.08-2.17 (m, 1H), 2.19 (s, 3H), 2.27 (s, |
| | 3H), 2.43-2.52 (m, 1H), 2.78-2.86 (m, 2H), 3.46 (d, J = 15.6 Hz, 1H), |
| | 3.64 (d, J = 15.6 Hz, 1H), 3.71 (s, 3H), 4.00 (d, J = 17.1 Hz, 1H), 4.18 |
| | (d, J = 17.1 Hz, 1H), 5.75 (s, 1H), 6.49 (t, J = 74.0 Hz), 7.03 (d, J = 8.5 |
| | Hz, 2H), 7.22 (d, J = 8.5 Hz, 2H). |
| I - 036 | 1H-NMR (CDCl3) δ: 1.02 (d, J = 6.8 Hz, 6H), 1.71-1.80 (m, 2H), 1.88- |
| | 1.98 (m, 2H), 2.00-2.15 (m, 1H), 2.85-2.94 (m, 2H), 2.98-3.10 (m, 2H), |
| | 3.71 (d, J = 6.5 Hz, 2H), 3.85 (s, 3H), 4.15 (s, 2H), 4.30 (s, 2H), 5.42 (br |
| | s, 1H), 6.01 (d, J = 2.3 Hz, 1H), 6.86 (d, J = 8.8 Hz, 2H), 7.24 (d, J = 8.5 |
| | Hz, 2H), 7.25-7.28 (m, 1H). |
| I - 037 | 1H-NMR (CDCl3) δ: 0.06-0.11 (m, 1H), 0.22-0.35 (m, 2H), 0.58-0.64 |
| | (m, 1H), 1.03 (t, J = 7.4 Hz, 3H), 1.76-1.89 (m, 2H), 1.86-1.91 (m, 2H), |
| | 3.01-3.12 (m, 2H), 3.33-3.43 (m, 3H), 3.63 (d, J = 15.6 Hz, 1H), 3.86 (s, |
| | 3H), 3.90 (t, J = 6.5 Hz, 2H), 4.06 (d, J = 17.2 Hz, 1H), 4.28 (d, J = 17.2 |
| | Hz, 1H), 6.01 (br s, 1H), 6.83 (d, J = 8.4 Hz, 2H), 7.16 (d, J = 8.4 Hz, |
| | 2H), 7.25 (br s, 1H). |
| I - 038 | 1H-NMR (CDCl3) δ: 0.06-0.12 (m, 1H), 0.23-0.36 (m, 2H), 0.58-0.65 |
| | (m, 1H), 0.74-0.87 (m, 4H), 1.86-1.92 (m, 2H), 2.12 (d, J = 13.1 Hz, |
| | 1H), 3.02-3.11 (m, 2H), 3.33-3.45 (m, 2H), 3.61-3.74 (m, 2H), 3.86 (s, |
| | 3H), 4.07 (d, J = 17.1 Hz, 1H), 4.29 (d, J = 17.1 Hz, 1H), 6.01 (br s, 1H), |
| | 6.97 (d, J = 8.5 Hz, 2H), 7.17 (d, J = 8.5 Hz, 2H), 7.25 (br s, 1H). |
| I - 039 | 1H-NMR (CDCl3) c: 1.03 (6H, d, J = 6.5 Hz), 2.00-2.13 (2H, m), 2.13- |
| | 2.31 (4H, m), 2.31-2.39 (2H, m), 2.42 (3H, s), 2.88-3.06 (2H, m), 3.71 |
| | (2H, d, J = 6.5 Hz), 4.17 (2H, s), 4.73 (2H, s), 6.86 (2H, d, J = 7.7 Hz), |
| | 7.12 (1H, s), 7.23 (2H, d, J = 7.7 Hz), 7.98 (1H, s). |

TABLE 28

| | |
|---|---|
| I - 041 | 1H-NMR (CDCl3) δ: 0.06-0.14 (m, 1H), 0.39-0.54 (m, 2H), 0.55-0.62 |
| | (m, 1H), 0.74-0.84 (m, 4H), 1.93-2.07 (m, 2H), 2.18 (s, 3H), 2.30-2.39 |
| | (m, 2H), 2.53-2.70 (m, 1H), 2.89-3.06 (m, 1H), 3.43 (d, J = 15.6 Hz, |
| | 1H), 3.61-3.73 (m, 2H), 3.86 (s, 3H), 4.04 (d, J = 17.1 Hz, 1H), 4.28 (d, |
| | J = 17.1 Hz, 1H), 6.01 (d, J = 2.0 Hz, 1H), 6.97 (d, J = 8.5 Hz, 2H), 7.16 |
| | (d, J = 8.5 Hz, 2H), 7.25 (d, J = 2.0 Hz, 1H). |
| I - 042 | 1H-NMR (CDCl3) δ: 0.21-0.32 (1H, m), 0.38-0.53 (2H, m), 1.03 (6H, d, |
| | J = 6.6 Hz), 1.79-1.92 (1H, m), 2.00-2.16 (1H, m), 2.16-2.75 (8H, m), |
| | 3.02-3.14 (1H, m), 3.70 (2H, d, J = 6.6 Hz), 4.30 (1H, d, J = 15.5 Hz), |
| | 4.47 (1H, d, J = 15.5 Hz), 4.77 (2H, q, J = 15.5 Hz), 6.74-6.87 (3H, m), |
| | 7.16 (2H, d, J = 8.0 Hz), 7.30 (1H, t, J = 9.0 Hz), 8.05 (1H, br s), 8.34 |
| | (1H, s). |
| I - 043 | 1H-NMR (CDCl3) δ: 0.17-0.29 (1H, m), 0.34-0.48 (2H, m), 0.76-0.91 |
| | (1H, m), 1.03 (8H, d, J = 6.5 Hz), 1.77-1.90 (1H, m), 2.05-2.66 (8H, m), |
| | 3.02-3.14 (1H, m), 3.72 (2H, d, J = 6.5 Hz), 3.84 (3H, s), 4.25 (2H, dd, |
| | J = 37.8, 15.4 Hz), 4.75 (2H, dd, J = 34.9, 14.6 Hz), 5.70 (1H, s), 6.87 |
| | (2H, d, J = 8.5 Hz), 7.10-7.29 (3H, m). |
| I - 044 | 1H-NMR (CDCl3) δ: 0.05-0.12 (m, 1H), 0.37-0.40 (m, 2H), 0.54-0.59 |
| | (m, 1H), 1.03 (t, J = 7.4 Hz, 3H), 1.76-1.94 (m, 2H), 1.88-1.94 (m, 2H), |
| | 2.11 (td, J = 12.7 Hz, 4.7 Hz, 1H), 2.26 (s, 3H), 2.47 (t, J = 11.0 Hz, 1H), |
| | 2.78-2.84 (m, 2H), 3.41 (d, J = 15.6 Hz, 1H), 3.62 (d, J = 15.6 Hz, 1H), |
| | 3.86 (s, 3H), 3.90 (t, J = 6.7 Hz, 2H), 4.03 (d, J = 17.1 Hz, 1H), 4.26 (d, |
| | J = 17.1 Hz, 1H), 6.02 (d, J = 2.3 Hz, 1H), 6.82 (d, J = 8.5 Hz, 2H), 7.15 |
| | (d, J = 8.5 Hz, 2H), 7.25 (d, J = 2.3 Hz, 1H). |
| I - 045 | 1H-NMR (CDCl3) δ: 0.05-0.12 (m, 1H), 0.37-0.40 (m, 2H), 0.54-0.59 |
| | (m, 1H), 1.03 (t, J = 7.4 Hz, 3H), 1.76-1.94 (m, 2H), 1.88-1.94 (m, 2H), |
| | 2.11 (td, J = 12.7 Hz, 4.7 Hz, 1H), 2.26 (s, 3H), 2.47 (t, J = 11.0 Hz, 1H), |
| | 2.78-2.84 (m, 2H), 3.41 (d, J = 15.6 Hz, 1H), 3.62 (d, J = 15.6 Hz, 1H), |
| | 3.86 (s, 3H), 3.90 (t, J = 6.7 Hz, 2H), 4.03 (d, J = 17.1 Hz, 1H), 4.26 (d, |
| | J = 17.1 Hz, 1H), 6.02 (d, J = 2.3 Hz, 1H), 6.82 (d, J = 8.5 Hz, 2H), 7.15 |
| | (d, J = 8.5 Hz, 2H), 7.25 (d, J = 2.3 Hz, 1H). |

TABLE 29

| | |
|---|---|
| I - 046 | 1H-NMR (CDCl3) δ: 1.03 (6H, d, J = 6.5 Hz), 1.76-1.82 (2H, m), 1.99- |
| | 2.14 (3H, m), 2.29 (3H, s), 2.49 (4H, br s), 3.65 (1H, s), 3.71 (2H, d, J = |
| | 6.5 Hz), 3.87 (3H, s), 4.14 (2H, s), 4.21 (2H, s), 6.87 (2H, d, J = 8.8 Hz), |
| | 7.16 (2H, d, J = 8.8 Hz), 7.33 (1H, s), 7.36 (1H, s). |
| I - 047 | 1H-NMR (CDCl3) δ: 1.83-1.98 (m, 2H), 2.00-2.10 (m, 2H), 2.32 (s, 3H), |
| | 2.42 (s, 3H), 2.48-2.63 (m, 4H), 4.03 (s, 2H), 4.31 (s, 2H), 4.34 (q, J = |
| | 8.3 Hz, 2H), 5.75 (br s, 1H), 6.90 (d, J = 8.5 Hz, 2H), 7.27 (d, J = 8.5 |
| | Hz, 2H), 7.27 (s, 1H). |
| I - 048 | 1H-NMR (CDCl3) δ: 1.02 (6H, d, J = 6.7 Hz), 1.77-1.84 (2H, m), 1.93- |
| | 1.99 (2H, m), 2.03-2.13 (1H, m), 2.42 (3H, s), 2.87-2.93 (2H, m), 3.03- |
| | 3.09 (2H, m), 3.71 (2H, d, J = 6.5 Hz), 4.04 (2H, s), 4.29 (2H, s), 5.71 |
| | (1H, s), 6.85 (2H, d, J = 8.7 Hz), 7.21 (2H, d, J = 8.7 Hz). |
| I - 049 | 1H-NMR (CDCl3) δ: 0.99-1.08 (m, 10H), 1.84-1.94 (m, 2H), 1.96-2.13 |
| | (m, 4H), 2.32 (s, 3H), 2.44-2.67 (m, 4H), 3.70 (d, J = 6.5 Hz, 2H), 3.99 |
| | (s, 2H), 4.27 (s, 2H), 5.86 (br s, 1H), 6.84 (d, J = 8.8 Hz, 2H), 7.18 (s, |
| | 1H), 7.20 (d, J = 8.8 Hz, 2H). |
| I - 050 | 1H-NMR (CDCl3) δ: 1.03 (d, J = 6.8 Hz, 6H), 1.80-1.91 (m, 2H), 1.98- |
| | 2.13 (m, 3H), 2.31 (s, 3H), 2.43-2.62 (m, 4H), 3.71 (d, J = 6.5 Hz, 2H), |
| | 4.18 (s, 3H), 4.33 (s, 2H), 4.80 (br s, 1H), 6.60 (t, J = 52.7 Hz, 1H), 6.86 |
| | (d, J = 8.5 Hz, 2H), 7.19 (d, J = 8.5 Hz, 2H), 7.53 (s, 1H). |
| I - 051 | 1H-NMR (CDCl3) δ: 7.41 (1H, s), 7.19 (2H, d, J = 8.7 Hz), 6.84 (2H, d, |
| | J = 8.7 Hz), 4.35 (2H, s), 4.13 (2H, s), 3.70 (2H, d, J = 6.5 Hz), 3.10 |
| | (3H, s), 3.03-2.92 (4H, m), 2.44 (3H, s), 2.11-2.02 (1H, m), 1.87-1.81 |
| | (2H, m), 1.78-1.76 (2H, m), 1.03 (3H, s), 1.01 (3H, s). |
| I - 052 | 1H-NMR (CDCl3) δ: 7.40 (1H, s), 7.19 (2H, d, J = 8.7 Hz), 6.84 (2H, d, |
| | J = 8.7 Hz), 4.34 (2H, s), 4.12 (2H, s), 3.70 (2H, d, J = 6.5 Hz), 3.09 |
| | (3H, s), 2.64-2.63 (2H, br m), 2.49-2.47 (2H, br m), 2.43 (3H, s), 2.32 |
| | (3H, s), 2.10-2.05 (1H, m), 1.97-1.94 (2H, br m), 1.84-1.80 (2H, br m), |
| | 1.03 (3H, s), 1.01 (3H, s). |

TABLE 30

| | |
|---|---|
| I - 053 | 1H-NMR (CDCl3) δ: 1.03 (6H, d, J = 6.8 Hz), 1.79-1.89 (2H, m), 1.99- |
| | 2.14 (3H, m), 2.30 (3H, s), 2.43 (3H, s), 2.47-2.57 (4H, m), 3.71 (2H, d, |
| | J = 6.8 Hz), 4.01 (1H, s), 4.23 (2H, s), 4.26 (2H, s), 6.78 (1H, s), 6.87 |
| | (2H, d, J = 8.5 Hz), 7.17 (2H, d, J = 8.5 Hz). |
| I - 054 | 1H-NMR (CDCl3) δ: 1.02 (6H, d, J = 6.6 Hz), 1.81-1.93 (2H, m), 1.99- |
| | 2.11 (3H, m), 2.14 (3H, d, J = 1.3 Hz), 2.31 (3H, s), 2.45-2.63 (4H, m), |
| | 3.70 (2H, d, J = 6.6 Hz), 4.24 (2H, s), 4.35 (2H, s), 5.01 (1H, s), 6.85 |
| | (2H, d, J = 8.7 Hz), 7.22 (2H, d, J = 8.7 Hz), 7.30 (1H, d, J = 1.3 Hz). |
| I - 055 | 1H-NMR (CDCl3) δ: 1.03 (6H, d, J = 6.6 Hz), 1.79-1.85 (2H, m), 1.99- |
| | 2.13 (3H, m), 2.31 (3H, s), 2.44-2.60 (4H, m), 3.71 (2H, d, J = 6.6 Hz), |
| | 3.92 (1H, s), 4.22 (2H, s), 4.28 (2H, s), 6.86 (2H, d, J = 8.5 Hz), 7.02 |
| | (2H, t, J = 8.6 Hz), 7.12 (2H, d, J = 8.5 Hz), 7.21 (2H, dd, J = 8.5, 5.5 |
| | Hz). |
| I - 056 | 1H-NMR (CDCl3) δ: 1.81-1.90 (2H, m), 1.98-2.06 (2H, m), 2.31 (3H, s), |
| | 2.46-2.59 (4H, m), 3.84 (3H, s), 3.98 (2H, s), 4.39 (2H, s), 5.64 (1H, s), |
| | 6.20 (1H, d, J = 2.3 Hz), 7.25-7.31 (6H, m), 7.47 (2H, d, J = 8.9 Hz), |
| | 9.24 (1H, s). |
| I - 057 | 1H-NMR (CDCl3) δ: 1.03 (6H, d, J = 6.6 Hz), 1.56-1.71 (2H, m), 2.01- |
| | 2.14 (1H, m), 2.14-2.30 (4H, m), 2.36 (3H, s), 2.67 (3H, s), 2.96-3.04 |
| | (2H, m), 3.71 (2H, d, J = 6.6 Hz), 4.32 (2H, s), 4.74 (2H, s), 6.69 (1H, s), |
| | 6.85 (2H, d, J = 8.5 Hz), 7.24 (2H, d, J = 8.5 Hz), 8.14 (1H, s). |
| I - 058 | 1H-NMR (CDCl3) δ: 1.03 (6H, d, J = 6.8 Hz), 1.49-1.57 (2H, m), 2.02- |
| | 2.13 (1H, m), 2.12-2.21 (4H, m), 2.22 (3H, s), 2.35 (3H, s), 2.91-3.02 |
| | (2H, m), 3.71 (3H, s), 3.72 (2H, d, J = 6.8 Hz), 4.18 (2H, s), 4.76 (2H, s), |
| | 5.58 (1H, s), 6.87 (2H, d, J = 8.7 Hz), 7.27 (2H, d, J = 8.7 Hz), 7.61 (1H, |
| | s). |
| I - 059 | 1H-NMR (CDCl3) δ: 1.03 (6H, d, J = 6.6 Hz), 1.43-1.54 (2H, m), 1.76- |
| | 1.91 (2H, m), 2.02-2.16 (3H, m), 2.24 (3H, s), 2.56 (3H, s), 2.82-2.91 |
| | (2H, m), 3.71 (2H, d, J = 6.6 Hz), 4.36 (2H, br s), 4.84 (2H, br s), 5.29 |
| | (1H, s), 6.89 (2H, d, J = 8.5 Hz), 7.03-7.14 (2H, m), 7.16 (1H, d, J = 8.5 |
| | Hz), 8.37 (1H, s). |

TABLE 31

| | |
|---|---|
| I - 060 | 1H-NMR (CDCl3) δ: 1.03 (6H, d, J = 6.6 Hz), 1.40-1.94 (4H, m), 1.96- |
| | 2.16 (3H, m), 2.24 (3H, s), 2.77-2.91 (2H, m), 3.72 (3H, d, J = 6.6 Hz), |
| | 4.54 (2H, s), 4.61 (2H, s), 5.37 (1H, br s), 6.47-6.54 (1H, m), 6.69 (1H, |
| | d, J = 9.5 Hz), 6.92 (2H, d, J = 8.3 Hz), 6.98-7.04 (1H, m), 7.09 (2H, d, |
| | J = 8.3 Hz). |
| I - 062 | 1H-NMR (CDCl3) δ: 1.03 (6H, d, J = 6.6 Hz), 1.54-1.63 (2H, m), 2.01- |
| | 2.13 (1H, m), 2.13-2.28 (4H, m), 2.34 (3H, s), 2.37 (3H, s), 2.96-3.05 |
| | (2H, m), 3.71 (2H, d, J = 6.6 Hz), 4.34 (2H, s), 4.77 (2H, s), 6.78 (1H, d, |
| | J = 7.5 Hz), 6.84 (2H, d, J = 8.4 Hz), 7.23 (2H, d, J = 8.4 Hz), 7.43 (1H, |
| | d, J = 7.5 Hz), 8.31 (1H, s), 8.84 (1H, s). |
| I - 063 | 1H-NMR (CDCl3) δ: 1.02 (6H, d, J = 6.6 Hz), 1.26-1.67 (2H, m), 1.95- |
| | 2.21 (5H, m), 2.29 (3H, s), 2.77-3.06 (2H, m), 3.71 (2H, d, J = 6.6 Hz), |
| | 4.12-5.08 (4H, br m), 5.82-6.34 (1H, m), 6.34-6.38 (1H, m), 6.87 (2H, d, |
| | J = 8.5 Hz), 6.96-7.35 (2H, m), 7.38-7.41 (1H, m). |
| I - 064 | 1H-NMR (CDCl3) δ: 1.85-1.95 (2H, m), 2.01-2.10 (3H, m), 2.32 (3H, s), |
| | 2.42 (3H, s), 2.51-2.62 (4H, m), 4.03 (2H, s), 4.31 (2H, s), 4.34 (2H, t, |
| | J = 11.8 Hz), 5.75 (1H, s), 5.91-6.22 (1H, m), 6.88 (2H, d, J = 8.5 Hz), |
| | 7.24-7.31 (3H, m). |
| I - 065 | 1H-NMR (CDCl3) δ: 1.77 (4H, t, J = 18.8 Hz), 1.86-1.97 (2H, m), 2.01- |
| | 2.11 (2H, m), 2.33 (3H, s), 2.42 (3H, s), 2.51-2.65 (4H, m), 4.03 (2H, s), |
| | 4.10 (2H, t, J = 11.3 Hz), 4.30 (2H, s), 5.75 (1H, s), 6.88 (2H, d, J = 8.8 |
| | Hz), 7.21-7.28 (3H, m). |
| I - 066 | 1H-NMR (CDCl3) δ: 1.86-1.93 (2H, m), 2.00-2.10 (2H, m), 2.32 (3H, s), |
| | 2.42 (3H, s), 2.50-2.61 (4H, m), 4.03 (2H, s), 4.18 (2H, td, J = 13.1, 4.1 |
| | Hz), 4.30 (2H, s), 5.73 (1H, s), 5.93-6.24 (1H, m), 6.87 (2H, d, J = 8.7 |
| | Hz), 7.22-7.29 (3H, m). |
| I - 067 | 1H-NMR (CDCl3) δ: 7.46 (1H, d, J = 1.8 Hz), 7.20 (2H, d, J = 8.8 Hz), |
| | 6.85 (2H, d, J = 8.8 Hz), 5.34 (2H, d, J = 47.2 Hz), 5.10 (1H, s), 4.31 |
| | (2H, s), 4.14 (2H, s), 3.71 (2H, d, J = 6.8 Hz), 2.53 (4H, s), 2.31 (3H, s), |
| | 2.17-2.02 (3H, m), 1.89-1.83 (2H, m), 1.03 (6H, d, J = 6.8 Hz). |

TABLE 32

| | |
|---|---|
| I - 068 | ¹H-NMR (CDCl₃) δ: 7.21 (2H, d, J = 8.8 Hz), 6.84 (2H, d, J = 8.8 Hz), |
| | 5.92 (1H, s), 4.29 (2H, s), 4.03 (2H, s), 3.71 (2H, d, J = 6.5 Hz), 2.74 |
| | (2H, q, J = 7.5 Hz), 2.55 (4H, s), 2.32 (3H, s), 2.11-2.05 (3H, m), 1.93- |
| | 1.86 (2H, m), 1.31 (3H, t, J = 7.6 Hz), 1.02 (6H, d, J = 6.8 Hz). |
| I - 069 | 1H-NMR (CDCl3) δ: 1.84-1.94 (2H, m), 2.00-2.09 (2H, m), 2.32 (3H, s), |
| | 2.43 (4H, s), 2.50-2.60 (4H, m), 4.11 (2H, s), 4.52 (2H, s), 5.57 (1H, s), |
| | 6.95 (1H, dd, J = 5.0, 3.4 Hz), 6.99-7.01 (1H, m), 7.25 (1H, d, J = 5.0 |
| | Hz), 7.32 (1H, s). |
| I - 070 | 1H-NMR (CDCl3) δ: 1.84-1.94 (2H, m), 2.00-2.09 (2H, m), 2.32 (3H, s), |
| | 2.43 (4H, s), 2.50-2.60 (4H, m), 4.11 (2H, s), 4.52 (2H, s), 5.57 (1H, s), |
| | 6.95 (1H, dd, J = 5.0, 3.4 Hz), 6.99-7.01 (1H, m), 7.25 (1H, d, J = 5.0 |
| | Hz), 7.32 (1H, s). |
| I - 071 | 1H-NMR (CDCl3) δ: 1.84-1.93 (2H, m), 2.01-2.09 (2H, m), 2.32 (3H, s), |
| | 2.42 (3H, s), 2.49-2.60 (4H, m), 3.80 (3H, s), 4.03 (2H, s), 4.30 (2H, s), |
| | 5.69 (1H, s), 6.86 (2H, d, J = 8.7 Hz), 7.22 (2H, d, J = 8.7 Hz), 7.26 (1H, |
| | s). |
| I - 072 | 1H-NMR (CDCl3) δ: 1.03 (7H, d, J = 6.6 Hz), 1.78-1.88 (2H, m), 1.99- |
| | 2.14 (3H, m), 2.30 (3H, s), 2.45-2.58 (4H, m), 3.71 (2H, d, J = 6.6 Hz), |
| | 4.15 (3H, s), 4.27 (4H, s), 4.57 (1H, s), 6.86 (2H, d, J = 8.5 Hz), 7.20 |
| | (2H, d, J = 8.5 Hz), 7.36 (1H, s). |
| I - 073 | 1H-NMR (CDCl3) δ: 1.85-1.92 (2H, m), 1.99-2.09 (2H, m), 2.32 (3H, s), |
| | 2.42 (3H, s), 2.49-2.61 (4H, m), 4.07 (3H, s), 4.36 (3H, s), 5.67 (1H, s), |
| | 7.05 (1H, d, J = 4.9 Hz), 7.17 (1H, d, J = 2.2 Hz), 7.26-7.30 (2H, m). |
| I - 074 | 1H-NMR (CDCl3) δ: 7.37-7.35 (2H, m), 7.09 (2H, d, J = 8.7 Hz), 7.04- |
| | 7.02 (2H, m), 6.82 (2H, d, J = 8.7 Hz), 5.08 (1H, q, J = 6.9 Hz), 4.19 |
| | (1H, d, J = 15.7 Hz), 4.05 (1H, d, J = 15.7 Hz), 3.70-3.68 (3H, m), 2.48- |
| | 2.46 (2H, br m), 2.34-2.31 (1H, br m), 2.25 (3H, s), 2.12-2.02 (1H, m), |
| | 1.98-1.90 (2H, m), 1.63-1.61 (3H, m), 1.55 (3H, d, J = 7.0 Hz), 1.02 |
| | (3H, s), 1.01 (3H, s). |
| I - 075 | 1H-NMR (CDCl3) δ: 1.02 (6H, d, J = 6.6 Hz), 1.78-1.84 (2H, m), 1.97- |
| | 2.13 (3H, m), 2.29 (3H, s), 2.40 (3H, d, J = 0.6 Hz), 2.44-2.58 (4H, m), |
| | 3.71 (2H, d, J = 6.6 Hz), 4.27 (2H, s), 4.29 (2H, s), 4.30 (1H, s), 5.92 |
| | (1H, s), 6.86 (2H, d, J = 8.7 Hz), 7.20 (2H, d, J = 8.7 Hz). |

TABLE 33

| | |
|---|---|
| I - 076 | 1H-NMR (CDCl3) δ: 1.03 (6H, d, J = 6.6 Hz), 1.78-1.87 (2H, m), 1.99- |
| | 2.13 (3H, m), 2.28 (3H, s), 2.30 (3H, s), 2.43-2.57 (4H, m), 3.71 (2H, d, |
| | J = 6.6 Hz), 4.04 (1H, s), 4.31 (4H, s), 6.00 (1H, s), 6.87 (2H, d, J = 8.6 |
| | Hz), 7.18 (2H, d, J = 8.6 Hz). |
| I - 077 | 1H-NMR (CDCl3) δ: 0.93 (3H, t, J = 7.3 Hz), 1.30-1.40 (2H, m), 1.54- |
| | 1.62 (2H, m), 1.83-1.93 (2H, m), 2.01-2.09 (2H, m), 2.31 (3H, s), 2.42 |
| | (3H, s), 2.49-2.59 (4H, m), 2.59 (2H, t, J = 7.7 Hz), 4.05 (2H, s), 4.33 (2H, |
| | s), 5.66 (1H, s), 7.13 (2H, d, J = 7.9 Hz), 7.20 (2H, d, J = 7.9 Hz), 7.25 |
| | (1H, s). |
| I - 078 | 1H-NMR (CDCl3) δ: 7.40 (1H, s), 7.19 (2H, d, J = 8.7 Hz), 6.84 (2H, d, |
| | J = 8.7 Hz), 4.34 (2H, s), 4.12 (2H, s), 3.70 (2H, d, J = 6.5 Hz), 3.09 (3H, |
| | s), 2.64-2.63 (2H, br m), 2.49-2.47 (2H, br m), 2.43 (3H, s), 2.32 (3H, s), |
| | 2.10-2.05 (1H, m), 1.97-1.94 (2H, br m), 1.84-1.80 (2H, br m), 1.03 (3H, |
| | s), 1.01 (3H, s). |
| I - 079 | 1H-NMR (CDCl3) δ: 0.32-0.38 (2H, m), 0.61-0.68 (2H, m), 1.22-1.32 (1H, |
| | m), 1.83-1.93 (2H, m), 2.00-2.10 (2H, m), 2.31 (3H, s), 2.42 (3H, s), 2.49- |
| | 2.61 (4H, m), 3.79 (2H, d, J = 6.9 Hz), 4.03 (2H, s), 4.29 (2H, s), 5.68 |
| | (1H, s), 6.85 (2H, d, J = 8.5 Hz), 7.21 (2H, d, J = 8.5 Hz), 7.25 (1H, s). |
| I - 081 | 1H-NMR (CDCl3) δ: 8.32 (1H, d, J = 2.0 Hz), 7.54 (1H, dd, J = 8.0, 2.0 |
| | Hz), 7.13-7.12 (3H, m), 6.87-6.85 (2H, m), 4.27 (2H, s), 4.21 (2H, s), 3.91 |
| | (1H, s), 3.71 (2H, d, J = 6.5 Hz), 2.55 (3H, s), 2.49 (4H, br s), 2.29 (3H, |
| | s), 2.11-2.02 (3H, m), 1.84-1.77 (2H, m), 1.04 (3H, s), 1.02 (3H, s). |
| I - 082 | 1H-NMR (CDCl3) δ: 1.86-1.93 (2H, m), 2.00-2.10 (2H, m), 2.32 (3H, s), |
| | 2.42 (3H, s), 2.50-2.61 (4H, m), 4.04 (2H, s), 4.31 (2H, s), 4.41 (2H, t, J = |
| | 12.3 Hz), 5.74 (1H, s), 6.90 (2H, d, J = 8.5 Hz), 7.25-7.29 (3H, m). |
| I - 083 | 1H-NMR (CDCl3) δ: 1.83-2.00 (6H, m), 2.00-2.09 (2H, m), 2.10-2.19 (2H, |
| | m), 2.31 (3H, s), 2.42 (3H, s), 2.49-2.62 (4H, m), 2.70-2.82 (1H, m), 3.91 |
| | (2H, d, J = 6.8 Hz), 4.03 (2H, s), 4.29 (2H, s), 5.68 (1H, s), 6.85 (2H, d, |
| | J = 8.7 Hz), 7.20 (2H, d, J = 8.7 Hz), 7.26 (1H, s). |
| I - 084 | 1H-NMR (CDCl3) δ: 1.84-1.94 (2H, m), 2.01-2.10 (2H, m), 2.32 (3H, s), |
| | 2.42 (3H, s), 2.44-2.80 (9H, m), 3.99 (2H, d, J = 6.1 Hz), 4.03 (2H, s), |
| | 4.29 (2H, s), 5.69 (1H, s), 6.85 (2H, d, J = 8.5 Hz), 7.22 (2H, d, J = 8.5 |
| | Hz), 7.27 (1H, s). |

TABLE 34

| | |
|---|---|
| I - 085 | 1H-NMR (CDCl3) δ: 7.26 (1H, s), 7.20 (2H, d, J = 8.5 Hz), 6.85 (2H, d, |
| | J = 8.8 Hz), 6.19 (1H, s), 4.53 (1H, d, J = 48.7 Hz), 4.28 (2H, dd, J = 23.3, |
| | 15.1 Hz), 4.04 (2H, d, J = 6.3 Hz), 3.71 (2H, d, J = 6.8 Hz), 3.21-3.16 (1H, |
| | m), 3.10-2.88 (2H, m), 2.42 (3H, s), 2.13-2.06 (1H, m), 1.99-1.94 (2H, m), |
| | 1.03 (3H, s), 1.02 (3H, s). |
| I - 086 | 1H-NMR (CDCl3) δ: 0.98-1.09 (4H, m), 1.85-1.95 (2H, m), 1.96-2.10 (3H, |
| | m), 2.32 (3H, s), 2.50-2.62 (4H, m), 4.00 (2H, s), 4.30 (2H, s), 4.34 (2H, |
| | q, J = 8.2 Hz), 5.90 (1H, s), 6.89 (2H, d, J = 8.7 Hz), 7.20 (1H, s), 7.26 |
| | (1H, d, J = 8.7 Hz). |
| I - 087 | 1H-NMR (CDCl3) δ: 0.94 (3H, t, J = 7.3 Hz), 1.57-1.69 (2H, m), 1.85- |
| | 1.96 (2H, m), 2.01-2.11 (2H, m), 2.33 (3H, s), 2.42 (3H, s), 2.51-2.63 (6H, |
| | m), 4.05 (2H, s), 4.33 (2H, s), 5.68 (1H, s), 7.13 (2H, d, J = 8.0 Hz), 7.20 |
| | (2H, d, J = 8.0 Hz), 7.25 (1H, s). |

TABLE 35

| | |
|---|---|
| I-088 | 1H-NMR (CDCl3) δ: 1.88-1.94 (2H, m), 2.02-2.10 (2H, m), 2.32 (3H, s), |
| | 2.38 (3H, s), 2.50-2.60 (4H, m), 3.85 (3H, s), 4.31 (2H, s), 4.62 (2H, s), |
| | 5.79 (1H, s), 6.94 (1H, dd, J = 8.9, 2.4 Hz), 7.17 (1H, d, J = 2.4 Hz), 7.38 |
| | (1H, d, J = 8.9 Hz), 7.42 (1H, s). |
| I-089 | 1H-NMR (CDCl3) δ: 1.86-1.96 (2H, m), 2.02-2.10 (2H, m), 2.32 (3H, s), |
| | 2.38 (3H, s), 2.48-2.61 (4H, m), 4.32 (2H, s), 4.65 (2H, s), 5.79 (1H, s), |
| | 7.32 (1H, dd, J = 8.7, 2.1 Hz), 7.42 (1H, s), 7.43 (1H, d, J = 8.7 Hz), 7.68 |
| | (1H, d, J = 2.1 Hz). |
| I-090 | 1H-NMR (CDCl3) δ: 1.87-1.96 (2H, m), 2.01-2.10 (2H, m), 2.32 (3H, s), |
| | 2.38 (3H, s), 2.48-2.62 (4H, m), 4.32 (2H, s), 4.65 (2H, s), 5.79 (1H, s), |
| | 7.38 (1H, d, J = 8.6 Hz), 7.42 (1H, s), 7.47 (1H, dd, J = 8.6, 1.9 Hz), 7.84 |
| | (1H, d, J = 1.9 Hz). |
| I-091 | 1H-NMR (CDCl3) δ: 1.87-1.96 (2H, m), 1.99-2.08 (2H, m), 2.33 (3H, s), |
| | 2.43 (3H, s), 2.50-2.63 (4H, m), 4.26 (2H, s), 4.47 (2H, s), 6.65 (1H, s), |
| | 6.77-6.81 (1H, m), 7.15-7.21 (1H, m), 7.51 (2H, d, J = 8.2 Hz), 7.52 (1H, |
| | s), 7.61 (1H, s), 8.08 (1H, d, J = 6.8 Hz). |
| I-092 | 1H-NMR (CDCl3) δ: 0.97 (3H, t, J = 7.3 Hz), 1.48 (2H, td, J = 15.0, 7.4 |
| | Hz), 1.72-1.79 (2H, m), 1.89-1.96 (2H, m), 2.02-2.09 (2H, m), 2.34 (3H, |
| | s), 2.42 (3H, s), 2.58 (4H, br s), 3.93 (2H, t, J = 6.5 Hz), 4.07 (2H, s), 4.41 |
| | (2H, s), 5.92 (1H, s), 6.83 (1H, dd, J = 8.6, 2.4 Hz), 7.07 (1H, d, J = 2.4 |
| | Hz), 7.34 (1H, s), 7.37 (1H, d, J = 8.5 Hz). |
| I-093 | 1H-NMR (CDCl3) δ: 1.89-1.95 (2H, m), 2.05-2.11 (2H, m), 2.33 (3H, s), |
| | 2.43 (3H, s), 2.51-2.62 (4H, m), 4.06 (2H, s), 4.31 (3H, s), 4.51 (2H, s), |
| | 5.91 (1H, s), 7.29 (1H, s), 7.48 (1H, d, J = 8.5 Hz), 7.59 (1H, dd, J = 8.5, |
| | 1.3 Hz), 7.89 (1H, s). |
| I-094 | 1H-NMR (CDCl3) δ: 1.87-1.95 (2H, m), 2.02-2.10 (2H, m), 2.32 (3H, s), |
| | 2.37 (3H, s), 2.49-2.61 (4H, m), 4.33 (2H, s), 4.66 (2H, s), 5.80 (1H, s), |
| | 7.33-7.36 (2H, m), 7.42 (1H, s), 7.49-7.53 (1H, m), 7.68-7.72 (1H, m). |
| I-095 | 1H-NMR (CDCl3) δ: 1.90-1.99 (2H, m), 2.05-2.14 (2H, m), 2.33 (3H, s), |
| | 2.42 (3H, s), 2.52-2.63 (4H, m), 4.12 (2H, s), 4.57 (2H, s), 5.87 (1H, s), |
| | 7.30 (1H, s), 7.56 (1H, t, J = 7.8 Hz), 7.71 (1H, ddd, J = 7.8, 2.1, 8.3 Hz), |
| | 7.81 (1H, d, J = 8.3 Hz), 8.10 (2H, d, J = 9.3 Hz), 8.83 (1H, d, J = 2.1 Hz). |

TABLE 36

| | |
|---|---|
| I-096 | 1H-NMR (CDCl3) δ: 1.88-1.97 (2H, m), 2.05-2.13 (2H, m), 2.33 (3H, s), |
| | 2.43 (3H, s), 2.52-2.63 (4H, m), 4.11 (2H, s), 4.56 (2H, s), 5.86 (1H, s), |
| | 7.28 (1H, s), 7.42 (1H, dd, J = 8.3, 4.2 Hz), 7.67 (1H, dd, J = 8.6, 1.7 Hz), |
| | 7.72 (1H, s), 8.07 (1H, d, J = 8.6 Hz), 8.13 (1H, d, J = 7.0 Hz), 8.91 (1H, |
| | dd, J = 4.2, 1.7 Hz). |
| I-097 | 1H-NMR (CDCl3) δ: 1.86-1.96 (2H, m), 2.04-2.12 (2H, m), 2.32 (3H, s), |
| | 2.42 (3H, s), 2.50-2.62 (4H, m), 4.09 (2H, s), 4.53 (2H, s), 5.81 (1H, s), |
| | 7.25 (1H, s), 7.43-7.51 (3H, m), 7.70 (1H, s), 7.78-7.85 (3H, m). |
| I-098 | 1H-NMR (CDCl3) δ: 1.89-2.01 (2H, m), 2.04-2.12 (2H, m), 2.34 (3H, s), |
| | 2.42 (3H, s), 2.53-2.66 (4H, m), 4.25 (2H, s), 4.76 (2H, s), 6.14 (1H, s), |
| | 7.40 (1H, t, J = 7.8 Hz), 7.44 (1H, s), 7.49 (1H, t, J = 7.4 Hz), 7.87 (1H, d, |
| | J = 7.4 Hz), 7.98 (1H, d, J = 8.3 Hz). |
| I-099 | 1H-NMR (CDCl3) δ: 1.87-1.94 (2H, m), 2.01-2.09 (2H, m), 2.32 (3H, s), |
| | 2.41 (3H, s), 2.50-2.60 (4H, m), 4.23 (2H, s), 4.49 (2H, s), 5.66 (1H, s), |
| | 6.68 (1H, s), 7.20-7.29 (2H, m), 7.39 (1H, s), 7.42 (1H, d, J = 8.3 Hz), |
| | 7.53 (1H, d, J = 6.8 Hz). |
| I-100 | 1H-NMR (CDCl3) δ: 0.97-1.08 (10H, m), 1.81-1.88 (2H, m), 1.98-2.14 |
| | (3H, m), 2.31 (3H, s), 2.45-2.62 (4H, m), 3.48-3.55 (1H, m), 3.71 (2H, d, |
| | J = 6.5 Hz), 4.13 (2H, s), 4.29 (2H, s), 5.46 (1H, s), 5.97 (1H, d, J = 2.3 Hz), |
| | 6.85 (2H, d, J = 8.7 Hz), 7.23 (2H, d, J = 8.7 Hz), 7.34 (1H, d, J = 2.3 Hz). |
| I-101 | 1H-NMR (CDCl3) δ: 1.87-2.13 (4H, m), 2.35 (3H, br s), 2.43 (3H, s), 2.46 |
| | (3H, s), 2.54-2.66 (4H, m), 4.15 (2H, s), 4.58 (2H, s), 5.64 (1H, br s), |
| | 7.14-7.18 (2H, m), 7.35 (1H, s), 7.56-7.61 (2H, m). |
| I-102 | 1H-NMR (CDCl3) δ: 1.88-1.98 (3H, m), 2.03-2.12 (3H, m), 2.33 (3H, s), |
| | 2.43 (3H, s), 2.51-2.64 (4H, m), 4.15 (2H, s), 4.58 (2H, s), 5.68 (1H, s), |
| | 7.18 (1H, s), 7.37 (2H, s), 7.44 (2H, d, J = 8.4 Hz), 7.56 (1H, d, J = 8.4 |
| | Hz), 7.91 (1H, s). |
| I-103 | 1H-NMR (CDCl3) δ: 1.84-1.91 (2H, m), 2.00-2.07 (2H, m), 2.33 (3H, s), |
| | 2.53-2.58 (4H, m), 4.16 (3H, s), 4.30 (2H, s), 4.33 (2H, s), 4.39-4.42 (2H, |
| | m), 4.69 (1H, s), 6.68-6.72 (2H, m), 7.39 (1H, dd, J = 8.5, 8.4 Hz), 7.45 |
| | (1H, s). |

TABLE 37

| | |
|---|---|
| I-104 | 1H-NMR (CDCl3) δ: 1.87-1.94 (2H, m), 2.04-2.07 (2H, m), 2.33 (3H, s), |
| | 2.42 (3H, s), 2.51-2.61 (4H, m), 4.07 (2H, s), 4.34 (2H, s), 4.38-4.41 (2H, |
| | m), 5.85 (1H, s), 6.66-6.70 (2H, m), 7.38 (1H, s), 7.46 (1H, dd, J = 8.5, |
| | 8.5 Hz). |
| I-106 | 1H-NMR (CDCl3) δ: 1.02 (6H, d, J = 6.8 Hz), 1.81-1.88 (2H, m), 2.00-2.11 |
| | (3H, m), 2.31 (3H, s), 2.47-2.58 (4H, m), 3.69 (2H, d, J = 6.7 Hz), 4.15 (3H, |
| | s), 4.29 (2H, s), 4.31 (2H, s), 4.65 (1H, s), 6.59-6.68 (2H, m), 7.29 (1H, t, |
| | J = 9.4 Hz), 7.48 (1H, s). |
| I-107 | 1H-NMR (CDCl3) δ: 1.04 (6H, d, J = 6.8 Hz), 1.81-1.88 (2H, m), 2.01-2.07 |
| | (2H, m), 2.09-2.16 (1H, m), 2.31 (3H, s), 2.48-2.57 (4H, m), 3.78 (2H, d, |
| | J = 6.7 Hz), 4.16 (3H, s), 4.26 (2H, s), 4.27 (2H, s), 4.57 (1H, s), 6.90 (1H, |
| | t, J = 8.3 Hz), 6.97 (1H, d, J = 8.7 Hz), 7.04 (1H, dd, J = 11.9, 1.9 Hz), 7.39 |
| | (1H, s). |
| I-108 | 1H-NMR (CDCl3) δ: 0.99-1.07 (4H, m), 1.84-1.94 (2H, m), 1.97-2.08 (3H, |
| | m), 2.32 (3H, s), 2.48-2.61 (4H, m), 4.04 (2H, s), 4.29-4.37 (4H, m), 5.99 |
| | (1H, s), 6.63-6.73 (2H, m), 7.30 (1H, s), 7.45 (1H, dd, J = 8.5, 8.5 Hz). |
| I-109 | 1H-NMR (CDCl3) δ: 0.94 (3H, t, J = 7.3 Hz), 1.61-1.64 (2H, m), 1.87- |
| | 1.93 (2H, m), 2.03-2.08 (2H, m), 2.32 (3H, s), 2.41 (3H, s), 2.55-2.57 (5H, |
| | m), 4.08 (2H, s), 4.36 (2H, s), 5.78 (1H, s), 6.85 (1H, dd, J = 11.2, 1.4 |
| | Hz), 6.92 (1H, dd, J = 7.8, 1.4 Hz), 7.34-7.36 (2H, m). |
| I-110 | 1H-NMR (CDCl3) δ: 0.94 (3H, t, J = 7.3 Hz), 1.57-1.66 (2H, m), 1.86- |
| | 1.93 (2H, m), 2.02-2.09 (2H, m), 2.32 (3H, s), 2.42 (3H, s), 2.51-2.61 (6H, |
| | m), 4.06 (2H, s), 4.34 (2H, s), 5.73 (1H, s), 6.93-6.99 (2H, m), 7.12 (1H, |
| | dd, J = 7.6, 7.6 Hz), 7.26-7.29 (1H, m). |
| I-111 | 1H-NMR (CDCl3) δ: 0.98 (3H, t, J = 7.3 Hz), 1.51 (2H, td, J = 15.0, 7.3 |
| | Hz), 1.77-1.88 (4H, m), 2.00-2.08 (2H, m), 2.31 (3H, s), 2.45-2.60 (4H, |
| | m), 4.03 (2H, t, J = 6.5 Hz), 4.16 (3H, s), 4.26 (2H, s), 4.27 (2H, s), 4.58 |
| | (1H, s), 6.91 (1H, dd, J = 8.4, 8.4 Hz), 6.98 (1H, dd, J = 8.4, 2.0 Hz), 7.04 |
| | (1H, dd, J = 11.9, 2.0 Hz), 7.39 (1H, s). |
| I-112 | 1H-NMR (CDCl3) δ: 0.98 (3H, t, J = 7.3 Hz), 1.42-1.52 (2H, m), 1.71- |
| | 1.89 (4H, m), 1.98-2.07 (2H, m), 2.31 (3H, s), 2.42-2.59 (4H, m), 3.94 |
| | (2H, t, J = 6.4 Hz), 4.15 (3H, s), 4.29 (2H, s), 4.31 (2H, s), 4.65 (1H, s), |
| | 6.61 (1H, d, J = 12.2 Hz), 6.66 (1H, d, J = 8.8 Hz), 7.30 (1H, d, J = 8.8 |
| | Hz), 7.44 (1H, s). |

TABLE 38

| | |
|---|---|
| I-115 | 1H-NMR (CDCl3) δ: 0.98 (3H, t, J = 7.4 Hz), 1.50 (2H, td, J = 14.9, 7.4 |
| | Hz), 1.76-1.83 (2H, m), 1.86-1.93 (2H, m), 2.01-2.10 (2H, m), 2.32 (3H, |
| | s), 2.42 (3H, s), 2.49-2.62 (4H, m), 4.02 (2H, t, J = 6.6 Hz), 4.04 (2H, s), |
| | 4.28 (2H, s), 5.73 (1H, s), 6.89 (1H, dd J = 8.4, 8.4 Hz), 6.98 (1H, d, J = |
| | 8.4 Hz), 7.03 (1H, dd, J = 11.9, 2.0 Hz), 7.29 (1H, s). |
| I-116 | 1H-NMR (CDCl3) δ: 1.00-1.08 (4H, m), 1.85-1.94 (2H, m), 1.97-2.10 (3H, |
| | m), 2.32 (3H, s), 2.49-2.62 (4H, m), 4.02 (2H, s), 4.29 (2H, s), 4.40 (2H, |
| | q, J = 8.2 Hz), 5.96 (1H, s), 6.97 (1H, dd, J = 8.0, 8.0 Hz), 7.01 (1H, d, J = |
| | 10.3 Hz), 7.09 (1H, d, J = 11.9 Hz), 7.24 (1H, s). |
| I-117 | 1H-NMR (CDCl3) δ: 1.02 (6H, d, J = 6.8 Hz), 1.88-1.91 (2H, m), 2.04- |
| | 2.10 (6H, m), 2.32 (3H, s), 2.35 (3H, s), 2.55 (4H, s), 3.70 (2H, d, J = 6.7 |
| | Hz), 3.96 (2H, s), 4.28 (2H, s), 6.14 (1H, s), 6.84 (2H, d, J = 8.5 Hz), 7.19 |
| | (2H, d, J = 8.5 Hz). |
| I-118 | 1H-NMR (CDCl3) δ: 1.03 (6H, d, J = 6.7 Hz), 1.91-1.97 (2H, m), 2.03- |
| | 2.16 (3H, m), 2.34 (3H, s), 2.58 (4H, br s), 3.71 (2H, d, J = 6.5 Hz), 4.14 |
| | (2H, s), 4.34 (2H, s), 5.81 (1H, s), 6.86 (2H, d, J = 5.7 Hz), 7.25 (2H, d, |
| | J = 5.7 Hz), 7.39 (1H, s), 7.43-7.53 (3H, m), 7.96-7.99 (2H, m). |
| I-119 | 1H-NMR (CDCl3) δ: 1.83-1.93 (2H, m), 2.00-2.08 (2H, m), 2.31 (3H, s), |
| | 2.41 (3H, s), 2.48-2.61 (4H, m), 3.97 (2H, s), 4.04 (2H, s), 4.32 (2H, s), |
| | 5.68 (1H, s), 7.13-7.31 (7H, m). |
| I-120 | 1H-NMR (CDCl3) δ: 1.86-1.95 (2H, m), 2.02-2.09 (2H, m), 2.32 (3H, s), |
| | 2.42 (3H, s), 2.49-2.62 (4H, m), 3.93 (3H, s), 4.02 (2H, s), 4.28 (2H, s), |
| | 5.83 (1H, s), 6.72 (1H, d, J = 8.6 Hz), 7.30 (1H, s), 7.63 (1H, dd, J = 8.6, |
| | 2.5 Hz), 8.02 (1H, d, J = 2.5 Hz). |
| I-121 | 1H-NMR (CDCl3) δ: 1.85-2.08 (4H, m), 2.32 (3H, s), 2.42 (3H, s), 2.50- |
| | 2.64 (4H, m), 3.86 (3H, s), 4.18 (2H, s), 4.39 (2H, s), 6.44 (1H, s), 7.16 |
| | (1H, dd, J = 8.5, 2.9 Hz), 7.31 (1H, d, J = 8.5 Hz), 7.44 (1H, s), 8.20 (1H, |
| | d, J = 2.9 Hz). |
| I-122 | 1H-NMR (CDCl3) δ: 1.87-2.07 (4H, m), 2.33 (3H, s), 2.36 (3H, s), 2.50- |
| | 2.63 (4H, m), 4.05 (2H, s), 4.62 (2H, s), 7.10 (1H, s), 7.25-7.38 (3H, m), |
| | 7.82-7.89 (2H, m). |

TABLE 39

| | |
|---|---|
| I-123 | 1H-NMR (CDCl3) δ: 1.87-1.96 (2H, m), 2.03-2.11 (2H, m), 2.32 (3H, s), |
| | 2.43 (3H, s), 2.50-2.63 (4H, m), 4.16 (2H, s), 4.61 (2H, s), 5.63 (1H, s), |
| | 7.22 (1H, s), 7.32 (2H, dd, J = 7.0, 7.2 Hz), 7.37 (1H, s), 7.71 (1H, d, J = |
| | 7.0 Hz), 7.79 (1H, d, J = 7.2 Hz). |
| I-124 | 1H-NMR (CDCl3) δ: 1.25 (3H, t, J = 7.0 Hz), 1.89-2.12 (4H, m), 2.35 (3H, |
| | s), 2.42 (3H, s), 2.53-2.69 (4H, m), 3.54 (2H, q, J = 7.0 Hz), 4.04 (2H, s), |
| | 4.35 (2H, s), 4.49 (2H, s), 5.76 (1H, s), 7.27-7.34 (5H, m). |
| I-125 | 1H-NMR (CDCl3) δ: 1.86-1.97 (2H, m), 2.02-2.10 (2H, m), 2.33-2.42 (2H, |
| | m), 2.33 (3H, s), 2.42 (3H, s), 2.52-2.63 (4H, m), 2.82-2.90 (2H, m), 4.05 |
| | (2H, s), 4.34 (2H, s), 5.75 (1H, s), 7.16 (2H, d, J = 8.0 Hz), 7.22-7.26 (3H, |
| | m). |
| I-126 | 1H-NMR (CDCl3) δ: 1.03 (3H, t, J = 7.4 Hz), 1.71-1.86 (2H, m), 1.84- |
| | 1.96 (2H, m), 1.97-2.12 (2H, m), 2.31 (3H, s), 2.42 (3H, s), 2.55 (4H, br |
| | s), 3.89 (2H, t, J = 6.5 Hz), 4.07 (2H, s), 4.41 (2H, s), 5.87 (1H, s), 6.78 |
| | (1H, dd, J = 8.5, 2.5 Hz), 6.89 (1H, d, J = 2.5 Hz), 7.30-7.35 (1H, m), |
| | 7.34-7.44 (1H, m). |
| I-127 | 1H-NMR (CDCl3) δ: 1.03 (6H, d, J = 6.8 Hz), 1.86-1.93 (2H, m), 2.03- |
| | 2.17 (3H, m), 2.31 (3H, s), 2.42 (3H, s), 2.55 (4H, br s), 3.77 (2H, d, J = |
| | 6.5 Hz), 4.04 (2H, s), 4.27 (2H, s), 5.73 (1H, s), 6.88 (1H, t, J = 8.3 Hz), |
| | 6.96-7.05 (2H, m), 7.30 (1H, s). |
| I-128 | 1H-NMR (CDCl3) δ: 1.02 (6H, d, J = 5.1 Hz), 1.85-1.92 (2H, m), 1.98- |
| | 2.14 (3H, m), 2.31 (3H, s), 2.41 (3H, s), 2.55 (4H, s), 3.69 (2H, d, J = 4.8 |
| | Hz), 4.06 (2H, s), 4.33 (2H, s), 5.77 (1H, s), 6.58 (1H, dd, J = 12.0, 2.5 |
| | Hz), 6.66 (1H, dd, J = 8.4, 2.4 Hz), 7.36 (2H, t, J = 8.7 Hz). |
| I-129 | 1H-NMR (CDCl3) δ: 1.03 (3H, t, J = 5.4 Hz), 1.73-1.88 (4H, m), 1.90- |
| | 2.01 (2H, m), 2.42 (3H, s), 2.84-2.95 (2H, m), 3.03-3.09 (2H, m), 3.89 |
| | (2H, t, J = 6.5 Hz), 4.08 (2H, s), 4.42 (2H, s), 5.91 (1H, s), 6.79 (1H, dd, |
| | J = 8.5, 2.5 Hz), 6.85-6.94 (1H, m), 7.33 (1H, s), 7.35-7.42 (1H, m). |
| I-130 | 1H-NMR (CDCl3) δ: 1.03 (6H, d, J = 4.8 Hz), 1.79-1.85 (2H, m), 1.88- |
| | 2.04 (2H, m), 2.04-2.21 (1H, m), 2.41 (3H, s), 2.89-2.94 (2H, m), 3.03- |
| | 3.09 (2H, m), 3.77 (2H, d, J = 5.1 Hz), 4.05 (2H, s), 4.29 (2H, s), 5.78 |
| | (1H, s), 6.89 (1H, t, J = 8.3 Hz), 6.97-7.09 (2H, m), 7.31 (1H, s). |

TABLE 40

| | |
|---|---|
| I-131 | 1H-NMR (CDCl3) δ: 1.02 (6H, d, J = 5.1 Hz), 1.78-1.84 (2H, m), 1.89- |
| | 2.00 (2H, m), 2.02-2.15 (1H, m), 2.43 (3H, s), 2.82-2.99 (2H, m), 3.03- |
| | 3.09 (2H, m), 3.69 (2H, d, J = 5.1 Hz), 4.06 (2H, s), 4.33 (2H, s), 5.81 |
| | (1H, s), 6.58 (1H, dd, J = 12.0, 2.5 Hz), 6.66 (1H, dd, J = 8.5, 2.3 Hz), |
| | 7.36 (2H, t, J = 6.9 Hz). |
| I-132 | 1H-NMR (CDCl3) δ: 1.83-1.92 (2H, m), 2.01-2.08 (2H, m), 2.31 (3H, s), |
| | 2.48-2.60 (4H, m), 4.14 (2H, s), 4.34 (2H, s), 4.35 (2H, q, J = 8.1 Hz), |
| | 5.16 (1H, s), 5.34 (2H, d, J = 47.3 Hz), 6.91 (2H, d, J = 8.7 Hz), 7.26 (2H, |
| | d, J = 8.7 Hz), 7.47 (1H, s). |
| I-133 | 1H-NMR (CDCl3) δ: 0.93 (3H, t, J = 7.4 Hz), 1.35 (2H, td, J = 14.8, 7.4 |
| | Hz), 1.54-1.63 (2H, m), 1.79-1.86 (2H, m), 1.99-2.07 (2H, m), 2.30 (3H, |
| | s), 2.46-2.55 (4H, m), 2.60 (2H, t, J = 7.8 Hz), 4.15 (3H, s), 4.29 (2H, s), |
| | 4.31 (2H, s), 4.56 (1H, s), 7.15 (2H, d, J = 7.8 Hz), 7.19 (2H, d, J = 7.8 |
| | Hz), 7.36 (1H, s). |
| I-134 | 1H-NMR (CDCl3) δ: 0.98 (3H, t, J = 7.4 Hz), 1.47-1.51 (2H, m), 1.76- |
| | 1.80 (4H, m), 1.95-1.97 (2H, m), 2.42 (3H, s), 2.89-2.92 (2H, m), 3.03- |
| | 3.07 (2H, m), 3.95 (2H, t, J = 6.5 Hz), 4.04 (2H, s), 4.29 (2H, s), 5.72 (1H, |
| | s), 6.83-6.86 (2H, m), 7.20-7.22 (2H, m), 7.26 (1H, s). |

The following is a description of a biological test example of the compound according to the present invention. The compounds of the present invention can be tested essentially as described in test examples below.

The compound represented by Formula (I), Formula (II), or Formula (III) according to the present invention may have serotonin 5-HT2A receptor antagonism and/or inverse agonism, and may antagonize the human serotonin 5-HT2A receptor.

The compound represented by Formula (I), Formula (II), or Formula (III) according to the present invention may have serotonin 5-HT2A and 5-HT2C receptor antagonism and/or inverse agonism, and may antagonize the human serotonin 5-HT2A and 5-HT2C receptors.

Specifically, in the evaluation method described below, the Ki value is preferably 5000 nM or less, more preferably 1000 nM or less, even more preferably 100 nM or less.

### Test Example 1: 5-HT2A Receptor Binding Inhibition Test

### (Each Experimental Condition)

Cell membrane: 15 μg of Jump-In HEK cell membrane (expressing human recombinant 5-HT2A receptor) per well

Assay buffer: Tris-HCl 50 mmol/L (pH 7.4) containing NaCl 120 mmol/L, MgCl₂·6H₂O 1 mmol/L, KCl 5 mmol/L, 0.1% BSA and CaCl₂ 2 mmol/L

Radioactive ligand: [³H]-Ketanserin with a final concentration of around Kd value calculated by the following method

Non-specific ligand: Serotonin HCl with a final concentration of 500 μmol/L

The Kd value is calculated when the lot of cell membrane is changed. In advance, 0.5 μL of a 1 mmol/L compound for non-specific binding calculation dissolved in DMSO or DMSO is dispensed into a microplate, and the cell membrane is diluted with an Assay buffer. The radioactive ligand solution is serially diluted and the count is confirmed with a liquid scintillator. Assay buffer containing diluted cell membrane is dispensed into a microplate at 50 μL/well. Then, the radioactive ligand solution is dispensed into a microplate at 50 μL/well, and the plate is sealed. It is allowed to stand at room temperature (25° C.) for 1.5 hours. During this period, 50 mmol/L Tris-HCl (pH 7.4) is dispensed into a GF/B UniFilter plate at 50 μL/well and allowed to stand at 4° C. for 1 hour or longer. After that, filtration is performed with Cell harvester (PerkinElmer). The radioactive ligand solution is dispensed into an empty well of the GF/B UniFilter plate at 10 μL/well. After the GF/B UniFilter plate is dried at room temperature, MicroScinti 20 is dispensed into the GF/B UniFilter plate at 50 μL/well to seal the plate. The GF/B UniFilter plate is allowed to stand overnight at room temperature. The radioactivity of [³H]-Ketanserin bound to the 5-HT2A receptor is measured using Microbeta2 (PerkinElmer) at a measurement time of 1 min/well. The Saturation curve is drawn from the measured value, and the Kd value is calculated from the slope of the Scatchard Plot.

### (Binding Test of the Compound According to the Present Invention)

In advance, 0.5 μL of the compound solution dissolved in DMSO is dispensed into a microplate, and the cell membrane and the hot ligand are diluted with Assay buffer, respectively. Then, the Assay buffer containing the diluted cell membrane is dispensed into a microplate at 50 μL/well. Then, the radioactive ligand solution is dispensed into a microplate at 50 μL/well, and the plate is sealed. Then, it is allowed to stand at room temperature (25° C.) for 1.5 hours. During this period, 50 mmol/L Tris-HCl (pH 7.4) is dispensed into a GF/B UniFilter plate at 50 μL/well and allowed to stand at 4° C. for 1 hour or longer. After that, filtration is performed with Cell harvester (PerkinElmer). After the GF/B UniFilter plate is dried at room temperature, MicroScinti 20 is dispensed into the GF/B UniFilter plate at 50 μL/well to seal the plate. The GF/B UniFilter plate is allowed to stand overnight at room temperature. The radioactivity of [³H]-Ketanserin bound to the 5-HT2A receptor is measured using Microbeta2 (PerkinElmer) at a measurement time of 1 min/well. The non-specific binding is calculated from the radioactivity of [³H]-Ketanserin in the presence of 500 μmol/L Serotonin HCl which is the unlabeled ligand, and the total binding is calculated from the radioactivity of [³H]-Ketanserin in the absence of the compound according to the present invention (Vehicle). Finally, the Ki value is calculated from the dose-response curve.

(The binding activity of the compound according to the present invention is calculated from the following binding inhibition rate (%).)

$$Inhibition rate ( \% ) = [ 1 ( c - a ) / ( b - a ) ] \times 100$$

a; mean cpm of non-specific binding
b; average cpm of total binding
c; cpm in the presence of the test compound

The compounds of the present invention were tested essentially as described above. Results are shown below.

### (Results)

The evaluation results regarding the human serotonin 5-HT2A receptor binding activity of the compound according to the present invention are shown below. In the table shown below, “A” means that the Ki value is less than 10 nM, “B” means that the Ki value is 10 nM or more and less than 100 nM, and “C” means that the Ki value is 100 nM or more and 500 nM or less.
Compound I-001: 4.82 nM
Compound I-002: 11.1 nM
Compound I-003: 19.8 nM
Compound I-005: 85.3 nM
Compound I-011: 1.22 nM
Compound I-027: 1.89 nM
Compound I-033: 10.1 nM
Compound I-049: 0.961 nM
Compound I-057: 5.20 nM
Compound I-067: 1.37 nM
Compound I-080: 0.823 nM
Compound I-087: 1.11 nM
Compound I-089: 1.49 nM
Compound I-099: 0.960 nM
Compound I-104: 1.56 nM
Compound I-105: 0.979 nM
Compound I-113: 1.20 nM
Compound I-114: 0.740 nM
Compound I-115: 0.703 nM
Compound I-125: 1.26 nM
Compound I-128: 1.36 nM
Compound I-130: 1.29 nM

TABLE 41

| Compound No. | h-5-HT2A Ki |
|---|---|
| I-004 | A |
| I-006 | C |
| I-007 | A |
| I-008 | B |
| I-009 | A |
| I-010 | A |
| I-012 | A |
| I-013 | B |
| I-014 | A |
| I-015 | A |
| I-016 | A |
| I-017 | A |
| I-018 | B |
| I-019 | A |
| I-020 | B |
| I-021 | A |
| I-022 | A |
| I-023 | B |
| I-024 | A |
| I-025 | B |
| I-026 | A |
| I-028 | A |
| I-029 | A |
| I-030 | A |
| I-031 | B |
| I-032 | B |
| I-034 | A |
| I-035 | A |
| I-036 | A |
| I-037 | A |
| I-038 | B |
| I-039 | B |
| I-040 | C |
| I-041 | A |
| I-042 | B |
| I-043 | B |
| I-044 | A |
| I-045 | B |
| I-046 | B |
| I-047 | A |
| I-048 | A |
| I-050 | A |
| I-051 | C |
| I-052 | C |
| I-053 | A |
| I-054 | A |
| I-055 | A |
| I-056 | C |
| I-058 | B |
| I-059 | B |
| I-060 | A |
| I-061 | B |
| I-062 | A |
| I-063 | A |
| I-064 | A |
| I-065 | A |
| I-066 | A |
| I-067 | A |
| I-068 | A |
| I-069 | B |
| I-070 | A |
| I-071 | A |
| I-072 | A |
| I-073 | B |
| I-074 | C |
| I-075 | A |
| I-076 | B |
| I-077 | A |
| I-078 | A |
| I-079 | A |
| I-080 | A |
| I-081 | A |
| I-082 | A |
| I-083 | A |
| I-084 | A |
| I-085 | A |
| I-086 | A |

TABLE 42

| Compound No. | h-5-HT2A Ki |
|---|---|
| I-088 | B |
| I-090 | A |
| I-091 | B |
| I-092 | A |
| I-093 | C |
| I-094 | B |
| I-095 | B |
| I-096 | A |
| I-097 | A |
| I-098 | A |
| I-100 | A |
| I-101 | A |
| I-102 | A |
| I-103 | A |
| I-106 | A |
| I-107 | A |
| I-108 | A |
| I-109 | A |
| I-110 | A |
| I-111 | A |
| I-112 | A |
| I-116 | A |
| I-117 | B |
| I-118 | C |
| I-119 | A |
| I-120 | B |
| I-121 | B |
| I-122 | A |
| I-123 | A |
| I-124 | B |
| I-126 | A |
| I-127 | A |
| I-129 | A |
| I-131 | A |
| I-132 | A |
| I-133 | A |
| I-134 | A |

### Test Example 2: 5-HT2C Receptor Binding Inhibition Test

### (Each Experimental Condition)

Cell membrane: 0.5 μg of Jump-In HEK cell membrane (expressing human recombinant 5-HT2C receptor) per well

Assay buffer: Tris-HCl 50 mmol/L (pH 7.4) containing NaCl 120 mmol/L, MgCl₂·6H₂O 1 mmol/L, KCl 5 mmol/L, 0.1% BSA and CaCl₂ 2 mmol/L

Radioactive ligand: [³H]-Mesulergine with a final concentration of around Kd value calculated by the following method

Non-specific ligand: Serotonin HCl with a final concentration of 500 gmol/L

The Kd value is calculated when the lot of cell membrane is changed. In advance, 0.5 μL of a 1 mmol/L compound for non-specific binding calculation dissolved in DMSO or DMSO is dispensed into a microplate, and the cell membrane is diluted with an Assay buffer. The radioactive ligand solution is serially diluted and the count is confirmed with a liquid scintillator. Assay buffer containing diluted cell membrane is dispensed into a microplate at 50 μL/well. Then, the radioactive ligand solution is dispensed into a microplate at 50 μL/well, and the plate is sealed. It is allowed to stand at 37° C. for 2 hours. During this period, 50 mmol/L Tris-HCl (pH 7.4) is dispensed into a GF/B UniFilter plate at 50 μL/well and allowed to stand at 4° C. for 1 hour or longer. After that, filtration is performed with Cell harvester (PerkinElmer). The radioactive ligand solution is dispensed into an empty well of the GF/B UniFilter plate at 10 μL/well. After the GF/B UniFilter plate is dried at room temperature, MicroScinti 20 is dispensed into the GF/B UniFilter plate at 50 μL/well to seal the plate. The GF/B UniFilter plate is allowed to stand overnight at room temperature. The radioactivity of [³H]-Mesulergine bound to the 5-HT2C receptor is measured using Microbeta2 (PerkinElmer) at a measurement time of 1 min/well. The Saturation curve is drawn from the measured value, and the Kd value is calculated from the slope of the Scatchard Plot.

### (Binding Test of the Compound According to the Present Invention)

In advance, 0.5 μL of the compound solution dissolved in DMSO is dispensed into a microplate, and the cell membrane and the hot ligand are diluted with Assay buffer, respectively. Then, the Assay buffer containing the diluted cell membrane is dispensed into a microplate at 50 μL/well. Then, the radioactive ligand solution is dispensed into a microplate at 50 μL/well, and the plate is sealed. Then, it is allowed to stand at 37° C. for 2 hours. During this period, 50 mmol/L Tris-HCl (pH 7.4) is dispensed into a GF/B UniFilter plate at 50 μL/well and allowed to stand at 4° C. for 1 hour or longer. After that, filtration is performed with Cell harvester (PerkinElmer). After the GF/B UniFilter plate is dried at room temperature, MicroScinti 20 is dispensed into the GF/B UniFilter plate at 50 μL/well, and the plate is sealed. The GF/B UniFilter plate is allowed to stand overnight at room temperature. The radioactivity of [³H]-Mesulergine bound to the 5-HT2C receptor is measured using Microbeta2 (PerkinElmer) at a measurement time of 1 min/well. The non-specific binding is calculated from the radioactivity of [31H]-Mesulergine in the presence of 500 μmol/L Serotonin HCl which is the unlabeled ligand, and the total binding is calculated from the radioactivity of [³H]-Mesulergine in the absence of the compound according to the present invention (Vehicle). Finally, the Ki value is calculated from the dose-response curve.

(The binding activity of the compound according to the present invention is calculated from the following binding inhibition rate (%).)

$$Inhibition rate ( \% ) = [ 1 - ( c - a ) / ( b - a ) ] \times 100$$

a; mean cpm of non-specific binding
b; average cpm of total binding
c; cpm in the presence of the test compound

The compounds of the present invention were tested essentially as described above. Results are shown below.

### (Results)

The evaluation results regarding the human serotonin 5-HT2C receptor binding activity of the compound according to the present invention are shown below. In the table shown below, “A” means that the Ki value is less than 10 nM, “B” means that the Ki value is 10 nM or more and less than 100 nM, and “C” means that the Ki value is 100 nM or more and 500 nM or less.
Compound I-001: 16.7 nM
Compound I-002: 27.1 nM
Compound I-003: 4.11 nM
Compound I-005: 219 nM
Compound I-011: 0.585 nM
Compound I-027: 2.21 nM
Compound I-033: 19.0 nM
Compound I-049: 0.524 nM
Compound I-057: 2.19 nM
Compound I-067: 0.950 nM
Compound I-080: 0.579 nM
Compound I-087: 0.787 nM
Compound I-089: 5.46 nM
Compound I-099: 3.08 nM
Compound I-104: 0.578 nM
Compound I-105: 1.13 nM
Compound I-113: 1.60 nM
Compound I-114: 0.543 nM
Compound I-115: 0.469 nM
Compound I-125: 0.694 nM
Compound I-128: 0.374 nM
Compound I-130: 0.535 nM

TABLE 43

| Compound No. | h-5-HT2C Ki |
|---|---|
| I-004 | B |
| I-007 | B |
| I-008 | C |
| I-009 | A |
| I-010 | A |
| I-012 | B |
| I-013 | B |
| I-014 | B |
| I-015 | B |
| I-016 | A |
| I-017 | A |
| I-018 | A |
| I-019 | C |
| I-020 | C |
| I-021 | B |
| I-022 | A |
| I-023 | B |
| I-024 | B |
| I-025 | B |
| I-026 | A |
| I-028 | A |
| I-029 | A |
| I-030 | B |
| I-031 | B |
| I-032 | B |
| I-034 | B |
| I-035 | A |
| I-036 | A |
| I-037 | A |
| I-038 | A |
| I-039 | A |
| I-040 | C |
| I-041 | A |
| I-042 | B |
| I-043 | B |
| I-044 | A |
| I-045 | C |
| I-046 | C |
| I-047 | A |
| I-048 | A |
| I-050 | A |
| I-051 | C |
| I-052 | C |
| I-053 | A |
| I-054 | B |
| I-055 | B |
| I-058 | C |
| I-060 | B |
| I-061 | B |
| I-062 | B |
| I-063 | B |
| I-064 | A |
| I-065 | A |
| I-066 | A |
| I-067 | A |
| I-068 | A |
| I-069 | A |
| I-070 | A |
| I-071 | A |
| I-072 | A |
| I-073 | A |
| I-074 | C |
| I-075 | C |
| I-076 | C |
| I-077 | A |
| I-078 | C |
| I-079 | A |
| I-080 | A |
| I-081 | C |
| I-082 | A |
| I-083 | A |
| I-084 | A |
| I-085 | A |
| I-086 | A |

TABLE 44

| Compound No. | h-5-HT2C Ki |
|---|---|
| I-088 | B |
| I-090 | A |
| I-091 | C |
| I-092 | A |
| I-093 | C |
| I-094 | B |
| I-095 | C |
| I-096 | B |
| I-097 | A |
| I-098 | B |
| I-100 | B |
| I-101 | A |
| I-102 | A |
| I-103 | A |
| I-106 | A |
| I-107 | A |
| I-108 | A |
| I-109 | A |
| I-110 | A |
| I-111 | A |
| I-112 | A |
| I-116 | A |
| I-117 | B |
| I-118 | C |
| I-119 | A |
| I-120 | B |
| I-121 | B |
| I-122 | A |
| I-123 | A |
| I-124 | B |
| I-126 | A |
| I-127 | A |
| I-129 | A |
| I-131 | A |
| I-132 | A |
| I-133 | A |
| I-134 | A |

### Test Example 3: hERG Test

For the purpose of evaluating risk of an electrocardiogram QT interval prolongation of the compound of the present invention, effects of the compound are studied by evaluating the activity of potassium channel using CHO cells expressing human ether-a-go-go related gene (hERG) channel.

Evaluation is performed using a FluxORII Green Potassium IonCgannel Assay kit

### (Invitrogen: Molecular Probes).

Cells are seeded in a 384-assay plate (8000 cells/well/40 μL) and incubated (37° C., 5% COD overnight. After replacing the medium with a Wash buffer (1×HBSS, 20 mM HEPES) with a microplate washer, a fluorescent indicator is added to the medium and allowed to incubate (37° C., 5% CO₂) for 1 hour for incorporation of the fluorescent indicator into cells.

A cell plate is placed on a cell-based kinetic assay system FLIPR (Molecular Devices, LLC.), and the compound is added to the cells so as to have a desired concentration and reacted for 10 minutes. When a mixed solution of potassium and thallium as a stimulator is added thereto, the potassium channel is opened, and thallium that flowed into the cells binds to the fluorescent indicator, so that the intracellular fluorescence signal increases, and the potassium channel current is detected as a fluorescence signal. For the inhibition rate at each concentration, the signal intensity when E-4031 is added to cells at a final concentration of 10.3 μmol/L is defined as an inhibition rate of 100%, and the signal intensity when DMSO is added to cells at a final concentration of 0.5% is defined as an inhibition rate of 0%. Then, the inhibition rate is calculated from the signal intensity at each concentration. IC₅₀ is calculated from the inhibition rate at each concentration.

The compounds of the present invention were tested essentially as described above. Results are shown below.

### (Results)

Compound I-067: IC₅₀=16.3 μM
Compound I-080: IC₅₀>52.0 μM
Compound I-104: IC₅₀=20.6 μM
Compound I-105: IC₅₀=17.9 μM
Compound I-113: IC₅₀>52.0 μM
Compound I-114: IC₅₀>52.0 μM
Compound I-115: IC₅₀>52.0 μM
Compound I-125: IC₅₀=20.0 μM
Compound I-128: IC₅₀>52.0 μM

### Test Example 4: BA Test Evaluation of Oral Absorption

### Materials and Methods for Experiments

(1) Animals used: Mice or rats are used.
(2) Rearing conditions: Mice or rats are allowed to freely take solid feed and sterilized tap water.
(3) Dose and grouping setting: A given dose is orally administered and intravenously administered. Grouping is set as below. The dosage is changed per compound as necessary.
   Oral administration 2 to 60 μmol/kg or 1 to 30 mg/kg (n=2 to 3)
   Intravenous administration 1 to 30 μmol/kg or 0.5 to 10 mg/kg (n=2 to 3)
(4) Preparation of administration solution: The test sample is administered as a solution or a suspension for the oral administration. Intravenous administration is performed after solubilization.
(5) Administration method: The test sample is forcedly administered into the stomach through an oral probe for the oral administration. Intravenous administration is performed from caudal vein by syringes with needle.
(6) Evaluation items: Blood is collected serially and concentration of a compound according to the present invention in plasma is measured by LC/MS/MS.
(7) Statistical analysis: About transition of concentration of a compound according to the present invention in plasma, the area under the plasma concentration versus time curve (AUC) is calculated by moment analysis method, and bioavailability (BA) of a compound according to the present invention is calculated from the dosage ratio and the AUC ratio between the oral administration group and the intravenous administration group.

The dilution concentration or the dilution solvent are changed as necessary.

The compounds of the present invention can be tested essentially as described above.

### Test Example 5: Clearance Evaluation Test

### Materials and Methods for Experiments

(1) Animals used: SD rats are used.
(2) Rearing conditions: SD rats are allowed to freely take solid feed and sterilized tap water.
(3) Dose and grouping setting: A given dose was intravenously administered. Grouping is set as below.
   Intravenous administration 1 μmol/kg (n=2)
(4) Preparation of administration solution: The test sample is solubilized using a solvent of dimethyl sulfoxide/propylene glycol=1/1 and administered.
(5) Administration method: The test sample is administered to the tail vein through a syringe with an injection needle.
(6) Evaluation items: Blood is collected serially and concentration of a compound according to the present invention in plasma is measured by LC/MS/MS.
(7) Statistical analysis: About transition of concentration of a compound according to the present invention in plasma, total clearance (CLtot) of a compound according to the present invention is calculated by the moment analysis method. The dilution concentration or the dilution solvent are changed as necessary.

The compounds of the present invention can be tested essentially as described above.

### Test Example 6: Metabolic Stability Test

Using commercially available pooled human liver microsomes, a compound of the present invention is reacted for a constant time, and a remaining rate is calculated by comparing a reacted sample and an unreacted sample, thereby, a degree of metabolism in liver is assessed.

A reaction is performed (oxidative reaction) at 37° C. for 0 minutes or 30 minutes in the presence of 1 mmol/L NADPH in 0.2 mL of a buffer (50 mmol/L Tris-HCl pH 7.4, 150 mmol/L potassium chloride, 10 mmol/L magnesium chloride) containing 0.5 mg protein/mL of human liver microsomes. After the reaction, 70 μL of the reaction solution is added to 140 μL of a solution of methanol/acetonitrile=1/1 (v/v), mixed and centrifuged at 3000 rpm for 15 minutes. The compound of the present invention in the centrifuged supernatant is quantified by LC/MS/MS or solid-phase extraction (SPE)/MS. The ratio of the amount of the compound after the reaction, with respect to the amount of the compound of the present invention at 0 minutes of the reaction defined as 100%, is shown as the remaining rate. Hydrolysis reaction is performed in the absence of NADPH, and glucuronidation reaction is performed in the presence of 5 mmol/L UDP-glucuronic acid instead of NADPH. Then, the same operation is carried out. The dilution concentration or the dilution solvent are changed as necessary.

The compounds of the present invention can be tested essentially as described above.

### Test Example 7: P-Gp Substrate Test

The compound according to the present invention is added to one side of a Transwell (registered trademark, CORNING) in which human MDR1-expressing cells or parent cells are cultured in a single layer, and reacted for a certain period of time. For MDR1-expressing cells and parent cells, the membrane permeability coefficients from the Apical side to the Basolateral side (A→B) and from the Basolateral side to the Apical side (B→A) are calculated, and Efflux Ratio (ER; ratio of membrane permeability coefficients of B→A and A→B) value of the MDR1-expressing cells and the parent cells are calculated. The Efflux Ratio (ER value) of the MDR1-expressing cells and the parent cells are compared to determine whether the compound according to the present invention is a P-gp substrate or not.

The compounds of the present invention can be tested essentially as described above.

### Test Example 8: CYP3A4 (MDZ) MBI Test

This test is a test to evaluate mechanism based inhibition (MBI) potency from enhancement of CYP3A4 inhibition of the compound of the present invention by a metabolism reaction. CYP3A4 inhibition is evaluated using pooled human liver microsomes by 1-hydroxylation reaction of midazolam (MDZ) as a marker reaction.

The reaction conditions are as follows: substrate, 10 μmol/L MDZ; pre-reaction time, 0 or 30 minutes; reaction time, 2 minutes; reaction temperature, 37° C.; pooled human liver microsomes, at pre-reaction 0.5 mg/mL, at reaction 0.05 mg/mL (at 10-fold dilution); concentrations of the compound of the present invention, 0.83, 5, 10, 20 μmol/L (four points).

Pooled human liver microsomes and a solution of the compound of the present invention in K-Pi buffer (pH 7.4) as a pre-reaction solution are added to a 96-well plate at the composition of the pre-reaction. A part of pre-reaction solution is transferred to another 96-well plate, and 1/10 diluted by K-Pi buffer containing a substrate. NADPH as a co-factor is added to initiate a reaction as a marker reaction (without preincubation). After a predetermined time of a reaction, methanol/acetonitrile=1/1 (V/V) solution is added to stop the reaction. In addition, NADPH is added to a remaining pre-reaction solution to initiate a pre-reaction (with preincubation). After a predetermined time of a pre-reaction, a part is transferred to another plate, and 1/10 diluted by K-Pi buffer containing a substrate to initiate a reaction as a marker reaction. After a predetermined time of a reaction, methanol/acetonitrile=1/1 (V/V) solution is added to stop the reaction. Each plate where the marker reaction has been performed is centrifuged at 3000 rpm for 15 minutes. Then, midazolam 1-hydroxide in the centrifuged supernatant is quantified by LC/MS/MS.

Only DMSO which is a solvent dissolving the compound of the present invention added to a reaction system is adopted as a control (100%). Remaining activity (%) is calculated at each concentration of the compound of the present invention added. IC is calculated by reverse-presumption by a logistic model using a concentration and an inhibition rate. Shifted IC value is calculated as “IC of preincubation at 0 min/IC of preincubation at 30 min”. When a shifted IC is 1.5 or more, this is defined as positive. When a shifted IC is 1.0 or less, this is defined as negative.

The compounds of the present invention were tested essentially as described above. Results are shown below.

### (Results)

Compound I-067: Negative
Compound I-080: Negative
Compound I-104: Negative
Compound I-105: Negative
Compound I-113: Negative
Compound I-114: Negative
Compound I-115: Negative
Compound I-125: Negative
Compound I-128: Negative

### Test Example 9: MK801-Induced Hyperactivity Inhibition Test

6 to 10-week-old Wistar male rats are used. For preparation of a test compound administration solution, 30 mmol/L HCl is used as a vehicle by dissolving the test compound, and for preparation of an MK801 administration solution, physiological saline is used as a vehicle by dissolving MK801. Using SCANET manufactured by Melquest Ltd., a data collection program SCL-40, and a transparent plastic cage, the MK801-induced hyperactivity inhibition test is performed as follows.

In the rearing room, the compound administration solution (vehicle or test compound solution) is subcutaneously administered, and returned to the rearing cage. After 30 minutes, the animals are brought into a laboratory and subjected to laboratory acclimation. 15 minutes after that, the rats are gently taken out, and the MK801 administration solution (vehicle or MK801 solution) is intraperitoneally administered and returned to the rearing cage. The rats are taken out 15 minutes after intraperitoneal administration, and placed gently into the SCANET, and measurement of the amount of motor activity is started. The measurement is ended 30 minutes after the start of the measurement, and the amounts of motor activity of the individuals for 30 minutes are summed.

Analysis of the test results is performed as follows.

Student-T test (significance level: two-sided 5%) is performed in the test compound administration group and the vehicle administration group. When significant suppression of the amount of motor activity is shown in the test compound administration group as compared with the vehicle administration group, it is determined that the test compound has an antipsychotic effect.

The compounds of the present invention can be tested essentially as described above.

The following formulation examples are merely examples, and are not intended to limit the scope of the invention.

The compound according to the present invention can be administered as a pharmaceutical composition by any conventional route, in particular enterally, for example, orally, for example, in the form of tablets or capsules, or parenterally, for example, in the form of injectable solutions or suspensions, topically, for example, in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets, granules, or capsules containing excipients, disintegrants, binders, lubricants and the like and active ingredients. Compositions for injection can be solutions or suspensions, may be sterilized, and may contain preservatives, stabilizers, buffering agents, and the like.

### INDUSTRIAL APPLICABILITY

The compound according to the present invention has serotonin 5-HT2A receptor antagonism and/or inverse agonism, and the compound is considered to be useful as a therapeutic and/or prophylactic agent for a disease or condition associated with a serotonin 5-HT2A receptor.

1. A compound represented by Formula (I): 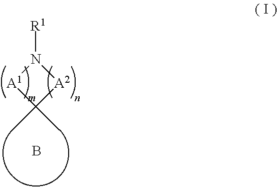
wherein R¹ is a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
A¹ is each independently CR²R^{2′};
A² is each independently CR³R^{3′};
R² is each independently a hydrogen atom, halogen, substituted or unsubstituted alkyl, or substituted or unsubstituted alkyloxy;
R^{2′} is each independently a hydrogen atom, halogen, substituted or unsubstituted alkyl, or substituted or unsubstituted alkyloxy;
R³ is each independently a hydrogen atom, halogen, substituted or unsubstituted alkyl, or substituted or unsubstituted alkyloxy;
R^{3′} is each independently a hydrogen atom, halogen, substituted or unsubstituted alkyl, or substituted or unsubstituted alkyloxy;
R² and R^{2′} and R³ and R^{3′} may be taken together with an identical carbon atom to which they are bonded to form a substituted or unsubstituted non-aromatic carbocycle or a substituted or unsubstituted non-aromatic heterocycle;
m and n are each independently 1, 2, or 3;
ring B is a ring represented by Formula: 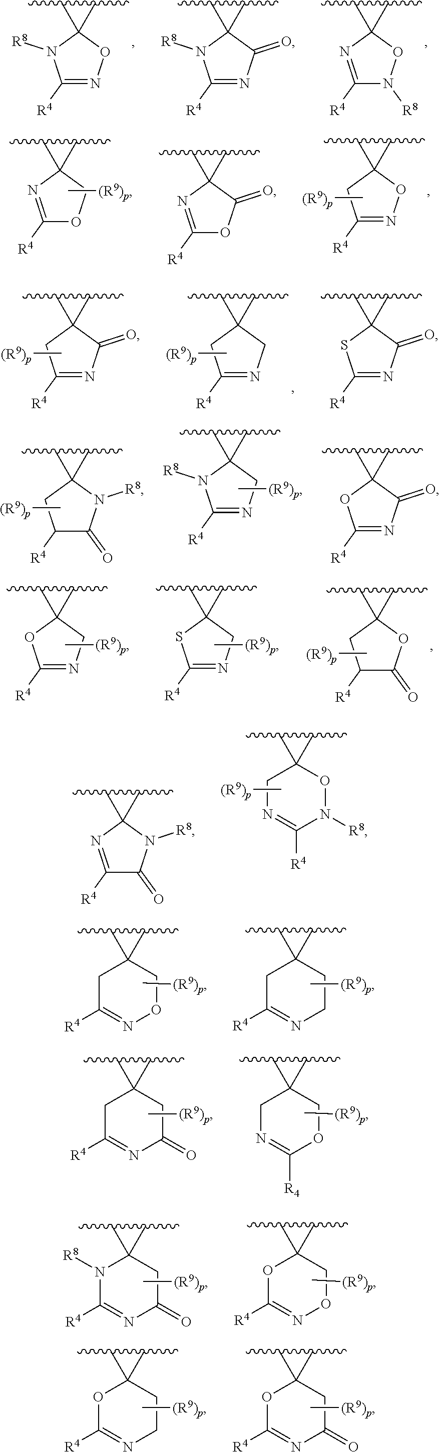 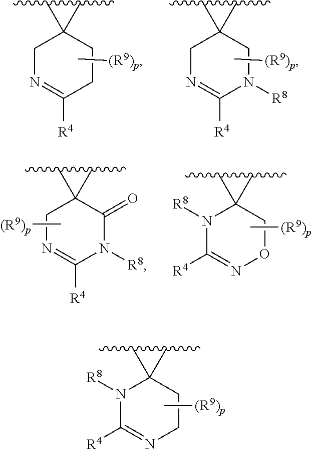
or
wherein R⁴ is a group represented by Formula: 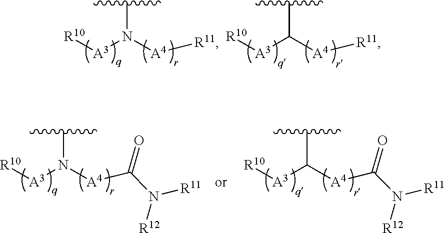
wherein A³ is each independently CR¹³R^{13′};
A⁴ is each independently CR¹⁴R^{14′};
R¹³ is each independently a hydrogen atom, halogen, substituted or unsubstituted alkyl, or substituted or unsubstituted alkyloxy;
R^{13′} is each independently a hydrogen atom, halogen, substituted or unsubstituted alkyl, or substituted or unsubstituted alkyloxy;
R¹⁴ is each independently a hydrogen atom, halogen, substituted or unsubstituted alkyl, or substituted or unsubstituted alkyloxy;
R^{14′} is each independently a hydrogen atom, halogen, substituted or unsubstituted alkyl, or substituted or unsubstituted alkyloxy;
q and r are each independently 0, 1, or 2;
q′ and r′ are each independently 1 or 2;
R¹⁰ and R¹¹ are each independently substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R¹² is a hydrogen atom or substituted or unsubstituted alkyl;
R⁸ is a hydrogen atom or substituted or unsubstituted alkyl;
R⁹ is each independently halogen or substituted or unsubstituted alkyl;
p is an integer of any of 0 to 6,
or a pharmaceutically acceptable salt thereof.

2. The compound according to claim 1, wherein R¹ is a hydrogen atom or substituted or unsubstituted alkyl, or a pharmaceutically acceptable salt thereof.

3. The compound according to claim 1, wherein m and n are each independently 1 or 2, or a pharmaceutically acceptable salt thereof.

4. The compound according to claim 1, wherein m and n are 2, or a pharmaceutically acceptable salt thereof.

5. The compound according to claim 1, wherein ring B is a ring represented by Formula: 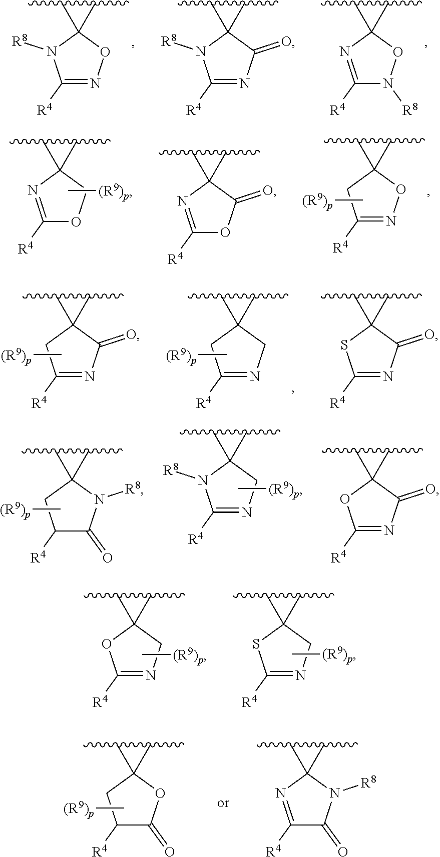
wherein symbols have the same meanings as those in claim 1, or a pharmaceutically acceptable salt thereof.

6. The compound according to claim 1, wherein ring B is a ring represented by Formula: 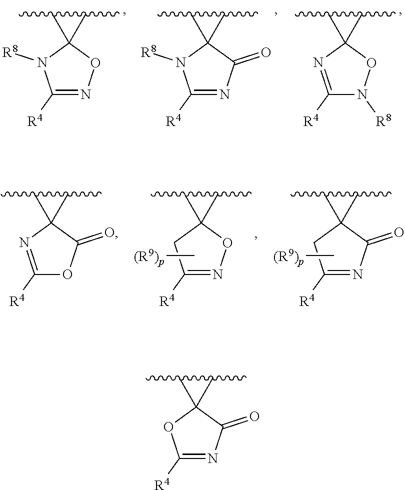
or
wherein symbols have the same meanings as those in claim 1, or a pharmaceutically acceptable salt thereof.

7. The compound according to claim 1, wherein R⁴ is a group represented by Formula: 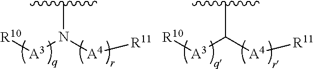
or
wherein symbols have the same meanings as those in claim 1, or a pharmaceutically acceptable salt thereof.

8. The compound according to claim 1, wherein R¹⁰ is substituted or unsubstituted aromatic carbocyclyl or substituted or unsubstituted aromatic heterocyclyl, or a pharmaceutically acceptable salt thereof.

9. The compound according to claim 1, wherein R¹⁰ is substituted or unsubstituted aromatic heterocyclyl, or a pharmaceutically acceptable salt thereof.

10. The compound according to claim 1, wherein R¹⁰ is substituted or unsubstituted 5-membered aromatic heterocyclyl, or a pharmaceutically acceptable salt thereof.

11. The compound according to claim 1, wherein R¹¹ is substituted or unsubstituted aromatic carbocyclyl, or a pharmaceutically acceptable salt thereof.

12. The compound according to claim 1, wherein q, r, q′, and r′ are 1, or a pharmaceutically acceptable salt thereof.

13. The compound according to claim 1, wherein the compound is represented by Formula (II): 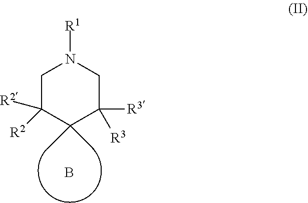
wherein R¹ is a hydrogen atom, substituted or unsubstituted alkyl, substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R² is a hydrogen atom, halogen, or substituted or unsubstituted alkyl;
R^{2′} is a hydrogen atom, halogen, or substituted or unsubstituted alkyl;
R³ is a hydrogen atom, halogen, or substituted or unsubstituted alkyl;
R^{3′} is a hydrogen atom, halogen, or substituted or unsubstituted alkyl; and
other symbols have the same meanings as those in claim 1,
or a pharmaceutically acceptable salt thereof.

14. The compound according to claim 1, wherein the compound is represented by Formula (II): 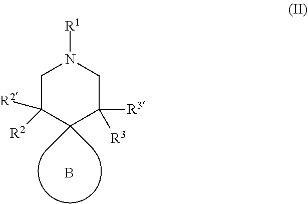
wherein R¹ is a hydrogen atom or alkyl;
R² is a hydrogen atom or halogen;
R^{2′} is a hydrogen atom;
R³ is a hydrogen atom;
R^{3′} is a hydrogen atom;
ring B is a ring represented by Formula: 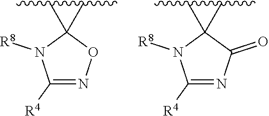
or
wherein R⁴ is a group represented by Formula: 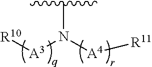
wherein A³ is CR¹³R^{13′};
A⁴ is CR¹⁴R^{14′};
R¹³ is a hydrogen atom;
R^{13′} is a hydrogen atom;
R¹⁴ is a hydrogen atom;
R^{14′} is a hydrogen atom;
q and r are each 1;
R¹⁰ is phenyl substituted with halogen, phenyl, 5-membered aromatic heterocyclyl substituted with one or more substituents selected from substituent group ω (substituent group ω: alkyl, haloalkyl, and non-aromatic carbocyclyl), or 6-membered aromatic heterocyclyl substituted with one or more substituents selected from substituent group ω′ (substituent group ω′: alkyl and halogen);
R¹¹ is a group represented by Formula: 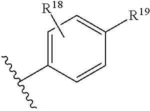
wherein R⁸ is a hydrogen atom or halogen;
R¹⁹ is alkyl, haloalkyl, alkyl substituted with aromatic carbocyclyl, alkyloxy, alkyloxy substituted with non-aromatic carbocyclyl, alkyloxy substituted with non-aromatic carbocyclyl substituted with halogen, or haloalkyloxy, 9-membered aromatic heterocyclyl, which is bicyclic, or 9-membered aromatic heterocyclyl, which is bicyclic, substituted with one or more substituents selected from substituent group ψ (substituent group ψ: halogen, alkyl, and alkyloxy);
R⁸ is a hydrogen atom,
or a pharmaceutically acceptable salt thereof.

15. A compound represented by Formula (III): 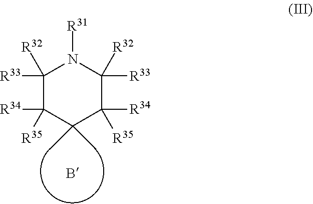
wherein R³¹ is a hydrogen atom or C1-C3 alkyl;
R³² is each independently a hydrogen atom or substituted or unsubstituted alkyl;
R³³ is each independently a hydrogen atom or substituted or unsubstituted alkyl;
R³⁴ is each independently a hydrogen atom, halogen, or substituted or unsubstituted alkyl;
R³⁵ is each independently a hydrogen atom, halogen, or substituted or unsubstituted alkyl;
R³² and R³³ and R³⁴ and R³⁵ may be taken together with an identical carbon atom to which they are bonded to form a substituted or unsubstituted non-aromatic carbocycle or a substituted or unsubstituted non-aromatic heterocycle;
ring B′ is a group represented by Formula: 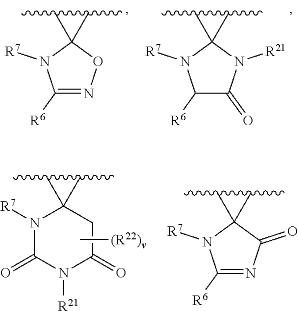
or
wherein R⁶ is a group represented by Formula: 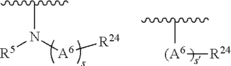
or
wherein A⁶ is each independently CR²⁵R^{25′};
R²⁵ is each independently a hydrogen atom, halogen, substituted or unsubstituted alkyl, or substituted or unsubstituted alkyloxy;
R^{25′} is each independently a hydrogen atom, halogen, substituted or unsubstituted alkyl, or substituted or unsubstituted alkyloxy;
s is 0 or 1;
s′ is 0, 1, or 2;
R²⁴ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁵ is a hydrogen atom or substituted or unsubstituted alkyl;
R^{6′} is a group represented by Formula: 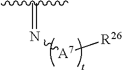
wherein A⁷ is CR²⁷R^{27′};
R²⁷ is a hydrogen atom, halogen, substituted or unsubstituted alkyl, or substituted or unsubstituted alkyloxy;
R^{27′} is a hydrogen atom, halogen, substituted or unsubstituted alkyl, or substituted or unsubstituted alkyloxy;
t is 0 or 1;
R²⁶ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl;
R⁷ is a group represented by Formula: 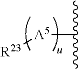
wherein A⁵ is each independently CR²⁸R^{28′};
R²⁸ is each independently a hydrogen atom, halogen, substituted or unsubstituted alkyl, or substituted or unsubstituted alkyloxy;
R^{28′} is each independently a hydrogen atom, halogen, substituted or unsubstituted alkyl, or substituted or unsubstituted alkyloxy;
u is 0, 1, or 2;
R²³ is substituted or unsubstituted aromatic carbocyclyl, substituted or unsubstituted non-aromatic carbocyclyl, substituted or unsubstituted aromatic heterocyclyl, or substituted or unsubstituted non-aromatic heterocyclyl,
R²¹ is a hydrogen atom or substituted or unsubstituted alkyl;
R²² is each independently halogen or substituted or unsubstituted alkyl;
v is 0, 1, or 2,
or a pharmaceutically acceptable salt thereof.

16. The compound according to claim 15, wherein ring B′ is a ring represented by Formula: 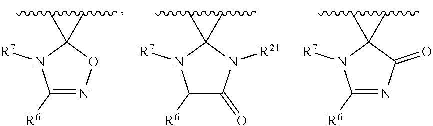
or
wherein symbols have the same meanings as those in claim 15, or a pharmaceutically acceptable salt thereof.

17. The compound according to claim 15, wherein R⁶ is a group represented by Formula: 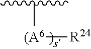
wherein symbols have the same meanings as those in claim 15, or a pharmaceutically acceptable salt thereof.

18. The compound according to claim 15, wherein s′ is 1, or a pharmaceutically acceptable salt thereof.

19. The compound according to claim 15, wherein R²⁴ is substituted or unsubstituted aromatic carbocyclyl, or a pharmaceutically acceptable salt thereof.

20. The compound according to claim 15, wherein u is 1, or a pharmaceutically acceptable salt thereof.

21. The compound according to claim 15, wherein R²³ is substituted or unsubstituted aromatic heterocyclyl, or a pharmaceutically acceptable salt thereof.

22. The compound according to claim 15, wherein R³² and R³³ are hydrogen atoms, or a pharmaceutically acceptable salt thereof.

23. The compound according to claim 1, wherein the compound is selected from the group consisting of compounds I-067, I-080, I-104, I-105, I-113, I-114, I-115, I-125, and I-128, or a pharmaceutically acceptable salt thereof.

24. A pharmaceutical composition comprising the compound according to claim 1 or a pharmaceutically acceptable salt thereof.

25. The pharmaceutical composition according to claim 24, wherein the pharmaceutical composition is a serotonin 5-HT2A receptor antagonist and/or inverse agonist.

26. The pharmaceutical composition according to claim 24, wherein the pharmaceutical composition is a serotonin 5-HT2A and 5-HT2C receptor antagonist and/or inverse agonist.

27. A method for treating and/or preventing a disease associated with a 5-HT2A receptor comprising administering the compound according to claim 1 or a pharmaceutically acceptable salt thereof.

28. A method for treating and/or preventing a disease associated with 5-HT2A and 5-HT2C receptors comprising administering the compound according to claim 1 or a pharmaceutically acceptable salt thereof.

29-30. (canceled)
